(19) United States
(12) Patent Application Publication
(10) Pub. No.: US 2025/0019712 A1
(43) Pub. Date: Jan. 16, 2025

(54) PLANT REGULATORY ELEMENTS AND USES THEREOF

(71) Applicant: Monsanto Technology LLC, St. Louis, MO (US)

(72) Inventors: Stanislaw Flasinski, Ballwin, MO (US); Barrett C. Foat, St. Louis, MO (US); Mohammed Oufattole, Wildwood, MO (US); Randall W. Shultz, St. Louis, MO (US); Xiaoping Wei, St. Louis, MO (US); Wei Wu, Chesterfield, MO (US); Shiaw-Pyng Yang, St. Louis, MO (US)

(21) Appl. No.: 18/905,108

(22) Filed: Oct. 2, 2024

Related U.S. Application Data
(62) Division of application No. 17/488,189, filed on Sep. 28, 2021.
Application No. 17/488,189 is a division of application No. 16/722,287, filed on Dec. 20, 2019, now Pat. No. 11,180,768.
Application No. 16/722,287 is a division of application No. 16/549,573, filed on Aug. 23, 2019, now Pat. No. 11,046,966.
Application No. 16/549,573 is a division of application No. 15/802,843, filed on Nov. 3, 2017, now Pat. No. 10,550,401.
Application No. 15/802,843 is a division of application No. 14/117,342, filed on Oct. 23, 2014, now Pat. No. 9,845,477.
(60) Provisional application No. 61/485,876, filed on May 13, 2011.

(51) Int. Cl. C12N 15/82

(52) U.S. Cl. CPC C12N 15/8216

(57) ABSTRACT

The invention provides DNA molecules and constructs, including their nucleotide sequences, useful for modulating gene expression in plants and plant cells. Transgenic plants, plant cells, plant parts, seeds, and commodity products comprising the DNA molecules operably linked to heterologous transcribable polynucleotides are also provided, as are methods of their use.

### CROSS REFERENCE TO RELATED APPLICATIONS

This application is a divisional of U.S. patent application Ser. No. 17/488,189,filed Sep. 28, 2021, which is a divisional of U.S. patent application Ser. No. 16/722,287,filed Dec. 20, 2019, now U.S. Pat. No. 11,180,768, which is a divisional of U.S. patent application Ser. No. 16/549,573, filed Aug. 23, 2019, now U.S. Pat. No. 11,046,966, which is a divisional of U.S. patent application Ser. No. 15/802,843, filed Nov. 3, 2017, now U.S. Pat. No. 10,550,401, which is a divisional of U.S. patent application Ser. No. 14/117,342, filed Oct. 23, 2014, now U.S. Pat. No. 9,845,477, which is a 371 National Stage application of International Application No. PCT/US2012/037561, filed May 11, 2012, which claims the benefit of priority to U.S. Provisional Application Ser. No. 61/485,876, filed May 13, 2011, and are herein incorporated by reference in their entireties.

### INCORPORATION OF SEQUENCE LISTING

The sequence listing that is contained in the file named “MONS304USD6_ST26.xml”, which is 537 kilobytes (as measured in Microsoft Windows®) and was created on Aug. 11, 2024, is filed herewith by electronic submission and is incorporated by reference herein.

### FIELD OF THE INVENTION

The invention relates to the field of plant molecular biology and plant genetic engineering, and DNA molecules useful for modulating gene expression in plants.

### BACKGROUND

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

### SUMMARY OF THE INVENTION

The present invention provides novel gene regulatory elements such as promoters, leaders and introns derived from Cucumis melo, a plant species commonly referred to as muskmelon, for use in plants. The present invention also provides DNA constructs, transgenic plant cells, plants, and seeds comprising the regulatory elements. The sequences may be provided operably linked to a transcribable polynucleotide molecule which may be heterologous with respect to a regulatory sequence provided herein. The present invention also provides methods of making and using the regulatory elements, the DNA constructs comprising the regulatory elements, and the transgenic plant cells, plants, and seeds comprising the regulatory elements operably linked to a transcribable polynucleotide molecule.

Thus, in one aspect, the present invention provides a DNA molecule, such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOs: 1-199, 211 and 212 exhibiting gene-regulatory activity, wherein said DNA molecule is operably linked to a heterologous transcribable polynucleotide molecule. In specific embodiments, a transcriptional regulatory expression element group, or promoter, or leader, or intron is at least 90 percent, at least 95 percent, at least 98 percent, or at least 99 percent identical to any of SEQ ID NOs: 1-199, 211 and 212. In particular embodiments, the heterologous transcribable polynucleotide molecule comprises a gene of agronomic interest, a gene capable of providing herbicide resistance in plants, or a gene capable of providing plant pest resistance in plants.

The invention also provides a transgenic plant cell containing a DNA molecule such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOs: 1-199, 211 and 212 exhibiting gene-regulatory activity, wherein said DNA molecule is operably linked to a heterologous transcribable polynucleotide molecule. Further, the transcriptional regulatory expression element group, or promoter, or leader, or intron regulates the expression of a gene. The transgenic plant cell can be a monocotyledonous or dicotyledonous plant cell.

Further provided by the invention is a transgenic plant, or part of the transgenic plant containing a DNA molecule such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOs: 1-199, 211 and 212 exhibiting gene-regulatory activity, wherein said DNA molecule is operably linked to a heterologous transcribable polynucleotide molecule. In specific embodiments, the transgenic plant may be a progeny plant of any generation that contains the transcriptional regulatory expression element group, or promoter, or leader, or intron.

Still further provided is a transgenic seed containing a DNA molecule such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOs: 1-199, 211 and 212 exhibiting gene-regulatory activity, wherein said DNA molecule is operably linked to a heterologous transcribable polynucleotide molecule.

In yet another aspect, the invention provides a method of producing a commodity product from the transgenic plant, transgenic plant part or transgenic seed which contains a DNA molecule such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOs: 1-199, 211 and 212 exhibiting gene-regulatory activity, wherein said DNA molecule is operably linked to a heterologous transcribable polynucleotide molecule. In one embodiment, the commodity product is protein concentrate, protein isolate, grain, starch, seeds, meal, flour, biomass, or seed oil.

In another aspect, the invention provides a commodity product comprising a DNA molecule such as a transcriptional regulatory expression element group, or promoter, or leader, or intron, comprising a polynucleotide sequence selected from the group consisting of: a) a sequence with at least 85 percent sequence identity to any of SEQ ID NOs: 1-199, 211 and 212; b) a sequence comprising any of SEQ ID NOs: 1-199, 211 and 212; and c) a fragment of any of SEQ ID NOS: 1-199, 211 and 212 exhibiting gene-regulatory activity, wherein said DNA molecule is operably linked to a heterologous transcribable polynucleotide molecule.

In still yet another aspect, the invention provides a method of expressing a transcribable polynucleotide molecule in a transgenic plant using a DNA molecule such as a transcriptional regulatory expression element group, or promoter, or leader, or intron which has a DNA sequence which is at least 85 percent identical to that of any of SEQ ID NOs: 1-199, 211 and 212, or contains any of SEQ ID NOs: 1-199, 211 and 212, or consists of a fragment of any of SEQ ID NOs: 1-199, 211 and 212; and cultivating the transgenic plant.

### BRIEF DESCRIPTION OF THE SEQUENCES

SEQ ID NOs: 1, 5, 7, 9, 11, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 159, 162, 167, 168, 172, 175, 176, 177, 178, 181, 182, 183, 184, 185, 188, 189, 190, 191, 192, 193, 194, 195, 196, 197, 198, 199, 211 and 212 are Cucumis transcriptional regulatory expression element groups or EXP sequences which are comprised of either a promoter element, operably linked to a leader clement; or a promoter element, operably linked to a leader element and an intron element, or a promoter clement, operably linked to a leader element, operably linked to an intron element, operably linked to a leader element.

SEQ ID NOs: 2, 6, 8, 10, 12, 163 and 169 are promoter elements.

SEQ ID NOs: 3, 164, 166 and 170 are leader sequences.

SEQ ID NOs: 4, 165 and 171 are intron sequences.

SEQ ID NOs: 157, 160, 173, 179 and 186 are sequences wherein a promoter is operably linked to a leader element.

SEQ ID NOs: 158, 161, 174, 180 and 187 are sequences wherein an intron is operably linked to a leader element.

### BRIEF DESCRIPTION OF THE DRAWINGS

FIGS. 1a-1f depict alignment of promoter variant segments corresponding to promoter elements isolated from the Cucumis melo. In particular, FIGS. 1a-1f show alignment of the 2068 bp promoter sequence P-CUCme.Ubq1-1:1:15 (SEQ ID NO: 2), found in the transcriptional regulatory expression element group EXP-CUCme.Ubq1:1:1 (SEQ ID NO: 1), vs. promoter sequences derived via 5′ deletions of the promoter, P-CUCme.Ubq1-1:1:15. Deletion, for instance of the 5′ end of P-CUCme.Ubq1-1:1:15, produced the promoters, P-CUCme.Ubq1-1:1:16 (SEQ ID NO: 6) a 1459 bp promoter which is found within EXP-CUCme.Ubq1:1:2 (SEQ ID NO: 5); P-CUCme.Ubq1-1:1:17 (SEQ ID NO: 8), a 964 bp sequence comprised within EXP-CUCme.Ubq1:1:3 (SEQ ID NO: 7); P-CUCme.Ubq1-1:1:18 (SEQ ID NO: 10), a 479 bp sequence comprised within EXP-CUCme.Ubq1:1:4 (SEQ ID NO: 9); and P-CUCme.Ubq1-1:1:19 (SEQ ID NO: 12), a 173 bp sequence comprised within EXP-CUCme.Ubq1:1:5 (SEQ ID NO: 11).

### DETAILED DESCRIPTION OF THE INVENTION

The invention disclosed herein provides polynucleotide molecules obtained from Cucumis melo having beneficial gene regulatory activity. The design, construction, and use of these polynucleotide molecules are described. The nucleotide sequences of these polynucleotide molecules are provided among SEQ ID NOs: 1-199, 211 and 212. These polynucleotide molecules are, for instance, capable of affecting the expression of an operably linked transcribable polynucleotide molecule in plant tissues, and therefore selectively regulating gene expression, or activity of an encoded gene product, in transgenic plants. The present invention also provides methods of modifying, producing, and using the same. The invention also provides compositions, transformed host cells, transgenic plants, and seeds containing the promoters and/or other disclosed nucleotide sequences, and methods for preparing and using the same.

The following definitions and methods are provided to better define the present invention and to guide those of ordinary skill in the art in the practice of the present invention. Unless otherwise noted, terms are to be understood according to conventional usage by those of ordinary skill in the relevant art.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods are known in the to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NOs: 1-199, 211 and 212.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a DNA molecule comprising a sequence that when optimally aligned to a reference sequence, provided herein as SEQ ID NOs: 1-199, 211 and 212, has at least about 85 percent identity at least about 90 percent identity at least about 95 percent identity, at least about 96 percent identity, at least about 97 percent identity, at least about 98 percent identity, or at least about 99 percent identity to the reference sequence. In particular embodiments such sequences may be defined as having gene-regulatory activity or encoding a peptide that functions to localize an operably linked polypeptide within a cell.

### Regulatory Elements

A regulatory clement is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory expression element group (EXP) may be comprised of expression elements, such as enhancers, promoters, leaders and introns, operably linked. Thus a transcriptional regulatory expression element group may be comprised, for instance, of a promoter operably linked 5′ to a leader sequence, which is in turn operably linked 5′ to an intron sequence. The intron sequence may be comprised of a sequence beginning at the point of the first intron/exon splice junction of the native sequence and further may be comprised of a small leader fragment comprising the second intron/exon splice junction so as to provide for proper intron/exon processing to facilitate transcription and proper processing of the resulting transcript. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, leaders, introns, and transcription termination regions are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory clement for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include any of SEQ ID NOs: 2, 6, 8, 10, 12, 163 and 169, or the promoter elements comprised within any of SEQ ID NOs: 13 through 199, 211 and 212, or fragments or variants thereof. In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for intiation of transcription.

In one embodiment, fragments of a promoter molecule are provided. Promoter fragments provide promoter activity, as described above, and may be useful alone or in combination with other promoters and promoter fragments, such as in constructing chimeric promoters. In specific embodiments, fragments of a promoter are provided comprising at least about 50, 95, 150, 250, 500, 750, or at least about 1000 contiguous nucleotides, or longer, of a polynucleotide molecule having promoter activity disclosed herein.

Compositions derived from any of the promoters presented as SEQ ID NOs: 2, 6, 8, 10, 12, 163 and 169, or the promoter elements comprised within SEQ ID NOs: 13 through 199, 211 and 212, such as internal or 5′ deletions, for example, can be produced to improve or alter expression, including by removing elements that have either positive or negative effects on expression; duplicating elements that have positive or negative effects on expression; and/or duplicating or removing elements that have tissue or cell specific effects on expression. Compositions derived from any of the promoters presented as SEQ ID NOs: 2, 6, 8, 10, 12, 163 and 169, or the promoter elements comprised within SEQ ID NOs: 13 through 199, 211 and 212 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. Any of the promoters presented as SEQ ID NOs: 2, 6, 8, 10, 12, 163 and 169, or the promoter elements comprised within SEQ ID NOs: 13 through 199, 211 and 212, and fragments or enhancers derived there from can be used to make chimeric transcriptional regulatory clement compositions comprised of any of the promoters presented as SEQ ID NOs: 2, 6, 8, 10, 12, 163 and 169, or the promoter elements comprised within SEQ ID NOs: 13 through 199, 211 and 212, and the fragments or enhancers derived therefrom operably linked to other enhancers and promoters. The efficacy of the modifications, duplications or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders useful in practicing the present invention include SEQ ID NOs: 3, 164, 166 and 170, or the leader element comprised within SEQ ID NOs: 13 through 199, 211 and 212, or fragments or variants thereof. In specific embodiments, such sequences may be provided defined as being capable of acting as a leader in a host cell, including, for example, a transgenic plant cell. In one embodiment such sequences are decoded as comprising leader activity.

The leader sequences (5′ UTR) presented as SEQ ID NOs: 3, 164, 166 and 170, or the leader element comprised within any of SEQ ID NOs: 13 through 199, 211 and 212 may be comprised of regulatory elements or may adopt secondary structures that can have an effect on transcription or translation of a transgene. The leader sequences presented as SEQ ID NOs: 3,164, 166 and 170, or the leader element comprised within SEQ ID NOs: 13 through 199, 211 and 212 can be used in accordance with the invention to make chimeric regulatory elements that affect transcription or translation of a transgene. In addition, the leader sequences presented as SEQ ID NOs: 3, 164, 166 and 170, or the leader element comprised within any of SEQ ID NOs: 13 through 199, 211 and 212 can be used to make chimeric leader sequences that affect transcription or translation of a transgene.

The introduction of a foreign gene into a new plant host does not always result in a high expression of the incoming gene. Furthermore, if dealing with complex traits, it is sometimes necessary to modulate several genes with spatially or temporarily different expression pattern. Introns can principally provide such modulation. However, multiple use of the same intron in one transgenic plant has shown to exhibit disadvantages. In those cases it is necessary to have a collection of basic control elements for the construction of appropriate recombinant DNA elements. As the available collection of introns known in the art with expression enhancing properties is limited, alternatives are needed.

Compositions derived from any of the introns presented as SEQ ID NOs: 4, 165 and 171 or the intron clement comprised within SEQ ID NOs: 13 through 199, 211 and 212 can be comprised of internal deletions or duplications of cis regulatory elements; and/or alterations of the 5′ and 3′ sequences comprising the intron/exon splice junctions can be used to improve expression or specificity of expression when operably linked to a promoter+leader or chimeric promoter+leader and coding sequence. Alterations of the 5′ and 3′ regions comprising the intron/exon splice junction can also be made to reduce the potential for introduction of false start and stop codons being produced in the resulting transcript after processing and splicing of the messenger RNA. The introns can be tested empirically as described in the working examples to determine the intron's effect on expression of a transgene.

In accordance with the invention a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants having a similar expression pattern to the original promoter.

As used herein, the term “enhancer” or “enhancer element” refers to a cis-acting transcriptional regulatory element, a.k.a. cis-clement, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter.cis-element, which confers an aspect of the overall modulation of gene expression. A promoter or promoter fragment may comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer clement may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-clement motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention.

In plants, the inclusion of some introns in gene constructs leads to increased mRNA and protein accumulation relative to constructs lacking the intron. This effect has been termed “intron mediated enhancement” (IME) of gene expression (Mascarenhas et al., (1990) Plant Mol. Biol. 15:913-920). Introns known to stimulate expression in plants have been identified in maize genes (e.g. tubA1, Adh1, Sh1, Ubi1 (Jeon et al. (2000) Plant Physiol. 123:1005-1014; Callis et al. (1987) Genes Dev. 1:1183-1200; Vasil et al. (1989) Plant Physiol. 91:1575-1579; Christiansen et al. (1992) Plant Mol. Biol. 18:675-689) and in rice genes (e.g. salt, tpi: McElroy et al., Plant Cell 2:163-171 (1990); Xu et al., Plant Physiol. 106:459-467 (1994)). Similarly, introns from dicotyledonous plant genes like those from petunia (e.g. rbcS), potato (e.g. st-ls1) and from Arabidopsis thaliana (e.g. ubq3 and pat1) have been found to elevate gene expression rates (Dean et al. (1989) Plant Cell 1:201-208; Leon et al. (1991) Plant Physiol. 95:968-972; Norris et al. (1993) Plant Mol Biol 21:895-906; Rose and Last (1997) Plant J. 11:455-464). It has been shown that deletions or mutations within the splice sites of an intron reduce gene expression, indicating that splicing might be needed for IME (Mascarenhas et al. (1990) Plant Mol Biol. 15:913-920; Clancy and Hannah (2002) Plant Physiol. 130:918-929). However, that splicing per se is not required for a certain IME in dicotyledonous plants has been shown by point mutations within the splice sites of the pat1 gene from A. thaliana (Rose and Beliakoff (2000) Plant Physiol. 122:535-542).

Enhancement of gene expression by introns is not a general phenomenon because some intron insertions into recombinant expression cassettes fail to enhance expression (e.g. introns from dicot genes (rbcS gene from pea, phascolin gene from bean and the stls-1 gene from Solanum tuberosum) and introns from maize genes (adh1 gene the ninth intron, hsp81 gene the first intron)) (Chee et al. (1986) Gene 41:47-57; Kuhlemeier et al. (1988) Mol Gen Genet 212:405-411; Mascarenhas et al. (1990) Plant Mol. Biol. 15:913-920; Sinibaldi and Mettler (1992) In WE Cohn, K Moldave, eds, Progress in Nucleic Acid Research and Molecular Biology, Vol 42. Academic Press, New York, pp 229-257; Vancanneyt et al. 1990 Mol. Gen. Genet. 220:245-250). Therefore, not each intron can be employed in order to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause IME.

As used herein, the term “chimeric” refers to a single DNA molecule produced by fusing a first DNA molecule to a second DNA molecule, where neither first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is in composition similar, but not identical to, a first DNA molecule and yet the second DNA molecule still maintains the general functionality, i.e. same or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion and/or insertion of one or more nucleotides of a reference sequence, wherein the derivative regulatory element has more or less or equivalent transcriptional or translational activity than the corresponding parent regulatory molecule. The regulatory element “variants” may also encompass variants arising from mutations that naturally occur in bacterial and plant cell transformation. In the present invention, a polynucleotide sequence provided as SEQ ID NOs: 1-199, 211 and 212 may be used to create variants similar in composition, but not identical to, the polynucleotide sequence of the original regulatory element, while still maintaining the general functionality of, i.e. same or similar expression pattern, the original regulatory element. Production of such variants of the present invention is well within the ordinary skill of the art in light of the disclosure and is encompassed within the scope of the present invention. “Varients” of chimeric regulatory element comprise the same constituent elements as a reference chimeric regulatory element sequence but the constituent elements comprising the chimeric regulatory element may be operatively linked by various methods known in the art such as, restriction enzyme digestion and ligation, ligation independent cloning, modular assembly of PCR products during amplification, or direct chemical synthesis of the chimeric regulatory element as well as other methods known in the art. The resulting “variant” chimeric regulatory element is comprised of the same, or variants of the same, constituent elements as the reference sequence but differ in the sequence or sequences that are used to operably link the constituent elements. In the present invention, the polynucleotide sequences provided as SEQ ID NOs: 1-199, 211 and 212 each provide a reference sequence wherein the constituent elements of the reference sequence may be joined by methods known in the art and may consist of substitutions, deletions and/or insertions of one or more nucleotides or mutations that naturally occur in bacterial and plant cell transformation.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known in the art of plant transformation can function in the present invention.

Methods are available for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells can be found in, for example, Molecular Cloning: A Laboratory Manual, 3^{rd} edition Volumes 1, 2, and 3 (2000) J. F. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061;and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ transcription termination molecule.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus 35S transcript promoter (see, U.S. Pat. No. 5,352,605). The expression properties imparted by such operable linkages of heterologous elements is not necessarily additive of the elucidated properties of each promoter and leader, but rather is determined through empirical analysis of expression driven by the operably linked heterologous promoter and leader.

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Introns useful in practicing the present invention include SEQ ID NOs: 4, 165 and 171 or the intron clement comprised within any of SEQ ID NOs: 13 through 199, 211 and 212.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules are the nopaline synthase 3′ region (see, Fraley, et al., Proc. Natl. Acad. Sci. USA, 80:4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

3′ UTRs typically find beneficial use for the recombinant expression of specific genes. In animal systems, a machinery of 3′ UTRs has been well defined (e.g. Zhao et al., Microbiol Mol Biol Rev 63:405-445 (1999); Proudfoot, Nature 322:562-565 (1986); Kim et al., Biotechnology Progress 19:1620-1622 (2003); Yonaha and Proudfoot, EMBO J. 19:3770-3777 (2000); Cramer et al., FEBS Letters 498:179-182 (2001); Kuerstem and Goodwin, Nature Reviews Genetics 4:626-637 (2003)). Effective termination of RNA transcription is required to prevent unwanted transcription of trait-unrelated (downstream) sequences, which may interfere with trait performance. Arrangement of multiple gene expression cassettes in local proximity to one another (e.g. within one T-DNA) may cause suppression of gene expression of one or more genes in said construct in comparison to independent insertions (Padidam and Cao, BioTechniques 31:328-334 (2001). This may interfere with achieving adequate levels of expression, for instance in cases were strong gene expression from all cassettes is desired.

In plants, clearly defined polyadenylation signal sequences are not known. Hasegawa et al., Plant J. 33:1063-1072, (2003)) were not able to identify conserved polyadenylation signal sequences in both in vitro and in vivo systems in Nicotiana sylvestris and to determine the actual length of the primary (non-polyadenylated) transcript. A weak 3′ UTR has the potential to generate read-through, which may affect the expression of the genes located in the neighboring expression cassettes (Padidam and Cao, BioTechniques 31:328-334 (2001)). Appropriate control of transcription termination can prevent read-through into sequences (e.g. other expression cassettes) localized downstream and can further allow efficient recycling of RNA polymerase, to improve gene expression. Efficient termination of transcription (release of RNA Polymerase II from the DNA) is pre-requisite for re-initiation of transcription and thereby directly affects the overall transcript level. Subsequent to transcription termination, the mature mRNA is released from the site of synthesis and template to the cytoplasm. Eukaryotic mRNAs are accumulated as poly(A) forms in vivo, so that it is difficult to detect transcriptional termination sites by conventional methods. However, prediction of functional and efficient 3′ UTRs by bioinformatics methods is difficult in that there are no conserved sequences which would allow easy prediction of an effective 3′ UTR.

From a practical standpoint, it is typically beneficial that a 3′ UTR used in a transgene cassette possesses the following characteristics. The 3′ UTR should be able to efficiently and effectively terminate transcription of the transgene and prevent read-through of the transcript into any neighboring DNA sequence which can be comprised of another transgene cassette as in the case of multiple cassettes residing in one T-DNA, or the neighboring chromosomal DNA into which the T-DNA has inserted. The 3′ UTR should not cause a reduction in the transcriptional activity imparted by the promoter, leader and introns that are used to drive expression of the transgene. In plant biotechnology, the 3′ UTR is often used for priming of amplification reactions of reverse transcribed RNA extracted from the transformed plant and used to (1) assess the transcriptional activity or expression of the transgene cassette once integrated into the plant chromosome; (2) assess the copy number of insertions within the plant DNA; and (3) assess zygosity of the resulting seed after breeding. The 3′ UTR is also used in amplification reactions of DNA extracted from the transformed plant to characterize the intactness of the inserted cassette.

3′ UTRs useful in providing expression of a transgene in plants may be identified based upon the expression of expressed sequence tags (ESTs) in cDNA libraries made from messenger RNA isolated from seed, flower and other tissues derived from Foxtail millet (Setaria italica (L.) Beauv). Libraries of cDNA are made from tissues isolated from selected plant species using flower tissue, seed, leaf and root. The resulting cDNAs are sequenced using various sequencing methods. The resulting ESTs are assembled into clusters using bioinformatics software such as clc_ref_assemble_complete version 2.01.37139 (CLC bio USA, Cambridge, Massachusetts 02142). Transcript abundance of each cluster is determined by counting the number of cDNA reads for each cluster. The identified 3′ UTRs may be comprised of sequence derived from cDNA sequence as well as sequence derived from genomic DNA. The cDNA sequence is used to design primers, which are then used with Genome Walker™ (Clontech Laboratories, Inc, Mountain View, CA) libraries constructed following the manufacturer's protocol to clone the 3′ region of the corresponding genomic DNA sequence to provide a longer termination sequence. Analysis of relative transcript abundance either by direct counts or normalized counts of observed sequence reads for each tissue library can be used to infer properties about patters of expression. For example, some 3′ UTRs may be found in transcripts seen in higher abundance in root tissue as opposed to leaf. This is suggestive that the transcript is highly expressed in root and that the properties of root expression may be attributable to the transcriptional regulation of the promoter, the lead, the introns or the 3′ UTR. Empirical testing of 3′ UTRs identified by the properties of expression within specific organs, tissues or cell types can result in the identification of 3′ UTRs that enhance expression in those specific organs, tissues or cell types.

Constructs and vectors may also include a transit peptide coding sequence that expresses a linked peptide that is useful for targeting of a protein product, particularly to a chloroplast, leucoplast, or other plastid organelle; mitochondria; peroxisome; vacuole; or an extracellular location. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (See, Klee et al., Mol. Gen. Genet. 210:437-442 (1987)) or the Petunia hybrida EPSPS CTP (CTP4) (See, della-Cioppa et al., Proc. Natl. Acad. Sci. USA 83:6873-6877 (1986)) has been show to target heterologous EPSPS protein sequences to chloroplasts in transgenic plants (See, U.S. Pat. Nos. 5,627,061; 5,633,435; and 5,312,910 and EP 0218571; EP 189707; EP 508909; and EP 924299).

### Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species or the same genes from different species. A promoter is thus heterologous with respect to an operably linked transcribable polynucleotide molecule if such a combination is not normally found in nature, i.e. that transcribable polynucleotide molecule is not naturally occurring operably linked in combination with that promoter molecule.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. Briefly, as the antisense transcribable polynucleotide molecule is transcribed, the RNA product hybridizes to and sequesters a complimentary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NOs: 1-199, 211 and 212, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product. In another embodiment, the transcribable polynucleotide molecule comprises an antisense region of a gene, and the promoter affects the transcription of an antisense RNA molecule, double stranded RNA or other similar inhibitory RNA molecule in order to inhibit expression of a specific RNA molecule of interest in a target host cell.

### Genes of Agronomic Interest

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism or may be act as a pesticidal agent in the diet of a pest that feeds on the plant.

In one embodiment of the invention, a promoter of the present invention is incorporated into a construct such that the promoter is operably linked to a transcribable polynucleotide molecule that is a gene of agronomic interest. The expression of the gene of agronomic interest is desirable in order to confer an agronomically beneficial trait. A beneficial agronomic trait may be, for example, but is not limited to, herbicide tolerance, insect control, modified yield, fungal disease resistance, virus resistance, nematode resistance, bacterial disease resistance, plant growth and development, starch production, modified oils production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production. Examples of genes of agronomic interest include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Patent Nos. U.S. RE 38,446; U.S. Pat. Nos. 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Patent Nos. U.S. RE 37,543; U.S. Pat. Nos. 6,228,623; and 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristic or phenotype by encoding a RNA molecule that causes the targeted modulation of gene expression of an endogenous gene, for example via antisense (sec e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

### Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding ß-glucuronidase (GUS described in U.S. Pat. No. 5,599,670), green fluorescent protein and variants thereof (GFP described in U.S. Pat. Nos. 5,491,084 and 6,146,826), proteins that confer antibiotic resistance, or proteins that confer herbicide tolerance. Useful antibiotic resistance markers include those encoding proteins conferring resistance to kanamycin (nptII), hygromycin B (aph IV), streptomycin or spectinomycin (aad, spec/strep) and gentamycin (aac3 and aacC4). Herbicides for which transgenic plant tolerance has been demonstrated and the method of the present invention can be applied, include, but are not limited to: amino-methyl-phosphonic acid, glyphosate, glufosinate, sulfonylureas, imidazolinones, bromoxynil, delapon, dicamba, cyclohezanedione, protoporphyrinogen oxidase inhibitors, and isoxasflutole herbicides. Transcribable polynucleotide molecules encoding proteins involved in herbicide tolerance include, but are not limited to, a transcribable polynucleotide molecule encoding 5-enolpyruvylshikimate-3-phosphate synthase (EPSPS for glyphosate tolerance described in U.S. Pat. Nos. 5,627,061; 5,633,435; 6,040,497; and 5,094,945); a transcribable polynucleotide molecule encoding a glyphosate oxidoreductase and a glyphosate-N-acetyl transferase (GOX described in U.S. Pat. No. 5,463,175; GAT described in U.S. Patent publication No. 20030083480, and dicamba monooxygenase U.S. Patent publication No. 20030135879); a transcribable polynucleotide molecule encoding bromoxynil nitrilase (Bxn for Bromoxynil tolerance described in U.S. Pat. No. 4,810,648); a transcribable polynucleotide molecule encoding phytoene desaturase (crtI) described in Misawa, et al., Plant Journal 4:833-840 (1993) and Misawa, et al., Plant Journal 6:481-489 (1994) for norflurazon tolerance; a transcribable polynucleotide molecule encoding acetohydroxyacid synthase (AHAS, aka ALS) described in Sathasiivan, et al., Nucl. Acids Res. 18:2188-2193 (1990) for tolerance to sulfonylurea herbicides; and the bar gene described in DeBlock, et al., EMBO Journal 6:2513-2519 (1987) for glufosinate and bialaphos tolerance. The promoter molecules of the present invention can express linked transcribable polynucleotide molecules that encode for phosphinothricin acetyltransferase, glyphosate resistant EPSPS, aminoglycoside phosphotransferase, hydroxyphenyl pyruvate dehydrogenase, hygromycin phosphotransferase, neomycin phosphotransferase, dalapon dehalogenase, bromoxynil resistant nitrilase, anthranilate synthase, aryloxyalkanoate dioxygenases, acetyl COA carboxylase, glyphosate oxidoreductase, and glyphosate-N-acetyl transferase.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present invention.

### Cell Transformation

The invention is also directed to a method of producing transformed cells and plants which comprise a promoter operably linked to a transcribable polynucleotide molecule.

The term “transformation” refers to the introduction of nucleic acid into a recipient host. As used herein, the term “host” refers to bacteria, fungi, or plant, including any cells, tissue, organs, or progeny of the bacteria, fungi, or plant. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules.

There are many methods for introducing polynucleic acid molecules into plant cells. The method generally comprises the steps of selecting a suitable host cell, transforming the host cell with a recombinant vector, and obtaining the transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42:205 (1991)).

Any transformation methods may be utilized to transform a host cell with one or more promoters and/or constructs of the present invention. Host cells may be any cell or organism such as a plant cell, algae cell, algae, fungal cell, fungi, bacterial cell, or insect cell. Preferred hosts and transformed cells include cells from: plants, Aspergillus, yeasts, insects, bacteria and algae.

Regenerated transgenic plants can be self-pollinated to provide homozygous transgenic plants. Alternatively, pollen obtained from the regenerated transgenic plants may be crossed with non-transgenic plants, preferably inbred lines of agronomically important species. Descriptions of breeding methods that are commonly used for different traits and crops can be found in one of several reference books, see, for example, Allard, Principles of Plant Breeding, John Wiley & Sons, NY, U. of CA, Davis, CA, 50-98 (1960); Simmonds, Principles of crop improvement, Longman, Inc., NY, 369-399 (1979); Sneep and Hendriksen, Plant breeding perspectives, Wageningen (ed), Center for Agricultural Publishing and Documentation (1979); Fehr, Soybeans: Improvement, Production and Uses, 2nd Edition, Monograph, 16:249 (1987); Fehr, Principles of variety development, Theory and Technique, (Vol. 1) and Crop Species Soybean (Vol 2), Iowa State Univ., Macmillan Pub. Co., NY, 360-376 (1987). Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immuno-precipitation, and ELISA.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Identification and Cloning of Regulatory Elements

Novel transcriptional regulatory elements, or transcriptional regulatory expression element group (EXP) sequences were identified and isolated from genomic DNA of the dicot species Cucumis melo WSH-39-1070AN.

Transcriptional regulatory elements were selected based upon proprietary and public microarray data derived from transcriptional profiling experiments conducted in soybean (Glycine max) and Arabidopsis as well as homology based searches using known dicot sequences as query against proprietary Cucumis melo sequences.

Using the identified sequences, a bioinformatic analysis was conducted to identify regulatory elements within the amplified DNA, followed by identification of the transcriptional start site (TSS) and any bi-directionality, introns, or upstream coding sequence present in the sequence. Using the results of this analysis, regulatory elements were defined within the DNA sequences and primers designed to amplify the regulatory elements. The corresponding DNA molecule for each regulatory element was amplified using standard polymerase chain reaction conditions with primers containing unique restriction enzyme sites and genomic DNA isolated from Cucumis melo. The resulting DNA fragments were ligated into base plant expression vectors using standard restriction enzyme digestion of compatible restriction sites and DNA ligation methods.

Analysis of the regulatory element TSS and intron/exon splice junctions can be performed using transformed plant protoplasts. Briefly, the protoplasts are transformed with the plant expression vectors comprising the cloned DNA fragments operably linked to a heterologous transcribable polynucleotide molecule and the 5′ RACE System for Rapid Amplification of cDNA Ends, Version 2.0 (Invtrogen, Carlsbad, California 92008) is used to confirm the regulatory element TSS and intron/exon splice junctions by analyzing the sequence of the mRNA transcripts produced thereby.

Sequences encoding ubiquitin 1 transcriptional regulatory expression element groups (EXP) were analyzed as described above and each transcriptional regulatory expression element groups (“EXP's”) was also broken down into the corresponding promoters, leaders and introns comprising each transcriptional regulatory expression element group. Sequences of the identified ubiquitin 1 transcriptional regulatory expression element groups (“EXP's”) are provided herein as SEQ ID NOs: 1, 5, 7, 9 and 11 and is listed in Table 1 below. The corresponding ubiquitin 1 promoters are provided herein as SEQ ID NOs: 2, 6, 8, 10 and 12. The ubiquitin 1 leader and intron are herein provided as SEQ ID NOs: 3 and 4, respectively.

Sequences encoding other Cucumis transcriptional regulatory expression element groups or EXP sequences which are comprised of either a promoter element, operably linked to a leader element; or a promoter element, operably linked to a leader element and an intron element, or a promoter element, operably linked to a leader element, operably linked to an intron element, operably linked to a leader element are provided as SEQ ID NOs: 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 159, 162, 167, 168, 172, 175, 176, 177, 178, 181, 182, 183, 184, 185, 188, 189, 190, 191, 192, 193, 194, 195, 196, 197, 198, 199, 211 and 212 and are also listed in Table 1 below. Additional promoter elements are provided as SEQ ID NOs: 163 and 169. Additional leader elements are provided as SEQ ID NOs: 164, 166 and 170. Additional intron elements are provided as SEQ ID NOs: 165 and 171. Elements wherein a promoter is operably linked to a leader element are provided as SEQ ID NOs: 157, 160, 173, 179 and 186. Elements wherein an intron is operably linked to a leader element are provided as SEQ ID NOs: 158, 161, 174, 180 and 187. With respect to the subset of sequences provided as SEQ ID NOs: 13 through 199, 211 and 212, these sequences were selected and cloned based upon the results of experiments such as transcript profiling or expression driven by promoters from homologous genes of a different species suggesting desirable patterns of expression such as constitutive expression, root expression, above ground expression or seed expression. The actual activity imparted by the Cucumis sequences is determined empirically and is not necessarily the same as that of a regulatory element derived from a homologous gene from a species other than Cucumis melo when used in a transformed plant host cell and whole transgenic plant.

TABLE 1
Transcriptional regulatory expression element groups, promoters, leaders and introns isolated from Cucumis melo.

| | SEQ | | Compo- | | | |
|---|---|---|---|---|---|---|
| | ID | | sition | Size | | Coordinates of |
| Annotation | NO: | Description | Type | (bp) | Composition | Elements within EXP |
| EXP-CUCme.Ubq1:1:1 | 1 | Ubiquitin 1 | EXP | 2611 | Promoter; Leader; | 1-2068; 2069- |
| | | | | | Intron | 2150; 2151-2608 |
| P-CUCme.Ubq1-1:1:15 | 2 | Ubiquitin 1 | P | 2068 | Promoter | |
| L-CUCme.Ubq1-1:1:1 | 3 | Ubiquitin 1 | L | 82 | Leader | |
| I-CUCme.Ubq1-1:1:1 | 4 | Ubiquitin 1 | I | 461 | Intron | |
| EXP-CUCme.Ubq1:1:2 | 5 | Ubiquitin 1 | EXP | 2002 | Promoter; Leader; | 1-1459; 1460- |
| | | | | | Intron | 1541; 1542-1999 |
| P-CUCme.Ubq1-1:1:16 | 6 | Ubiquitin 1 | P | 1459 | Promoter | |
| EXP-CUCme.Ubq1:1:3 | 7 | Ubiquitin 1 | EXP | 1507 | Promoter; Leader; | 1-964; 965-1046; |
| | | | | | Intron | 1047-1504 |
| P-CUCme.Ubq1-1:1:17 | 8 | Ubiquitin 1 | P | 964 | Promoter | |
| EXP-CUCme.Ubq1:1:4 | 9 | Ubiquitin 1 | EXP | 1022 | Promoter; Leader; | 1-479; 480-561; 562- |
| | | | | | Intron | 1019 |
| P-CUCme.Ubq1-1:1:18 | 10 | Ubiquitin 1 | P | 479 | Promoter | |
| EXP-CUCme.Ubq1:1:5 | 11 | Ubiquitin 1 | EXP | 716 | Promoter; Leader; | 1-173; 174-255; |
| | | | | | Intron | 256-713 |
| P-CUCme.Ubq1-1:1:19 | 12 | Ubiquitin 1 | P | 173 | Promoter | |
| P-CUCme.1-1:1:1 | 13 | Phosphatase 2A | EXP | 2000 | Promoter; Leader; | Reverse compliment; |
| | | | | | Intron; Leader | see SEQ ID NO: 155 |
| P-CUCme.2-1:1:1 | 14 | Actin 1 | EXP | 2000 | Promoter; Leader; | 1-964; 965-1028; 1029- |
| | | | | | Intron; Leader | 1991; 1992-2003 |
| P-CUCme.3-1:1:3 | 15 | Actin 2 | EXP | 1990 | Promoter; Leader; | 1-1243; 1244- |
| | | | | | Intron; Leader | 1319; 1320-1982; |
| | | | | | | 1983-1990 |
| P-CUCme.4-1:1:2 | 16 | Ubiquitin 2 | EXP | 2005 | Promoter; Leader; | 1-1646; 1647- |
| | | | | | Intron; Leader | 1704; 1705-2005; |
| | | | | | | 2006-2008 |
| P-CUCme.5-1:1:2 | 17 | Ubiquitin 3 | EXP | 2004 | Promoter; Leader; | 1-748; 749-819; |
| | | | | | Intron | 820-2004 |
| P-CUCme.6-1:1:1 | 18 | Tubulin beta chain | EXP | 1935 | Promoter; Leader; | 1-1436; 1437- |
| | | | | | Intron; Leader | 1482; 1483-1919; |
| | | | | | | 1920-1935 |
| P-CUCme.8-1:1:2 | 19 | Tubulin beta chain | EXP | 1606 | Promoter; Leader | 1-1527; 1528-1606 |
| P-CUCme.9-1:1:2 | 20 | Tubulin beta chain | EXP | 1487 | Promoter; Leader | 1-1384; 1385-1487 |
| P-CUCme.10-1:1:1 | 21 | Tubulin beta chain | EXP | 1448 | Promoter; Leader | 1-1363; 1364-1448 |
| P-CUCme.11-1:1:2 | 22 | Elongation Factor 1 | EXP | 1235 | Promoter; Leader; | 1-617; 618-677; 678- |
| | | alpha | | | Intron | 1213; 1214-1235 |
| P-CUCme.15-1:1:2 | 23 | Elongation Factor 1 | EXP | 2003 | Promoter; Leader; | 1-1330; 1331- |
| | | alpha | | | Intron; Leader | 1435; 1430-1975; |
| | | | | | | 1976-2002 |
| P-CUCme.16a-1:1:2 | 24 | Ubiquitin 7 | EXP | 2015 | Promoter; Leader | |
| P-CUCme.16b-1:1:1 | 25 | Ubiquitin 6 | EXP | 2006 | Promoter; Leader | |
| P-CUCme.17-1:1:2 | 26 | ubiquitin-40S | EXP | 2017 | Promoter; Leader | 1-1969; 1970-2017 |
| | | ribosomal protein | | | | |
| | | S27a | | | | |
| P-CUCme.18-1:1:2 | 27 | ubiquitin-40S | EXP | 1353 | Promoter; Leader | 1-1308; 1309-1353 |
| | | ribosomal protein | | | | |
| | | S27a | | | | |
| P-CUCme.19-1:1:2 | 28 | Chloropyll a/b | EXP | 2005 | Promoter; Leader | 1-1960; 1961-2005 |
| | | binding protein | | | | |
| P-CUCme.20-1:1:2 | 29 | Chloropyll a/b | EXP | 1445 | Promoter; Leader | 1-1390; 1391-1445 |
| | | binding protein | | | | |
| P-CUCme.21-1:1:1 | 30 | Chloropyll a/b | EXP | 1282 | Promoter; Leader | 1-1233; 1234-1282 |
| | | binding protein | | | | |
| P-CUCme.22-1:1:3 | 31 | Elongation Factor 4 | EXP | 2002 | | |
| | | alpha | | | | |
| P-CUCme.24-1:1:2 | 32 | S- | EXP | 2003 | Promoter; Leader; | 1-1067; 1068- |
| | | Adenosylmethionine | | | Intron; Leader | 1165; 1166-2001; |
| | | Synthetase | | | | 2002-2003 |
| P-CUCme.26-1:1:2 | 33 | Stress responsive | EXP | 1372 | Promoter; Leader; | 1-577; 578-654; 655- |
| | | protein | | | Intron; Leader | 1366; 1367-1372 |
| P-CUCme.28-1:1:2 | 34 | Ribosomal protein | EXP | 1122 | | |
| | | S5a | | | | |
| P-CUCme.29-1:1:2 | 35 | Ribosomal protein | EXP | 2017 | Promoter; Leader; | 1-490; 491-571; 572- |
| | | S5a | | | Intron; Leader | 2012; 2013-2017 |
| CumMe_WSM_SF143981.G5150 | 36 | LHCB6 (LIGHT | EXP | 2000 | | |
| | | HARVESTING | | | | |
| | | COMPLEX PSII | | | | |
| | | SUBUNIT 6) | | | | |
| CumMe_WSM_SF144839.G5080 | 37 | EIF2 GAMMA | EXP | 1760 | | |
| | | translation initiation | | | | |
| | | factor | | | | |
| CumMe_WSM_SF146040.G5050 | 38 | EIF2 translation | EXP | 1767 | | |
| | | initiation factor | | | | |
| CumMe_WSM_SF16408.G5350 | 39 | elongation factor Tu | EXP | 2000 | | |
| CumMe_WSM_SF16429.G5670 | 40 | unknown protein | EXP | 2000 | | |
| CumMe_WSM_SF16444.G5140 | 41 | histone H4 | EXP | 2000 | Promoter; Leader | 1-1947; 1948-2000 |
| CumMe_WSM_SF16530.G6000 | 42 | HMGB2 (HIGH | EXP | 2000 | | |
| | | MOBILITY GROUP | | | | |
| | | B 2) transcription | | | | |
| | | factor | | | | |
| CumMe_WSM_SF16553.G5090 | 43 | PBG1; threonine- | EXP | 1115 | | |
| | | type endopeptidase | | | | |
| CumMe_WSM_SF16563.G5560 | 44 | ATARFB1A (ADP- | EXP | 2000 | Promoter; Leader; | 1-1329; 1330- |
| | | ribosylation factor | | | Intron; Leader | 1427; 1428-1988; 1989- |
| | | B1A) | | | | 2000 |
| CumMe_WSM_SF16675.G5720 | 45 | chromatin protein | EXP | 2000 | | |
| | | family | | | | |
| CumMe_WSM_SF16920.G5650 | 46 | CSD1 | EXP | 2000 | | |
| | | (COPPER/ZINC | | | | |
| | | SUPEROXIDE | | | | |
| | | DISMUTASE 1) | | | | |
| CumMe_WSM_SF16953.G5180 | 47 | SCE1 (SUMO | EXP | 2000 | | |
| | | CONJUGATION | | | | |
| | | ENZYME 1); | | | | |
| | | SUMO ligase | | | | |
| CumMe_WSM_SF17051.G5470 | 48 | 60S ribosomal | EXP | 2000 | | |
| | | protein L9 | | | | |
| | | (RPL90D) | | | | |
| CumMe_WSM_SF17111.G5790 | 49 | ubiquinol- | EXP | 2000 | Promoter; Leader | 1-1895; 1896-2000 |
| | | cytochrome C | | | | |
| | | reductase complex | | | | |
| | | ubiquinone-binding | | | | |
| | | protein | | | | |
| CumMe_WSM_SF17142.G5920 | 50 | peptidyl-prolyl cis- | EXP | 2000 | | |
| | | trans isomerase, | | | | |
| | | chloroplast | | | | |
| CumMe_WSM_SF17190.G6200 | 51 | PRK | EXP | 2000 | | |
| | | (PHOS- | | | | |
| | | PHORIBULOKINASE) | | | | |
| CumMe_WSM_SF17250.G5910 | 52 | LHCB5 (LIGHT | EXP | 2000 | | |
| | | HARVESTING | | | | |
| | | COMPLEX OF | | | | |
| | | PHOTOSYSTEM II | | | | |
| | | 5) | | | | |
| CumMe_WSM_SF17252.G7330 | 53 | nascent polypeptide- | EXP | 2000 | Promoter; Leader; | 1-1195; 1196- |
| | | associated complex | | | Intron | 1297; 1298-2000 |
| | | (NAC) domain- | | | | |
| | | containing protein | | | | |
| CumMe_WSM_SF17253.G5150 | 54 | RPS9 | EXP | 1547 | | |
| | | (RIBOSOMAL | | | | |
| | | PROTEIN S9) | | | | |
| CumMe_WSM_SF17322.G5110 | 55 | 60S ribosomal | EXP | 2000 | | |
| | | protein L22 | | | | |
| | | (RPL22A) | | | | |
| CumMe_WSM_SF17349.G5770 | 56 | PGRL1B (PGR5- | EXP | 2000 | | |
| | | Like B) | | | | |
| CumMe_WSM_SF17357.G5630 | 57 | 40S ribosomal | EXP | 2000 | | |
| | | protein S10 | | | | |
| | | (RPS10B) | | | | |
| CumMe_WSM_SF17494.G5140 | 58 | MEE34 (maternal | EXP | 1591 | | |
| | | effect embryo arrest | | | | |
| | | 34) | | | | |
| CumMe_WSM_SF17524.G6410 | 59 | SUS2 | EXP | 2000 | | |
| | | (ABNORMAL | | | | |
| | | SUSPENSOR 2) | | | | |
| CumMe_WSM_SF17672.G5610 | 60 | PSAK (photosystem | EXP | 2000 | | |
| | | I subunit K) | | | | |
| CumMe_WSM_SF17773.G6620 | 61 | aconitase C-terminal | EXP | 2000 | | |
| | | domain-containing | | | | |
| | | protein | | | | |
| CumMe_WSM_SF17866.G6050 | 62 | ATPDIL5-1 (PDI- | EXP | 2000 | | |
| | | like 5-1) | | | | |
| CumMe_WSM_SF18004.G6600 | 63 | hydroxyproline-rich | EXP | 2000 | | |
| | | glycoprotein family | | | | |
| | | protein | | | | |
| CumMe_WSM_SF18045.G6670 | 64 | | EXP | 2000 | | |
| CumMe_WSM_SF18053.G5410 | 65 | endomembrane | EXP | 2000 | | |
| | | protein 70 | | | | |
| CumMe_WSM_SF18287.G5380 | 66 | CP12-1 | EXP | 2000 | | |
| CumMe_WSM_SF18488.G5340 | 67 | caffeoyl-CoA 3-O- | EXP | 2000 | Promoter; Leader | 1-1923; 1924-2000 |
| | | methyltransferase | | | | |
| CumMe_WSM_SF18504.G5090 | 68 | vacuolar ATP | EXP | 2000 | | |
| | | synthase subunit H | | | | |
| | | family protein | | | | |
| CumMe_WSM_SF18530.G5750 | 69 | GUN5 (GENOMES | EXP | 2000 | | |
| | | UNCOUPLED 5); | | | | |
| | | magnesium chelatase | | | | |
| CumMe_WSM_SF18536.G6480 | 70 | MBF1A | EXP | 2000 | | |
| | | (MULTIPROTEIN | | | | |
| | | BRIDGING | | | | |
| | | FACTOR 1A) | | | | |
| | | transcription | | | | |
| | | coactivator | | | | |
| CumMe_WSM_SF18575.G6410 | 71 | unknown protein | EXP | 2000 | | |
| CumMe_WSM_SF18634.G5190 | 72 | 60S ribosomal | EXP | 2000 | Promoter; Leader | 1-1971; 1972-2000 |
| | | protein L23 | | | | |
| | | (RPL23A) | | | | |
| CumMe_WSM_SF18645.G5380 | 73 | GS2 (GLUTAMINE | EXP | 2000 | | |
| | | SYNTHETASE 2) | | | | |
| CumMe_WSM_SF18716.G5860 | 74 | 40S ribosomal | EXP | 2000 | Promoter; Leader | Reverse compliment; |
| | | protein S12 | | | | see SEQ ID NO: 184 |
| | | (RPS12A); reverse | | | | |
| | | compliment: Auxin- | | | | |
| | | induced protein | | | | |
| | | x10A-like | | | | |
| CumMe_WSM_SF18801.G5040 | 75 | | EXP | 2000 | | |
| CumMe_WSM_SF18806.G6220 | 76 | unknown protein | EXP | 2000 | | |
| CumMe_WSM_SF18850.G5630 | 77 | PAC1; threonine- | EXP | 2000 | | |
| | | type endopeptidase | | | | |
| CumMe_WSM_SF18863.G7550 | 78 | ATP synthase | EXP | 2000 | | |
| | | gamma chain, | | | | |
| | | mitochondrial | | | | |
| | | (ATPC) | | | | |
| CumMe_WSM_SF18986.G6110 | 79 | GER1 (GERMIN- | EXP | 2000 | | |
| | | LIKE PROTEIN 1); | | | | |
| | | oxalate oxidase | | | | |
| CumMe_WSM_SF19064.G5690 | 80 | histone H3.2 | EXP | 2000 | Promoter; Leader; | 1-1581; 1582- |
| | | | | | Intron | 1670; 1671-2000 |
| CumMe_WSM_SF19323.G5120 | 81 | chloroplast outer | EXP | 2000 | | |
| | | envelope GTP- | | | | |
| | | binding protein, | | | | |
| | | putative | | | | |
| CumMe_WSM_SF19452.G5090 | 82 | glucan | EXP | 1072 | | |
| | | phosphorylase, | | | | |
| | | putative | | | | |
| CumMe_WSM_SF19631.G5170 | 83 | RuBisCO activase, | EXP | 1730 | | |
| | | putative | | | | |
| CumMe_WSM_SF19647.G5760 | 84 | 6-phosphogluconate | EXP | 2000 | Promoter; Leader; | 1-936; 937-1021; 1022- |
| | | dehydrogenase | | | Intron; Leader | 1992; 1993-2000 |
| | | family protein | | | | |
| CumMe_WSM_SF19839.G5090 | 85 | ATPDX1.1 | EXP | 1020 | Promoter; Leader | 1-928; 929-1020 |
| | | (pyridoxine | | | | |
| | | biosynthesis 1.1) | | | | |
| CumMe_WSM_SF19850.G5130 | 86 | HMGB2 (HIGH | EXP | 2000 | | |
| | | MOBILITY GROUP | | | | |
| | | B 2) transcription | | | | |
| | | factor | | | | |
| CumMe_WSM_SF19902.G5260 | 87 | universal stress | EXP | 2000 | | |
| | | protein (USP) family | | | | |
| | | protein/early | | | | |
| | | nodulin ENOD18 | | | | |
| | | family protein | | | | |
| CumMe_WSM_SF19992.G6100 | 88 | unknown protein | EXP | 2000 | | |
| CumMe_WSM_SF20132.G5560 | 89 | peroxidase 21 | EXP | 2000 | Promoter; Leader | 1-1962; 1963-2000 |
| CumMe_WSM_SF20147.G7910 | 90 | CSD1 | EXP | 2000 | | |
| | | (COPPER/ZINC | | | | |
| | | SUPEROXIDE | | | | |
| | | DISMUTASE 1) | | | | |
| CumMe_WSM_SF20355.G5130 | 91 | ATP synthase family | EXP | 2000 | | |
| CumMe_WSM_SF20359.G5870 | 92 | NADH-ubiquinone | EXP | 2000 | | |
| | | oxidoreductase 20 | | | | |
| | | kDa subunit, | | | | |
| | | mitochondrial | | | | |
| CumMe_WSM_SF20368.G5700 | 93 | PGR5 (proton | EXP | 2000 | | |
| | | gradient regulation | | | | |
| | | 5) | | | | |
| CumMe_WSM_SF20409.G5240 | 94 | elongation factor 1B | EXP | 2000 | | |
| | | alpha-subunit 1 | | | | |
| | | (eEF1 Balpha1) | | | | |
| CumMe_WSM_SF20431.G6340 | 95 | DHS2 (3-deoxy-d- | EXP | 2000 | | |
| | | arabino- | | | | |
| | | heptulosonate 7- | | | | |
| | | phosphate synthase) | | | | |
| CumMe_WSM_SF20505.G5440 | 96 | THIC (ThiaminC); | EXP | 1373 | | |
| | | ADP-ribose | | | | |
| | | pyrophosphohydrolase | | | | |
| CumMe_WSM_SF20509.G5920 | 97 | Y14; RNA binding/ | EXP | 2000 | | |
| | | protein binding | | | | |
| CumMe_WSM_SF206458.G5970 | 98 | FAD2 (FATTY | EXP | 2000 | Promoter | 1-2000 |
| | | ACID | | | | |
| | | DESATURASE 2) | | | | |
| CumMe_WSM_SF206534.G5200 | 99 | unknown protein | EXP | 2000 | | |
| CumMe_WSM_SF20997.G6990 | 100 | ALD1 (AGD2-LIKE | EXP | 2000 | | |
| | | DEFENSE | | | | |
| | | RESPONSE | | | | |
| | | PROTEIN1) | | | | |
| CumMe_WSM_SF21035.G5090 | 101 | sodium/calcium | EXP | 1078 | | |
| | | exchanger family | | | | |
| | | protein | | | | |
| CumMe_WSM_SF21117.G5370 | 102 | 30S ribosomal | EXP | 2000 | | |
| | | protein, putative | | | | |
| CumMe_WSM_SF21141.G5630 | 103 | 40S ribosomal | EXP | 2000 | | |
| | | protein S24 | | | | |
| | | (RPS24A) | | | | |
| CumMe_WSM_SF21198.G5180 | 104 | | EXP | 1974 | | |
| CumMe_WSM_SF21366.G5980 | 105 | GRF12 (GENERAL | EXP | 2000 | | |
| | | REGULATORY | | | | |
| | | FACTOR 12) | | | | |
| CumMe_WSM_SF21828.G5150 | 106 | cpHsc70-1 | EXP | 1643 | | |
| | | (chloroplast heat | | | | |
| | | shock protein 70-1) | | | | |
| CumMe_WSM_SF21886.G5080 | 107 | NPQ4 | EXP | 2000 | | |
| | | (NONPHOTO- | | | | |
| | | CHEMICAL | | | | |
| | | QUENCHING) | | | | |
| CumMe_WSM_SF22008.G5670 | 108 | NAP1; 2 | EXP | 2000 | | |
| | | (NUCLEOSOME | | | | |
| | | ASSEMBLY | | | | |
| | | PROTEIN 1; 2) | | | | |
| CumMe_WSM_SF22070.G5280 | 109 | fructose- | EXP | 2000 | | |
| | | bisphosphate | | | | |
| | | aldolase, putative | | | | |
| CumMe_WSM_SF22097.G5540 | 110 | APX3 | EXP | 2000 | | |
| | | (ASCORBATE | | | | |
| | | PEROXIDASE 3) | | | | |
| CumMe_WSM_SF22254.G5760 | 111 | 40S ribosomal | EXP | 2000 | | |
| | | protein S7 (RPS7B) | | | | |
| CumMe_WSM_SF22275.G5780 | 112 | ribosomal protein | EXP | 1027 | | |
| | | L17 family protein | | | | |
| CumMe_WSM_SF22355.G5310 | 113 | | EXP | 2000 | | |
| CumMe_WSM_SF22531.G5120 | 114 | eukaryotic | EXP | 2000 | Promoter; Leader; | 1-759; 760-858; 859- |
| | | translation initiation | | | Intron; Leader | 1979; 1980-2000 |
| | | factor 1A, putative | | | | |
| CumMe_WSM_SF22870.G5370 | 115 | ATSARA1A | EXP | 2000 | | |
| | | (ARABIDOPSIS | | | | |
| | | THALIANA | | | | |
| | | SECRETION- | | | | |
| | | ASSOCIATED RAS | | | | |
| | | SUPER FAMILY 1) | | | | |
| CumMe_WSM_SF22934.G5290 | 116 | T-complex protein 1 | EXP | 2000 | | |
| | | epsilon subunit, | | | | |
| | | putative | | | | |
| CumMe_WSM_SF23181.G5100 | 117 | CEV1 | EXP | 1025 | | |
| | | (CONSTITUTIVE | | | | |
| | | EXPRESSION OF | | | | |
| | | VSP 1) | | | | |
| CumMe_WSM_SF23186.G6160 | 118 | ubiquinol- | EXP | 2000 | | |
| | | cytochrome C | | | | |
| | | reductase complex | | | | |
| | | 14 kDa protein, | | | | |
| | | putative | | | | |
| CumMe_WSM_SF23397.G5210 | 119 | RPL27 | EXP | 2000 | | |
| | | (RIBOSOMAL | | | | |
| | | PROTEIN LARGE | | | | |
| | | SUBUNIT 27) | | | | |
| CumMe_WSM_SF23760.G5200 | 120 | NDPK1; ATP | EXP | 2000 | Promoter; Leader | 1-1901; 1902-2000 |
| | | binding/nucleoside | | | | |
| | | diphosphate kinase | | | | |
| CumMe_WSM_SF23906.G6180 | 121 | PSBX (photosystem | EXP | 2000 | | |
| | | II subunit X) | | | | |
| CumMe_WSM_SF24040.G5450 | 122 | RPS17 | EXP | 2000 | | |
| | | (RIBOSOMAL | | | | |
| | | PROTEIN S17) | | | | |
| CumMe_WSM_SF24045.G5400 | 123 | EXL3 (EXORDIUM | EXP | 2000 | | |
| | | LIKE 3) | | | | |
| CumMe_WSM_SF24117.G5600 | 124 | 60S ribosomal | EXP | 2000 | | |
| | | protein L26 | | | | |
| | | (RPL26A) | | | | |
| CumMe_WSM_SF25084.G5580 | 125 | | EXP | 2000 | | |
| CumMe_WSM_SF25141.G5160 | 126 | isocitrate | EXP | 1397 | Promoter; Leader | 1-1322; 1323-1397 |
| | | dehydrogenase, | | | | |
| | | putative | | | | |
| CumMe_WSM_SF25355.G5000 | 127 | LOS1; copper ion | EXP | 2000 | Promoter; Leader; | 1-734; 735-811; 812- |
| | | binding translation | | | Intron; Leader; | 1340; 1341-1360; |
| | | elongation factor | | | CDS | 1361-2000 |
| CumMe_WSM_SF25370.G5000 | 128 | PSBP-1 | EXP | 1657 | | |
| | | (PHOTOSYSTEM II | | | | |
| | | SUBUNIT P-1) | | | | |
| CumMe_WSM_SF25455.G5370 | 129 | GLY3 | EXP | 2000 | | |
| | | (GLYOXALASE II | | | | |
| | | 3) | | | | |
| CumMe_WSM_SF25936.G5450 | 130 | mitochondrial | EXP | 2000 | Promoter; Leader | 1-1878; 1879-2000 |
| | | substrate carrier | | | | |
| | | family protein | | | | |
| CumMe_WSM_SF27080.G5510 | 131 | LIP1 (LIPOIC ACID | EXP | 2000 | | |
| | | SYNTHASE 1) | | | | |
| CumMe_WSM_SF27222.G5150 | 132 | DRT112; copper ion | EXP | 2000 | | |
| | | binding/electron | | | | |
| | | carrier | | | | |
| CumMe_WSM_SF27957.G5450 | 133 | SMAP1 (SMALL | EXP | 2000 | | |
| | | ACIDIC PROTEIN | | | | |
| | | 1) | | | | |
| CumMe_WSM_SF28729.G5340 | 134 | RNA-binding protein | EXP | 1696 | | |
| | | cp29, putative | | | | |
| CumMe_WSM_SF28805.G6200 | 135 | unknown protein | EXP | 2000 | | |
| CumMe_WSM_SF31264.G5380 | 136 | ATPH1 | EXP | 2000 | | |
| | | (ARABIDOPSIS | | | | |
| | | THALIANA | | | | |
| | | PLECKSTRIN | | | | |
| | | HOMOLOGUE 1) | | | | |
| CumMe_WSM_SF35856.G5150 | 137 | TIP4; 1 (tonoplast | EXP | 1575 | | |
| | | intrinsic protein 4; 1) | | | | |
| CumMe_WSM_SF40859.G5250 | 138 | SMT2 (STEROL | EXP | 2000 | | |
| | | METHYLTRANS- | | | | |
| | | FERASE 2) | | | | |
| CumMe_WSM_SF41124.G5080 | 139 | 40S ribosomal | EXP | 1006 | Promoter; Leader | 1-883; 884-1006 |
| | | protein S2 (RPS2C) | | | | |
| CumMe_WSM_SF41128.G5410 | 140 | CRY2 | EXP | 2000 | | |
| | | (CRYPTOCHROME | | | | |
| | | 2) | | | | |
| CumMe_WSM_SF41254.G5160 | 141 | GDP-D-glucose | EXP | 1556 | | |
| | | phosphorylase | | | | |
| CumMe_WSM_SF41588.G5470 | 142 | PRPL11 (PLASTID | EXP | 2000 | | |
| | | RIBOSOMAL | | | | |
| | | PROTEIN L11) | | | | |
| CumMe_WSM_SF41644.G6400 | 143 | SHD (SHEPHERD) | EXP | 2000 | | |
| CumMe_WSM_SF41983.G5000 | 144 | catalytic/coenzyme | EXP | 1337 | | |
| | | binding | | | | |
| CumMe_WSM_SF42075.G5100 | 145 | CPN60B | EXP | 2000 | | |
| | | (CHAPERONIN 60 | | | | |
| | | BETA) | | | | |
| CumMe_WSM_SF42141.G5110 | 146 | cathepsin B-like | EXP | 1212 | | |
| | | cysteine protease, | | | | |
| | | putative | | | | |
| CumMe_WSM_SF44933.G5290 | 147 | EBF1 (EIN3- | EXP | 2000 | | |
| | | BINDING F BOX | | | | |
| | | PROTEIN 1) | | | | |
| | | ubiquitin-protein | | | | |
| | | ligase | | | | |
| CumMe_WSM_SF44977.G5000 | 148 | PAP26 (PURPLE | EXP | 1254 | | |
| | | ACID | | | | |
| | | PHOSPHATASE | | | | |
| | | (26) | | | | |
| CumMe_WSM_SF45441.G5510 | 149 | GAPA-2 | EXP | 2000 | | |
| | | (GLYCERALDEHYDE | | | | |
| | | 3-PHOSPHATE | | | | |
| | | DEHYDROGENASE | | | | |
| | | A SUBUNIT 2) | | | | |
| CumMe_WSM_SF45882.G5120 | 150 | fructose-1,6- | EXP | 1680 | | |
| | | bisphosphatase, | | | | |
| | | putative | | | | |
| CumMe_WSM_SF47806.G5070 | 151 | ATP synthase | EXP | 1524 | | |
| | | epsilon chain, | | | | |
| | | mitochondrial | | | | |
| CumMe_WSM_SF53106.G5190 | 152 | CPN60A | EXP | 1851 | | |
| | | (CHAPERONIN- | | | | |
| | | 60ALPHA) | | | | |
| CumMe_WSM_SF65588.G5230 | 153 | vacuolar calcium- | EXP | 2000 | | |
| | | binding protein- | | | | |
| | | related | | | | |
| CumMe_WSM_SF9060.G5120 | 154 | APE2 | EXP | 1288 | | |
| | | (ACCLIMATION | | | | |
| | | OF | | | | |
| | | PHOTOSYNTHESIS | | | | |
| | | TO | | | | |
| | | ENVIRONMENT 2) | | | | |
| P-CUCme.1-1:1:1rc | 155 | Phosphatase 2A | EXP | 2000 | Promoter; Leader; | 1-1135; 1136- |
| | | | | | Intron; Leader | 1249; 1250-1990; |
| | | | | | | 1991-2000 |
| EXP-CUCme.4:1:1 | 156 | Ubiquitin 2 | EXP | 2011 | Promoter; Leader; | 1-1646; 1647- |
| | | | | | Intron; Leader | 1704; 1705-2005; |
| | | | | | | 2006-2008 |
| P-CUCme.4-1:1:4 | 157 | Ubiquitin 2 | P; L | 1698 | Promoter; Leader | |
| I-CUCme.4-1:1:1 | 158 | Ubiquitin 2 | I; L | 313 | Intron; Leader | |
| EXP-CUCme.5:1:1 | 159 | Ubiquitin 3 | EXP | 2010 | Promoter; Leader; | 1-748; 749-819; 820- |
| | | | | | Intron; Leader | 2004; 2005-2007 |
| P-CUCme.5-1:1:3 | 160 | Ubiquitin 3 | P; L | 1107 | Promoter; Leader | |
| I-CUCme.5-1:1:1 | 161 | Ubiquitin 3 | I; L | 903 | Intron; Leader | |
| EXP-CUCme.eEF1a:1:1 | 162 | Elongation Factor 1 | EXP | 1235 | Promoter; Leader; | 1-617; 618-677; 678- |
| | | alpha | | | Intron; Leader | 1213; 1214-1235 |
| P-CUCme.eEF1a-1:1:1 | 163 | Elongation Factor 1 | P | 617 | Promoter | |
| | | alpha | | | | |
| L-CUCme.eEF1a-1:1:1 | 164 | Elongation Factor 1 | L | 54 | Leader | |
| | | alpha | | | | |
| I-CUCme.eEF1a-1:1:1 | 165 | Elongation Factor 1 | I | 545 | Intron | |
| | | alpha | | | | |
| L-CUCme.eEF1a-1:1:2 | 166 | Elongation Factor 1 | L | 19 | Leader | |
| | | alpha | | | | |
| P-CUCme.19-1:1:3 | 167 | Chloropyll a/b | EXP | 2003 | Promoter; Leader | 1-1958; 1959-2003 |
| | | binding protein | | | | |
| EXP-CUCme.SAMS2:1:1 | 168 | S- | EXP | 2004 | Promoter; Leader; | 1-1067; 1068- |
| | | Adenosylmethionine | | | Intron | 1165; 1166-2003 |
| | | Synthetase | | | | |
| P-CUCme.SAMS2-1:1:1 | 169 | S- | P | 1067 | Promoter | |
| | | Adenosylmethionine | | | | |
| | | Synthetase | | | | |
| L-CUCme.SAMS2-1:1:1 | 170 | S- | L | 92 | Leader | |
| | | Adenosylmethionine | | | | |
| | | Synthetase | | | | |
| I-CUCme.SAMS2-1:1:1 | 171 | S- | I | 845 | Intron | |
| | | Adenosylmethionine | | | | |
| | | Synthetase | | | | |
| EXP-CUCme.29:1:1 | 172 | Ribosomal protein | EXP | 2018 | Promoter; Leader; | 1-490; 491-571; 572- |
| | | S5a | | | Intron; Leader | 2012; 2013-2018 |
| P-CUCme.29-1:1:4 | 173 | Ribosomal protein | P; L | 565 | Promoter; Leader | |
| | | S5a | | | | |
| I-CUCme.29-1:1:1 | 174 | Ribosomal protein | I; L | 1453 | Intron; Leader | |
| | | S5a | | | | |
| P- | 175 | histone H4 | EXP | 1999 | Promoter; Leader; | 1-1946; 947-1999 |
| CUCme.CumMe_WSM_SF16444.G5140- | | | | | Intron | |
| 1:1:1 | | | | | | |
| P- | 176 | ATARFB1A (ADP- | EXP | 2004 | Promoter; Leader; | 1-1331; 1332- |
| CUCme.CumMe_WSM_SF16563.G5560- | | ribosylation factor | | | Intron; Leader | 1429; 1430-1992; 1993- |
| 1:1:1 | | B1A) | | | | 2004 |
| P- | 177 | ubiquinol- | EXP | 2005 | Promoter; Leader | 1-1901; 1902-2005 |
| CUCme.CumMe_WSM_SF17111.G5790- | | cytochrome C | | | | |
| 1:1:1 | | reductase complex | | | | |
| | | ubiquinone-binding | | | | |
| | | protein | | | | |
| EXP- | 178 | nascent polypeptide- | EXP | 1978 | Promoter; Leader; | 1-1167; 1168- |
| CumMe.WSM_SF17252.G7330:1:1 | | associated complex | | | Intron; Leader | 1269; 1270-1972; 1973- |
| | | (NAC) domain- | | | | 1975 |
| | | containing protein | | | | |
| P-CUCme.WSM_SF17252.G7330- | 179 | nascent polypeptide- | P; L | 1263 | Promoter; Leader | |
| 1:1:1 | | associated complex | | | | |
| | | (NAC) domain- | | | | |
| | | containing protein | | | | |
| I-CUCme.WSM_SF17252.G7330- | 180 | nascent polypeptide- | I; L | 715 | Intron; Leader | |
| 1:1:1 | | associated complex | | | | |
| | | (NAC) domain- | | | | |
| | | containing protein | | | | |
| P- | 181 | caffeoyl-CoA 3-O- | EXP | 2000 | Promoter; Leader | 1-923; 1924-2000 |
| CUCme.CumMe_WSM_SF18488.G5340- | | methyltransferase | | | | |
| 1:1:1 | | | | | | |
| P- | 182 | MBF1A | EXP | 2000 | Promoter; Leader; | |
| CUCme.CumMe_WSM_SF18536.G6480- | | (MULTIPROTEIN | | | Intron | |
| 1:1:1 | | BRIDGING | | | | |
| | | FACTOR 1A) | | | | |
| | | transcription | | | | |
| | | coactivator | | | | |
| P- | 183 | 60S ribosomal | EXP | 1989 | Promoter; Leader | 1-1960; 1961-1989 |
| CUCme.CumMe_WSM_SF18634.G5190- | | protein L23 | | | | |
| 1:1:1 | | (RPL23A) | | | | |
| P- | 184 | Auxin-induced | EXP | 1463 | Promoter; Leader | 1-1392; 1393-1463 |
| CUCme.CumMe_WSM_SF18716.G5860- | | prtoein X10A-like | | | | |
| 1:1:1 | | | | | | |
| EXP- | 185 | histone H3.2 | EXP | 2006 | Promoter; Leader; | 1-1581; 1582- |
| CUCme.WSM_SF19064.G5690:1:1 | | | | | Intron; Leader | 1670; 1671-2000; 2001- |
| | | | | | | 2003 |
| P-CUCme.WSM_SF19064.G5690- | 186 | histone H3.2 | P; L | 1664 | Promoter; Leader | |
| 1:1:1 | | | | | | |
| I-CUCme.WSM_SF19064.G5690- | 187 | histone H3.2 | I; L | 342 | Intron; Leader | |
| 1:1:1 | | | | | | |
| P- | 188 | 6-phosphogluconate | EXP | 2003 | Promoter; Leader; | 1-939; 940-1024; 1025- |
| CUCme.CumMe_WSM_SF19647.G5760- | | dehydrogenase | | | Intron; Leader | 1995; 1996-2003 |
| 1:1:1 | | family protein | | | | |
| P- | 189 | ATPDX1.1 | EXP | 1024 | Promoter; Leader | 1-904; 905-1024 |
| CUCme.CumMe_WSM_SF19839.G5090- | | (pyridoxine | | | | |
| 1:1:1 | | biosynthesis 1.1) | | | | |
| P- | 190 | peroxidase 21 | EXP | 2001 | Promoter; Leader | 1-1962; 1963-2001 |
| CUCme.CumMe_WSM_SF20132.G5560- | | | | | | |
| 1:1:1 | | | | | | |
| P- | 191 | FAD2 (FATTY | EXP | 4175 | Promoter; Leader; | 1-2171; 2172- |
| CUCme.CumMe_WSM_SF206458.G5970- | | ACID | | | Intron; Leader | 2325; 2326-4155; 4156- |
| 1:1:1 | | DESATURASE 2) | | | | 4175 |
| P- | 192 | eukaryotic | EXP | 1999 | Promoter; Leader; | 1-759; 760-858; 859- |
| CUCme.CumMe_WSM_SF22531.G5120- | | translation initiation | | | Intron; Leader | 1978; 1979-1999 |
| 1:1:1 | | factor 1A, putative | | | | |
| P- | 193 | NDPK1; ATP | EXP | 2000 | Promoter; Leader | 1-1901; 1902-2000 |
| CUCme.CumMe_WSM_SF23760.G5200- | | binding/nucleoside | | | | |
| 1:1:1 | | diphosphate kinase | | | | |
| P- | 194 | PSBX (photosystem | EXP | 2000 | Promoter; Leader | |
| CUCme.CumMe_WSM_SF23906.G6180- | | II subunit X) | | | | |
| 1:1:1 | | | | | | |
| P- | 195 | isocitrate | EXP | 1400 | Promoter; Leader | 1-1325; 1326-1400 |
| CUCme.CumMe_WSM_SF25141.G5160- | | dehydrogenase, | | | | |
| 1:1:2 | | putative | | | | |
| P- | 196 | LOS1; copper ion | EXP | 2019 | Promoter; Leader; | 1-734; 735-811; 812- |
| CUCme.CumMe_WSM_SF25355.G5000- | | binding translation | | | Intron; Leader; CDS | 1340; 1341-1360; 1361- |
| 1:1:1 | | elongation factor | | | | 2019 |
| P- | 197 | mitochondrial | EXP | 1999 | Promoter; Leader | 1-1877; 1878-1999 |
| CUCme.CumMe_WSM_SF25936.G5450- | | substrate carrier | | | | |
| 1:1:1 | | family protein | | | | |
| P- | 198 | TIP4; 1 (tonoplast | EXP | 1578 | | |
| CUCme.CumMe_WSM_SF35856.G5150- | | intrinsic protein 4; 1) | | | | |
| 1:1:1 | | | | | | |
| P- | 199 | 40S ribosomal | EXP | 1023 | Promoter; Leader | 1-945; 946-1023 |
| CUCme.CumMe_WSM_SF41124.G5080- | | protein S2 (RPS2C) | | | | |
| 1:1:1 | | | | | | |
| P-CUCme.20-1:3 | 211 | Chloropyll a/b | EXP | 1446 | Promoter; Leader | 1-1390; 1391-1446 |
| | | binding protein | | | | |
| EXP-CUCme.29:1:2 | 212 | Ribosomal protein | EXP | 2018 | Promoter; Leader; | 1-490; 491-571; 572- |
| | | S5a | | | Intron; Leader | 2011; 2013-2018 |

As shown in Table 1, for example, the transcriptional regulatory expression element group (EXP) designated EXP-CUCme.Ubq1:1:1 (SEQ ID NO: 1), with components isolated from C. melo, comprises a 2068 base pair sized (bp) promoter element, P-CUCme.Ubq1-1:1:15 (SEQ ID NO: 2), operably linked 5′ to a leader element, L-CUCme.Ubq1-1:1:1 (SEQ ID NO: 3), operably linked 5′ to an intron element, I-CUCme.Ubq1-1:1:1 (SEQ ID NO: 4). The transcriptional regulatory expression element group (EXP) designated EXP-CUCme.Ubq1:1:2(SEQ ID NO: 5), with components isolated from C. melo, comprises a 1459 bp promoter element, P-CUCme.Ubq1-1:1:16 (SEQ ID NO: 6), operably linked 5′ to a leader element, L-CUCme.Ubq1-1:1:1 (SEQ ID NO: 3), operably linked 5′ to an intron element, I-CUCme.Ubq1-1:1:1 (SEQ ID NO: 4). The transcriptional regulatory expression element group (EXP) designated EXP-CUCme.Ubq1:1:3 (SEQ ID NO: 7), with components isolated from C. melo, comprises a 964 bp promoter element, P-CUCme.Ubq1-1:1:17 (SEQ ID NO: 8), operably linked 5′ to a leader element, L-CUCme.Ubq1-1:1:1 (SEQ ID NO: 3), operably linked 5′ to an intron element, I-CUCme.Ubq1-1:1:1 (SEQ ID NO: 4). The transcriptional regulatory expression element group (EXP) designated EXP-CUCme.Ubq1:1:4 (SEQ ID NO: 9), with components isolated from C. melo, comprises a 479 bp promoter element, P-CUCme.Ubq1-1:1:18 (SEQ ID NO: 10), operably linked 5′ to a leader element, L-CUCme.Ubq1-1:1:1 (SEQ ID NO: 3), operably linked 5′ to an intron element, I-CUCme.Ubq1-1:1:1 (SEQ ID NO: 4). The transcriptional regulatory expression element group (EXP) designated EXP-CUCme.Ubq1:1:5 (SEQ ID NO: 11), with components isolated from C. melo, comprises a 173 bp promoter clement, P-CUCme.Ubq1-1:1:19 (SEQ ID NO: 12), operably linked 5′ to a leader element, L-CUCme.Ubq1-1:1:1 (SEQ ID NO: 3), operably linked 5′ to an intron element, I-CUCme.Ubq1-1:1:1 (SEQ ID NO: 4).

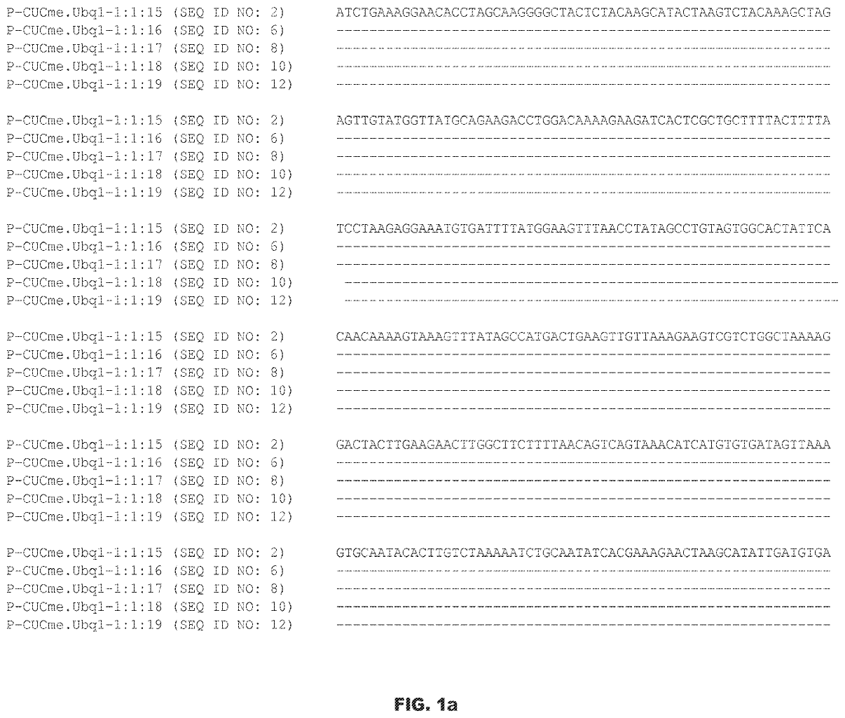

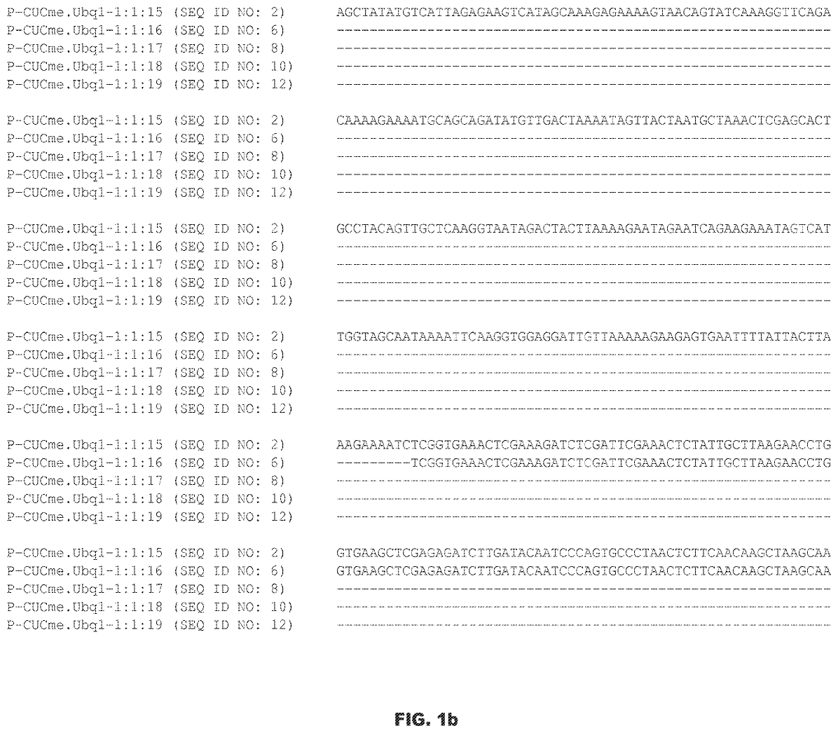

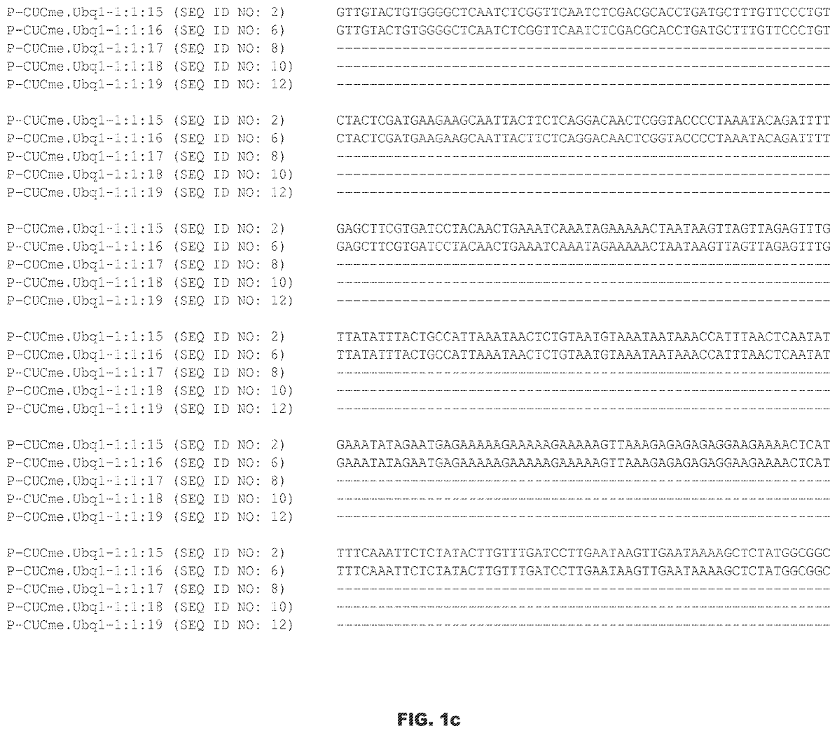

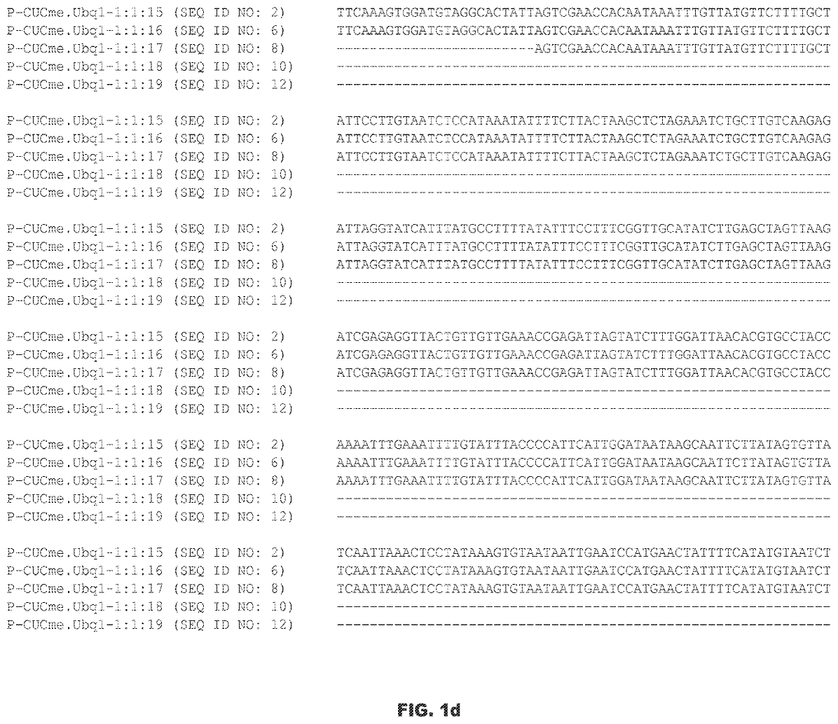

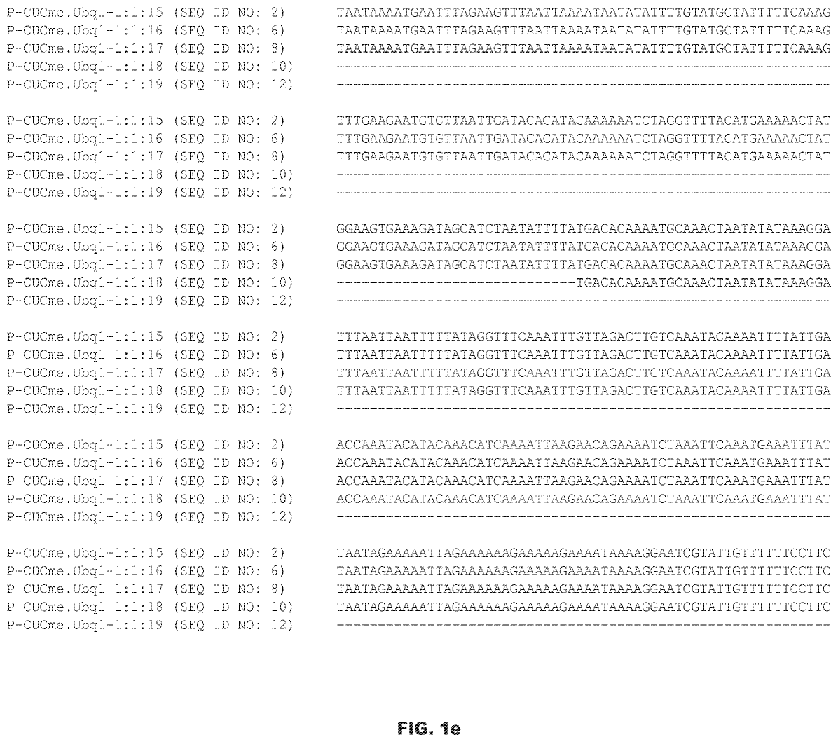

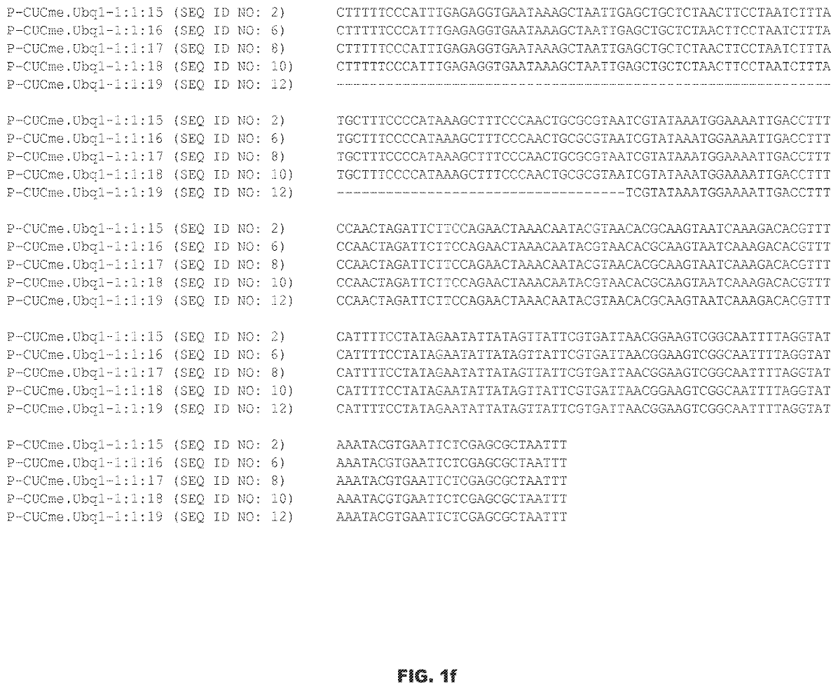

An alignment of the ubiquitin 1 promoter sequences is provided in FIGS. 1a-1f. The promoter elements, P-CUCme.Ubq1-1:1:16 (SEQ ID NO: 6), P-CUCme.Ubq1-1:1:17 (SEQ ID NO: 8), P-CUCme.Ubq1-1:1:18 (SEQ ID NO: 10) and P-CUCme.Ubq1-1:1:19 (SEQ ID NO: 12) were built by introducing varying lengths of deletions from the 5′ end of the promoter, P-CUCme.Ubq1-1:1:15 (SEQ ID NO: 2).

### Example 2: Analysis of Regulatory Elements Driving GUS in Soy Cotyledon Protoplasts

Soybean cotyledon protoplasts were transformed with plant expression vectors containing a test transcriptional regulatory expression element group driving expression of the ß-glucuronidase (GUS) transgene and compared to GUS expression in leaf protoplasts in which expression of GUS is driven by known constitutive promoters.

Expression of a transgene driven by EXP-CUCme.Ubq1:1:1 (SEQ ID NO: 1), EXP-CUCme.Ubq1:1:2 (SEQ ID NO: 5), EXP-CUCme.Ubq1:1:3 (SEQ ID NO: 7), EXP-CUCme.Ubq1:1:4 (SEQ ID NO: 9) and EXP-CUCme.Ubq1:1:5 (SEQ ID NO: 11) was compared with expression from known constitutive promoters. Each plant expression vector was comprised of a right border region from Agrobacterium tumefaciens, a first transgene cassette comprised of an EXP sequence or known constitutive promoter operably linked 5′ to a coding sequence for ß-glucuronidase (GUS, SEQ ID NO: 206) containing a processable intron derived from the potato light-inducible tissue-specific ST-LS1 gene (Genbank Accession: X04753), operably linked 5′ to a 3′ termination region from the Gossypium barbadense E6 gene (T-Gb.E6-3b:1:1, SEQ ID NO: 204), the Pisum sativum RbcS2-E9 gene (T-Ps.RbcS2-E9-1:1:6, SEQ ID NO: 203), or the Gossypium barbadense FbLate-2 gene (T-Gb.FbL2-1:1:1, SEQ ID NO: 205); a second transgene selection cassette used for selection of transformed plant cells that either confers resistance to the herbicide glyphosate (driven by the Arabidopsis Actin 7 promoter) or the antibiotic, kanamycin and a left border region from A. tumefaciens. A promoterless control plant expression vector (pMON124912) served as a negative control for expression. The foregoing test and constitutive expression element groups were cloned into plant expression vectors as shown in Table 2 below.

TABLE 2
Plant expression vectors and corresponding
expression element group and 3′ UTR.

| Expression | Regulatory | SEQ ID | |
|---|---|---|---|
| Vector | Element | NO: | 3′ UTR |
| pMON80585 | EXP-At.Atntt1:1:2 | 200 | T-Ps.RbcS2-E9- |
| | | | 1:1:6 |
| pMON109584 | EXP-CaMV.35S- | 201 | T-Gb.E6-3b:1:1 |
| | enh + Ph.DnaK:1:3 | | |
| pMON118756 | EXP-At.Act7:1:11 | 202 | T-Gb.E6-3b:1:1 |
| pMON124912 | No promoter | | T-Gb.FbL2-1:1:1 |
| pMON138776 | EXP-CUCme.Ubq1:1:1 | 1 | T-Gb.FbL2-1:1:1 |
| pMON138777 | EXP-CUCme.Ubq1:1:2 | 5 | T-Gb.FbL2-1:1:1 |
| pMON138778 | EXP-CUCme.Ubq1:1:3 | 7 | T-Gb.FbL2-1:1:1 |
| pMON138779 | EXP-CUCme.Ubq1:1:4 | 9 | T-Gb.FbL2-1:1:1 |
| pMON138780 | EXP-CUCme.Ubq1:1:5 | 11 | T-Gb.FbL2-1:1:1 |

Two plasmids, for use in co-transformation and normalization of data, were also constructed. One transformation control plasmid was comprised of a constitutive promoter, driving the expression of the firefly (Photinus pyralis) luciferase coding sequence (FLuc, SEQ ID NO: 207), operably linked 5′ to a 3′ termination region from the Agrobacterium tumefaciens nopaline synthase gene (T-AGRtu.nos-1:1:13, SEQ ID NO: 209). The other transformation control plasmid was comprised of a constitutive promoter, driving the expression of the sea pansy (Renilla reniformis) luciferase coding sequence (RLuc, SEQ ID NO: 208), operably linked 5′ to a 3′ termination region from the Agrobacterium tumefaciens nopaline synthase gene.

The plant expression vectors, pMON80585, pMON109584, pMON118756, pMON124912, pMON138776, pMON138777, pMON138778, pMON138779 and pMON138780 were used to transform soybean cotyledon protoplast cells using PEG transformation methods. Protoplast cells were transformed with equimolar amounts of each of the two transformation control plasmids and a test plant expression vector. GUS and luciferase activity was assayed. Measurements of both GUS and luciferase were conducted by placing aliquots of a lysed preparation of cells transformed as above into two different small-well trays. One tray was used for GUS measurements, and a second tray was used to perform a dual luciferase assay using the dual luciferase reporter assay system (Promega Corp., Madison, WI; see for example, Promega Notes Magazine, No: 57, 1996, p. 02). Sample measurements were made using 3 or 4 replicates per transformation. The average GUS and luciferase values are presented in Table 3 below.

TABLE 3
Average GUS and luciferase expression values and GUS/luciferase ratios.

| | | SEQ | | | | | |
|---|---|---|---|---|---|---|---|
| | | ID | Average | Average | Average | GUS/ | GUS/ |
| Construct | Regulatory Element | NO: | GUS | FLuc | RLuc | FLuc | RLuc |
| pMON80585 | EXP-At.Atntt1:1:2 | 200 | 55173 | 6498 | 30503 | 8.49 | 1.81 |
| pMON109584 | EXP-CaMV.35S- | 200 | 24940 | 5050.75 | 35495 | 4.94 | 0.70 |
| | enh + Ph.DnaK:1:3 | | | | | | |
| pMON118756 | EXP-At.Act7:1:11 | 201 | 9871 | 6880 | 40850 | 1.43 | 0.24 |
| pMON124912 | No promoter | | 2000 | 11670 | 73187 | 0.17 | 0.03 |
| pMON138776 | EXP- | 1 | 26972 | 6467.25 | 37200 | 4.17 | 0.73 |
| | CUCme.Ubq1:1:1 | | | | | | |
| pMON138777 | EXP- | 5 | 41307 | 5902.5 | 24396 | 7.00 | 1.69 |
| | CUCme.Ubq1:1:2 | | | | | | |
| pMON138778 | EXP- | 7 | 90140 | 10710.5 | 60983 | 8.42 | 1.48 |
| | CUCme.Ubq1:1:3 | | | | | | |
| pMON138779 | EXP- | 9 | 35526 | 5590 | 28001 | 6.36 | 1.27 |
| | CUCme.Ubq1:1:4 | | | | | | |
| pMON138780 | EXP- | 11 | 23298 | 4483.25 | 19075 | 5.20 | 1.22 |
| | CUCme.Ubq1:1:5 | | | | | | |

To compare the relative activity of each promoter in soybean cotyledon protoplasts, GUS values were expressed as a ratio of GUS to luciferase activity and normalized with respect to the expression levels observed for the constitutive expression element groups, EXP-At.Act7:1:11 and EXP-CaMV.35S-enh+Ph.DnaK:1:3. Table 4 below shows the GUS to firefly luciferase (FLuc) ratios normalized with respect to EXP-At.Act7:1:11 and EXP-CaMV.35S-enh+Ph.DnaK:1:3. Table 5 below shows the GUS to renilla luciferase (RLuc) ratios normalized with respect to EXP-At.Act7:1:11 and EXP-CaMV.35S-enh+Ph.DnaK:1:3.

TABLE 4
GUS to firefly luciferase (FLuc) ratios normalized with respect
to EXP-At.Act7:1:11 and EXP-CaMV.35S-enh + Ph.DnaK:1:3.

| | | | GUS/FLuc | GUS/FLuc |
|---|---|---|---|---|
| | | | normalized | normalized with |
| | | | with respect | respect to EXP- |
| | | SEQ ID | to EXP- | CaMV.35S- |
| Construct | Regulatory Element | NO: | At.Act7:1:11 | enh + Ph.DnaK:1:3 |
| pMON80585 | EXP-At.Atntt1:1:2 | 200 | 5.92 | 1.72 |
| pMON109584 | EXP-CaMV.35S- | 201 | 3.44 | 1.00 |
| | enh + Ph.DnaK:1:3 | | | |
| pMON118756 | EXP-At.Act7:1:11 | 202 | 1.00 | 0.29 |
| pMON124912 | No promoter | | 0.12 | 0.03 |
| pMON138776 | EXP-CUCme.Ubq1:1:1 | 1 | 2.91 | 0.84 |
| pMON138777 | EXP-CUCme.Ubq1:1:2 | 5 | 4.88 | 1.42 |
| pMON138778 | EXP-CUCme.Ubq1:1:3 | 7 | 5.87 | 1.70 |
| pMON138779 | EXP-CUCme.Ubq1:1:4 | 9 | 4.43 | 1.29 |
| pMON138780 | EXP-CUCme.Ubq1:1:5 | 11 | 3.62 | 1.05 |

TABLE 5
GUS to renilla luciferase (RLuc) ratios normalized with respect
to EXP-At.Act7:1:11 and EXP-CaMV.35S-enh + Ph.DnaK:1:3.

| | | | GUS/RLuc | GUS/RLuc |
|---|---|---|---|---|
| | | | normalized | normalized with |
| | | | with respect | respect to EXP- |
| | | SEQ ID | to EXP- | CaMV.35S- |
| Construct | Regulatory Element | NO: | At.Act7:1:11 | enh + Ph.DnaK:1:3 |
| pMON80585 | EXP-At.Atntt1:1:2 | 200 | 7.49 | 2.57 |
| pMON109584 | EXP-CaMV.35S- | 201 | 2.91 | 1.00 |
| | enh + Ph.DnaK:1:3 | | | |
| pMON118756 | EXP-At.Act7:1:11 | 202 | 1.00 | 0.34 |
| pMON124912 | No promoter | | 0.11 | 0.04 |
| pMON138776 | EXP-CUCme.Ubq1:1:1 | 1 | 3.00 | 1.03 |
| pMON138777 | EXP-CUCme.Ubq1:1:2 | 5 | 7.01 | 2.41 |
| pMON138778 | EXP-CUCme.Ubq1:1:3 | 7 | 6.12 | 2.10 |
| pMON138779 | EXP-CUCme.Ubq1:1:4 | 9 | 5.25 | 1.81 |
| pMON138780 | EXP-CUCme.Ubq1:1:5 | 11 | 5.05 | 1.74 |

As can be seen in Tables 4 and 5 above, each of the expression element groups EXP-CUCme.Ubq1:1:1 (SEQ ID NO: 1), EXP-CUCme.Ubq1:1:2 (SEQ ID NO: 5), EXP-CUCme.Ubq1:1:3 (SEQ ID NO: 7), EXP-CUCme.Ubq1:1:4 (SEQ ID NO: 9) and EXP-CUCme.Ubq1:1:5 (SEQ ID NO: 11) demonstrated the ability of driving transgene expression in soybean cotyledon protoplasts. Expression levels were greater than that of EXP-At.Act7:1:11 and was 2.9 to 5.8 (FLuc) or 3 to 7 (RLuc) fold higher than EXP-At.Act7:1:11 in this assay. Expression was equivalent or higher than expression observed for EXP-CaMV.35S-enh+Ph.DnaK:1:3. Expression levels were 0.8 to 1.7 (FLuc) or 1 to 2.4 (RLuc) fold higher than expression observed for EXP-CaMV.35S-enh+Ph.DnaK:1:3.

### Example 3: Analysis of Regulatory Elements Driving GUS in Bombarded Soybean Leaves and Roots

Soybean leaves and roots were transformed with plant expression vectors containing a test transcriptional regulatory expression element group driving expression of the ß-glucuronidase (GUS) transgene and compared to GUS expression in roots and leaves in which expression of GUS is driven by known constitutive promoters.

Expression of a transgene driven by EXP-CUCme.Ubq1:1:1 (SEQ ID NO: 1), EXP-CUCme.Ubq1:1:2 (SEQ ID NO: 5), EXP-CUCme.Ubq1:1:3 (SEQ ID NO: 7), EXP-CUCme.Ubq1:1:4 (SEQ ID NO: 9) and EXP-CUCme.Ubq1:1:5 (SEQ ID NO: 11) was compared with expression from known constitutive promoters in particle bombarded soybean leaves and roots. The plant expression vectors used for transformation of leaves and roots was the same as those presented in Table 2 of Example 2 above.

The plant expression vectors, pMON80585, pMON109584, pMON118756, pMON124912, pMON138776, pMON138777, pMON138778, pMON138779 and pMON138780 were used to transform soybean leaves and roots using particle bombardment transformation methods.

Briefly, A3244 soybean seeds were surface sterilized and allowed to germinate in trays with a photoperiod of 16 hours light and 8 hours of darkness. After approximately 13 days, leaf and root tissue was harvested under sterile conditions from the seedlings and used for bombardment. The tissue samples were randomly placed on a petri dish containing plant culture medium. Ten micrograms of plasmid DNA was used to coat 0.6 micron gold particles (Catalog #165-2262 Bio-Rad, Hercules, CA) for bombardment. Macro-carriers were loaded with the DNA-coated gold particles (Catalog #165-2335 Bio-Rad, Hercules CA). A PDS 1000/He biolistic gun was used for transformation (Catalog #165-2257 Bio-Rad, Hercules CA). The bombarded root and leaf tissues were allowed to incubate in the dark for 24 hours at 26 degrees Celsius. Following this overnight incubation, the tissues were stained in solution for GUS expression overnight at 37 degrees Celsius. After staining overnight, the tissues were soaked in 70% ethanol overnight to remove chlorophyll and reveal the GUS staining. The tissues were then photographed and a rating scale of “0”, “+” to “++++++” reflecting the level of GUS expression is assigned to each construct (0—no expression, + to ++++++—low to high, respectively).

Expression of the GUS transgene demonstrated in each tissue is used to infer the relative potential level and specificity of each element's capacity to drive transgene expression in stably transformed corn plants. Average GUS expression ratings are provided in Table 6 below.

TABLE 6
GUS expression ratings for particle bombarded leaf and root.

| | | SEQ ID | Leaf Expression | Root Expression |
|---|---|---|---|---|
| Construct | Regulatory Element | NO: | Rating | Rating |
| pMON80585 | EXP-At.Atntt1:1:2 | 200 | ++++ | ++ |
| pMON109584 | EXP-CaMV.35S- | 201 | +++++ | +++ |
| | enh + Ph.DnaK:1:3 | | | |
| pMON118756 | EXP-At.Act7:1:11 | 202 | ++++ | ++ |
| pMON124912 | No promoter | | 0 | 0 |
| pMON138776 | EXP-CUCme.Ubq1:1:1 | 1 | ++++ | +++ |
| pMON138777 | EXP-CUCme.Ubq1:1:2 | 5 | +++ | ++ |
| pMON138778 | EXP-CUCme.Ubq1:1:3 | 7 | +++ | ++ |
| pMON138779 | EXP-CUCme.Ubq1:1:4 | 9 | +++ | ++ |
| pMON138780 | EXP-CUCme.Ubq1:1:5 | 11 | ++ | + |

As can be seen in Table 6 above, each of the expression element groups EXP-CUCme.Ubq1:1:1 (SEQ ID NO: 1), EXP-CUCme.Ubq1:1:2 (SEQ ID NO: 5), EXP-CUCme.Ubq1:1:3 (SEQ ID NO: 7), EXP-CUCme.Ubq1:1:4 (SEQ ID NO: 9) and EXP-CUCme.Ubq1:1:5 (SEQ ID NO: 11) demonstrated the ability of driving transgene expression in particle bombarded transformed leaf and root tissues.

### Example 4: Analysis of Regulatory Elements Driving GUS in Soy Cotyledon Protoplasts

Soybean cotyledon protoplasts were transformed with plant expression vectors containing a test transcriptional regulatory expression element group driving expression of the ß-glucuronidase (GUS) transgene and compared to GUS expression in leaf protoplasts in which expression of GUS is driven by known constitutive promoters.

Expression of a transgene driven by P-CUCme.1-1:1:1rc (SEQ ID NO: 155), P-CUCme.2-1:1:1 (SEQ ID NO: 14), P-CUCme.3-1:1:3 (SEQ ID NO: 15), EXP-CUCme.4:1:1 (SEQ ID NO: 156), EXP-CUCme.5:1:1 (SEQ ID NO: 159), P-CUCme.6-1:1:1 (SEQ ID NO: 18), P-CUCme.8-1:1:2 (SEQ ID NO: 19), P-CUCme.9-1:1:2 (SEQ ID NO: 20), P-CUCme.10-1:1:1 (SEQ ID NO: 21), EXP-CUCme.eEF1a:1:1 (SEQ ID NO: 162), P-CUCme.15-1:1:2 (SEQ ID NO: 23), P-CUCme.16a-1:1:2 (SEQ ID NO: 24), P-CUCme.17-1:1:2 (SEQ ID NO: 26), P-CUCme.18-1:1:2 (SEQ ID NO: 27), P-CUCme.19-1:1:3 (SEQ ID NO: 167), P-CUCme.20-1:3 (SEQ ID NO: 211), P-CUCme.21-1:1:1 (SEQ ID NO: 30), P-CUCme.22-1:1:3 (SEQ ID NO: 31), EXP-CUCme.SAMS2:1:1 (SEQ ID NO: 168), P-CUCme.26-1:1:2 (SEQ ID NO: 33), P-CUCme.28-1:1:2 (SEQ ID NO: 34) and EXP-CUCme.29:1:2 (SEQ ID NO: 212) was compared with expression from known constitutive expression element groups. Each plant expression vector was comprised of a right border region from Agrobacterium tumefaciens, a first transgene cassette comprised of a test promoter or known constitutive promoter operably linked 5′ to a coding sequence for ß-glucuronidase (GUS, SEQ ID NO: 206) containing a processable intron derived from the potato light-inducible tissue-specific ST-LS1 gene (Genbank Accession: X04753), operably linked 5′ to a 3′ termination region from the Gossypium barbadense E6 gene (T-Gb.E6-3b:1:1, SEQ ID NO: 204), the Pisum sativum RbcS2-E9 gene (T-Ps.RbcS2-E9-1:1:6, SEQ ID NO: 203), or the Gossypium barbadense FbLate-2 gene (T-Gb.FbL2-1:1:1, SEQ ID NO: 205); a second transgene selection cassette used for selection of transformed plant cells that either confers resistance to the herbicide glyphosate (driven by the Arabidopsis Actin 7 promoter) or the antibiotic, kanamycin and a left border region from A. tumefaciens. A promoterless control plant expression vector (pMON124912) served as a negative control for expression. The foregoing test and constitutive expression element groups were cloned into plant expression vectors as shown in Table 7 below.

TABLE 7
Plant expression vectors and corresponding expression element group and 3′ UTR.

| | | SEQ ID | |
|---|---|---|---|
| Construct | Regulatory Element | NO: | 3′ UTR |
| pMON80585 | EXP-At.Atntt1:1:2 | 200 | T-Ps.RbcS2-E9-1:1:6 |
| pMON109584 | EXP-CaMV.35S- | 201 | T-Gb.E6-3b:1:1 |
| | enh + Ph.DnaK:1:3 | | |
| pMON118756 | EXP-At.Act7:1:11 | 202 | T-Gb.E6-3b:1:1 |
| pMON124912 | Promoterless | | T-Gb.FbL2-1:1:1 |
| pMON140818 | P-CUCme.1-1:1:1rc | 155 | T-Gb.FbL2-1:1:1 |
| pMON140819 | P-CUCme.2-1:1:1 | 14 | T-Gb.FbL2-1:1:1 |
| pMON140820 | P-CUCme.3-1:1:3 | 15 | T-Gb.FbL2-1:1:1 |
| pMON140821 | EXP-CUCme.4:1:1 | 156 | T-Gb.FbL2-1:1:1 |
| pMON140822 | EXP-CUCme.5:1:1 | 159 | T-Gb.FbL2-1:1:1 |
| pMON140823 | P-CUCme.6-1:1:1 | 18 | T-Gb.FbL2-1:1:1 |
| pMON140824 | P-CUCme.8-1:1:2 | 19 | T-Gb.FbL2-1:1:1 |
| pMON140825 | P-CUCme.9-1:1:2 | 20 | T-Gb.FbL2-1:1:1 |
| pMON140826 | P-CUCme.10-1:1:1 | 21 | T-Gb.FbL2-1:1:1 |
| pMON140827 | EXP-CUCme.eEF1a:1:1 | 162 | T-Gb.FbL2-1:1:1 |
| pMON140828 | P-CUCme.15-1:1:2 | 23 | T-Gb.FbL2-1:1:1 |
| pMON140829 | P-CUCme.16a-1:1:2 | 24 | T-Gb.FbL2-1:1:1 |
| pMON140830 | P-CUCme.17-1:1:2 | 26 | T-Gb.FbL2-1:1:1 |
| pMON140831 | P-CUCme.18-1:1:2 | 27 | T-Gb.FbL2-1:1:1 |
| pMON140832 | P-CUCme.19-1:1:3 | 167 | T-Gb.FbL2-1:1:1 |
| pMON140833 | P-CUCme.20-1:3 | 211 | T-Gb.FbL2-1:1:1 |
| pMON140834 | P-CUCme.21-1:1:1 | 30 | T-Gb.FbL2-1:1:1 |
| pMON140835 | P-CUCme.22-1:1:3 | 31 | T-Gb.FbL2-1:1:1 |
| pMON140836 | EXP-CUCme.SAMS2:1:1 | 168 | T-Gb.FbL2-1:1:1 |
| pMON140837 | P-CUCme.26-1:1:2 | 33 | T-Gb.FbL2-1:1:1 |
| pMON140838 | P-CUCme.28-1:1:2 | 34 | T-Gb.FbL2-1:1:1 |
| pMON140839 | EXP-CUCme.29:1:2 | 212 | T-Gb.FbL2-1:1:1 |

Two plasmids, for use in co-transformation and normalization of data, were also constructed. One transformation control plasmid was comprised of a constitutive promoter, driving the expression of the firefly (Photinus pyralis) luciferase coding sequence (FLuc, SEQ ID NO: 207), operably linked 5′ to a 3′ termination region from the Agrobacterium tumefaciens nopaline synthase gene (T-AGRtu.nos-1:1:13, SEQ ID NO: 209). The other transformation control plasmid was comprised of a constitutive promoter, driving the expression of the sea pansy (Renilla reniformis) luciferase coding sequence (RLuc, SEQ ID NO: 208), operably linked 5′ to a 3′ termination region from the Agrobacterium tumefaciens nopaline synthase gene.

The plant expression vectors, pMON80585, pMON109584, pMON118756, pMON124912, pMON140818, pMON140819, pMON140820, pMON140821, pMON140822, pMON140823, pMON140824, pMON140825, pMON140826, pMON140827, pMON140828, pMON140829, pMON140830, pMON140831, pMON140832, pMON140833, pMON140834, pMON140835, pMON140836, pMON140837, pMON140838 and pMON140839 were used to transform soybean cotyledon protoplast cells using PEG transformation methods. Protoplast cells were transformed with equimolar amounts of each of the two transformation control plasmids and a test plant expression vector. GUS and luciferase activity was assayed. Measurements of both GUS and luciferase were conducted by placing aliquots of a lysed preparation of cells transformed as above into two different small-well trays. One tray was used for GUS measurements, and a second tray was used to perform a dual luciferase assay using the dual luciferase reporter assay system (Promega Corp., Madison, WI; see for example, Promega Notes Magazine, No: 57, 1996, p. 02). Sample measurements were made using 3 or 4 replicates per transformation. The average GUS and luciferase values are presented in Table 8 below.

TABLE 8
Average GUS and luciferase expression values and GUS/luciferase ratios.

| | | SEQ ID | Average | Average | Average | GUS/ | GUS/ |
|---|---|---|---|---|---|---|---|
| Construct | Regulatory Element | NO: | GUS | FLuc | RLuc | FLuc | RLuc |
| pMON80585 | EXP-At.Atntt1:1:2 | 200 | 586 | 5220.7 | 8323 | 0.1100 | 0.0700 |
| pMON109584 | EXP-CaMV.35S- | 201 | 5768 | 4275 | 15098 | 1.3500 | 0.3800 |
| | enh + Ph.DnaK:1:3 | | | | | | |
| pMON118756 | EXP-At.Act7:1:11 | 202 | 773 | 7722 | 10545 | 0.1000 | 0.0700 |
| pMON124912 | Promoterless | | 48 | 9746.5 | 13905 | 0.0000 | 0.0000 |
| pMON140818 | P-CUCme.1-1:1:1rc | 155 | 194 | 4772 | 6363 | 0.0400 | 0.0300 |
| pMON140819 | P-CUCme.2-1:1:1 | 14 | 171 | 6855 | 10123 | 0.0200 | 0.0200 |
| pMON140820 | P-CUCme.3-1:1:3 | 15 | 37 | 7089.3 | 9593 | 0.0100 | 0.0000 |
| pMON140821 | EXP-CUCme.4:1:1 | 156 | 4211 | 7626.8 | 13935 | 0.5500 | 0.3000 |
| pMON140822 | EXP-CUCme.5:1:1 | 159 | 626 | 15609.3 | 21140 | 0.0400 | 0.0300 |
| pMON140823 | P-CUCme.6-1:1:1 | 18 | 331 | 15178.5 | 22818 | 0.0200 | 0.0100 |
| pMON140824 | P-CUCme.8-1:1:2 | 19 | 238 | 17514.5 | 28429 | 0.0100 | 0.0100 |
| pMON140825 | P-CUCme.9-1:1:2 | 20 | 510 | 13208 | 19567 | 0.0400 | 0.0300 |
| pMON140826 | P-CUCme.10-1:1:1 | 21 | 352 | 14805.3 | 22200 | 0.0200 | 0.0200 |
| pMON140827 | EXP-CUCme.eEF1a:1:1 | 162 | 724 | 9326.8 | 14476 | 0.0800 | 0.0500 |
| pMON140828 | P-CUCme.15-1:1:2 | 23 | 304 | 11798 | 17486 | 0.0300 | 0.0200 |
| pMON140829 | P-CUCme.16a-1:1:2 | 24 | 88 | 5429 | 9596 | 0.0200 | 0.0100 |
| pMON140830 | P-CUCme.17-1:1:2 | 26 | 180 | 10477.8 | 15291 | 0.0200 | 0.0100 |
| pMON140831 | P-CUCme.18-1:1:2 | 27 | 111 | 5059.3 | 6778 | 0.0200 | 0.0200 |
| pMON140832 | P-CUCme.19-1:1:3 | 167 | 121 | 3765 | 6032 | 0.0300 | 0.0200 |
| pMON140833 | P-CUCme.20-1:3 | 211 | 155 | 10458.8 | 14748 | 0.0100 | 0.0100 |
| pMON140834 | P-CUCme.21-1:1:1 | 30 | 582 | 7760 | 11440 | 0.0800 | 0.0500 |
| pMON140835 | P-CUCme.22-1:1:3 | 31 | 400 | 11393.8 | 18654 | 0.0400 | 0.0200 |
| pMON140836 | EXP- | 168 | 568 | 9466.3 | 13962 | 0.0600 | 0.0400 |
| | CUCme.SAMS2:1:1 | | | | | | |
| pMON140837 | P-CUCme.26-1:1:2 | 33 | 87 | 6683 | 8494 | 0.0100 | 0.0100 |
| pMON140838 | P-CUCme.28-1:1:2 | 34 | 171 | 19104.8 | 29619 | 0.0100 | 0.0100 |
| pMON140839 | EXP-CUCme.29:1:2 | 212 | 90 | 11247.3 | 15919 | 0.0100 | 0.0057 |

To compare the relative activity of each promoter in soybean cotyledon protoplasts, GUS values were expressed as a ratio of GUS to luciferase activity and normalized with respect to the expression levels observed for the constitutive expression element groups, EXP-At.Act7:1:11 and EXP-CaMV.35S-enh+Ph.DnaK:1:3. Table 9 below shows the GUS to firefly luciferase (FLuc) ratios normalized with respect to EXP-At.Act7:1:11 and EXP-CaMV.35S-enh+Ph.DnaK:1:3. Table 10 below shows the GUS to renilla luciferase (RLuc) ratios normalized with respect to EXP-At.Act7:1:11 and EXP-CaMV.35S-enh+Ph.DnaK:1:3.

TABLE 9
GUS to firefly luciferase (FLuc) ratios normalized with respect
to EXP-At.Act7:1:11 and EXP-CaMV.35S-enh + Ph.DnaK:1:3.

| | | | GUS/FLuc | GUS/FLuc |
|---|---|---|---|---|
| | | | normalized | normalized with |
| | | | with respect to | respect to EXP- |
| | | SEQ ID | EXP- | CaMV.35S- |
| Construct | Regulatory Element | NO: | At.Act7:1:11 | enh + Ph.DnaK:1:3 |
| pMON80585 | EXP-At.Atntt1:1:2 | 200 | 1.12 | 0.08 |
| pMON109584 | EXP-CaMV.35S-enh + Ph.DnaK:1:3 | 201 | 13.48 | 1.00 |
| pMON118756 | EXP-At.Act7:1:11 | 202 | 1.00 | 0.07 |
| pMON124912 | Promoterless | | 0.05 | 0.00 |
| pMON140818 | P-CUCme.1-1:1:1rc | 155 | 0.41 | 0.03 |
| pMON140819 | P-CUCme.2-1:1:1 | 14 | 0.25 | 0.02 |
| pMON140820 | P-CUCme.3-1:1:3 | 15 | 0.05 | 0.00 |
| pMON140821 | EXP-CUCme.4:1:1 | 156 | 5.52 | 0.41 |
| pMON140822 | EXP-CUCme.5:1:1 | 159 | 0.40 | 0.03 |
| pMON140823 | P-CUCme.6-1:1:1 | 18 | 0.22 | 0.02 |
| pMON140824 | P-CUCme.8-1:1:2 | 19 | 0.14 | 0.01 |
| pMON140825 | P-CUCme.9-1:1:2 | 20 | 0.39 | 0.03 |
| pMON140826 | P-CUCme.10-1:1:1 | 21 | 0.24 | 0.02 |
| pMON140827 | EXP-CUCme.eEF1a:1:1 | 162 | 0.78 | 0.06 |
| pMON140828 | P-CUCme.15-1:1:2 | 23 | 0.26 | 0.02 |
| pMON140829 | P-CUCme.16a-1:1:2 | 24 | 0.16 | 0.01 |
| pMON140830 | P-CUCme.17-1:1:2 | 26 | 0.17 | 0.01 |
| pMON140831 | P-CUCme.18-1:1:2 | 27 | 0.22 | 0.02 |
| pMON140832 | P-CUCme.19-1:1:3 | 167 | 0.32 | 0.02 |
| pMON140833 | P-CUCme.20-1:3 | 211 | 0.15 | 0.01 |
| pMON140834 | P-CUCme.21-1:1:1 | 30 | 0.75 | 0.06 |
| pMON140835 | P-CUCme.22-1:1:3 | 31 | 0.35 | 0.03 |
| pMON140836 | EXP-CUCme.SAMS2:1:1 | 168 | 0.60 | 0.04 |
| pMON140837 | P-CUCme.26-1:1:2 | 33 | 0.13 | 0.01 |
| pMON140838 | P-CUCme.28-1:1:2 | 34 | 0.09 | 0.01 |
| pMON140839 | EXP-CUCme.29:1:2 | 212 | 0.08 | 0.01 |

TABLE 10
GUS to renilla luciferase (RLuc) ratios normalized with respect
to EXP-At.Act7:1:11 and EXP-CaMV.35S-enh + Ph.DnaK:1:3.

| | | | GUS/RLuc | GUS/RLuc |
|---|---|---|---|---|
| | | | normalized | normalized with |
| | | | with respect to | respect to EXP- |
| | | SEQ ID | EXP- | CaMV.35S- |
| Construct | Regulatory Element | NO: | At.Act7:1:11 | enh + Ph.DnaK:1:3 |
| pMON80585 | EXP-At.Atntt1:1:2 | 200 | 0.96 | 0.18 |
| pMON109584 | EXP-CaMV.35S-enh + Ph.DnaK:1:3 | 201 | 5.21 | 1.00 |
| pMON118756 | EXP-At.Act7:1:11 | 202 | 1.00 | 0.19 |
| pMON124912 | Promoterless | | 0.05 | 0.01 |
| pMON140818 | P-CUCme.1-1:1:1rc | 155 | 0.42 | 0.08 |
| pMON140819 | P-CUCme.2-1:1:1 | 14 | 0.23 | 0.04 |
| pMON140820 | P-CUCme.3-1:1:3 | 15 | 0.05 | 0.01 |
| pMON140821 | EXP-CUCme.4:1:1 | 156 | 4.12 | 0.79 |
| pMON140822 | EXP-CUCme.5:1:1 | 159 | 0.40 | 0.08 |
| pMON140823 | P-CUCme.6-1:1:1 | 18 | 0.20 | 0.04 |
| pMON140824 | P-CUCme.8-1:1:2 | 19 | 0.11 | 0.02 |
| pMON140825 | P-CUCme.9-1:1:2 | 20 | 0.36 | 0.07 |
| pMON140826 | P-CUCme.10-1:1:1 | 21 | 0.22 | 0.04 |
| pMON140827 | EXP-CUCme.eEF1a:1:1 | 162 | 0.68 | 0.13 |
| pMON140828 | P-CUCme.15-1:1:2 | 23 | 0.24 | 0.05 |
| pMON140829 | P-CUCme.16a-1:1:2 | 24 | 0.13 | 0.02 |
| pMON140830 | P-CUCme.17-1:1:2 | 26 | 0.16 | 0.03 |
| pMON140831 | P-CUCme.18-1:1:2 | 27 | 0.22 | 0.04 |
| pMON140832 | P-CUCme.19-1:1:3 | 167 | 0.27 | 0.05 |
| pMON140833 | P-CUCme.20-1:3 | 211 | 0.14 | 0.03 |
| pMON140834 | P-CUCme.21-1:1:1 | 30 | 0.69 | 0.13 |
| pMON140835 | P-CUCme.22-1:1:3 | 31 | 0.29 | 0.06 |
| pMON140836 | EXP-CUCme.SAMS2:1:1 | 168 | 0.55 | 0.11 |
| pMON140837 | P-CUCme.26-1:1:2 | 33 | 0.14 | 0.03 |
| pMON140838 | P-CUCme.28-1:1:2 | 34 | 0.08 | 0.02 |
| pMON140839 | EXP-CUCme.29:1:2 | 212 | 0.08 | 0.01 |

As can be seen in Tables 9 and 10. most of the expression element groups tested. demonstrated the ability to drive transgene expression in soybean cotyledon protoplast cells. One expression element group, EXP-CUCme.4:1:1 (SEQ ID NO: 156) demonstrated levels of transgene expression higher than that of EXP-At.Act7:1:11 in this assay.

### Example 5: Analysis of Regulatory Elements Driving GUS in Bombarded Soybean Leaves and Roots

Soybean leaves and roots were transformed with plant expression vectors containing a test transcriptional regulatory expression clement group driving expression of the ß-glucuronidase (GUS) transgene and compared to GUS expression in roots and leaves in which expression of GUS is driven by known constitutive promoters.

Expression of a transgene driven by P-CUCme.1-1:1:1rc (SEQ ID NO: 155), P-CUCme.2-1:1:1 (SEQ ID NO: 14), P-CUCme.3-1:1:3 (SEQ ID NO: 15), EXP-CUCme.4:1:1 (SEQ ID NO: 156), EXP-CUCme.5:1:1 (SEQ ID NO: 159), P-CUCme.6-1:1:1 (SEQ ID NO: 18), P-CUCme.8-1:1:2 (SEQ ID NO: 19), P-CUCme.9-1:1:2 (SEQ ID NO: 20), P-CUCme.10-1:1:1 (SEQ ID NO: 21), EXP-CUCme.eEF1a:1:1 (SEQ ID NO: 162), P-CUCme.15-1:1:2 (SEQ ID NO: 23), P-CUCme.16a-1:1:2 (SEQ ID NO: 24), P-CUCme.17-1:1:2 (SEQ ID NO: 26), P-CUCme.18-1:1:2(SEQ ID NO: 27), P-CUCme.19-1:1:3 (SEQ ID NO: 167), P-CUCme.20-1:3 (SEQ ID NO: 211), P-CUCme.21-1:1:1 (SEQ ID NO: 30), P-CUCme.22-1:1:3 (SEQ ID NO: 31), EXP-CUCme.SAMS2:1:1 (SEQ ID NO: 168), P-CUCme.26-1:1:2 (SEQ ID NO: 33), P-CUCme.28-1:1:2 (SEQ ID NO: 34) and EXP-CUCme.29:1:2 (SEQ ID NO: 212) was compared with expression from known constitutive expression element groups in particle bombarded soybean leaves and roots. The plant expression vectors used for transformation of leaves and roots was the same as those presented in Table 7 of Example 4 above.

The plant expression vectors, pMON80585, pMON109584, pMON118756, pMON124912, pMON140818, pMON140819, pMON140820, pMON140821, pMON140822, pMON140823, pMON140824, pMON140825, pMON140826, pMON140827, pMON140828, pMON140829, pMON140830, pMON140831, pMON140832, pMON140833, pMON140834, pMON140835, pMON140836, pMON140837, pMON140838 and pMON140839 were used to transform soybean leaves and roots using particle bombardment transformation methods.

Briefly, A3244 soybean seeds were surface sterilized and allowed to germinate in trays with a photoperiod of 16 hours light and 8 hours of darkness. After approximately 13 days, leaf and root tissue was harvested under sterile conditions from the seedlings and used for bombardment. The tissue samples were randomly placed on a petri dish containing plant culture medium. Ten micrograms of plasmid DNA was used to coat 0.6 micron gold particles (Catalog #165-2262 Bio-Rad, Hercules, CA) for bombardment. Macro-carriers were loaded with the DNA-coated gold particles (Catalog #165-2335 Bio-Rad, Hercules CA). A PDS 1000/He biolistic gun was used for transformation (Catalog #165-2257 Bio-Rad, Hercules CA). The bombarded root and leaf tissues were allowed to incubate in the dark for 24 hours at 26 degrees Celsius. Following this overnight incubation, the tissues were stained in solution for GUS expression overnight at 37 degrees Celsius. After staining overnight, the tissues were soaked in 70% ethanol overnight to remove chlorophyll and reveal the GUS staining. The tissues were then photographed and a rating scale of “0”, “+” to “++++++” reflecting the level of GUS expression is assigned to each construct (0—no expression, + to ++++++—low to high, respectively).

Expression of the GUS transgene demonstrated in each tissue is used to infer the relative potential level and specificity of each element's capacity to drive transgene expression in stably transformed corn plants. Average GUS expression ratings are provided in Table 11 below.

TABLE 11
GUS expression ratings for particle bombarded leaf and root.

| | | SEQ ID | Leaf | Root |
|---|---|---|---|---|
| Construct | Regulatory Element | NO: | Expression | Expression |
| pMON80585 | EXP-At.Atntt1:1:2 | 200 | +++ | +++ |
| pMON109584 | EXP-CaMV.35S- | 201 | +++++ | ++ |
| | enh + Ph.DnaK:1:3 | | | |
| pMON118756 | EXP-At.Act7:1:11 | 202 | ++++ | +++ |
| pMON124912 | Promoterless | | 0 | 0 |
| pMON140818 | P-CUCme.1-1:1:1rc | 155 | +++ | + |
| pMON140819 | P-CUCme.2-1:1:1 | 14 | ++ | + |
| pMON140820 | P-CUCme.3-1:1:3 | 15 | 0 | 0 |
| pMON140821 | EXP-CUCme.4:1:1 | 156 | ++++++ | +++ |
| pMON140822 | EXP-CUCme.5:1:1 | 159 | ++ | + |
| pMON140823 | P-CUCme.6-1:1:1 | 18 | ++ | + |
| pMON140824 | P-CUCme.8-1:1:2 | 19 | + | + |
| pMON140825 | P-CUCme.9-1:1:2 | 20 | ++ | + |
| pMON140826 | P-CUCme.10-1:1:1 | 21 | +++ | +++ |
| pMON140827 | EXP-CUCme.eEF1a:1:1 | 162 | ++++ | +++ |
| pMON140828 | P-CUCme.15-1:1:2 | 23 | + | + |
| pMON140829 | P-CUCme.16a-1:1:2 | 24 | + | − |
| pMON140830 | P-CUCme.17-1:1:2 | 26 | ++++ | + |
| pMON140831 | P-CUCme.18-1:1:2 | 27 | +++ | + |
| pMON140832 | P-CUCme.19-1:1:3 | 167 | + | + |
| pMON140833 | P-CUCme.20-1:3 | 211 | + | + |
| pMON140834 | P-CUCme.21-1:1:1 | 30 | + | + |
| pMON140835 | P-CUCme.22-1:1:3 | 31 | ++++ | + |
| pMON140836 | EXP-CUCme.SAMS2:1:1 | 168 | +++++ | +++ |
| pMON140837 | P-CUCme.26-1:1:2 | 33 | + | + |
| pMON140838 | P-CUCme.28-1:1:2 | 34 | + | + |
| pMON140839 | EXP-CUCme.29:1:2 | 212 | + | + |

As can be seen in Table 11 above, all but one of the expression element groups demonstrated the ability to drive transgene expression in particle bombarded soybean leaf and root tissue. Two expression element groups, P-CUCme.28-1:1:2 (SEQ ID NO: 34) and EXP-CUCme.4:1:1 (SEQ ID NO: 156) demonstrated similar or higher levels of expression relative to expression driven by EXP-CaMV.35S-enh+Ph.DnaK:1:3 in this assay.

### Example 6: Analysis of Regulatory Elements Driving GUS in Soy Cotyledon Protoplast Using Transgene Cassette Amplicons

Soybean cotyledon protoplasts were transformed with transgene cassette amplicons containing a transcriptional regulatory expression element group driving expression of the ß-glucuronidase (GUS) transgene and compared to GUS expression in leaf protoplasts in which expression of GUS is driven by known constitutive promoters. The transgene cassette amplicons were comprised of an EXP sequence, operably linked to a GUS coding sequence (GUS, SEQ ID NO: 206), operably linked to a 3′ UTR (T-Gb.FbL2-1:1:1, SEQ ID NO: 205). Average GUS expression was compared to the control EXP elements, P-CaMV.35S-enh-1:1:102/L-CaMV.35S-1:1:2 (SEQ ID NO: 210) and EXP-At.Atntt1:1:2 (SEQ ID NO: 200).

A plasmid, for use in co-transformation and normalization of data was also used in a similar manner as that described above in Example 2. The transformation control plasmid was comprised of a constitutive promoter, driving the expression of the firefly (Photinus pyralis) luciferase coding sequence (FLuc, SEQ ID NO: 205), operably linked 5′ to a 3′ termination region from the Agrobacterium tumefaciens nopaline synthase gene (T-AGRtu.nos-1:1:13, SEQ ID NO: 209).

Table 12 below shows the mean GUS expression values conferred by each transgene amplicon. Table 13 below shows the GUS to firefly luciferase (FLuc) ratios normalized with respect to EXP-At.Atntt1:1:2 and P-CaMV.35S-enh-1:1:102/L-CaMV.35S-1:1:2

TABLE 12
Average GUS and luciferase expression values and GUS/luciferase ratios.

| | | SEQ ID | Mean | Mean | |
|---|---|---|---|---|---|
| Amplicon ID | Regulatory Element | NO: | GUS | Fluc | GUS/Fluc |
| No DNA | | | 0.00 | 0.00 | 0.00 |
| pMON124912 | No promoter | | 54.67 | 34905.00 | 0.00 |
| pMON33449 | P-CaMV.35S-enh-1:1:102/L-CaMV.35S-1:1:2 | 210 | 107064.67 | 21757.67 | 4.92 |
| pMON80585 | EXP-At.Atntt1:1:2 | 200 | 4962.33 | 40778.67 | 0.12 |
| 56969 | CumMe_WSM_SF16429.G5670 | 40 | 283.67 | 53452.00 | 0.01 |
| 56877 | P-CUCme.CumMe_WSM_SF16444.G5140-1:1:1 | 175 | 5297.67 | 46576.67 | 0.11 |
| 56749 | P-CUCme.CumMe_WSM_SF16563.G5560-1:1:1 | 176 | 280.67 | 41958.33 | 0.01 |
| 56918 | CumMe_WSM_SF17051.G5470 | 48 | 1088.00 | 36321.00 | 0.03 |
| 56849 | P-CUCme.CumMe_WSM_SF17111.G5790-1:1:1 | 177 | 196.00 | 48128.00 | 0.00 |
| 56754 | P-CUCme.WSM_SF17252.G7330-1:1:1 | 179 | 175.67 | 45427.00 | 0.00 |
| 56892 | CumMe_WSM_SF17349.G5770 | 56 | 34.00 | 38016.00 | 0.00 |
| 56477 | CumMe_WSM_SF17866.G6050 | 62 | 862.00 | 52203.33 | 0.02 |
| 56842 | P-CUCme.CumMe_WSM_SF18488.G5340-1:1:1 | 181 | 2892.67 | 49144.33 | 0.06 |
| 56852 | P-CUCme.CumMe_WSM_SF18536.G6480-1:1:1 | 182 | 3462.67 | 46549.33 | 0.07 |
| 56497 | CumMe_WSM_SF18575.G6410 | 71 | 92.67 | 47628.33 | 0.00 |
| 56847 | P-CUCme.CumMe_WSM_SF18634.G5190-1:1:1 | 183 | 122.33 | 36815.33 | 0.00 |
| 56746 | P-CUCme.CumMe_WSM_SF18716.G5860-1:1:1 | 184 | 14.33 | 62483.33 | 0.00 |
| 56883 | CumMe_WSM_SF18986.G6110 | 79 | 863.33 | 54379.33 | 0.02 |
| 56734 | EXP-CUCme.WSM_SF19064.G5690:1:1 | 185 | 142.00 | 46962.67 | 0.00 |
| 56912 | P-CUCme.CumMe_WSM_SF19647.G5760-1:1:1 | 188 | 7659.00 | 46935.67 | 0.16 |
| 56482 | P-CUCme.CumMe_WSM_SF19839.G5090-1:1:1 | 189 | 3279.00 | 37070.67 | 0.09 |
| 56963 | CumMe_WSM_SF19902.G5260 | 87 | 1629.00 | 55649.00 | 0.03 |
| 56747 | P-CUCme.CumMe_WSM_SF20132.G5560-1:1:1 | 190 | 340.33 | 40577.00 | 0.01 |
| 56479 | CumMe_WSM_SF20359.G5870 | 92 | 192.00 | 61341.67 | 0.00 |
| 56744 | CumMe_WSM_SF206458.G5970 | 98 | 154.67 | 33139.33 | 0.00 |
| 56948 | CumMe_WSM_SF206534.G5200 | 99 | 62.00 | 52118.00 | 0.00 |
| 56896 | CumMe_WSM_SF22008.G5670 | 108 | 1585.00 | 53540.00 | 0.03 |
| 56919 | CumMe_WSM_SF22275.G5780 | 112 | 8.33 | 48546.33 | 0.00 |
| 56967 | CumMe_WSM_SF22355.G5310 | 113 | 74.33 | 36202.67 | 0.00 |
| 56837 | P-CUCme.CumMe_WSM_SF22531.G5120-1:1:1 | 192 | 1526.67 | 52799.33 | 0.03 |
| 56940 | CumMe_WSM_SF22870.G5370 | 115 | 14.67 | 53663.33 | 0.00 |
| 56495 | P-CUCme.CumMe_WSM_SF23760.G5200-1:1:1 | 193 | 196.33 | 49870.67 | 0.00 |
| 56868 | P-CUCme.CumMe_WSM_SF23906.G6180-1:1:1 | 194 | 1584.33 | 42532.33 | 0.04 |
| 56998 | CumMe_WSM_SF24045.G5400 | 123 | 80.67 | 47553.00 | 0.00 |
| 56976 | P-CUCme.CumMe_WSM_SF25141.G5160-1:1:2 | 195 | 4506.00 | 57213.00 | 0.08 |
| 56742 | P-CUCme.CumMe_WSM_SF25355.G5000-1:1:1 | 196 | 4.00 | 41114.33 | 0.00 |
| 56915 | P-CUCme.CumMe_WSM_SF25936.G5450-1:1:1 | 197 | 965.33 | 34494.67 | 0.03 |
| 56854 | CumMe_WSM_SF28729.G5340 | 134 | 208.33 | 53956.00 | 0.00 |
| 56936 | CumMe_WSM_SF31264.G5380 | 136 | 292.67 | 42320.67 | 0.01 |
| 56863 | P-CUCme.CumMe_WSM_SF35856.G5150-1:1:1 | 198 | 125.00 | 48705.33 | 0.00 |
| 56751 | P-CUCme.CumMe_WSM_SF41124.G5080-1:1:1 | 199 | 31.33 | 53595.00 | 0.00 |
| 56921 | CumMe_WSM_SF41254.G5160 | 141 | 11.67 | 52643.67 | 0.00 |
| 56884 | CumMe_WSM_SF42141.G5110 | 146 | 48.33 | 40556.67 | 0.00 |

TABLE 13
GUS to firefly luciferase (FLuc) ratios normalized with respect
to EXP-At.Atntt1:1:2 and P-CaMV.35S-enh-1:1:102/L-CaMV.35S-1:1:2.

| | | | | GUS/Fluc |
|---|---|---|---|---|
| | | | GUS/Fluc | normalized |
| | | | normalized | with respect |
| | | | with respect | to P-CaMV.35S- |
| | | SEQ ID | to EXP- | enh-1:1:102/L- |
| Amplicon ID | Regulatory Element | NO: | At.Atntt1:1:2 | CaMV.35S-1:1:2 |
| No DNA | | | 0.00 | 0.00 |
| pMON124912 | No promoter | | 0.01 | 0.00 |
| pMON33449 | P-CaMV.35S-enh-1:1:102/L-CaMV.35S-1:1:2 | 210 | 40.44 | 1.00 |
| pMON80585 | EXP-At.Atntt1:1:2 | 200 | 1.00 | 0.02 |
| 56969 | CumMe_WSM_SF16429.G5670 | 40 | 0.04 | 0.00 |
| 56877 | P-CUCme.CumMe_WSM_SF16444.G5140-1:1:1 | 175 | 0.93 | 0.02 |
| 56749 | P-CUCme.CumMe_WSM_SF16563.G5560-1:1:1 | 176 | 0.05 | 0.00 |
| 56918 | CumMe_WSM_SF17051.G5470 | 48 | 0.25 | 0.01 |
| 56849 | P-CUCme.CumMe_WSM_SF17111.G5790-1:1:1 | 177 | 0.03 | 0.00 |
| 56754 | P-CUCme.WSM_SF17252.G7330-1:1:1 | 179 | 0.03 | 0.00 |
| 56892 | CumMe_WSM_SF17349.G5770 | 56 | 0.01 | 0.00 |
| 56477 | CumMe_WSM_SF17866.G6050 | 62 | 0.14 | 0.00 |
| 56842 | P-CUCme.CumMe_WSM_SF18488.G5340-1:1:1 | 181 | 0.48 | 0.01 |
| 56852 | P-CUCme.CumMe_WSM_SF18536.G6480-1:1:1 | 182 | 0.61 | 0.02 |
| 56497 | CumMe_WSM_SF18575.G6410 | 71 | 0.02 | 0.00 |
| 56847 | P-CUCme.CumMe_WSM_SF18634.G5190-1:1:1 | 183 | 0.03 | 0.00 |
| 56746 | P-CUCme.CumMe_WSM_SF18716.G5860-1:1:1 | 184 | 0.00 | 0.00 |
| 56883 | CumMe_WSM_SF18986.G6110 | 79 | 0.13 | 0.00 |
| 56734 | EXP-CUCme.WSM_SF19064.G5690:1:1 | 185 | 0.02 | 0.00 |
| 56912 | P-CUCme.CumMe_WSM_SF19647.G5760-1:1:1 | 188 | 1.34 | 0.03 |
| 56482 | P-CUCme.CumMe_WSM_SF19839.G5090-1:1:1 | 189 | 0.73 | 0.02 |
| 56963 | CumMe_WSM_SF19902.G5260 | 87 | 0.24 | 0.01 |
| 56747 | P-CUCme.CumMe_WSM_SF20132.G5560-1:1:1 | 190 | 0.07 | 0.00 |
| 56479 | CumMe_WSM_SF20359.G5870 | 92 | 0.03 | 0.00 |
| 56744 | CumMe_WSM_SF206458.G5970 | 98 | 0.04 | 0.00 |
| 56948 | CumMe_WSM_SF206534.G5200 | 99 | 0.01 | 0.00 |
| 56896 | CumMe_WSM_SF22008.G5670 | 108 | 0.24 | 0.01 |
| 56919 | CumMe_WSM_SF22275.G5780 | 112 | 0.00 | 0.00 |
| 56967 | CumMe_WSM_SF22355.G5310 | 113 | 0.02 | 0.00 |
| 56837 | P-CUCme.CumMe_WSM_SF22531.G5120-1:1:1 | 192 | 0.24 | 0.01 |
| 56940 | CumMe_WSM_SF22870.G5370 | 115 | 0.00 | 0.00 |
| 56495 | P-CUCme.CumMe_WSM_SF23760.G5200-1:1:1 | 193 | 0.03 | 0.00 |
| 56868 | P-CUCme.CumMe_WSM_SF23906.G6180-1:1:1 | 194 | 0.31 | 0.01 |
| 56998 | CumMe_WSM_SF24045.G5400 | 123 | 0.01 | 0.00 |
| 56976 | P-CUCme.CumMe_WSM_SF25141.G5160-1:1:2 | 195 | 0.65 | 0.02 |
| 56742 | P-CUCme.CumMe_WSM_SF25355.G5000-1:1:1 | 196 | 0.00 | 0.00 |
| 56915 | P-CUCme.CumMe_WSM_SF25936.G5450-1:1:1 | 197 | 0.23 | 0.01 |
| 56854 | CumMe_WSM_SF28729.G5340 | 134 | 0.03 | 0.00 |
| 56936 | CumMe_WSM_SF31264.G5380 | 136 | 0.06 | 0.00 |
| 56863 | P-CUCme.CumMe_WSM_SF35856.G5150-1:1:1 | 198 | 0.02 | 0.00 |
| 56751 | P-CUCme.CumMe_WSM_SF41124.G5080-1:1:1 | 199 | 0.00 | 0.00 |
| 56921 | CumMe_WSM_SF41254.G5160 | 141 | 0.00 | 0.00 |
| 56884 | CumMe_WSM_SF42141.G5110 | 146 | 0.01 | 0.00 |

As can be seen in Table 12 above, not all EXP sequences demonstrated the ability to drive transgene expression when compared to the promoterless control. However, the EXP sequences, CumMe_WSM_SF16429.G5670 (SEQ ID NO: 40), P-CUCme.CumMe_WSM_SF16444.G5140-1:1:1 (SEQ ID NO: 175), P-CUCme.CumMe_WSM_SF16563.G5560-1:1:1 (SEQ ID NO: 176), CumMe_WSM_SF17051.G5470 (SEQ ID NO: 48), P-CUCme.CumMe_WSM_SF17111.G5790-1:1:1 (SEQ ID NO: 177), P-CUCme.WSM_SF17252.G7330-1:1:1 (SEQ ID NO: 179), CumMe_WSM_SF17866.G6050 (SEQ ID NO: 62), P-CUCme.CumMe_WSM_SF18488.G5340-1:1:1 (SEQ ID NO: 181), P-CUCme.CumMe_WSM_SF18536.G6480-1:1:1 (SEQ ID NO: 182), CumMe_WSM_SF18575.G6410 (SEQ ID NO: 71), P-CUCme.CumMe_WSM_SF18634.G5190-1:1:1 (SEQ ID NO: 183), CumMe_WSM_SF18986.G6110 (SEQ ID NO: 79), EXP-CUCme. WSM_SF19064.G5690:1:1 (SEQ ID NO: 185), P-CUCme.CumMe_WSM_SF19647.G5760-1:1:1 (SEQ ID NO: 188), P-CUCme.CumMe_WSM_SF19839.G5090-1:1:1 (SEQ ID NO: 189), CumMe_WSM_SF19902.G5260 (SEQ ID NO: 87), P-CUCme.CumMe_WSM_SF20132.G5560-1:1:1 (SEQ ID NO: 190), CumMe_WSM_SF20359.G5870 (SEQ ID NO: 92), CumMe_WSM_SF206458.G5970 (SEQ ID NO: 98), CumMe_WSM_SF206534.G5200 (SEQ ID NO: 99), CumMe_WSM_SF22008.G5670 (SEQ ID NO: 108), CumMe_WSM_SF22355.G5310 (SEQ ID NO: 113), P-CUCme.CumMe_WSM_SF22531.G5120-1:1:1 (SEQ ID NO: 192), EXP-CUCme.WSM_SF19064.G5690:1:1 (SEQ ID NO: 193), P-CUCme.CumMe_WSM_SF23906.G6180-1:1:1 (SEQ ID NO: 194), CumMe_WSM_SF24045.G5400 (SEQ ID NO: 123), P-CUCme.CumMe_WSM_SF25141.G5160-1:1:2 (SEQ ID NO: 195), P-CUCme.CumMe_WSM_SF25936.G5450-1:1:1 (SEQ ID NO: 197), CumMe_WSM_SF28729.G5340 (SEQ ID NO: 134), CumMe_WSM_SF31264.G5380 (SEQ ID NO: 136) and P-CUCme.CumMe_WSM_SF35856.G5150-1:1:1 (SEQ ID NO: 198) demonstrated the ability to drive trangene expression in soybean cotyledon protoplasts at a level similar or greater than EXP-At.Atntt1:1:2. As shown in Table 13 above, the EXP sequence P-CUCme.CumMe_WSM_SF19647.G5760-1:1:1 (SEQ ID NO: 188) demonstrated the ability to drive transgene expression in this assay at a level greater than EXP-At.Atntt1:1:2.

### Example 7: Analysis of Regulatory Elements Driving GUS in Cotton Leaf Protoplasts

Cotton leaf protoplasts were transformed with plant expression vectors containing a test transcriptional regulatory expression clement group driving expression of the ß-glucuronidase (GUS) transgene and compared to GUS expression in leaf protoplasts in which expression of GUS is driven by known constitutive promoters.

Expression of a transgene driven by P-CUCme.1-1:1:1rc (SEQ ID NO: 155), P-CUCme.2-1:1:1 (SEQ ID NO: 14), P-CUCme.3-1:1:3 (SEQ ID NO: 15), EXP-CUCme.4:1:1 (SEQ ID NO: 156), P-CUCme.6-1:1:1 (SEQ ID NO: 18), P-CUCme.8-1:1:2 (SEQ ID NO: 19), P-CUCme.9-1:1:2 (SEQ ID NO: 20), P-CUCme.10-1:1:1 (SEQ ID NO: 21), EXP-CUCme.eEF1a:1:1 (SEQ ID NO: 162), P-CUCme.15-1:1:2 (SEQ ID NO: 23), P-CUCme.16a-1:1:2 (SEQ ID NO: 24), P-CUCme.17-1:1:2 (SEQ ID NO: 26), P-CUCme.18-1:1:2 (SEQ ID NO: 27), P-CUCme.19-1:1:3 (SEQ ID NO: 167), P-CUCme.20-1:3 (SEQ ID NO: 211), P-CUCme.21-1:1:1 (SEQ ID NO: 30), P-CUCme.22-1:1:3 (SEQ ID NO: 31), EXP-CUCme.SAMS2:1:1 (SEQ ID NO: 168), P-CUCme.26-1:1:2 (SEQ ID NO: 33), P-CUCme.28-1:1:2 (SEQ ID NO: 34) and EXP-CUCme.29:1:2 (SEQ ID NO: 212) was compared with expression from known constitutive expression element groups. Each plant expression vector was comprised of a right border region from Agrobacterium tumefaciens, a first transgene cassette comprised of a test promoter or known constitutive promoter operably linked 5′ to a coding sequence for ß-glucuronidase (GUS, SEQ ID NO: 206) containing a processable intron derived from the potato light-inducible tissue-specific ST-LS1 gene (Genbank Accession: X04753), operably linked 5′ to a 3′ termination region from the Gossypium barbadense E6 gene (T-Gb.E6-3b:1:1, SEQ ID NO: 204), the Pisum sativum RbcS2-E9 gene (T-Ps.RbcS2-E9-1:1:6, SEQ ID NO: 203), or the Gossypium barbadense FbLate-2 gene (T-Gb.FbL2-1:1:1, SEQ ID NO: 205); a second transgene selection cassette used for selection of transformed plant cells that either confers resistance to the herbicide glyphosate (driven by the Arabidopsis Actin 7 promoter) or the antibiotic, kanamycin and a left border region from A. tumefaciens. A promoterless control plant expression vector (pMON124912) served as a negative control for expression. The foregoing test and constitutive expression element groups were cloned into plant expression vectors as shown in Table 14 below.

TABLE 14
Plant expression vectors and corresponding expression element group and 3′ UTR.

| | | SEQ ID | |
|---|---|---|---|
| Construct | Regulatory Element | NO: | 3′ UTR |
| pMON109584 | EXP-CaMV.35S-enh + Ph.DnaK:1:3 | 201 | T-Gb.E6-3b:1:1 |
| pMON118756 | EXP-At.Act7:1:11 | 202 | T-Gb.E6-3b:1:1 |
| pMON124912 | Promoterless | | T-Gb.FbL2-1:1:1 |
| pMON140818 | P-CUCme.1-1:1:1rc | 155 | T-Gb.FbL2-1:1:1 |
| pMON140819 | P-CUCme.2-1:1:1 | 14 | T-Gb.FbL2-1:1:1 |
| pMON140820 | P-CUCme.3-1:1:3 | 15 | T-Gb.FbL2-1:1:1 |
| pMON140821 | EXP-CUCme.4:1:1 | 156 | T-Gb.FbL2-1:1:1 |
| pMON140823 | P-CUCme.6-1:1:1 | 18 | T-Gb.FbL2-1:1:1 |
| pMON140824 | P-CUCme.8-1:1:2 | 19 | T-Gb.FbL2-1:1:1 |
| pMON140825 | P-CUCme.9-1:1:2 | 20 | T-Gb.FbL2-1:1:1 |
| pMON140826 | P-CUCme.10-1:1:1 | 21 | T-Gb.FbL2-1:1:1 |
| pMON140827 | EXP-CUCme.eEF1a:1:1 | 162 | T-Gb.FbL2-1:1:1 |
| pMON140828 | P-CUCme.15-1:1:2 | 23 | T-Gb.FbL2-1:1:1 |
| pMON140829 | P-CUCme.16a-1:1:2 | 24 | T-Gb.FbL2-1:1:1 |
| pMON140830 | P-CUCme.17-1:1:2 | 26 | T-Gb.FbL2-1:1:1 |
| pMON140831 | P-CUCme.18-1:1:2 | 27 | T-Gb.FbL2-1:1:1 |
| pMON140832 | P-CUCme.19-1:1:3 | 167 | T-Gb.FbL2-1:1:1 |
| pMON140833 | P-CUCme.20-1:3 | 211 | T-Gb.FbL2-1:1:1 |
| pMON140834 | P-CUCme.21-1:1:1 | 30 | T-Gb.FbL2-1:1:1 |
| pMON140835 | P-CUCme.22-1:1:3 | 31 | T-Gb.FbL2-1:1:1 |
| pMON140836 | EXP-CUCme.SAMS2:1:1 | 168 | T-Gb.FbL2-1:1:1 |
| pMON140837 | P-CUCme.26-1:1:2 | 33 | T-Gb.FbL2-1:1:1 |
| pMON140838 | P-CUCme.28-1:1:2 | 34 | T-Gb.FbL2-1:1:1 |
| pMON140839 | EXP-CUCme.29:1:2 | 212 | T-Gb.FbL2-1:1:1 |

Two plasmids, for use in co-transformation and normalization of data, were also constructed. One transformation control plasmid was comprised of a constitutive promoter, driving the expression of the firefly (Photinus pyralis) luciferase coding sequence (FLuc, SEQ ID NO: 205), operably linked 5′ to a 3′ termination region from the Agrobacterium tumefaciens nopaline synthase gene (T-AGRtu.nos-1:1:13, SEQ ID NO: 209). The other transformation control plasmid was comprised of a constitutive promoter, driving the expression of the sea pansy (Renilla reniformis) luciferase coding sequence (RLuc, SEQ ID NO: 206), operably linked 5′ to a 3′ termination region from the Agrobacterium tumefaciens nopaline synthase gene.

The plant expression vectors, pMON80585, pMON109584, pMON118756, pMON124912, pMON140818, pMON140819, pMON140820, pMON140821, pMON140823, pMON140824, pMON140825, pMON140826, pMON140827, pMON140828, pMON140829, pMON140830, pMON140831, pMON140832, pMON140833, pMON140834, pMON140835, pMON140836, pMON140837, pMON140838 and pMON140839 were used to transform cotton leaf protoplast cells using PEG transformation methods. Protoplast cells were transformed with equimolar amounts of each of the two transformation control plasmids and a test plant expression vector. GUS and luciferase activity was assayed. Measurements of both GUS and luciferase were conducted by placing aliquots of a lysed preparation of cells transformed as above into two different small-well trays. One tray was used for GUS measurements, and a second tray was used to perform a dual luciferase assay using the dual luciferase reporter assay system (Promega Corp., Madison, WI; see for example, Promega Notes Magazine, No: 57, 1996, p. 02). Sample measurements were made using 4 replicates per transformation. The average GUS and luciferase values are presented in Table 15 below.

TABLE 15
Average GUS and luciferase expression values and GUS/luciferase ratios.

| | | SEQ | | | | | |
|---|---|---|---|---|---|---|---|
| | | ID | Average | Average | Average | GUS/ | GUS/ |
| Construct | Regulatory Element | NO: | GUS | FLuc | RLuc | FLuc | RLuc |
| pMON109584 | EXP-CaMV.35S-enh + | 201 | 5322.8 | 14842.8 | 27990.5 | 0.3586 | 0.1902 |
| | Ph.DnaK:1:3 | | | | | | |
| pMON118756 | EXP-At.Act7:1:11 | 202 | 1006.3 | 19746.8 | 25582.3 | 0.0510 | 0.0393 |
| pMON124912 | Promoterless | | 21 | 19248.5 | 25012 | 0.0011 | 0.0008 |
| pMON140818 | P-CUCme.1-1:1:1rc | 155 | 170.3 | 17796.8 | 22026.3 | 0.0096 | 0.0077 |
| pMON140819 | P-CUCme.2-1:1:1 | 14 | 34.8 | 16326.3 | 21407.5 | 0.0021 | 0.0016 |
| pMON140820 | P-CUCme.3-1:1:3 | 15 | 51.5 | 17356.8 | 21523.8 | 0.0030 | 0.0024 |
| pMON140821 | EXP-CUCme.4:1:1 | 156 | 3497.8 | 18745.3 | 26065.3 | 0.1866 | 0.1342 |
| pMON140823 | P-CUCme.6-1:1:1 | 18 | 40.8 | 19533.8 | 26361.5 | 0.0021 | 0.0015 |
| pMON140824 | P-CUCme.8-1:1:2 | 19 | 22 | 19701 | 26278 | 0.0011 | 0.0008 |
| pMON140825 | P-CUCme.9-1:1:2 | 20 | 372.5 | 21972.3 | 28755 | 0.0170 | 0.0130 |
| pMON140826 | P-CUCme.10-1:1:1 | 21 | 198 | 21362.8 | 28902 | 0.0093 | 0.0069 |
| pMON140827 | EXP-CUCme.eEF1a:1:1 | 162 | 725 | 21589 | 27635.3 | 0.0336 | 0.0262 |
| pMON140828 | P-CUCme.15-1:1:2 | 23 | 55.3 | 17706 | 28846 | 0.0031 | 0.0019 |
| pMON140829 | P-CUCme.16a-1:1:2 | 24 | 14 | 23289.5 | 30190 | 0.0006 | 0.0005 |
| pMON140830 | P-CUCme.17-1:1:2 | 26 | 155.5 | 23178.3 | 31602.8 | 0.0067 | 0.0049 |
| pMON140831 | P-CUCme.18-1:1:2 | 27 | 86.8 | 19085.8 | 22396.5 | 0.0045 | 0.0039 |
| pMON140832 | P-CUCme.19-1:1:3 | 167 | 130 | 21520.3 | 27270.5 | 0.0060 | 0.0048 |
| pMON140833 | P-CUCme.20-1:3 | 211 | 88.5 | 22223.8 | 30786 | 0.0040 | 0.0029 |
| pMON140834 | P-CUCme.21-1:1:1 | 30 | 98.5 | 18579 | 20506.3 | 0.0053 | 0.0048 |
| pMON140835 | P-CUCme.22-1:1:3 | 31 | 363 | 21780.3 | 28816.3 | 0.0167 | 0.0126 |
| pMON140836 | EXP-CUCme.SAMS2:1:1 | 168 | 515 | 17906 | 23031 | 0.0288 | 0.0224 |
| pMON140837 | P-CUCme.26-1:1:2 | 33 | 125 | 15529.3 | 15169.3 | 0.0080 | 0.0082 |
| pMON140838 | P-CUCme.28-1:1:2 | 34 | 115.8 | 17013.5 | 22236.5 | 0.0068 | 0.0052 |
| pMON140839 | EXP-CUCme.29:1:2 | 212 | 15.5 | 16370.3 | 20409 | 0.0009 | 0.0008 |

To compare the relative activity of each promoter in cotton leaf protoplasts, GUS values were expressed as a ratio of GUS to luciferase activity and normalized with respect to the expression levels observed for the constitutive expression element groups, EXP-At.Act7:1:11 and EXP-CaMV.35S-enh+Ph.DnaK:1:3. Table 16 below shows the GUS to firefly luciferase (FLuc) ratios normalized with respect to EXP-At.Act7:1:11 and EXP-CaMV.35S-enh+Ph.DnaK:1:3. Table 17 below shows the GUS to Renilla luciferase (RLuc) ratios normalized with respect to EXP-At.Act7:1:11 and EXP-CaMV.35S-enh+Ph.DnaK:1:3.

TABLE 16
GUS to firefly luciferase (FLuc) ratios normalized with respect
to EXP-At.Act7:1:11 and EXP-CaMV.35S-enh + Ph.DnaK:1:3.

| | | | GUS/FLuc | GUS/FLuc |
|---|---|---|---|---|
| | | | normalized | normalized with |
| | | | with respect | respect to EXP- |
| | | SEQ ID | to EXP- | CaMV.35S- |
| Construct | Regulatory Element | NO: | At.Act7:1:11 | enh + Ph.DnaK:1:3 |
| pMON109584 | EXP-CaMV.35S- | 201 | 7.037 | 1.000 |
| | enh + Ph.DnaK:1:3 | | | |
| pMON118756 | EXP-At.Act7:1:11 | 202 | 1.000 | 0.142 |
| pMON124912 | Promoterless | | 0.021 | 0.003 |
| pMON140818 | P-CUCme.1-1:1:1rc | 155 | 0.188 | 0.027 |
| pMON140819 | P-CUCme.2-1:1:1 | 14 | 0.042 | 0.006 |
| pMON140820 | P-CUCme.3-1:1:3 | 15 | 0.058 | 0.008 |
| pMON140821 | EXP-CUCme.4:1:1 | 156 | 3.662 | 0.520 |
| pMON140823 | P-CUCme.6-1:1:1 | 18 | 0.041 | 0.006 |
| pMON140824 | P-CUCme.8-1:1:2 | 19 | 0.022 | 0.003 |
| pMON140825 | P-CUCme.9-1:1:2 | 20 | 0.333 | 0.047 |
| pMON140826 | P-CUCme.10-1:1:1 | 21 | 0.182 | 0.026 |
| pMON140827 | EXP-CUCme.eEF1a:1:1 | 162 | 0.659 | 0.094 |
| pMON140828 | P-CUCme.15-1:1:2 | 23 | 0.061 | 0.009 |
| pMON140829 | P-CUCme.16a-1:1:2 | 24 | 0.012 | 0.002 |
| pMON140830 | P-CUCme.17-1:1:2 | 26 | 0.132 | 0.019 |
| pMON140831 | P-CUCme.18-1:1:2 | 27 | 0.089 | 0.013 |
| pMON140832 | P-CUCme.19-1:1:3 | 167 | 0.119 | 0.017 |
| pMON140833 | P-CUCme.20-1:3 | 211 | 0.078 | 0.011 |
| pMON140834 | P-CUCme.21-1:1:1 | 30 | 0.104 | 0.015 |
| pMON140835 | P-CUCme.22-1:1:3 | 31 | 0.327 | 0.046 |
| pMON140836 | EXP-CUCme.SAMS2:1:1 | 168 | 0.564 | 0.080 |
| pMON140837 | P-CUCme.26-1:1:2 | 33 | 0.158 | 0.022 |
| pMON140838 | P-CUCme.28-1:1:2 | 34 | 0.134 | 0.019 |
| pMON140839 | EXP-CUCme.29:1:2 | 212 | 0.019 | 0.003 |

TABLE 17
GUS to renilla luciferase (RLuc) ratios normalized with respect
to EXP-At.Act7:1:11 and EXP-CaMV.35S-enh + Ph.DnaK:1:3.

| | | | | GUS/RLuc |
|---|---|---|---|---|
| | | | GUS/RLuc | normalized with |
| | | | normalized with | respect to EXP- |
| | | SEQ ID | respect to EXP- | CaMV.35S- |
| Construct | Regulatory Element | NO: | At.Act7:1:11 | enh + Ph.DnaK:1:3 |
| pMON109584 | EXP-CaMV.35S-enh + Ph.DnaK:1:3 | 201 | 4.83 | 1.00 |
| pMON118756 | EXP-At.Act7:1:11 | 202 | 1.00 | 0.21 |
| pMON124912 | Promoterless | | 0.02 | 0.00 |
| pMON140818 | P-CUCme.1-1:1:1rc | 155 | 0.20 | 0.04 |
| pMON140819 | P-CUCme.2-1:1:1 | 14 | 0.04 | 0.01 |
| pMON140820 | P-CUCme.3-1:1:3 | 15 | 0.06 | 0.01 |
| pMON140821 | EXP-CUCme.4:1:1 | 156 | 3.4 | 0.71 |
| pMON140823 | P-CUCme.6-1:1:1 | 18 | 0.04 | 0.01 |
| pMON140824 | P-CUCme.8-1:1:2 | 19 | 0.02 | 0.00 |
| pMON140825 | P-CUCme.9-1:1:2 | 20 | 0.33 | 0.07 |
| pMON140826 | P-CUCme.10-1:1:1 | 21 | 0.17 | 0.04 |
| pMON140827 | EXP-CUCme.eEF1a:1:1 | 162 | 0.67 | 0.14 |
| pMON140828 | P-CUCme.15-1:1:2 | 23 | 0.05 | 0.01 |
| pMON140829 | P-CUCme.16a-1:1:2 | 24 | 0.01 | 0.00 |
| pMON140830 | P-CUCme.17-1:1:2 | 26 | 0.13 | 0.03 |
| pMON140831 | P-CUCme.18-1:1:2 | 27 | 0.10 | 0.02 |
| pMON140832 | P-CUCme.19-1:1:3 | 167 | 0.12 | 0.03 |
| pMON140833 | P-CUCme.20-1:3 | 211 | 0.07 | 0.02 |
| pMON140834 | P-CUCme.21-1:1:1 | 30 | 0.12 | 0.03 |
| pMON140835 | P-CUCme.22-1:1:3 | 31 | 0.32 | 0.07 |
| pMON140836 | EXP-CUCme.SAMS2:1:1 | 168 | 0.57 | 0.12 |
| pMON140837 | P-CUCme.26-1:1:2 | 33 | 0.21 | 0.04 |
| pMON140838 | P-CUCme.28-1:1:2 | 34 | 0.13 | 0.03 |
| pMON140839 | EXP-CUCme.29:1:2 | 212 | 0.02 | 0.00 |

As can be seen in Tables 16 and 17. most of the expression element groups tested. demonstrated the ability to drive transgene expression in cotton leaf protoplast cells. One expression element group, EXP-CUCme.4:1:1 (SEQ ID NO: 156) demonstrated levels of transgene expression higher than that of EXP-At.Act7:1:11 in this assay.

### Example 8: Analysis of Regulatory Elements Driving GUS in Cotton Leaf Protoplasts Using Transgene Cassette Amplicons

Cotton leaf protoplasts were transformed with transgene cassette amplicons containing a transcriptional regulatory expression element group driving expression of the ß-glucuronidase (GUS) transgene and compared to GUS expression in leaf protoplasts in which expression of GUS is driven by known constitutive promoters. The transgene cassette amplicons were comprised of an EXP sequence, operably linked to a GUS coding sequence (GUS, SEQ ID NO: 206), operably linked to a 3′ UTR (T-Gb.FbL2-1:1:1, SEQ ID NO: 205). Average GUS expression was compared to the control EXP elements, P-CaMV.35S-enh-1:1:102/L-CaMV.35S-1:1:2 (SEQ ID NO: 210) and EXP-At.Atntt1:1:2 (SEQ ID NO: 200).

A plasmid, for use in co-transformation and normalization of data was also used in a similar manner as that described above in Example 2. The transformation control plasmid was comprised of a constitutive promoter, driving the expression of the firefly (Photinus pyralis) luciferase coding sequence (FLuc, SEQ ID NO: 205), operably linked 5′ to a 3′ termination region from the Agrobacterium tumefaciens nopaline synthase gene (T-AGRtu.nos-1:1:13, SEQ ID NO: 209).

Table 18 below shows the mean GUS expression values conferred by each transgene amplicon. Table 19 below shows the GUS to firefly luciferase (FLuc) ratios normalized with respect to EXP-At.Atntt1:1:2 and P-CaMV.35S-enh-1:1:102/L-CaMV.35S-1:1:2.

TABLE 18
Average GUS and luciferase expression values and GUS/luciferase ratios.

| | | SEQ ID | Mean | Mean | |
|---|---|---|---|---|---|
| Amplicon ID | Regulatory Element | NO: | GUS | Fluc | GUS/Fluc |
| Empty Vector | No DNA | | 32.8 | 14087.5 | 0.002 |
| pMON124912 | No promoter | | 12 | 20486.3 | 0.001 |
| pMON80585 | EXP-At.Atntt1:1:2 | 200 | 55.5 | 18811 | 0.003 |
| pMON33449 | P-CaMV.35S-enh-1:1:102/L-CaMV.35S-1:1:2 | 210 | 12472.5 | 19126.3 | 0.652 |
| 56741 | CumMe_WSM_SF143981.G5150 | 36 | 5.8 | 17449.5 | 0.000 |
| 56492 | CumMe_WSM_SF144839.G5080 | 37 | 27.5 | 16674 | 0.002 |
| 56877 | P-CUCme.CumMe_WSM_SF16444.G5140-1:1:1 | 175 | 96.3 | 17237.8 | 0.006 |
| 56485 | CumMe_WSM_SF16530.G6000 | 42 | 27.3 | 17858.5 | 0.002 |
| 56844 | CumMe_WSM_SF16953.G5180 | 47 | 22.3 | 19398.5 | 0.001 |
| 56500 | CumMe_WSM_SF17250.G5910 | 52 | 12.3 | 23980.3 | 0.001 |
| 56754 | P-CUCme.WSM_SF17252.G7330-1:1:1 | 179 | 16 | 13848.8 | 0.001 |
| 56740 | CumMe_WSM_SF17672.G5610 | 60 | 12 | 16646.8 | 0.001 |
| 56870 | CumMe_WSM_SF18287.G5380 | 66 | 39.3 | 13930.5 | 0.003 |
| 56478 | CumMe_WSM_SF18504.G5090 | 68 | 11.8 | 15830.5 | 0.001 |
| 56481 | CumMe_WSM_SF18530.G5750 | 69 | 6.5 | 15211.3 | 0.000 |
| 56498 | CumMe_WSM_SF18645.G5380 | 73 | 36 | 14569.8 | 0.002 |
| 56746 | P-CUCme.CumMe_WSM_SF18716.G5860-1:1:1 | 184 | 11 | 18054.5 | 0.001 |
| 56490 | CumMe_WSM_SF18801.G5040 | 75 | 21.5 | 14147.3 | 0.002 |
| 56488 | CumMe_WSM_SF19323.G5120 | 81 | 15.3 | 11985.3 | 0.001 |
| 56499 | CumMe_WSM_SF19631.G5170 | 83 | 12.5 | 20140.5 | 0.001 |
| 56482 | P-CUCme.CumMe_WSM_SF19839.G5090-1:1:1 | 189 | 75 | 18690.5 | 0.004 |
| 56489 | CumMe_WSM_SF19850.G5130 | 86 | 38.3 | 19756.5 | 0.002 |
| 56476 | CumMe_WSM_SF20355.G5130 | 91 | 10.5 | 27901.8 | 0.000 |
| 56895 | CumMe_WSM_SF20431.G6340 | 95 | 34.8 | 16283.8 | 0.002 |
| 56744 | CumMe_WSM_SF206458.G5970 | 98 | 11 | 19659 | 0.001 |
| 56480 | CumMe_WSM_SF21366.G5980 | 105 | 10.8 | 17367 | 0.001 |
| 56930 | CumMe_WSM_SF22070.G5280 | 109 | 25.3 | 14210.5 | 0.002 |
| 56484 | CumMe_WSM_SF23181.G5100 | 117 | 20.3 | 13506 | 0.002 |
| 56495 | P-CUCme.CumMe_WSM_SF23760.G5200-1:1:1 | 193 | 7.8 | 15138.5 | 0.001 |
| 56971 | CumMe_WSM_SF25084.G5580 | 125 | 16 | 16135.3 | 0.001 |
| 56742 | P-CUCme.CumMe_WSM_SF25355.G5000-1:1:1 | 196 | 18 | 13782.8 | 0.001 |
| 56494 | CumMe_WSM_SF25455.G5370 | 129 | 10.5 | 16089.8 | 0.001 |
| 56751 | P-CUCme.CumMe_WSM_SF41124.G5080-1:1:1 | 199 | 24.3 | 17884.3 | 0.001 |
| 56483 | CumMe_WSM_SF41644.G6400 | 143 | 14.5 | 13130.5 | 0.001 |
| 56904 | CumMe_WSM_SF44933.G5290 | 147 | 33 | 13369 | 0.002 |
| 56743 | CumMe_WSM_SF9060.G5120 | 154 | 11.3 | 15230.8 | 0.001 |

TABLE 19
GUS to firefly luciferase (FLuc) ratios normalized with respect
to EXP-At.Atntt1:1:2 and P-CaMV.35S-enh-1:1:102/L-CaMV.35S-1:1:2.

| | | | | GUS/Fluc |
|---|---|---|---|---|
| | | | GUS/Fluc | normalized with |
| | | | normalized | respect to P- |
| | | | with respect | CaMV.35S-enh- |
| | | SEQ ID | to EXP- | 1:1:102/L- |
| Amplicon ID | Regulatory Element | NO: | At.Atntt1:1:2 | CaMV.35S-1:1:2 |
| Empty Vector | No DNA | | | |
| pMON124912 | No promoter | | | |
| pMON80585 | EXP-At.Atntt1:1:2 | 200 | 1.000 | 0.005 |
| pMON33449 | P-CaMV.35S-enh-1:1:102/L-CaMV.35S-1:1:2 | 210 | 221.025 | 1.000 |
| 56741 | CumMe_WSM_SF143981.G5150 | 36 | 0.113 | 0.001 |
| 56492 | CumMe_WSM_SF144839.G5080 | 37 | 0.559 | 0.003 |
| 56877 | P-CUCme.CumMe_WSM_SF16444.G5140-1:1:1 | 175 | 1.893 | 0.009 |
| 56485 | CumMe_WSM_SF16530.G6000 | 42 | 0.518 | 0.002 |
| 56844 | CumMe_WSM_SF16953.G5180 | 47 | 0.390 | 0.002 |
| 56500 | CumMe_WSM_SF17250.G5910 | 52 | 0.174 | 0.001 |
| 56754 | P-CUCme.WSM_SF17252.G7330-1:1:1 | 179 | 0.392 | 0.002 |
| 56740 | CumMe_WSM_SF17672.G5610 | 60 | 0.244 | 0.001 |
| 56870 | CumMe_WSM_SF18287.G5380 | 66 | 0.956 | 0.004 |
| 56478 | CumMe_WSM_SF18504.G5090 | 68 | 0.253 | 0.001 |
| 56481 | CumMe_WSM_SF18530.G5750 | 69 | 0.145 | 0.001 |
| 56498 | CumMe_WSM_SF18645.G5380 | 73 | 0.837 | 0.004 |
| 56746 | P-CUCme.CumMe_WSM_SF18716.G5860-1:1:1 | 184 | 0.207 | 0.001 |
| 56490 | CumMe_WSM_SF18801.G5040 | 75 | 0.515 | 0.002 |
| 56488 | CumMe_WSM_SF19323.G5120 | 81 | 0.433 | 0.002 |
| 56499 | CumMe_WSM_SF19631.G5170 | 83 | 0.210 | 0.001 |
| 56482 | P-CUCme.CumMe_WSM_SF19839.G5090-1:1:1 | 189 | 1.360 | 0.006 |
| 56489 | CumMe_WSM_SF19850.G5130 | 86 | 0.657 | 0.003 |
| 56476 | CumMe_WSM_SF20355.G5130 | 91 | 0.128 | 0.001 |
| 56895 | CumMe_WSM_SF20431.G6340 | 95 | 0.724 | 0.003 |
| 56744 | CumMe_WSM_SF206458.G5970 | 98 | 0.190 | 0.001 |
| 56480 | CumMe_WSM_SF21366.G5980 | 105 | 0.211 | 0.001 |
| 56930 | CumMe_WSM_SF22070.G5280 | 109 | 0.603 | 0.003 |
| 56484 | CumMe_WSM_SF23181.G5100 | 117 | 0.509 | 0.002 |
| 56495 | P-CUCme.CumMe_WSM_SF23760.G5200-1:1:1 | 193 | 0.175 | 0.001 |
| 56971 | CumMe_WSM_SF25084.G5580 | 125 | 0.336 | 0.002 |
| 56742 | P-CUCme.CumMe_WSM_SF25355.G5000-1:1:1 | 196 | 0.443 | 0.002 |
| 56494 | CumMe_WSM_SF25455.G5370 | 129 | 0.221 | 0.001 |
| 56751 | P-CUCme.CumMe_WSM_SF41124.G5080-1:1:1 | 199 | 0.461 | 0.002 |
| 56483 | CumMe_WSM_SF41644.G6400 | 143 | 0.374 | 0.002 |
| 56904 | CumMe_WSM_SF44933.G5290 | 147 | 0.837 | 0.004 |
| 56743 | CumMe_WSM_SF9060.G5120 | 154 | 0.251 | 0.001 |

As can be seen in Table 18 above, not all EXP sequences demonstrated the ability to drive transgene expression when compared to the promoterless control. However, the EXP sequences, P-CUCme.CumMe_WSM_SF16444.G5140-1:1:1 (SEQ ID NO: 175) and P-CUCme.CumMe_WSM_SF19839.G5090-1:1:1 (SEQ ID NO: 189) demonstrated the ability to drive trangene expression in soybean cotyledon protoplasts at a level similar or greater than EXP-At.Atntt1:1:2. As shown n in Table 19 above, EXP sequence, P-CUCme.CumMe_WSM_SF19839.G5090-1:1:1 (SEQ ID NO: 189) demonstrated the ability to drive transgene expression in this assay at a level greater than EXP-At.Atntt1:1:2.

### Example 9: Analysis of Regulatory Elements Driving GUS in Stably Transformed Soybean

Soybean plants were transformed with plant expression vectors containing an EXP sequence driving expression of the ß-glucuronidase (GUS) transgene.

Expression of the GUS transgene driven by EXP-CUCme.Ubq1:1:1 (SEQ ID NO: 1), EXP-CUCme.Ubq1:1:3 (SEQ ID NO: 7), P-CUCme.1-1:1:1rc (SEQ ID NO: 155), P-CUCme.2-1:1:1 (SEQ ID NO: 14), P-CUCme.3-1:1:3 (SEQ ID NO: 15), EXP-CUCme.4:1:1 (SEQ ID NO: 156), EXP-CUCme.5:1:1 (SEQ ID NO: 159), P-CUCme.6-1:1:1 (SEQ ID NO: 18), P-CUCme.8-1:1:2 (SEQ ID NO: 19), P-CUCme.9-1:1:2 (SEQ ID NO: 20), P-CUCme.10-1:1:1 (SEQ ID NO: 21), EXP-CUCme.eEF1a: 1:1 (SEQ ID NO: 162), P-CUCme.15-1:1:2 (SEQ ID NO: 23), P-CUCme.17-1:1:2 (SEQ ID NO: 26), P-CUCme.18-1:1:2 (SEQ ID NO: 27), P-CUCme.19-1:1:3 (SEQ ID NO: 167), P-CUCme.20-1:3 (SEQ ID NO: 211), P-CUCme.21-1:1:1 (SEQ ID NO: 30), EXP-CUCme.SAMS2:1:1 (SEQ ID NO: 168), P-CUCme.26-1:1:2 (SEQ ID NO: 33), EXP-CUCme.29:1:2 (SEQ ID NO: 212), P-CUCme.CumMe_WSM_SF25355.G5000-1:1:1 (SEQ ID NO: 196), P-CUCme.CumMe_WSM_SF17111.G5790-1:1:1 (SEQ ID NO: 177), P-CUCme.CumMe_WSM_SF22531.G5120-1:1:1 (SEQ ID NO: 192), P-CUCme.CumMe_WSM_SF18488.G5340-1:1:1 (SEQ ID NO: 181), P-CUCme.CumMe_WSM_SF23760.G5200-1:1:1 (SEQ ID NO: 193), EXP-CUCme.WSM_SF19064.G5690:1:1 (SEQ ID NO: 185), P-CUCme.WSM_SF17252.G7330-1:1:1 (SEQ ID NO: 179), P-CUCme.CumMe_WSM_SF18634.G5190-1:1:1 (SEQ ID NO: 183), P-CUCme.CumMe_WSM_SF19647.G5760-1:1:1 (SEQ ID NO: 188), P-CUCme.CumMe_WSM_SF25936.G5450-1:1:1 (SEQ ID NO: 197), P-CUCme.CumMe_WSM_SF19839.G5090-1:1:1 (SEQ ID NO: 189), CumMe_WSM_SF206458.G5970 (SEQ ID NO: 98) and P-CUCme.CumMe_WSM_SF18716.G5860-1:1:1 (SEQ ID NO: 184) assayed both qualitatively through inspection of stained tissue sections and quantitatively. Each plant expression vector was comprised of a right border region from Agrobacterium tumefaciens, a first transgene cassette comprised of an EXP sequence operably linked 5′ to a coding sequence for ß-glucuronidase (GUS, SEQ ID NO: 206) containing a processable intron derived from the potato light-inducible tissue-specific ST-LS1 gene (Genbank Accession: X04753), operably linked 5′ to a 3′ termination region from the the Gossypium barbadense FbLate-2 gene (T-Gb.FbL2-1:1:1, SEQ ID NO: 205); a second transgene selection cassette used for selection of transformed plant cells that confered resistance to the herbicide glyphosate (driven by the Arabidopsis Actin 7 promoter) and a left border region from A. tumefaciens.

The foregoing EXP sequences were cloned into plant expression constructs as shown in Tables 20 through 23 below and used to transform soybean plants using an agrobacterium mediated transformation method. Expression of GUS was assayed qualitatively using histological sections of selected tissues and quantitatively.

Histochemical GUS analysis was used for qualitative expression analysis of transformed plants. Whole tissue sections were incubated with GUS staining solution X-Gluc (5-bromo-4-chloro-3-indolyl-b-glucuronide) (1 milligram/milliliter) for an appropriate length of time, rinsed, and visually inspected for blue coloration. GUS activity was qualitatively determined by direct visual inspection or inspection under a microscope using selected plant organs and tissues. The R₀ generation plants were inspected for expression in Vn5 Root, R1Root, Vn5 Sink Leaf, Vn5 Source Leaf, R1 Source Leaf, R1 Petiole, Yellow Pod Embryo, Yellow Pod Cotyledon, R3 Immature Seed, R3 Pod, R5 Cotyledon and R1 Flower.

For quantitative analysis, total protein was extracted from selected tissues of transformed corn plants. One microgram of total protein was used with the fluorogenic substrate 4-methyleumbelliferyl-β-D-glucuronide (MUG) in a total reaction volume of 50 microliters. The reaction product, 4-methlyumbelliferone (4-MU), is maximally fluorescent at high pH, where the hydroxyl group is ionized. Addition of a basic solution of sodium carbonate simultaneously stops the assay and adjusts the pH for quantifying the fluorescent product. Fluorescence was measured with excitation at 365 nm, emission at 445 nm using a Fluoromax-3 (Horiba; Kyoto, Japan) with Micromax Reader, with slit width set at excitation 2 nm and emission 3 nm.

Tables 20 and 21 below show the mean quantitative expression levels measured in the R₀ generation plant tissues. Those tissued not assayed are shown as blank cells in both tables.

TABLE 20
Mean GUS expression in Vn5 Root, R1 Root, Vn5 Sink Leaf, Vn5 Source Leaf,
R1 Source Leaf and R1 Petiole of R₀ generation transformed soybean plants

| | | SEQ | | | Vn5_{—} | Vn5_{—} | R1_{—} | |
|---|---|---|---|---|---|---|---|---|
| | | ID | Vn5_{—} | R1_{—} | Sink_{—} | Source | Source_{—} | |
| Construct | Regulatory Element | NO: | Root | Root | Leaf | Leaf | Leaf | R1_Petiole |
| pMON138776 | EXP-CUCme.Ubq1:1:1 | 1 | 4 | | | | 4 | 4 |
| pMON138778 | EXP-CUCme.Ubq1:1:3 | 7 | 16 | | 1 | 2 | 13 | 23 |
| pMON140818 | P-CUCme.1-1:1:1rc | 155 | 48.21 | | 22.35 | 20.24 | 33.01 | 78.17 |
| pMON140819 | P-CUCme.2-1:1:1 | 14 | | | | | | |
| pMON140820 | P-CUCme.3-1:1:3 | 15 | | | | | | |
| pMON140821 | EXP-CUCme.4:1:1 | 156 | 96.82 | | 28.32 | 39.17 | 322.98 | 280.03 |
| pMON140822 | EXP-CUCme.5:1:1 | 159 | 28.88 | | | | 41.11 | |
| pMON140823 | P-CUCme.6-1:1:1 | 18 | 23.94 | | | | 32.14 | 30.22 |
| pMON140824 | P-CUCme.8-1:1:2 | 19 | | | | | | |
| pMON140825 | P-CUCme.9-1:1:2 | 20 | 22.06 | | | | 21.22 | 23.08 |
| pMON140826 | P-CUCme.10-1:1:1 | 21 | | | | | | |
| pMON140827 | EXP-CUCme.eEF1a:1:1 | 162 | 189.24 | 153.52 | 59.6 | 37.44 | 103.01 | 130.6 |
| pMON140828 | P-CUCme.15-1:1:2 | 23 | 30.53 | | | | | |
| pMON140830 | P-CUCme.17-1:1:2 | 26 | 51.62 | | 30.07 | 31.08 | 30.49 | 60.14 |
| pMON140831 | P-CUCme.18-1:1:2 | 27 | 57.38 | | | | | 30.03 |
| pMON140832 | P-CUCme.19-1:1:3 | 167 | 23.07 | | 50.21 | 59.73 | 65.58 | 137.42 |
| pMON140833 | P-CUCme.20-1:3 | 211 | 23.15 | | 61.6 | 118.76 | 502.55 | 119.46 |
| pMON140834 | P-CUCme.21-1:1:1 | 30 | | | | | 25.49 | |
| pMON140836 | EXP-CUCme.SAMS2:1:1 | 168 | 230.89 | 184.88 | 65.44 | 53.36 | 118.82 | 351.49 |
| pMON140837 | P-CUCme.26-1:1:2 | 33 | 56.21 | | 26.81 | 45.07 | 51.61 | 47.42 |
| pMON140839 | EXP-CUCme.29:1:2 | 212 | 82.17 | | 45.2 | 28.27 | 64.96 | 109.9 |
| pMON144926 | P- | 196 | 28.53 | | | | | |
| | CUCme.CumMe_WSM_SF25355.G5000- | | | | | | | |
| | 1:1:1 | | | | | | | |
| pMON144927 | P- | 177 | 23.62 | | | | | |
| | CUCme.CumMe_WSM_SF17111.G5790- | | | | | | | |
| | 1:1:1 | | | | | | | |
| pMON144928 | P- | 192 | 75.62 | | 23 | 20.46 | 21.78 | 39.77 |
| | CUCme.CumMe_WSM_SF22531.G5120- | | | | | | | |
| | 1:1:1 | | | | | | | |
| pMON144931 | P- | 181 | 43.2 | | | | | 52.55 |
| | CUCme.CumMe_WSM_SF18488.G5340- | | | | | | | |
| | 1:1:1 | | | | | | | |
| pMON144933 | P- | 193 | 25.61 | | 20.45 | 0 | 0 | 28.69 |
| | CUCme.CumMe_WSM_SF23760.G5200- | | | | | | | |
| | 1:1:1 | | | | | | | |
| pMON146941 | EXP- | 185 | 33.5 | | 0 | 0 | 24.27 | 47.82 |
| | CUCme.WSM_SF19064.G5690:1:1 | | | | | | | |
| pMON144932 | P-CUCme.WSM_SF17252.G7330- | 179 | 32.54 | | 23.76 | 21.5 | 0 | 22.21 |
| | 1:1:1 | | | | | | | |
| pMON146940 | P- | 183 | 0 | | 0 | 0 | 0 | 0 |
| | CUCme.CumMe_WSM_SF18634.G5190- | | | | | | | |
| | 1:1:1 | | | | | | | |
| pMON147340 | P- | 188 | 28.9 | | 0 | 0 | 29.77 | 25.82 |
| | CUCme.CumMe_WSM_SF19647.G5760- | | | | | | | |
| | 1:1:1 | | | | | | | |
| pMON147342 | P- | 197 | 50.15 | | 24.26 | 0 | 29.38 | 29.91 |
| | CUCme.CumMe_WSM_SF25936.G5450- | | | | | | | |
| | 1:1:1 | | | | | | | |
| pMON147343 | P- | 189 | 36.05 | | 25.7 | 27.54 | 22.85 | 37.15 |
| | CUCme.CumMe_WSM_SF19839.G5090- | | | | | | | |
| | 1:1:1 | | | | | | | |
| pMON144929 | CumMe_WSM_SF206458.G5970 | 98 | | | | | | |
| pMON147304 | P- | 184 | 35.01 | | 21.17 | 21.23 | 22 | 44.57 |
| | CUCme.CumMe_WSM_SF18716.G5860- | | | | | | | |
| | 1:1:1 | | | | | | | |

TABLE 21
Mean GUS expression in Yellow Pod Embryo, Yellow Pod Cotyledon, R3 Immature Seed,
R3 Pod, R5 Cotyledon and R1 Flower of R₀ generation transformed soybean plants

| | | SEQ | Yellow_{—} | Yellow_{—} | R3_{—} | | | |
|---|---|---|---|---|---|---|---|---|
| | | ID | Pod_{—} | Pod_{—} | Immature_{—} | R3_{—} | R5_{—} | R1_{—} |
| Construct | Regulatory Element | NO: | Embryo | Cotyledon | Seed | Pod | Cotyledon | Flower |
| pMON138776 | EXP-CUCme.Ubq1:1:1 | 1 | 12 | 9 | 13 | 11 | 10 | 7 |
| pMON138778 | EXP-CUCme.Ubq1:1:3 | 7 | 3 | 1 | 13 | 9 | 13 | 27 |
| pMON140818 | P-CUCme.1-1:1:1rc | 155 | 100.79 | 117.5 | 38.31 | 84.72 | 132.27 | 66.8 |
| pMON140819 | P-CUCme.2-1:1:1 | 14 | | | | | 20.35 | 36.18 |
| pMON140820 | P-CUCme.3-1:1:3 | 15 | | | | | | |
| pMON140821 | EXP-CUCme.4:1:1 | 156 | 86.68 | 225.53 | 105.62 | 342.07 | 119.08 | 184.92 |
| pMON140822 | EXP-CUCme.5:1:1 | 159 | 21.48 | 32.27 | 21.47 | 21.66 | | 36.88 |
| pMON140823 | P-CUCme.6-1:1:1 | 18 | 38.75 | | 23.03 | | 25.32 | 58.7 |
| pMON140824 | P-CUCme.8-1:1:2 | 19 | | | | | 90.33 | 25.77 |
| pMON140825 | P-CUCme.9-1:1:2 | 20 | 132.04 | | | 20.56 | 34.78 | |
| pMON140826 | P-CUCme.10-1:1:1 | 21 | | | | | 22.34 | |
| pMON140827 | EXP-CUCme.eEF1a:1:1 | 162 | 200.28 | 291.26 | 58.21 | 131.17 | 114.29 | 130.38 |
| pMON140828 | P-CUCme.15-1:1:2 | 23 | | | 142.24 | 26.2 | | |
| pMON140830 | P-CUCme.17-1:1:2 | 26 | 343.34 | 302.94 | 65.55 | 80.94 | 137.02 | 62.7 |
| pMON140831 | P-CUCme.18-1:1:2 | 27 | 103.17 | 135.97 | 30 | 34.62 | 88.14 | 23.73 |
| pMON140832 | P-CUCme.19-1:1:3 | 167 | 30.96 | 64.46 | | 316.66 | | 53.46 |
| pMON140833 | P-CUCme.20-1:3 | 211 | 174.62 | 524.88 | | 222.04 | 59.43 | 124.68 |
| pMON140834 | P-CUCme.21-1:1:1 | 30 | | | 28.15 | 20.52 | 23.89 | |
| pMON140836 | EXP-CUCme.SAMS2:1:1 | 168 | 110.23 | 159.43 | 61.99 | 248.96 | 49.17 | 224.24 |
| pMON140837 | P-CUCme.26-1:1:2 | 33 | 56.73 | 50.06 | 70 | 143.05 | 25.06 | 49.92 |
| pMON140839 | EXP-CUCme.29:1:2 | 212 | 251.76 | 237.2 | 49.16 | 89.28 | 114.92 | 57.84 |
| pMON144926 | P- | 196 | | | 21.41 | | 22.23 | |
| | CUCme.CumMe_WSM_SF25355.G5000- | | | | | | | |
| | 1:1:1 | | | | | | | |
| pMON144927 | P- | 177 | 58.84 | 28.94 | | | 20.97 | |
| | CUCme.CumMe_WSM_SF17111.G5790- | | | | | | | |
| | 1:1:1 | | | | | | | |
| pMON144928 | P- | 192 | 135.62 | 152.48 | 30.45 | 51.71 | 129.72 | 42.2 |
| | CUCme.CumMe_WSM_SF22531.G5120- | | | | | | | |
| | 1:1:1 | | | | | | | |
| pMON144931 | P- | 181 | 866.94 | | 23.26 | 21.49 | | |
| | CUCme.CumMe_WSM_SF18488.G5340- | | | | | | | |
| | 1:1:1 | | | | | | | |
| pMON144933 | P- | 193 | | | 29.03 | 34.9 | 69.63 | 24.42 |
| | CUCme.CumMe_WSM_SF23760.G5200- | | | | | | | |
| | 1:1:1 | | | | | | | |
| pMON146941 | EXP- | 185 | | | 36.69 | 83.08 | 89.81 | 33.99 |
| | CUCme.WSM_SF19064.G5690:1:1 | | | | | | | |
| pMON144932 | P-CUCme.WSM_SF17252.G7330- | 179 | | | 34.29 | 39.89 | 113.83 | 0 |
| | 1:1:1 | | | | | | | |
| pMON146940 | P- | 183 | | | 30.25 | 0 | 0 | 0 |
| | CUCme.CumMe_WSM_SF18634.G5190- | | | | | | | |
| | 1:1:1 | | | | | | | |
| pMON147340 | P- | 188 | | | 25.73 | 28.28 | 24.04 | 23.35 |
| | CUCme.CumMe_WSM_SF19647.G5760- | | | | | | | |
| | 1:1:1 | | | | | | | |
| pMON147342 | P- | 197 | | | 104.02 | 80.27 | 31.06 | 26.8 |
| | CUCme.CumMe_WSM_SF25936.G5450- | | | | | | | |
| | 1:1:1 | | | | | | | |
| pMON147343 | P- | 189 | | | | | | 29.09 |
| | CUCme.CumMe_WSM_SF19839.G5090- | | | | | | | |
| | 1:1:1 | | | | | | | |
| pMON144929 | CumMe_WSM_SF206458.G5970 | 98 | | | 24.42 | 25.33 | | |
| pMON147304 | P- | 184 | | | | 283.49 | | 61.43 |
| | CUCme.CumMe_WSM_SF18716.G5860- | | | | | | | |
| | 1:1:1 | | | | | | | |

As can be seen in Tables 20 and 21, the EXP sequences, EXP-CUCme.Ubq1:1:1(SEQ ID NO: 1), EXP-CUCme.Ubq1:1:3 (SEQ ID NO: 7), P-CUCme.1-1:1:1rc (SEQ ID NO: 155), P-CUCme.2-1:1:1 (SEQ ID NO: 14), EXP-CUCme.4:1:1 (SEQ ID NO: 156), EXP-CUCme.5:1:1 (SEQ ID NO: 159), P-CUCme.6-1:1:1 (SEQ ID NO: 18), P-CUCme.8-1:1:2 (SEQ ID NO: 19), P-CUCme.9-1:1:2 (SEQ ID NO: 20), P-CUCme.10-1:1:1 (SEQ ID NO: 21), EXP-CUCme.eEF1a:1:1 (SEQ ID NO: 162), P-CUCme.15-1:1:2 (SEQ ID NO: 23), P-CUCme.17-1:1:2 (SEQ ID NO: 26), P-CUCme.18-1:1:2 (SEQ ID NO: 27), P-CUCme.19-1:1:3 (SEQ ID NO: 167), P-CUCme.20-1:3 (SEQ ID NO: 211), P-CUCme.21-1:1:1 (SEQ ID NO: 30), EXP-CUCme.SAMS2:1:1 (SEQ ID NO: 168), P-CUCme.26-1:1:2 (SEQ ID NO: 33), EXP-CUCme.29:1:2 (SEQ ID NO: 212), P-CUCme.CumMe_WSM_SF25355.G5000-1:1:1 (SEQ ID NO: 196), P-CUCme.CumMe_WSM_SF17111.G5790-1:1:1 (SEQ ID NO: 177), P-CUCme.CumMe_WSM_SF22531.G5120-1:1:1 (SEQ ID NO: 192), P-CUCme.CumMe_WSM_SF18488.G5340-1:1:1 (SEQ ID NO: 181), P-CUCme.CumMe_WSM_SF23760.G5200-1:1:1 (SEQ ID NO: 193), EXP-CUCme.WSM_SF19064.G5690:1:1 (SEQ ID NO: 185), P-CUCme.WSM_SF17252.G7330-1:1:1 (SEQ ID NO: 179), P-CUCme.CumMe_WSM_SF18634.G5190-1:1:1 (SEQ ID NO: 183), P-CUCme.CumMe_WSM_SF19647.G5760-1:1:1 (SEQ ID NO: 188), P-CUCme.CumMe_WSM_SF25936.G5450-1:1:1 (SEQ ID NO: 197), P-CUCme.CumMe_WSM_SF19839.G5090-1:1:1 (SEQ ID NO: 189), CumMe_WSM_SF206458.G5970 (SEQ ID NO: 98) and P-CUCme.CumMe_WSM_SF18716.G5860-1:1:1 (SEQ ID NO: 184) demonstrated quantitatively the capacity to drive transgene expression in some or all tissues assayed, depending upon the EXP sequence used to drive expression.

Histological analysis of selected tissue sections provided further evidence of expression for many of the EXP sequences. EXP-CUCme.Ubq1:1:1 (SEQ ID NO: 1) and EXP-CUCme.Ubq1:1:3 (SEQ ID NO: 7) demonstrated a constitutive expression pattern with staining observed in all tissues, even though quantitative analysis showed fairly low levels of expression. This type of expression pattern can be most adventitious to driving expression of transgenes that require a low level of constitutive expression. Expression driven by P-CUCme.1-1:1:1rc (SEQ ID NO: 155) demonstrated expression in sink and source leaf vascular bundles and xylem and in the root cortex, phloem, xylem, endodermis, stele and tip. Expression driven by EXP-CUCme.4:1:1(SEQ ID NO: 156) was observed in all tissues with the highest expression observed in the reproductive phase of the plant. Expression driven by P-CUCme.10-1:1:1 (SEQ ID NO: 21) was observed only in in V5 Sink Leaf and R1 Flower anthers. Expression driven by EXP-CUCme.eEF1a:1:1 (SEQ ID NO: 162) demonstrated a consititutive expression pattern with highest expression being observed in yellow pod embryo and cotyledon. The yellow pod embryo activity was 5 fold higher in the R1 generation than in the R0 generation (see Table 23 below). Expression driven by P-CUCme.15-1:1:2 (SEQ ID NO: 23), P-CUCme.17-1:1:2 (SEQ ID NO: 26) and P-CUCme.18-1:1:2 (SEQ ID NO: 27) demonstrated a constitutive level of expression histologically. Expression driven by P-CUCme.19-1:1:3 (SEQ ID NO: 167) demonstrated a constitutive pattern of expression histologically with the exception of the V5 root and R1 petiole. R3 pod showed the highest expression.

Expression driven by P-CUCme.20-1:3 (SEQ ID NO: 211) demonstrated a constitutive expression pattern histologically with the exception of expression in V5 root. Expression was highest in the R8 stage cotyledon. Expression driven by EXP-CUCme.SAMS2:1:1 (SEQ ID NO: 168) demonstrated a constitutive pattern of expression with expression observed histologically in all tissues. GUS expression was observed to increase in the R1 generation (see Tables 22 and 23 below). The R1 stage flowers and petioles demonstrated the highest levels of in expression soybean. Expression driven by P-CUCme.CumMe_WSM_SF22531.G5120-1:1:1 (SEQ ID NO: 192) demonstrated a constitutive pattern of expression histologically with highest expression in the R8 stage cotyledon and embryo. Expression driven by P-CUCme.CumMe_WSM_SF18488.G5340-1:1:1 (SEQ ID NO: 181) demonstrated a constitutive level of expression while quantitatively high expression was observed in the yellow pod embryo.

R₀ generation plants transformed with the plasmid constructs comprising EXP-CUCme.eEF1a:1:1 (SEQ ID NO: 162) and EXP-CUCme.SAMS2:1:1 (SEQ ID NO: 168) were allowed to set seed and the R₁ generation plants analyzed for GUS expression. The R₁ generation plants were analyzed for expression in Vn5 Root, Vn5 Sink Leaf, Vn5 Source Leaf, R1 Source Leaf, R1 Petiole Yellow Pod Embryo, Yellow Pod Cotyledon, R3 Immature Seed, R3 Pod, R5 Cotyledon and R1 Flower. Tables 22 and 23 show the mean GUS expression measured in each tissue of the R₁ generation transformed plants.

TABLE 22
Mean GUS expression in Vn5 Root, Vn5 Sink Leaf, Vn5 Source Leaf, R1
Source Leaf, R1 Petiole of R₁ generation transformed soybean plants

| | | SEQ | | Vn5_{—} | Vn5_{—} | R1_{—} | |
|---|---|---|---|---|---|---|---|
| | | ID | Vn5_{—} | Sink_{—} | Source | Source_{—} | R1_{—} |
| Construct | Regulatory Element | NO: | Root | Leaf | Leaf | Leaf | Petiole |
| pMON140827 | EXP-CUCme.eEF1a:1:1 | 162 | 145.84 | 50.24 | 43.73 | 107.98 | 357.67 |
| pMON140836 | EXP-CUCme.SAMS2:1:1 | 168 | 260.41 | 65.52 | 51.12 | 129.86 | 623.42 |

TABLE 23
Mean GUS expression in Yellow Pod Embryo, Yellow Pod Cotyledon, R3 Immature Seed,
R3 Pod, R5 Cotyledon, R1 Flower of R₁ generation transformed soybean plants

| | | SEQ | Yellow_{—} | Yellow_{—} | R3_{—} | | | |
|---|---|---|---|---|---|---|---|---|
| | | ID | Pod_{—} | Pod_{—} | Immature_{—} | R3_{—} | R5_{—} | R1_{—} |
| Construct | Regulatory Element | NO: | Embryo | Cotyledon | Seed | Pod | Cotyledon | Flower |
| pMON140827 | EXP-CUCme.eEF1a:1:1 | 162 | 1098.51 | 764.83 | 288.77 | 214.6 | 459.62 | 394.77 |
| pMON140836 | EXP-CUCme.SAMS2:1:1 | 168 | 219.04 | 291.58 | 241.48 | 382.73 | 397.91 | 653.23 |

As can be seen in Tables 22 and 23 above expression driven in R₁ generation by EXP-CUCme.eEF1a:1:1 (SEQ ID NO: 162) and EXP-CUCme.SAMS2:1:1 (SEQ ID NO: 168) shows a constitutive level of expression with increase in expression observed in many tissues at R₁ generation relative to R₀ generation.

Having illustrated and described the principles of the present invention, it should be apparent to persons skilled in the art that the invention can be modified in arrangement and detail without departing from such principles. All modifications that are within the spirit and scope of the claims are intended to be included within the scope of the present invention. All publications and published patent documents cited herein are hereby incorporated by reference to the same extent as if each individual publication or patent application is specifically and individually indicated to be incorporated by reference.

What is claimed is:

1. A DNA molecule exhibiting a gene regulatory functional activity comprising a polynucleotide sequence selected from the group consisting of:
a) a sequence with at least 85 percent sequence identity to any of SEQ ID NOS: 1-199, 211 and 212;
b) a sequence comprising any of SEQ ID NOs: 1-199, 211 and 212; and
c) a fragment of any of SEQ ID NOs: 1-199, 211 and 212 exhibiting gene-regulatory activity;
wherein said DNA molecule is operably linked to a heterologous transcribable polynucleotide molecule.

2. The DNA molecule of claim 1, wherein said polynucleotide sequence has at least 90 percent sequence identity to the polynucleotide sequence as set forth in any of SEQ ID NOs: 1-199, 211 and 212.

3. The DNA molecule of claim 1, wherein said polynucleotide sequence has at least 95 percent sequence identity to the polynucleotide sequence as set forth in any of SEQ ID NOs: 1-199, 211 and 212.

4. The DNA molecule of claim 1, wherein the heterologous transcribable polynucleotide molecule comprises a gene of agronomic interest.

5. The DNA molecule of claim 4, wherein the gene of agronomic interest confers herbicide tolerance in plants.

6. The DNA molecule of claim 4, wherein the gene of agronomic interest confers pest resistance in plants.

7. A transgenic plant cell comprising a heterologous DNA molecule exhibiting a gene regulatory functional activity comprising a polynucleotide sequence selected from the group consisting of:
a) a sequence with at least 85 percent sequence identity to any of SEQ ID NOs: 1-199, 211 and 212;
b) a sequence comprising any of SEQ ID NOs: 1-199, 211 and 212; and
c) a fragment of any of SEQ ID NOs: 1-199, 211 and 212 exhibiting gene-regulatory activity;
wherein said DNA molecule is operably linked to a heterologous transcribable polynucleotide molecule.

8. The transgenic plant cell of claim 7, wherein said transgenic plant cell is a monocotyledonous plant cell.

9. The transgenic plant cell of claim 7, wherein said transgenic plant cell is a dicotyledonous plant cell.

10. A transgenic plant, or part thereof, comprising a DNA molecule exhibiting a gene regulatory functional activity comprising a polynucleotide sequence selected from the group consisting of:
a) a sequence with at least 85 percent sequence identity to any of SEQ ID NOS: 1-199, 211 and 212;
b) a sequence comprising any of SEQ ID NOs: 1-199, 211 and 212; and
c) a fragment of any of SEQ ID NOs: 1-199, 211 and 212 exhibiting gene-regulatory activity;
wherein said DNA molecule is operably linked to a heterologous transcribable polynucleotide molecule.

11. A progeny plant of the transgenic plant of claim 10, or part thereof, wherein the progeny plant or part thereof comprises said DNA molecule exhibiting a gene regulatory functional activity.

12. A transgenic seed comprising a DNA molecule exhibiting a gene regulatory functional activity comprising a polynucleotide sequence selected from the group consisting of:
a) a sequence with at least 85 percent sequence identity to any of SEQ ID NOs: 1-199, 211 and 212;
b) a sequence comprising any of SEQ ID NOs: 1-199, 211 and 212; and
c) a fragment of any of SEQ ID NOs: 1-199, 211 and 212 exhibiting gene-regulatory activity;
wherein said DNA molecule is operably linked to a heterologous transcribable polynucleotide molecule.

13. A method of producing a commodity product comprising:
a) obtaining a transgenic plant or part thereof comprising a DNA molecule exhibiting a gene regulatory functional activity comprising a polynucleotide sequence selected from the group consisting of:
1) a sequence with at least 85 percent sequence identity to any of SEQ ID NOs: 1-199, 211 and 212;
2) a sequence comprising any of SEQ ID NOs: 1-199, 211 and 212; and
3) a fragment of any of SEQ ID NOs: 1-199, 211 and 212 exhibiting gene-regulatory activity;
wherein said DNA molecule is operably linked to a heterologous transcribable polynucleotide molecule; and
b) producing the commodity product therefrom.

14. The method of claim 13, wherein the commodity product is protein concentrate, protein isolate, grain, starch, seeds, meal, flour, biomass, or seed oil.

15. A commodity product comprising a DNA molecule exhibiting a gene regulatory functional activity comprising a polynucleotide sequence selected from the group consisting of:
a) a sequence with at least 85 percent sequence identity to any of SEQ ID NOs: 1-199, 211 and 212;
b) a sequence comprising any of SEQ ID NOs: 1-199, 211 and 212; and
c) a fragment of any of SEQ ID NOs: 1-199, 211 and 212 exhibiting gene-regulatory activity;
wherein said DNA molecule is operably linked to a heterologous transcribable polynucleotide molecule.

16. A method of expressing a transcribable polynucleotide molecule comprising:
a) obtaining a transgenic plant comprising a DNA molecule exhibiting a gene regulatory functional activity comprising a polynucleotide sequence selected from the group consisting of:
1) a sequence with at least 85 percent sequence identity to any of SEQ ID NOs: 1-199, 211 and 212;
2) a sequence comprising any of SEQ ID NOs: 1-199, 211 and 212; and
3) a fragment of any of SEQ ID NOs: 1-199, 211 and 212 exhibiting gene-regulatory activity;
wherein said DNA molecule is operably linked to a heterologous transcribable polynucleotide molecule; and
b) cultivating said transgenic plant, wherein the transcribable polynucleotide is expressed.


SEQUENCE LISTING (212 sequences)

SEQ ID NO: 1 - DNA, 2611 bp, Cucumis melo
  source 1..2611: mol_type = unassigned DNA
atctgaaaggaacacctagcaaggggctactctacaagcatactaagtctacaaagctagagttgtatggttatgcagaagacctggacaaaagaagatcactcgctgcttttacttttatcctaagaggaaatgtgattttatggaagtttaacctatagcctgtagtggcactattcacaacaaaagtaaagtttatagccatgactgaagttgttaaagaagtcgtctggctaaaaggactacttgaagaacttggcttcttttaacagtcagtaaacatcatgtgtgatagttaaagtgcaatacacttgtctaaaaatctgcaatatcacgaaagaactaagcatattgatgtgaagctatatgtcattagagaagtcatagcaaagagaaaagtaacagtatcaaaggttcagacaaaagaaaatgcagcagatatgttgactaaaatagttactaatgctaaactcgagcactgcctacagttgctcaaggtaatagactacttaaaagaatagaatcagaagaaatagtcattggtagcaataaaattcaaggtggaggattgttaaaaagaagagtgaattttattacttaaagaaaatctcggtgaaactcgaaagatctcgattcgaaactctattgcttaagaacctggtgaagctcgagagatcttgatacaatcccagtgccctaactcttcaacaagctaagcaagttgtactgtggggctcaatctcggttcaatctcgacgcacctgatgctttgttccctgtctactcgatgaagaagcaattacttctcaggacaactcggtacccctaaatacagattttgagcttcgtgatcctacaactgaaatcaaatagaaaaactaataagttagttagagtttgttatatttactgccattaaataactctgtaatgtaaataataaaccatttaactcaatatgaaatatagaatgagaaaaagaaaaagaaaaagttaaagagagagaggaagaaaactcattttcaaattctctatacttgtttgatccttgaataagttgaataaaagctctatggcggcttcaaagtggatgtaggcactattagtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattttccatacagactcgaaatactctaaactttctcatcgcgctttattcctatttcgtaattcgctcttcttcaacctctcaaggttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 2 - DNA, 2068 bp, Cucumis melo
  source 1..2068: mol_type = unassigned DNA
atctgaaaggaacacctagcaaggggctactctacaagcatactaagtctacaaagctagagttgtatggttatgcagaagacctggacaaaagaagatcactcgctgcttttacttttatcctaagaggaaatgtgattttatggaagtttaacctatagcctgtagtggcactattcacaacaaaagtaaagtttatagccatgactgaagttgttaaagaagtcgtctggctaaaaggactacttgaagaacttggcttcttttaacagtcagtaaacatcatgtgtgatagttaaagtgcaatacacttgtctaaaaatctgcaatatcacgaaagaactaagcatattgatgtgaagctatatgtcattagagaagtcatagcaaagagaaaagtaacagtatcaaaggttcagacaaaagaaaatgcagcagatatgttgactaaaatagttactaatgctaaactcgagcactgcctacagttgctcaaggtaatagactacttaaaagaatagaatcagaagaaatagtcattggtagcaataaaattcaaggtggaggattgttaaaaagaagagtgaattttattacttaaagaaaatctcggtgaaactcgaaagatctcgattcgaaactctattgcttaagaacctggtgaagctcgagagatcttgatacaatcccagtgccctaactcttcaacaagctaagcaagttgtactgtggggctcaatctcggttcaatctcgacgcacctgatgctttgttccctgtctactcgatgaagaagcaattacttctcaggacaactcggtacccctaaatacagattttgagcttcgtgatcctacaactgaaatcaaatagaaaaactaataagttagttagagtttgttatatttactgccattaaataactctgtaatgtaaataataaaccatttaactcaatatgaaatatagaatgagaaaaagaaaaagaaaaagttaaagagagagaggaagaaaactcattttcaaattctctatacttgtttgatccttgaataagttgaataaaagctctatggcggcttcaaagtggatgtaggcactattagtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 3 - DNA, 82 bp, Cucumis melo
  source 1..82: mol_type = unassigned DNA
tccatacagactcgaaatactctaaactttctcatcgcgctttattcctatttcgtaattcgctcttcttcaacctctcaag

SEQ ID NO: 4 - DNA, 461 bp, Cucumis melo
  source 1..461: mol_type = unassigned DNA
gttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 5 - DNA, 2002 bp, Cucumis melo
  source 1..2002: mol_type = unassigned DNA
tcggtgaaactcgaaagatctcgattcgaaactctattgcttaagaacctggtgaagctcgagagatcttgatacaatcccagtgccctaactcttcaacaagctaagcaagttgtactgtggggctcaatctcggttcaatctcgacgcacctgatgctttgttccctgtctactcgatgaagaagcaattacttctcaggacaactcggtacccctaaatacagattttgagcttcgtgatcctacaactgaaatcaaatagaaaaactaataagttagttagagtttgttatatttactgccattaaataactctgtaatgtaaataataaaccatttaactcaatatgaaatatagaatgagaaaaagaaaaagaaaaagttaaagagagagaggaagaaaactcattttcaaattctctatacttgtttgatccttgaataagttgaataaaagctctatggcggcttcaaagtggatgtaggcactattagtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattttccatacagactcgaaatactctaaactttctcatcgcgctttattcctatttcgtaattcgctcttcttcaacctctcaaggttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 6 - DNA, 1459 bp, Cucumis melo
  source 1..1459: mol_type = unassigned DNA
tcggtgaaactcgaaagatctcgattcgaaactctattgcttaagaacctggtgaagctcgagagatcttgatacaatcccagtgccctaactcttcaacaagctaagcaagttgtactgtggggctcaatctcggttcaatctcgacgcacctgatgctttgttccctgtctactcgatgaagaagcaattacttctcaggacaactcggtacccctaaatacagattttgagcttcgtgatcctacaactgaaatcaaatagaaaaactaataagttagttagagtttgttatatttactgccattaaataactctgtaatgtaaataataaaccatttaactcaatatgaaatatagaatgagaaaaagaaaaagaaaaagttaaagagagagaggaagaaaactcattttcaaattctctatacttgtttgatccttgaataagttgaataaaagctctatggcggcttcaaagtggatgtaggcactattagtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 7 - DNA, 1507 bp, Cucumis melo
  source 1..1507: mol_type = unassigned DNA
agtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattttccatacagactcgaaatactctaaactttctcatcgcgctttattcctatttcgtaattcgctcttcttcaacctctcaaggttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 8 - DNA, 964 bp, Cucumis melo
  source 1..964: mol_type = unassigned DNA
agtcgaaccacaataaatttgttatgttcttttgctattccttgtaatctccataaatattttcttactaagctctagaaatctgcttgtcaagagattaggtatcatttatgccttttatatttcctttcggttgcatatcttgagctagttaagatcgagaggttactgttgttgaaaccgagattagtatctttggattaacacgtgcctaccaaaatttgaaattttgtatttaccccattcattggataataagcaattcttatagtgttatcaattaaactcctataaagtgtaataattgaatccatgaactattttcatatgtaatcttaataaaatgaatttagaagtttaattaaaataatatattttgtatgctatttttcaaagtttgaagaatgtgttaattgatacacatacaaaaaatctaggttttacatgaaaaactatggaagtgaaagatagcatctaatattttatgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 9 - DNA, 1022 bp, Cucumis melo
  source 1..1022: mol_type = unassigned DNA
tgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattttccatacagactcgaaatactctaaactttctcatcgcgctttattcctatttcgtaattcgctcttcttcaacctctcaaggttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 10 - DNA, 479 bp, Cucumis melo
  source 1..479: mol_type = unassigned DNA
tgacacaaaatgcaaactaatatataaaggatttaattaatttttataggtttcaaatttgttagacttgtcaaatacaaaattttattgaaccaaatacatacaaacatcaaaattaagaacagaaaatctaaattcaaatgaaatttattaatagaaaaattagaaaaaagaaaaagaaaataaaaggaatcgtattgttttttccttcctttttcccatttgagaggtgaataaagctaattgagctgctctaacttcctaatctttatgctttccccataaagctttcccaactgcgcgtaatcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 11 - DNA, 716 bp, Cucumis melo
  source 1..716: mol_type = unassigned DNA
tcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattttccatacagactcgaaatactctaaactttctcatcgcgctttattcctatttcgtaattcgctcttcttcaacctctcaaggttttcatcttttctctatcttctgttttcagattgcatcttttccccctcctgttcgattaattgatgtttgaattttcgagaaacgatttgaagtctttgttgtatttttcatttctgttcgttaggtaggtcgatttttaatcgtgatgtccgacgttgttcggatgattcacatttggtttttgtcatcttctttctatgttgtgattatcatgatttttatctttttttcttctcaagatttgtaatttatcgattccccatggttcttggttttttatacatgtattgaatctggttactagaattatgttcttcgacggacgtctttcagatttaaattgcattgtaggaaatatgatttgctatctgagtaacgtttttccagagtattcttgattgcgcgatctatcttcaattgttaaattgtttttgtttaattggggtcatgacaggtg

SEQ ID NO: 12 - DNA, 173 bp, Cucumis melo
  source 1..173: mol_type = unassigned DNA
tcgtataaatggaaaattgacctttccaactagattcttccagaactaaacaatacgtaacacgcaagtaatcaaagacacgtttcattttcctatagaatattatagttattcgtgattaacggaagtcggcaattttaggtataaatacgtgaattctcgagcgctaattt

SEQ ID NO: 13 - DNA, 2000 bp, Cucumis melo
  source 1..2000: mol_type = unassigned DNA
cttattcagcgctttcctgtaaaattaaagacttgatgagggagaaaaagaaaaccggttcgcagcttcaagaagacggcttccgaataagaatcagatactcgatgatggggaaacaataacaaagatatcaaaagaaatcatgaaacatagcataagaacgaaaacccagaggtgaagaacagtgcccaaacgcaactttacccaaagaacatgtataaaacgtcttccagacgttcaaaataagaaagtggacaaaatcaaagctacaaacgatctccaataactagatggaaaacactaattgcactagagattttgaatgctttgttgttgatttataatcctcgacttccaagaaaaagtaacaagtagaaatgaacgaatcagatccgcaatcgaagatctgaaggcaagataaggtaaggctaaagaaccataggaaaacggtaaaaacgtccaaaacagtgtgagaaatatcgcagattcaaaggtccgaaccctaagaacggtgttatgcagctataaaggtgagaatcaaaaccctctatccataacgtggacggcgcggttgaatcattgtcttgttccttgaaactgaaggtatgcgagacatagaattcgatctcactattatcttctaatcaacgacgaagtaaagaagtgaaatccagaacaaagaatggagaattggaaatgacaagaaaaacggcagaggaaagtggaaaagtgaaagcggactcacctagatcaatgcccttggctggtcgagcttcaggaacctgtcgtcggagagaaagagaaagagaaaagagcaagagagagagagagagagcacaaggagaagagaacgaggacaatggaggcttttgtttcgatactccctgatctggaattctataataacataactataaacttctctgggttggcccatcatcacgtatattgggcttttagcccaattatttgttcactgctcatgggccggtgattttgggctttcttctgggccttggtacataacaacccagtatatgacgtattttcggtgatagctattttcaagaacaccaacttttttgttcaacaatgtggagatcaaataacagtatgtatatatacacaaacatatgctcatttatgaaaaatagaaagaaaaagaatgttggtaatttgttacaaaattataatttctctctctttgtttgatttcatgaacggtgtgttctatataaaacaatgaaataacataattattaaaatgattcttaaaacatgatgatttcaatattcatggtttacatttggtgggatgattcgtttaattattattgataatgtatagttattgtgtgtcccgttttctttttctttggtggaagaaaagaaaaaagtaggaaggcatgtaatattgcgatccttcacgggacagatccattttccaatgtgatcgagtactagttaggtggagagtggaagaatcttcgtgcatgcataaatcaagtcacaacttgccaatttggaaagaatcatgttatattctacctttactttcaagtagggttaagtgaattagaccacaacgaagcaatcaagtccaaccaaactcacttaggtcaagcagtttagtgatatagacaggtcagtggtcgtttttttaatactaagaaatgtcaattctatctagttgactattattcatatagaaggagaaaaatgataactatgattgtcccacaaacaacaactaccgatcgatctaaaccaacaagtcgatgattggtgccactttaaatttaaatctgacgccactcaattgatgatcacccctattcaagcacacaagaacgcatgctcttaaaacatttggtaatatgattggaattagtacaaaatgtttattcgatctatacaaacaactcctttttaacacaaatgttttattgtactttcccgtgaaatggggttagtaaaactatggagttaataaaacataa

SEQ ID NO: 14 - DNA, 2000 bp, Cucumis melo
  source 1..2000: mol_type = unassigned DNA
tataacaaaatatgtgaaattagccattatgtttgtcctttcgttcttcttattcacttcgttgcgatttctttctatcgtctatcgtctttcttcttttttctgttgaaatttattttcatcgtttttcttctttttccatcgtgaaaaaaatagtcaaatctaaatgatcgtgtataaagaataaacgatcgtgtagacaaatctaaatggtcgaatattaagaaaattgataggaaaatttattcattagaaaaattttagtaagaaaaattagaaatgaaagggttgaaccagaaagaaataaaagtaatagacaaatgaaaattttaaataaaaagaaatttgggatgggtgcatttactatttagtcttgagttttaattcttttattactttacataagatgtattaaattaaagaggtaagatagaatttttttttaaaaaaaactatcattagtaaatttaacaaaagtgacatagcaccattttcgttaaaagaataattgttttatgtagtaaaattggtagaaatattttttaagtatagcaaaatatctttgtctttttatatctttcactgacagataataataatttattaatatatcatttatatagtcccattttcggtaaattttaatatttgaacataaaacactatttaaaataatgaaaaaaaactttacaaacttttttatttttattatatttgtaaatatttctaaaaaattttacatttaaaataatattttcaaggttaatacagaagaaaaaaaacaaaaaaagaggaaaaggcaatttaagaagaatgacaagaaaatcgggaggtggtgtggctaagaggaagaagggaccggttcttcaagatccaacgctccacattcaatctcacttccttcttcaattccgtcttctccgtttcctcctttatatgcttctctctttccctccctttctttctctccttcaatcaatcaatcaatcaatcaatcaatcctcccattcccattacattgccaaaaggttctattctcattctctacatccatttccctttctttccttcttcttcctctgtttcttcttcgtttccttgattcatttctctttgtacgttccttccttccttctgcattttgattattttcttttgttttacgtccggaattgcaatgtggtttatctttatttctgtttttggacgtcaagatgcctgttgtttttaacattttgatttgattcatcgttcatggcgtaatcatgtcttttggaattgtttgaaatccaaggatcacattgatttcactattgtttcatttgttcttttttgttaattttgtataatgaatcgtataggggatcatttttccattggttctcttgaaaatctttaagagttgcattatgtatactaagtctctcttatggcgtctgtttgagtgagaattgataaaagatccatgggaggaagaagttttctttcatgaggcttggttttattcagctgtttcttctcgttgcaatttgttgaagaagggacatgggtatcttttacatcaaagtataactaactatataattcaatttggttgataaagtagatacatgtaggagtcaaccgatttgagtgtataataatgttgttatgtcccttgcaacttaatgtagtgcatatttgggagtgattataaaattgtataaatcattttatgtttagaatcatcttgaaacacgttttttagtatttaaaaactaatttaatatttagttttgcacttttaaatgaaatttttgtttcactaatattagttttgattcattaaatgcatgctccatcgtaatattaaaagtaactagtgattttaaccattttataatcacatgtctgtgatatagtgaagtgtacggctgccttgttgagaattgttaccctttagaagaaacacaggatgtatttgatgtttaacttgcattttcttctggcaggcttagaaa

SEQ ID NO: 15 - DNA, 1990 bp, Cucumis melo
  source 1..1990: mol_type = unassigned DNA
tatttgtacaatgaaaatatttatttcttttctcgattctttaacaaaagttcaaaatcttttatcataaatacaaatatttagtaatttaagtttagactaggtgtatcagatgtgcaccaagtgtatattacgtgcatcaagtatatatcaaatgtgtaccaaacgtatatcacgtgtatcaagtgtgtatcaagtgtctatttgaagtcaagtgcattaagaatatatcacatgtgtaccaaatatatcaaaataaatactgattgagcatcaagtatgtctattagtagtgtatcaagtgtattaattaagtttgtagcaaaggtgtatcatgatgtataacgtgtattatgtgggttggtttttttttttttttgtcatttttgcaaaagtaattaagtttgtgttatgaacctaattttttaaatttctttttgtcacgtataagagacttgaaaataggtttaaaaggtcttaagggtattttagtttgacttttttaaaaagtatttatatgatatttaaaaattagaattttttagaaagaataggagttttataaattattcttttaagaaaaattgcatcagatgacaaaaaaaatttagaaataagcagcccataataactctttaaatttgctatcagacgactatccgagggttatcatcttttaaatttgctacttttacaatttagaaaatgtagtgacatggaccctattatcataagatttttttttgctatttttgcaaacacatgttcttttaaaatgacataattatttaaaataaaaatataaagttatttgatggatcttttgaacctatttttaaaagctaaagtactaaaaagatacatattgaaaacttgaggtcaaatgggctattattataaatatgtggactaaaaatgtacatttctaaaacttagagactaaatgcacatatttaaaaaagcatgtgaactaaaaaagtcgtttttcctaatatttttttacaacaatgactaaattgaacctcaaatttgaagggtggaaaaccatactaattattcactaatgaactaaactcatttgatgatttcaagacatatgaggttcattgagtagttgggtttgaggggatgaaatgagtggtggaagaaagtttatgtaacgacccaacgaaataggaaggtcatcccaaggaagtcgcacatccaatgagtaattaccacaaaacaacctctccttttttctcaaattcccttttaataaataatttgattccccattccttcctttctcccttggcagccttctccttttttcaaaggtttttgttttttcttttcttttttaaatttcattcctttgtttctctctttctttcttcattaacattcttcttatttcctcattactgatcatctccttttcttggtattattcttctttcttttctcaaagttttgtttttcattgatgtagatgtttttgtatcaatcaatggaaatttgagtttttcttatctcattgtatcatcattgagtgtgtgtttatgttagggatccattattaggatggatgagaatcataatttcattgctaatctatgaaccatgaataaagaaatctaaatccaacatagaagatagaacatttgcattgtgttatgagtaaccagctctgtcacttcaattggttcttctacacatttgatggcaatggctttgtttgatattcgtgatggcatctaagcattggttcttcctatgtttttcgttggctcttggtttgatttgcaattagtgaagagcatgtttggaatgaatgagttgaaatcacctttaacatttttaaaatcactttaaatattaaattaattttgagtgataaaagtaattttaacaatgataaaattactttcaaatgtgggccgaatcaaattgtctagaatgtttagggttctccaactaatagcaatttatccaaacagggtaaaaa

SEQ ID NO: 16 - DNA, 2005 bp, Cucumis melo
  source 1..2005: mol_type = unassigned DNA
ttgttagaggagttagcttttcagggagtcggttacaacttacaagacagacaaccatactgtacaatcaatcactactgcctcaaagtcgaaaccatttaataccagttgagcctcattgtgactgcataattttgacccctaccacgaggtaatttcagttcaaatcaattgtatctctcttctggatatcagtcaagaatctcatgttctcaagttatagtacattgataattactgtgcatttttattaatttatgagttaaaatcctgctgattaattcaactaaaggaataaaatagttattggcatttggatgagaatagaatgtgaatcccatcattcatcgctagattggaccgtaattataagtgagagggagaaacttctgttgctattccctttttatttcttaattcatttataaattgtttttaggccttttatatatatatatttctaccatttttacatttaaaattcttttaactttattatgtatggactcaaactaacaagctttatttgataaaattgttcaaactattatattagttttatatttgtaaaccataaaacaaatccataaaattccacctgcatcactcactcatgttgttgaatggtacgaaataaacaatacatgtgaaagatgaaaataagaattgttctcttattaaatctaaaatctagattttctttttagtacatttaacacttcaatgaaaggtctaaaacattattgaatgacgtcacgaactattacaaaagattattccgatttatctcaaaaggggtctatttcactaattttggtgtcccacatctgtaaagagaattttcgtgatatgtgtagatatttaaatataattgaattcgatgaaagcaaagcaagaatcgatatccgtagttattttgatatagatcggtgataaataaaagacaatatgcataaagtttgtgggtacagagtcggctagttgcaatgaggggtatggccccaagacttttccccaaccccaacggtcaattaatcaccccaatggggcatcgaatcttccccaccattttccttttcttcgccgactcttctacccatctcttttgccgactctttctcacaggtttgattaaatcccattcatattcagatacactatttcaaaataactcgcaaattaatttgttttttaaatattggtataataataaaattaattataaattgtgacctaaaaagtactttgtggaagaggggatatagtctgtctaatatattattgattaaatatataatagaaccataaatgcaaaccactagaattgttaataaaaatcaacgctcttttaagtctttccatagaaagaaaggcaaagacgcgcaacgtctagaattttctttataacggaagctaactctgttataacggccgtccacataatatggtagatttaaaatgacgtcagcatgtcgcttctaaatttgggtccaaaggggggattcatttatagggaagggaaacggcaaatgcaagcatagtgagtaaatacgtggcggcaaagtaatggataattctgcctctgccgtgacgacattgcccttccatttcactataaatacacctcctctatccctcgatttcttcgcaattgaatttctcttctcatctctgtcgtagcaaaccaaatcgatttcttcaaaggtatttcttcctttcctttttttttttttttttttttttttaaatcatgttgttcaaactttgagagatgaaatgattaggggctttcaaagtggttttcgtttgatatgtttcttagatcgatagggtttagaatcgagcatccttgtaggtatcctgaggtttggtggttggatctgcttaatttttatgtggttgcatggaaaattgggattttttttttctaattacgtgattctggaaatattgatctgtggttcagatggaattgaatctgatctttctgttttgttctgtatag

SEQ ID NO: 17 - DNA, 2004 bp, Cucumis melo
  source 1..2004: mol_type = unassigned DNA
tagaaaaaaattgaagatcttcttaatgctaataataaaagcatatataaaaaggcttcatattacacaagcatcttaaaaccaaaccgaacttgatttgaaattatcccactaattctgtagatttcaccaaagtccttaacctttatactaacatctgcatttgactctttcatttgacctccaacatattctttctcattttccttccattcaccacaaaaaccaacaaatacaaaaaaccacaaatacatagaaaattaataaaaaaatcaaaatttcgagatgaaatcattataaactcatccgataactttgagatttgaaaccttacactatataaagaaactcatccgataactttgaattcgcatcgaaattacgtaagtgaagacgaaaatgtaaatgattagatgcgaataacaaaaaaaacataatttctaaacgtaaaacactatattcaccttattatacttgattattttggaaaagtatgaaatttgagtgtgggagaggagccaaagaattggaaacttgttattaggagtcgttatgaaacattttcaacaagccaatattctttcacatactataatgatatacatagaaataatacaataatatttttgaaattgaggcatttttgtcgtaatttatctaaaaatgtcagggtagattcatcatgtatacaaatctctcgctatcaaaactatataaaaatcttgtgaatgatttcaatcgaaatggaccgagaaaaaacatcgtaaccacctctaaaatcgataaatttgtttcaatttcaaatccctaaaactaaaggtgctactttgtacaatttcccctgattagggtgctaaagttaaaccctaaataaaggtgtgtacgtttccggaagtttctagaatccccagcgaagttctccaaatcgtggcttgcagaccaaggacccccattaaaattcgtttcttcctctaaacctcctcccttaattttggcattttggtattttggctcctataaattcaccccctccttatccctaatcctttgtcttccaaattttccttcaaagcctgcttttcccatttcgtcgtgctttttcttcatctaaaggtatatttcagttctagttttctttctctgttgatctcttggatttgagggacgtttgaagttggctttgtttaattctttgttattcaatctctttttttgttagagttgttgtttaatcgtttcccttgttgtttttctcccttctagttcgattttagaacgctttttgtgggttgattttaatttctccgttttcttacatctttcacaaagaaacgattgaaatcgtgtttgttttttttcccacggcatacgttattagatcttgtagataatgatctcaatctattgtttagtttttgcaaataagaagttggttttttatctccaacttttatatattcgattcgatgagatgttctacaccgttaggatggaaccaagaagtgaggtaagggtgtttgattgaaaaattgaactgagaagttaaagttccttcctaactttttaatggattgtataattcgttcaattccttgtcgttccatttttatttctgtttcgtttttcgtgttgctgcgtatcgcttcccttgttgttttcctcccctattgattttgcgtttcttggagtttctctgttttctctcttcatttttctacaaaaatcaattctatttttattcgttttcaattcccgagctccttggaatgttatccttttctcctgtgtaaataagaacccgtattcaatcccagttcatagtttggctttcccaaataagagcaaaaagattgtactgagaagttgaagatttcaaaattttgtacatgatttcttctaatttatcaatttgattggactttttgtatatagatttggttcttgagctatttatgttatgacgttttcatattgaggccatgcgttgaattggtttcttaacag

SEQ ID NO: 18 - DNA, 1935 bp, Cucumis melo
  source 1..1935: mol_type = unassigned DNA
tatacaaatgacaaaatatcgttgaagtccaaaaaagatttattgttggtaaatatcgttaaagttagtaaatagattttagagaaaggagatatagcccttgtagtagaaacacacacacaaattgaattagatgtgtttaatgtttaattaaattagatatgagtcaacttatatctaatataggacattattaaacaaataagaaataagaaacatgaaacaagaaaaacaagaaatagaaacaatatcaaacacattcctatttcttgttctaaaaaaagaaaaaacatggtacaagaaataggaaacggaaaagaggaaacaaggaacaaatgctaccaaacgggcctaagtttctaacaaaatgagctaggtgtagtttattggtatagatagtgactttcaattattttaaatttttttatccatacctccacgtctttagaatctttcttatttatatgtgatcttaattcattcatgtctcaatcttaaaattagaacattacatgttcatcattttttccttttgttactgtgtttaatctttcctaacaagacaaatagtttaaccttaatccacacattattataaccaaattaaaataatctaccttcaaagaaaacattattataatcttatattaaccacaaattataataccaaactctaacgctccaacccaacctaggaagaatgacaaggctgtcataatttagttggtttggcacgttgttggaagttctcaaaattatggaaatatttatttccttcttctttatccatcatcctccttgggagggtgaatttgtgttaaaaaagaatagaaactaaagtctaagtggcaggacttacattatgtgtgtatgtggaagtaaaattgcagtaacagtttacaaaaacaactcatccatgattcataaccaacttaaatgaatataattttttgcctaaagattttaaattaatatataagcggaagaattaacctataacttcaagtttaacaacacaaatattatatcatactgattaattattggaatgatgtttaggctttaaacataaagtattgagaggctaatttgagtttaactcactaaactatcattaccctttcaaaatagatccaatcatccatttattataatactcaatgaaataaagcaaaagatgagtaaaataattcaccatgaacattgataattaattttcccactaagataaactactactcctcaaatcttcatatgtgtttttcctttttgagttgcactcaaattttcatagttgaaatttacccatcaaaacaaccaacaatctttcaaattcaacaaacatttgaccttacaccctttgatgccaaatccttaccctctccctcttccataaaaattcttatataaaccaccatcactctcacttctcaattcactctcttctctactcccaatcacctgacttgcctcttactccaccgccaggttccgccccaacttccccggtaagttccagttcttcagatctggttaccacatttgatttcttgcttgtatttgacgtgggaattttcatatcggcgttttttcgaactgggttttgctttatgatcatattcttgtagtaaaatgccatgaatctgttatttgattccgttttttttggagatcggtctagctttatggccatattctggcatttaaatgccatgaatccgtgatttggttgaatttcacttccgatccaatgtttatgctgatattgacatctttgcattcaatgcagaggagttttgtttcgatttattactgatctcatcacactgatcttgaattttttatacttttatgtgtgtgtgtgtattttctttaatatctatgccaattgaactatgtggttaacttcagagtgttcttgtgggcagtgagaag

SEQ ID NO: 19 - DNA, 1606 bp, Cucumis melo
  source 1..1606: mol_type = unassigned DNA
atatattgtatcgattctttagttgctctatgtttttgtttgcttcatttgtcgattaaactgtaaaattaatttctttgacaaggaaaaagatataatttaattctataatttattacaatctaatccatatggtttaataaaacactgaaaattgtttatgaaaattttatcgaactacaagaactattaataaagtttttttaaaccgtaaattgaatgaattttctccacggtgtaaatttgaaaacattaattaattaattaattaattttaatttcaaggttttttctgacccatgaacctattttatgatataagttgttcaggggttgcaatagtaaccaaataaagttgatcagaaaaggttaacaactcatgaaaacttccaaatgcatttgtgtttcaattattttcttaaccctctttttttggtaattttagtttaaaaagtgagtcggttgatcattattgttctttaatttcttgggagaaaaatattaatgttgattatggtgatgagttaagtccaattcttcatcaaatcataccaaattaggaacaaaaaaaacatcaattttaaggtgcaaatccatttctaatggctaaaatgtcaagcatcccaccaaaccaacaatctctaaacccatttttactccactaatctaatgtttaataataatcaacaaggttttgctcattccttttttagttaataatcatttaacaccaaagctcaaaagtacccacccaatggatcaaaatcgagaatatatagcatttaaggatataaagactagagataataataacctagcttagagcttaaagggatacactagccatcaagtcaatttggtagacaatctaaaaacaaataattcgatgaaaataaagttgtatttttgtgttttcaaacatgttttaagacgaaggtttttgataaatttgatctcaataggtaaacaatggtaattactcgattataattactcactaaataccaaatcgaatataaattattactaattaattatgaacatgttttacattttaaaaaatgaataattttttttttagaatttgtgttattgaaaataattttcaaaacaatattgaatgaatcttaagtgaaatcaatgtattaaaagaacataaaacataatctagatggtctatcgaacaagctagaaaatatcttccataaatccaatgattaagacaggcaggcaggcatgaagataagaggattggattaattggtgattttaagttatgaataaagacacaagaactagcagctctcctcttcttgtcaccttcctttgtcatccagctcacacaactccaacttggaatttgacaggtctctcttcactcatacattcccacatgaaattattaattgaatcttcaacattgtctttgattcttcagctgcactgtccttttccaccatttttttcttcaagataaagactaataaactccttatatattcctctcttcccattcacctgtgcatactcacaaagcaactgccatttccttcttgtttatctctgtttttttcttacacatttgttgaactttccctctgaaaaa

SEQ ID NO: 20 - DNA, 1487 bp, Cucumis melo
  source 1..1487: mol_type = unassigned DNA
taatgaatttgtatttgttagtggattgagtttatatgatattaattttgacccaaacagttgagtacgtaattaatgtggcttgcattgaaagtgatatgggcatatagtatgtgtagaatgtagctgacacaacacattaacaaaacccaattttaactttttctttttctttttcttttaattttatatggatcagatcacatgtcattttccattacaactcactctctaccaatcatcccatcccataggccataccccataacatccctttctaaatatctaaatcatctccctaaattattacattttttttctctcaaatataactattcaattcataaatattattctttttttagctcttattatttcaattatgattttaaatattccttttcaatttacgaccttttatttaccatatcaacattttaattctactcaattaaagatcattataatgaaatttcaggtataacaaaataaataggtgtgatataatgatggactactaatttcactaatttcgtcatctgaaataaggacaagttccaactatcactattgtgaaaacctcataactcctaaaagtgttaaaattggaccctcaagtttataataattttgcaaattgaatcccaaaattaaataatcagtataatttatacgttttgagagtcaaatttaatatttgaataagcttgaatacttaacttctaattttgaaaatttaaaaatgcaactgcgagagtaacttttgcaattagccgtcgaaacaattaattatattggttaatttatgtctcattctcttttgatgaccataaagataaacccatttataatataaatatcaagcaaagctaaaacaaaatcttttttttttcaaattagatctaaatatgaataaaagcagaactttctagaagtacaaatttgattatttttcttgagataaaattttcgctatgaacctttttataataggaaaaagagaaaaaggatggttttatataaatgtatgataaaaaggtaataatatccattgtaatagtaaaaaagaaaaaaagaaaaaaagaaaaagcaattttctttttcatgattaggaaatataaaaacaaaaattggctcccaattgacatctttaatcttctttttctttttcttagaaaataaaattagtgagagaaggaaaaaaacgaagggttgagagatagagagagaaaaaattgatttttaatttagtttattttccttttttggagcacaaaataaatagataaataaaatattagtttgcaaaaaagcccctcgagtttatcttatttgctcaaaaaagcaaggataaatacctcccgacatccctgtttatccctctcagtttcataattccattggttcgataagaaaacaattctcccaatattcccgctgtagatctcgtcgattttccgtttgtttcccgggaagatcaatcaaag

SEQ ID NO: 21 - DNA, 1448 bp, Cucumis melo
  source 1..1448: mol_type = unassigned DNA
ggtgtcttgttgtaaggaaatggaaagaaaagagaaaggctcttgttgttgtccttgttctgtgtatcgatgaaaatggatcgacgcgaagaagatgaaggacgagagtggggattataagacagagaaactccgaaatttgagggctaatatggtaataacaaatggcgggatactttcaatggacgtggacccattgcttctttaactcaccgtctgatctttattttacggtcatgatttccctctttccccaatatttttgggagggaaaaccaactttgtttttgtaattttaatcatttttcctcaaatcgtaaaaaaaaaattatagattttttcaaaaatagaaaaaattcatataagaaaaccaagataaaatattttgaaaaatatcctattttttacttcttaaaaataattcataaaagaattattataaatattaaaaaatatcagtaccactatagacaactatttatatagcacatatagatatatttgttggtttttctatttagtatttgaaaacaactccaaaaacaatacatttcaatatacctacgaagcatacaaatataattattaattttaataagttcaaaaatatctaatggcatccttatttaatcaattttttcatcgacgttatacacggtaaggatgtcctaatccttgaccattgaaagacgtttgttttgataattatatcttttgatatatacaaacatttatctcatgattagaatagtcacctttttatttgatttaacgattatacataatatttgaaaatttttaaatccatcaacacaatcaaaccaaaaatttcctaactacataatctacaagagatttaccatcttctttaaacaattggtcattacgtttgttaatgtttaaaattaaatgcaaccatattgggtgtaaaagccaaacattgatttgattattaaagttttttctatatagacttgatgtgtaaacctaataaccaacttgagctaaataactttaatttctaaaattcattaaactgtcctcatccaaattataatatcaaagatttttgaaatatttaaaaattccgaacatgggaactactggaacttggcaataaattcaagcaagaaagaggaaaacgatataatcaaacaattaaaaaacaacagaaatttatttaatcaaaggaataatctcatcttttatttattgggttttacttttaatactgtgagtgatgattggaacattaattaacatttaagacattaatttgcaacaatcaatcaaaattgtataaatccacttgttttgatttatttgaaccatcacttttttttttatatatatatataatatgggagtgaaagatcaaacgtataatcatgaaatgaaagatgggatatcattgaacttaattaaatatcattgaactgcaatttttt

SEQ ID NO: 22 - DNA, 1235 bp, Cucumis melo
  source 1..1235: mol_type = unassigned DNA
aaattttaataattaaaatgaacaatttttcaagagtaatagagtttgagagatgtcagagaagtttgaggaagaagataacaagtgggagaagagaataagtttgttgtgtgaaagagaaggggaaatttcattcaagggtatattgaactttttactcaaattttgtaagtctattttttccgatcaatcctaaaatcacacacacccttaaaaaatggattatatttggcaattttccatgataaactcatttttaatttagagttattttttcaacgagatattaacagttttagttcatatactaattgtaagaatagtttcttttaagttgaatagaatttttgaaacttttaatagttcaaaaggtatttttgaaacaaaataagaatgtttttgaactttttataaaaagaattgagatttttttgaaatttttgataaagagaaaagaaaagaagaaagaaaaaagaaaaacaagtttgtagaactccgtgggaaaatcgtcgagggccctgtgaaggaattttgaaattataatgagggtattttcgtcaacaagggaatttagacatcgtatataagcatcctcaaaccctataattaagcccttcaatccaattgccattctccatctctcgccgcaagggtttaagagcagcttctctcctcaggttggggtttccccctatcttcttcattcttcctcttctcgatttctttcttctatttgctcgatagtctcttatttcttgagcttttgctgtttttctcctgtacatcctaacatgaattataacttggttttgattttgtcttttacttctgtattaaacaacttttcttacccttttattcttctcttcttcttcgtgtccctgcccttttgtttttatgctaattttatgtttctgtttatcaatctatcgaggcgtgacctgtcgttcttccaatagcgtagatctgcacttaatctattctagctgattggattggtcgtttttcgtttttttaatttattttctctgttctagttccgataaatttttttatatataattaacaagttctccagccaaaagggttaatattgcgttggatattttaatttttacgttatttagatgtgtgaatctaataaaattagggttattcataaatttcagtaatgatattttggttatctgttcttgctgttcctgtttcgcagttcttttacctaatattcaagc

SEQ ID NO: 23 - DNA, 2003 bp, Cucumis melo
  source 1..2003: mol_type = unassigned DNA
ctagacatttttgtctaacctttcaaatgttttgttttaatcttccctctcccaaatagtgaaggacatccagtgtcaaccgtgaacgcatactgtgtcattcatgaaacaaatcttttttgtagtgggcattgtcagcatacatagcatgtagaagctatagacagatgttgctttgagttgtatttagttctctttaaaggaactttgtacaaagtactgaatgtactctgttatttgaaatatcaatgaagtcctcttaattcttttgagttcccattccacgtttaagttgttagttgtattcattttcgcttactaggtgttctgcatgtatctcacagagagactcacgtgaaatgtttaggcggtcacatccctaatgacttttgaaggggtgtgacacgatcatttgaatatatcacttatctaatagtgacagtggtctatatctttgtctatctgtatagatcattggttgtttagatattggtacactattgtgtagtgaaaaaagaagaagaagaagaaaaatataatacttgataatgagaaaagaataagaaaaaatattgtctttatgaaagatgaaaaaatgatgctgaagacgagaaatgacggaaaaggaataaattctagatgaagagatgaagaaattctagaagaacaaatctagaatttataaatgggataacaataaagataaatgggataacaaagaaaaaaaatcaagaaattacctaaatgtttcaatcttgctacgccttaattagaaaaagaaaagaaacaaaaaagaaaatgcacaaaaatatctatatatatatacacacacaagcacaagaaaaaaatgaatattggaaaaagacgaaaatgcattattttttatttgcgttagcgagttgttgtgattttgtgagcaagaaaaggatatgcaggagaattaagataaataaggaagattgaatagagattaaaagagaaatatgggaatagagtggggatgaaaggtttaaagatagggagggagggagcgagagagaggagaaacaaacataccttgagaaagggagaatgagagagataataaataaatacggtgatttggaactcataaaaagattaaaaaaaaaaaccttagagtaaagacttttccatgcatttcgagaaaatggaaaagaatattctattctatttgcttggacaccaagttcctttttgtcgcatgcatacgtctatttatttctgcttgcttgcataggcagtttttgtccaaggaaattcagcaaaggcggtatcaatttcgtcaacttagaatccactcagtactatttgaagttcctcactaccaatttgcaccatccaatctcttctctctccaacttcctgccagggcttaacctctcttaattccttatccttacttgttaccttacctggttccactcttcacgtctctctattctatattgtttttttttcattcataattttgttactctcttctctgtcccctttgtcttggattttatctctccatatattcattggaataatttaagttctttgtagattttatgaaattaccaatttaatttttcaaacagtttttggatttgtttaatttctccttctctaaatcgcgttgactttatgttatttcgcccttgctctgttttctctgatcataaagtatgtacttgattttatggtgaatgctttcttgatttaacaaccctggtgctgaaatcttttttaaatcctacttttgttgttttacatatgttcttactctaaaatgagcgacttatttccttttattcttccttcttgattaaggatttaatcgttgaagtatgcttatattgtggaaatttggttttaattgatcatacgagctagtattactagcttctcggtttctttggatgagttatatgcatatgatgatttcaattccaatttttattttgcaacagattgttttttgtggctgaaattcaagt

SEQ ID NO: 24 - DNA, 2015 bp, Cucumis melo
  source 1..2015: mol_type = unassigned DNA
gatcagagtagcagttgagcaaacccaaaccaaaccctttatctatacaatcctctcaaaataaaattattgtttaattattcccatatctattatcatttttccataattgcattgagagaaaaaaaaaaaattctaggagacctaaatacaacaacaactatttaataatagccccatgtcacattaaataaaactaacaaaaagtttaatacgtcaagaaacgatacttgtggatattgaggcatgggtccctccttctttgtatattcaaatctgtggtctgccatcagataaggcctttaccatataataagttttcaaaaaagtaagcaccacttgctgcttttttaatttaattatatttaatctaattattgaaacttatagttgttttctatccttattcttttcttctcttcaaacaccctcctattaatttaaaataaccaaacaacctttttctttacatagacaaacttaattagattaaatataacttgtaattagattaataatatagtttaaaaagaattttattttaagtagaattattagtaaaaatgaattttgtggatagatacttggaatttaagagaaagttaaaagagagaaaaatatgaaaaggaattaaatgattaaagttgaatgtaagaaatcaataaacataaattccatgtattaaatttttgtcggtgtgtgaataaataaatatctattactattagattacccagctttgtttataaaaagaaaaagaaaaagtttttaaaatattggaaaattttgtataattattgaagaaattgcgtggtctttgcaatttgggcatcgttcttatcgcttccaatgaaggggccgtttacctccaccactatttccaacttgtttttgtaccattctctatatttctttgacacctatattacacgtgtctttaatccattggaccttcgtcctactatatttttacccgaaatgacgaatctgtccttctcatccacctataaattcacctctccggctccttccctttcattcagttttcctctattcttctctctatacgtcatattcatttcttccaaggttcgtcctccttttatctttcttctttctttcactttttttcgcttttttcttttctttcggtttttgttcttttaatttcattcgtttctttttgttatatggtatgtggtatttgttgaattgagatgttttagggtttcgatttaggttttatttcttatcctacttaagggctattgtgattttggagaaaggagttcttatttgtttttttttttttcctttttcttatctggcagatgcaaatcttcgttaaaaccctaaccggtaagacaatcacccttgaggttgagtcgtctgatacgatcgacaacgtcaaggccaagatccaggacaaggaagggattcccccggatcagcaacgtctcatcttcgccggtaaacaactcgaggatggccgtaccttggccgactacaacatccagaaggagtccaccctccaccttgtcctccgtcttcgtggtggcatgcagattttcgtgaagaccctgaccggaaagaccatcacccttgaggttgagtcgtctgacaccattgacaacgtgaaggccaagatccaggacaaagaaggcattcccccagaccaacagcgtcttatcttcgctggaaagcaactcgaggatggccgcactttggccgactacaacatccagaaggagtctaccctccacttggtcctccgtcttcgtggtggtatgcaaattttcgttaagaccctgacgggtaaaaccatcaccctcgaggtcgaatcctctgataccatcgataacgtcaaggcaaagatccaggacaaggagggaattcccccagaccaacaaagactcatctttgctggtaagcaattagaggacggccgtacccttgccgattacaacatccagaaggagtccaccctccaccttgtgttgcgtcttcgtggtggt

SEQ ID NO: 25 - DNA, 2006 bp, Cucumis melo
  source 1..2006: mol_type = unassigned DNA
accaccacgaagacgcaacacaaggtggagggtggactccttctggatgttgtaatcggcaagggtacggccgtcctctaattgcttaccagcaaagatgagtctttgttggtctgggggaattccctccttgtcctggatctttgccttgacgttatcgatggtatcagaggattcgacctcgagggtgatggttttacccgtcagggtcttaacgaaaatttgcataccaccacgaagacggaggaccaagtggagggtagactccttctggatgttgtagtcggccaaagtgcggccatcctcgagttgctttccagcgaagataagacgctgttggtctgggggaatgccttctttgtcctggatcttggccttcacgttgtcaatggtgtcagacgactcaacctcaagggtgatggtctttccggtcagggtcttcacgaaaatctgcatgccaccacgaagacgcaacacaaggtggagggtggactccttctggatgttgtaatcggcaagggtacggccgtcctctaattgcttaccagcaaagatgagtctttgttggtctgggggaattccctccttgtcctggatctttgccttgacgttatcgatggtatcagaggattcgacctcgagggtgatggttttacccgtcagggtcttaacgaaatttgcataccaccacgaagacggaggaccaagtggagggtagactccttctggatgttgtagtcggccaaagtgcggccatcctcgagttgcttttccagcgaagataagacgctgtttggtctgggggaatgcctttctttgtcctgggatcttggccttaaaagaacaaaaaccgaaagaaaagaaaaaagcgaaaaaaagtgaaagaaagaagaaagataaaaggaggacgaaccttggaagaaatgaatatgacgtatagagagaagaatagaggaaaactgaatgaaagggaaggagccggagaggtgaatttataggtggatgagaaggacagattcgtcatttcgggtaaaaatatagtaggacgaaggtccaatggattaaagacacgtgtaatataggtgtcaaagaaatatagagaatggtacaaaaacaagttggaaatagtggtggaggtaaacggccccttcaattggaaagcgataagaacgatgcccaaaattgcaaaagacccacgcaatttcttcaataattatacaaaattttcccaatattaaaaacttttctttttctttttataaacaaagctgggtaatctaatagtaatagatatttatttattcacacaccgacaaaaatttaatacatggaatttatgtttattgatttcttacattcaactttaatcatttaattccttttcatatttttctctcttttaactttctcttaaattccaagtatctatccacaaaattcatttttactaataattctacttaaaataaaattctttttaaactatattattaatctaattacaagttatatttaatctaattaagtttgtctatgtaaagaaaaaggttgtttggttattttaaattaataggagggtgtttgaagagaagaaaagaataaggatagaaaacaactataagtttcaataattagattaaatataattaaattaaaaaagcagcaagtggtgcttacttttttgaaaacttattatatggtaaaggccttatctgatggcagaccacagatttgaatatacaaagaaggagggacccatgcctcaatatccacaagtatcggtttcttgacgtattaaactttttgttagttttatttaatgtgacatggggctattattaaatagttgttgttgtatttaggtctcctagaattttttttttttctctcaatgcaattatggaaaaatgataatagatatgggaataattaaacaataattttattttgagaggattgtatagataaagggtttggtttgggtttgctcaactgctactctgatc

SEQ ID NO: 26 - DNA, 2017 bp, Cucumis melo
  source 1..2017: mol_type = unassigned DNA
atggataaggcagagcttaccactaaccttctaagatattttcgtcgctcggcatttattcttggagggaaccacaccaactccaaaatacccatgaaacataggaaaaaatggttcatagtctaagtttccatttcgattcggtttggttcggtcttttattttaaaaacaataaatataaacctactaatttgatgatgacaagtttactaatgttaagtaagaattcatcaatacctaagaatttgcaagtttttcttaagtttgatggtaaggatttcgtaatccttgaaatacaacaattgtatagaaatgaagcgttgcatttttaacgtctatataggaacactattttactccaatcaagttgtaatttgatagataatagtttgtataacttaatgatgaagagcttttttttttatatataatttttattaatacgtatagttcaaaattggaattagctatcactaacacgtgcttgcgatagaaacaacaataaattcaattagtgtcgcatgtatttcatatggtattgatgacataagagtagtttgatacgatgggttacatggagtgacatgataattgtattaaatttcaatagttatgatctcaagtttgggttgtgtctcactttgagctttttgagaaattggcctcaagactcgcctaatttaatgttgcttcaagctatagatgcttacatcgtgtgtatgaaacatattgcactttgatgcttaaagttaatatagtgagtaactaaccagatattacacgctactcttttaaaatggtcaaataagaacatttattagtatgtgatataacacgtaccctccaattacatacaataattgatcaacccaaatcttgaggtatttaataataacaaatacaaaatagatggattatatatctgaatagctaaagaataaagaatatgtgttatgttgtagttacatagtacaataagtcctctcaaaattagaatggtataataaaaaataagaggtacattcttaaagaaaatgttatcaaaactgttgcatcataggcattttggcaggaagaatagtggaagaaaattcttaaacctaaattctatcgatattaaatagattttataagggataattgcaaatgtagcaattatatttaaaataattaagtatatagcaacattttaaaaaaatggcaaatatagcaaaatttgtcaaaatctatcgatgaccgatagatcatgtaagtctatcactgataaaccataggagtttatcaacgatagaagtctatcaccgataaattttgttatatttataatttttttaaaatattgctacatagttaataattattctaaaaattgctattaccaccggtttttaaataggacctaaatttaaggtatttgacataaattttgatgaaccaaactagcccaaatcaaagaagtttgggcccaaagcccaacgaatccacaacaaacaaagcccacacaacacttcatgaaaatgattttttcaaattttagaaaaaggttataaaatataaaaaaaataatcaaactatccctggtagctaagtagttattattatttttatggatacgaattgagtagtatttattttaaaataggataattgatcttagtttcacttgtgatgaactatttcactttattatttgtttgtaattcaataaaattagggtttgattgtcaatgataattattacaacctcaatattatactcagtaaagaaaaataaaaatttaaaattgagaaattaataccaattttttttgtgaaataaaaggaaaagtaagtaaatattataaaattttggacttggaaattaaaatgcattaataataatatttagtattattgaattaaaatggacaccggaaaccctaaaagagggagtggccacctataaaagggaagcactcatctcacccaaacccttgttattcccaattggccgtgcggcaaagaagcctctcaacc

SEQ ID NO: 27 - DNA, 1353 bp, Cucumis melo
  source 1..1353: mol_type = unassigned DNA
tgagggtcaaaggaggaggaagaacaagaagtaaatgaagtggagtcatgggaaaaggaaaacaaatgtgagaaaagaaagaaagccagagagggaacataaaattattagtcagaattacaacagaaaatttctgaagaattgagtttgtatgcagcaataatatattgaacaaataaggagagaaggaggaggggaaattcaataaacagcagaggaagaagaatggcgaaaacccaatatctaaaactagttaattcaacaagaagcaacacaatcatttcattaaaaaaagaaaaggtaaagagaaattcccagattcgttactctagattggtccaatggagtggaaagggatgcaatgaaatcagtaatagaaaagaaaagagttaaagtagtattggtaggtaccgattaaaaatggaaggcgtcggaaggaaacggagagttcaataaaaggaagattctttgcttcctccggccatttgatgagaaacaaaaactccgcacctccaagttccttccgggggaaggagaagactcttctattctggggtacacaccctcccttcctgctacagaatcaaatctaaattattttggattggaatggcatgggattggtctaacttccaatttctcgacacacaaccccaatctacccgccacctgtacccagttttcccaaaacgcaactcacattgcaattgcaattcttgtctttaataaatacaaattgatttttctttttctttttttttttttttaataacgattaaccctaaaaaaaataaagaaaagaaagccgatcctaaaagtagaattactttttttttgtttttcaaggttcacgtctgtgtttgcatagacgtgttgtagtcggtgggtgtgtaaattagagtttgtttttctcatctcttgttctttttaacgaaatttcaaagatacaaaagcataatgaagaaaagtatacaaagcaacgtaaacttagcattttgcacatgatacaaatttagtcaaactcaaaccctggacaacctagcactctcttgggcacgtggtagatttatgtgaatttccctatttttcttttgaactcacaaatgggcaaataataataataaaatttattgttgatttttcttatatttcaatttattacctctagttttaacctaaagtttagatgtatataattataaatgagcggtgaaacgggcactgattgatgaatatattgggccttgggttggcccaacaaacctaatgcccaaatataaaactttggcaaccatagttaaccctaatctgtcaatctactctcctcgactcggtaaacctgcgactcccaca

SEQ ID NO: 28 - DNA, 2005 bp, Cucumis melo
  source 1..2005: mol_type = unassigned DNA
cagtgtgctggaattcgcccttatccaaggagattaatgtcgagagattattatcgaggtttgaatttattttgtccaatcatatgattccaagagctgaccatcaattcaacagaacatgaaccggaacctcatacctattgtaatggttcacagcatcctaatacagaacatgaaccgaaacctcttacccattgtaatggttcacagcatctttatacgtattataggtagtaccattgaagatgcatttaaatgctgtccatgctctgttctctaaaaagttggacttggacttggacgtcagctgaaagtatgaaatgcactgtagccaacgaagctatgttttcaggcttcaacatggttttaggaaagtggaggctctttggttgaagggttgaatgaatgcttttctaattccagcatgatcttcaaatttcgacacaaaaagcttaagtattttgttccgttattcttttaatccttgtattgttatatattcttttctctgaactgaatgtacgatgattgcaggggtcgagagcaagtccgatataatgaaacacgtaaggacgtgattgaatgaaaaactatgagcagagatacaaagtctaacttacgggatgaacgatgagaggtttgaccaagagctgtgacgcctgtatatttcaacaaaagttgatgactaacatcacatgtcagagtaatcaaagaaatgcagccgcacatatatatatctatatatatatcgtttagtttttttttttttttttttatttttttttttatctaattatattttaattctattttcctctgccctcctccccctcctcttcccccacccttcttctgcacatagtagccaaggattgatcggtttcttttgattcggggggaaaatgttgtacaatttttgcttccatagaagcttgaaagttttgcagattatgttgtaaaattacccttgtgtactcacactagttcttctcgtggaaacttatattacaatggttgagttttaaggggcatattcacactggtaactaccattttctaatttatgaatgccgagtttctctccatgaaagacctttcaaatgccctttcctccgcggtgcgtttgttgttgtaaatgtgcagtgtcgttggatacacgattgtgtgaaagggaaaagggaatacgattaactcttaaattcaacccctatctccatcagtatcaatcacatttcagcaactagctcttgaataacattgagattcttgtttaatccacgtactactactactattactactatttgacagccgatatctcaaataacatccatatttatcaaattggtattttaaggacttttaatttcttcgtacatatttcattataatttaactactctgaccatcattgaaaatttcacaaagaagacattttaaattgaattgagttgaattaagttgatataatggttgaacgttggatttaatttataatttagtggtgtatgggtccattgtaataattcttaaaaaaaatatcatattctgaattctaaagaaccatctaagaccaaaactaaggggtcaccaatgagtatggtaaagtcaacaaagtttgtctacttttcttatccttatcatcaagagtgcaatatgatatcaaagataaattgtacgtgggcgtcatccattgggtaagaccaagaagcaaaatatcatagagaagttgttttagtagccataggaaggaaggaagcaaaataataatatagatttgaaattgtggatgataaactgccaaatgggaattcaaaataaactaaataaataaaataaaaagagaaatcttgggagtttccattttagccaatgaggaaacagatagagatctcatcaagataaggaccctattctcttcttcatctataaaacaaaaacaaatcaaaccctcatttcactcattcaaaacaaaaagtactccaaagtcaaactaacaaatacg

SEQ ID NO: 29 - DNA, 1445 bp, Cucumis melo
  source 1..1445: mol_type = unassigned DNA
tcccttcagccacttaacacttaaaaatcttaggaaactccattggctcctctttctccaatgaaattttgacatctgtgttgttgatagctcctatatcctttgagaatttgatatacacctgtcatttgctgatttgtggataactgttagccttttgatattgatacgttccttttttatgaatttttgtaaatccattcaattttaatgctgtcgtaaatgaaaagccctttcattaatgttgtttatatacatattttaaaattaattcaataacaagtttagttctgttagcttctaggtttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagataagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaaca

SEQ ID NO: 30 - DNA, 1282 bp, Cucumis melo
  source 1..1282: mol_type = unassigned DNA
tgcaattaagaataagctaatcttaatgaagaaaagaaaatgttctttgtatttgataaatggtggcgttttgggggactttatatgctttttttttcccatgagattggttatcttcatttccgtcatgatgtcgccaagtggcgcttcattgatgatatcttaaaatctataatgatcatcctctttgccaatggtgtggtgacacgtggaaggactctccatcttctaaaagattcttcaaataaaaataaataaataaagaaaaaacttgtaagaagatacatatgtacatttttatatgaaattaatatgagaaataatcgactttacagtgacttgatcaaactttcttatttgtttcatatgttaggttaaattactaatcaattcacgtactttactagatgagatttcacgtactttactcattgagtccaacggttgattaacttatttcaagaaaattgattcattcaaggatgtttccaactctcatataatttccatgttgttccacttctatcaagtacaatcctatcgaacacaagtttgtttaactgaagttcaataatcgagatcaagataggccttattatttcttctagaggttcaagtgatcaatcaaaaaaggtttatcacatgattcattccaattcaactaagctaataagtggtgttgcatgatagagtatcggactagctcgaacccctatcaatatgataaatgtctatgtatataaataggtacttaacccaacgaacaatgtgtcttacgtgagaaagcttttttctaatatacataaaaagcttgcatgactttttgatgaattgtgttttgataaaacatatttgtgagtatattatctttataaatttaagttataacaacaatgtataggtgtgagtatgcttttaaacttaataaaaaaattagaaaaaattacctttttagtatgaaagttttaatgatatatcaatttgtgtctttatgatcaaaatgtatacttttagtctcaaatgtttataagaattaactccttaataattatcctaaacaatcatgttcaaacttggattcttattgacacatatttcattttaatctaagtttagaaatgaagataattaggataaggatctttagcttatgatatcttatccaatatcttaaataaatcttcaacaccaagaaatttccctattgcggatatttcaatatcgaatgccttggagtatcaaaggcattggataacaagtgggacataattgcgataaaaaa

SEQ ID NO: 31 - DNA, 2002 bp, Cucumis melo
  source 1..2002: mol_type = unassigned DNA
ccgtagattgaacttatggcttcggtcagttattgagattttaattctctttaacattaggctaatccatgagtttacgtgtgctaacatgttaatatgaaaagtatagtagaaaagtaaaataatataaataaataaattggattgttttgaaaagttgaaagattagaatatacataagattctgaaatatctcaaatttttgacccagcaaactgaaattagccaaagtaggttgtgttgtaaataattatactttatattgttctttttgtataagcttttatgtgtcaatgacaattttaacagctaaataatttaaacagaatattgccaagatgggtggctacaaaaataattgtaaatagaacccaataataattagtttaatcaattatgtttttattaacttgtaaattaaatttacactgaaaagttgaaagagtttggaaaatattttatttgaataaatcaaacaattgaatactaatttgcgtaaaatacgtagtttaaaatatatatatatgtatatatatatatatagtgtaattttcaagtaataaataaaatgaaaattaaaggtttaaaaataagctaatgggtgcttaaagtatctacggaaacgagattgcattcgactcacgtacgacatgaaaaagatataaatgaattttacattaaaactattaaattgcacatatgattgtccaacaagtaagaagaatcacaatcaaagtaaaaagaatcacaatcaaaagagaatgtatctaatggatgatgacaatttacttaagatttaagaattaatctaaaaatttagagagaggggtaaagatatcaacttttatttaccagaactaaaaattatccttaggcctcaattgctttagtaatggatatatatatatatatatacacatctacctaacaaagctttaataatagtaataataaaaatttaaataataaataaaagaaatcgaccaatataaaaacatataaaaaatgtatagttaaaaagaaagagagaaagagagaaagagagaagagtacatgcaagagatttgatttggaaggagcacataataggacaagagaagggtaattttggaatttgggtcaattattcttagtccaagggttacactacaaaaacctaacagccttcacaaatttttccctctttcgctcgcttcgctttgcccaaacactcgcctccaactccacggatcagatccgaagagtttggcaaaccctagcttcctctcttcaatctccatctttttcttctctaacaatccacaggtttgtttttcattcctttctctttcgattttgccttcctcttctacttattcgactgcacgaatatggttgtatgtatgtttccgccctcttttcatatccctttttgttcctttagccttgaactactctgggttttcttttctttttttacttttttctattattgtatatctcaagatttgacgctaatctggtctgtggttgtgggttgagttcgtttttattcgtttgtttgtttgtttgtttatggccatggcttgtaattgcttctgtaatctacgtgaatctgtttttgctttggaacgtttttgttgttcaactcatacgagaatcgtcgtctatagttgggttgggttttttttttcagtagcatcttgctttgggaaaaggttaatgcggtgtcttttttttttttttggagaaaaaaagttattagacatccctcaactccttttcctacattgagacagaagtttaatgcttgttttcctctttatctggattgcaagtttggcttttctgttacagatttcctttctcaggatagctttgaacagatttgtaatgttgttctgtttattccttggtggggttgataaaatggttatgattttttgtttgttggcggcataattctggatatttttatctgtttggtctgtgttcatatttgcattgttttccacttacagct

SEQ ID NO: 32 - DNA, 2003 bp, Cucumis melo
  source 1..2003: mol_type = unassigned DNA
tggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtccttgtaccgttgagcttcgccttctaatagagctctggttcggttggcgtattagctcgaattctttctctcttccagatctacgctgccgatttcatcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagag

SEQ ID NO: 33 - DNA, 1372 bp, Cucumis melo
  source 1..1372: mol_type = unassigned DNA
aaataaaacgcggagacaaacttggacttccattcccttcttcttcctttttcttgtaggaattcttctttcttccttataaaattctcggaccctttttttttcctttttaattttatttttccttctgtagttcgtttcttgatttagattttcgacaaaggtaccttttacaggtttgttccttctcttcatcgtttactccgattgatgcatttcctctattttcacttttggattggaattattacgatctatgttcaatatcgtttgatccattccctagatggaaattatgtctctgtaattatacatagtgtttgatttgtttgggaaattttgtttctttgtgataatgtcttcatcgatttgatgatgtatttgtttttttttttttggtggaatcgatatgatttatgatttgggtgtttttttgcttttgagaattatgatttgatcagagtttttcttattatttctgttgttttgtttcatttcctgccgtttttaaagatgtgtttagattctggttgtttttgtccttttgattatgtttttatttttcatgtagttggaaatcaataggatttcagataattcatttggttgcatagggatttgaggattggaagttcggcactctataactttgcagtgaatgatttgggtgaagtttttcctcttgtttgtgctttcatgcttcagttgcctcaaccaatatcgctttttggaagtcttgaaaatctgtagctttgagcttgtgtttagttcgcaactgaagcttcaaggaaaaagtaatttctttcgattttcgtaaaaggggggaaaaaggaagtaattctactaaaattttctcctatgaactcgtaggtcacatagttgttatttggtcagttgacactctagactatcttgttaccattccacataactcaaaggttttaagaataaactcaatatgggaatggtttcattaggattgcagagtcaggaacaagagaggttgctttgcacaagttacatactttctattcttagggagaaaagccagttgtcattgttcagggagaagattaatttggttggaaagatttattgtccttctgtctttaggttgtcattggtttgttataattaaagtttcttgtttcctagaaaatagaagtttttccctatgagtaatgttatacttcattgtcttttattttgtgacaagcaaacagtgatttattggatgaactacagttaaattctgaatccattaatttttctgaaatccattgtgattagaatcatgcaatgccaactgaagaaattttcaccaattattaaatgaatatgtttatttgcagggtgttttaaatagatcaag

SEQ ID NO: 34 - DNA, 1122 bp, Cucumis melo
  source 1..1122: mol_type = unassigned DNA
atatatttattgctagggttttccggttcctgtttgctccactatttcagccgcctagggttgaaacaactcattcctccgatttcaggattactatcttcctcctcgaccttctccggtaatactttctcttcacaccccttttgttgtttgtgatttttaacttcctttggattgaaatgcgagatctgtgtgtttctaccactcttctttcttaacttttcgatagtattgcatgttccttacttatggagaggataatgtgtacttagggatatcaattttcgttcacagtattcaatattcatgacttactgaggtgtgaggagttttcatttcatagaccgactgatgctatgatctcaagccgagtttgacccctgtttttctttttatattctttttcttatttttgtgtcaatatattaggtgatcaatgacatcctaatctattattagtgaattgagtaataagaagtaaagtcttgtttatccaattttttggtttggatttattactattttgttggaatgcttgaatgaattctaatggagtccgtagaaatttgtttcaggcgtgcgccttttcttctcactaaatttttcattaggaatgggtgtatttattttcaggagaatttgtcgattggcgatagttgtcttgttctttttcatttcctttataaattctttatggaaaaaatgtatttgctgcaacctctgtcttattacccctatttgaatcaatagagttcctgatccttcctacgatgtggtttctggggatttctctctgggttcgtgtgatagatgggtgaccgagggaacaccctttattggaaatgctcctattcttcagagtcggtttctcattttctcacctttacgctttgctgctgctctcattgacagtcgaaccgttttggaattcgtgatattgtgtgtattttggggatgaaagttttctttaataagactagtgacagttcattattgattgtggagaaatttatgaccatctaattttaatttgaacaagggaggatgaaaatgattgggcgcattgcatgttttatccaactagtactcattttttctttgttctgatattcttcaggaaca

SEQ ID NO: 35 - DNA, 2017 bp, Cucumis melo
  source 1..2017: mol_type = unassigned DNA
actattggagacgacgaaggaaaactctaatgtgagcttagaattgaagatggtgaacaattgggagtcttttttaaaaatctttcgtcggtatattgaaatttccttttacactcaaataaccccccttttcacgcgttcaacgtcttcaaaccttcctttttcaattattttttcgtttacattaatgacatttaggtaataagataaattagtggattcttttgtgaaaaatgttagcccatttaagacttttatgaaatatattagaaaaaaatcatatagcaatttatattattctagttaaaagcccataatagaaactaacccaacctaaagtaacgtaaacattcaataagagagacaaattttaaaataatttctaattaaaaaaaaaattgtcaagaccgtccgggtcggatccaaaatataacccgaaccggccggtttagcatctatataaatacacctttagggtttttattctctcaagacttcaccgcatttccacttctctgaggccacggtcagccattggagcagctcaataatcctttgactccctactacggtaagtcgaccttactgctttcggcttctagttttttcaatcctgtcattagtcctttggagttcttctgtacatttatgacgttttcggctcgtgttttgtttcgcctgtatgtagtgggtttttcgagttttgtttttacttttttttatacttgcaggaattagttgaaatctatgtacttcatgccttggataatactcttgatctgttgtgttattcaaaaatgaattgttttaagatggtatttgagaatggtcatgtgagttttgcctacttggttattaaaatgaattgttttaggatggtatttgagaatggtcttctgggtatttggttggaacctttgtgctctgctatgaattagggtgttctccccgtttttttttttttttttcttttggttattaatatatcttttatgactacttattcatatatgatatcttttactcgtaaattttgactcatttgaaagttttatccttagtcctttctcattcagggtgtaaaggtatgttgttagggttaaaatagcctatgcaggaaagttctgtatttgttctaattattgcatttgtgtgcatttgtatctagtttatttcttgctgagagtatgcttcattttttagtacacatcacttgtgccactttattatagttgcacatttttgtttatggagaggatgaatagcatttagggatgtcaattttttattgagaaaaccctctctcctacttaagcttggggaatttttgttctaaatgtggtaaacataatacttcttcttattttaatttgaatggaaggggaagacgaatactaatattttcaacgaaccttcacaacttttttttcttatttaggaagccatgtttttcaaaattgtactgtgtgatccacatatttatcgattattagtgaatcgaataataattagagttttattggtataattttgaagttcagacttattacatttgtggaaagtttggttacaattttcaattttattggaatcctaagaactttgtgttaacatatattgagttttcttctctttttttttactcattaagttctctattaggaatgtttggttcaatgtcacatagtcgatagctaagaccagtgacccacaaagctatgattgaacgaaaaacaagcctttcacatcttggtaggaatttgttatttctcaatagatttacagagctgtttcatgtgatcacaatttttttctatttttctgaagttctctattaggaatgggctatctggttagttgcttttgagagaacatgtggattggtgttgctcggtttccttgcctttgtaattttgtccttggaaaaagcaaaatgattaggtatcctgatatgcataacatgtttaagccaactagttctcacttttttagtgcaaataattgatcttcaggaatc

SEQ ID NO: 36 - DNA, 2000 bp, Cucumis melo
  source 1..2000: mol_type = unassigned DNA
aagttgttgaggctttcaatccaaccaaataattgtttcgttttccactacaatttcctagtactaaggtagcaatggatcgatccatagagaaccatttgatttttcactaaaatcaatggttgctaagtaaccaagggaggattggttgaattgattcctaatttcacttcaataattaaagcaatggcaataaaacaaaaattggaagattgttgaattaaatttagcaatgagataaatacctaggccaagacttacgtaggttactttagattcacaactcaattattgattcataatgatattagattccttgcaacatatgaacaaaatcttagttgaccacgtctagagaagctaatgtgatgttctataaatcaaatcaatccttatgtctagattaaaagcatcctagagatgaaaatcaattggcattaaggtttgaggctaaagctaagtcgatcaaacaatttggagttgtctaattgattgttcgatgtgatacaattctaaactagttagataaacgtaattagaatggaattgtcaattcaataaatgattctaacttagcttatgttatcttgcagtctaaaaataacaattacatattagatctagatctataacaattaattaaacatgcttggaaaatcgccaatatttccgaacacactcaatcaaagaaataagtccaaggaaagaattcattaaatcttaagattcacaggatgaaaatgttcataacatcacacaagtgtgtgaatcaaaagataagactagaatctcgagataatagtaccttagctatgatacatcctcgaaaacatccaacaaaatcaatgaaagtcttgagtcaattcgtctagtaaaatacgaagagttcaagagaaaatgcctaaaatttagtgccaaaaattgtgtaaaaagtgttggcggctagggtaataatgcaaaattaagtcacagcaccgcaacaacgtgcaaaacacatgtgctatactctcgaaaaactctatagcatcgcagtcaacacgataccgctacacaacacgttgtagggctgaggtgtttgcatgaaattagaccattctaccttacagcatcgtgccttcttcgttccatttcaattttcttgccccagttgacacactaaacctccaattaatctcgtttaatataaaagataattatgattttctttatctacgaacaacattattgtgaaaagatataaggatgatatatcacaatttttagggaaaaaaggaaaatatattggcatttattatctctatcaaatagatgattttacaattatatgttaagatgttttaatccttgctaatgtgaatatttattttatttttgttcacatgaaacaatggtattttgtacactccaagtacaatagtttctttaaaaaaatttaaaatgatacgtaaattatctaaattgacatcttaactaagcaaacaaaaatagttgtttgaaaactagacttatttagtttacaaaaacatgcaccagatatcctcacttaatcactagctctacacccaaaatatagactaaataacttcacatataatatacaaatttaccaaactcaattcggcatctcaattggcgaaagatctttttaacccaaaagaagacgttggggcattaacttttcaaaatgaactttggcttcatagtaggaaattgggagtgaaacatgaggctgaaaaagggctaacaaagagagcgatcgtccacgtggttggcagtcaagaggtctttatagaagaggatgaagaacttgtttcccattggtccgaaatctatccaacaccctcctattagatttcccttccagattctcatcttcatgattcctacttggctccatttaaacccacaattcaattcacaatatctcccacacatcctcttcttcatcatatcataaaacacagtccgttacatacctgaaatcttccatctcaaaaacc

SEQ ID NO: 37 - DNA, 1760 bp, Cucumis melo
  unsure 179..195: note = unsure at all n locations
  source 1..1760: mol_type = unassigned DNA
ataatgaatagcaaactacgtaagttaagttttggttactcaaatttaaacgacgtaaaaaaaagaagaagaaaagaaaaaatacgatggaaagaaatcacagagaaaaaaagaaaggaaaaaaagaaagacgatggaaagattaaacgacgtatagaaagaagaagaaaagaagaaannnnnnnnnnnnnnnnngcagcgaaaaaaaaagaggaaaacaataaagatgacaaaagaaatcggagcgaagagaagaaaaagatggaagaataaacttgaattggacaaattttatggactttttacatagacactaatttggtttttttgttagcttcctacaaattttcctcttttattttatttttgtaaaagtaaataaatatgtgtcattagtctaattttttgaacttattttgggagagatagaggaagactttaaaaaattattattactctccattttaattttgagaagagattgtgttgtaccattcctcttattgcttccaatttctttgagggcagccctagcctttgtacaacgcaagcttcctgtagtatctctatctctctctctccctcttccgacggtgatctcttttctctctctcacattcatcacccgccgccgccggtagcttctctttctctgacgccaccgccgccggtaatctctcactcgtcgctctcaacacagagaaatttctgattgagcatcaccaggtccggcaactaacattccttgcttctgcatctctttttcttcaatttctggtatagttttgatggatggattgtgtgtattcaatcatttattgtgttttgatggatgaacccgtttattattcttttttatgacttcaagtaattgcaactgggtacttctatctgcaacttcttggctgaagtaatttttagttaagtgcaaacggacgggctgggaccgagccaatctaacgcttattttatcgaattttgaggagttggttttgtttgggtttatagcttaggaagggtttttggtttcgaagaacctaccatttgaagggttgggtctaaaatgtcgcttaattcgacccaatatgactctgaatgttaaatattgaatagaaaagaaatgaaatactatccctaacctgtctgccaatttcgtgcaaaaaagcctaatagccagttttttctcgccggcagtacattcgccttccccttccaagcgctacggactgttgctcaatctccagaatctctcaattcgcagggggcaagttctttccatcaatcattttatgtatttttgcttctgccctagatcgttcatctaaagttctttacctttttcttctgttttgttttttggtgtataacttatttgatggtgatggattatgattcagtatcattttcttattttatatcagcaacaaatttggatttgaaatcattttttaaataccttttgatgttaagggtttaggcttattattatgattcagagtcattttctacgtgttaaattagtttactttccaagtatgcagttatgttcaagcagttatgcagtcattttctgatgtgggagatagtgctgttttccttaaatgttttctatttaaaccattgtgcgcttggttggtggccgtgcagataattgcatttctttttttggattggggcaggttggttactctctggtttaactttcacaaagaaccaagacagacatccgtaacttgtttgcataaagacattcaaccaag

SEQ ID NO: 38 - DNA, 1767 bp, Cucumis melo
  unsure 456..459: note = unsure at all n locations
  source 1..1767: mol_type = unassigned DNA
  unsure 832..834: note = unsure at all n locations
aatataaataaatctcattactctttatgagctagaaaggatgcctaatggacctacagactagaagctacaacgatatgagattaattggctaaactcattaaccacattatgatatatttgttaactgtgtgtacactccactaaagactcgcagctgaactcttctcactgtagatatatttatgtgtccacggatatagaccaataccaataagttagtccttcacaagtgttcataacactagctgggtcaaattactgttttccccttgggttacttctagtccttaaataccaatgctcctctaatgaacaacctgtttaatgtccaaccactaaacagaatcctttctcatgccatagagagggtaagaccttcaagtcctggatacaccatttaaaggagcgcttatctatttaccataaagtcaagaaggagtgaattccatcttnnnngattatgttcccagctccccacccggttttgtcctcaaaatgataaatatattgagttgacaatctgaccactctcacccgtacaaatcaaaagacaatccctcgcgaataggagttcataatatactcataattaagactaagttatccatgtcattctaatgaaatagaaacccaactagttaatggagttacatcttgtggttactatttcgtggtcgggtcttatgcaaactcattacatacgataccctcactcgcatgtcgcttacttgaacatgttgaataaatgcatttatattagatacaaagtaagtcgtatccatagtgttaccaggataagttacctagccttaaccctatactatagacnnnttaagctgatcttgaacattgtttcctgtatgtctctacatactgttcaagactcatcaaacaactcaagatgttaatttattggatttaggttattaagataaaacgaataatataattaataacacttcttgaaattataataatataacactttattaataactaccaatgaattatatttactatatacgagttttaagacataaaatccaatataagggtgtatgaactgttaaagatgatgtgctattcttgttggatattataggaggtatttagtggattatttgtgaaagaataaggaggtacttatgggaagactgctggaggttagggaggatctttgaaaattaggaagtagggatcaacaaaaaaaacgaaagggaaagcttaaagcttaaaaaagaaacgaaataaagaaaaatgatttagaccagcatactaaaatggcaatgtaatctgaggctaatgtatcaattgagaactttgtagtcataatgattaatcccaaacaaattagttttcaagaaatcaaccccaaataaaatgacttaaatattgaagagtttaaatggtctaaaattattgttactgttttttatttttggaaaagagacgaaaaaggaaaaataagaaacgcccaccgtggggctcgaacccacgaccacaaggttaagagccttgcgctctaccgactgagctagacgggcttggtgtccaaaaatccaataatattgaaaataccatatagtttaatgaactgggcaattggaataggcccaatatattagatatagcgacccaattgttaggcgtgtcttcttccaaaaattggaggcaaaacacaaaccctagcatccgcttctgctcctttatcgtttctctcggcgatcaattttcacggagctaggtttaatcaagcttcaagca

SEQ ID NO: 39 - DNA, 2000 bp, Cucumis melo
  source 1..2000: mol_type = unassigned DNA
tttaaataaaaataaaaaccatctctttattttaagtagttaaatgattgtcgtttactaaattaactctagcctattttaagacggtctggtcaaaaaatcgattacgaccgaccaatattcatctaacggtcttattatttttaaaagatatagaaatgtatctcgttaataaagccacgacggtctttttctaataaaaattcaactaaaccatataacaaaattattgtaccatgaaaaacactttcatacataatgcaaaacaacaatagcaaaaaaccaaagaggaaggggacaatttggggaaaagtaatctcaaatttccctttttgactttgttctaaattagtttattgaaataaaatctactaatttcccatttccaaattaaaatgcttaatctttgtctcattagcattgctagaacaaattgtcttctcaaaataaaaataaaaatacaatatcaactatttatacttaattatctaacatttctccaacataaaaagagttatatatatatcctattttgttctctaatttttcctctttttttggtaattaattataatattgtcctaacatattatattagatagcttcgacaaaccgttgcttaaaaaaagaaaagagaaatccaacctaactcaatccgaaaatatacaaagtacaaaataattataataaggtagatggtatatgcatcaatgaaataatattgtcaactttcctcgatgatgatggtaataataataataattttatatttattaggcgtaatattttcctcaattttagtgtttgtatatactttcatatgtttaatttaagttttaaaatttagtccctcaattaacttgaaattaattaaagaatgtgaaaatgttaatgggtgaaataaataatttagagaaaaaccaaaataaattagaggtagggagtaattttagaagttcaaaaaaaaaaaaaaaataaattagatgtttgaaagtacagatttgtttaaatatgaaccaacttcgaatagtctttccattttttcttataaaaagtctttctgatgtggatactagttagagtatcctatcaactcatcgatccaaagaacatactttcaatcgtaagtcgtccattctacttcgatctaaaatgatgctaggtttgcttcaccttcacccttcacaaagacaagtgcaggtgtgcttcgctctatcacatgattttgattatgtcttcaagaacttcacagcggttttaaaaaaacaagaaagaaaagagtgagagtgtttttatgtcagaaacatatgcccaagcttatgaaacttgttgatcttgtagcgattgaataacaaatggaaagtatctcatacaatttctctatttttcacttttatcgaagaactttgtctcactaactcgtaatctaaaatacaaactcttcgactctaatatattaactccaaacttcatttttcacatctatggaacagataaaggtctaattttttaaaaatatgatgggaattaaagtatagtaaagagattagcttcatcaatgggcttggattggagtccaaagggttagcccaaacccaaaacatagtaaatccaagccctggaacaatgaatagcacggaaagtttgtgctgccggaggagcgtattggaaatgaagggtttaggatagttatggagcagaaaacgacaccgcatcattaaggacggatttgggattttaagaatatattagggacagaataggaatttgaaaagtagccctagccactcaatttggtaacagtagcacaaaaattggaggatacctaaggtaagcgacatggggtaatacacagaattgtggctatggcagaattggatagaactcccatttgaggctctcttttctcttaccatttctacaagataacactactcttcttcactctccaaaaccccatcttcttcttcttctcttaggttcctctctcccttcctcca

SEQ ID NO: 40 - DNA, 2000 bp, Cucumis melo
  source 1..2000: mol_type = unassigned DNA
aattggaacctgctgatatgaaatgcataagagaactgaacctatcagtatcatgcgaaacctgcagcatagacatactaccgtgatgtttcaatttttcaagcaaacaaaatatccaaaaacacacaaaatagaggaaaataaggcgaaattacaaacctctctaggattttcaagatcttccatattcctctcagatccgggtgtaaagacaaccagttcgaagttccaaagctgttgcaatactacttgtctaaacgtcatagcaagtatatttttggacgaggtacttgaatggaaatcttgagcgagagactttctgagcttcgtggccttttccttgacttctctggcaggtaaaaactgttaacacagtcaacttaggaatgacaaatacaatcggatagctaaattttatctaacgacaatattccagagaggggagagagacacattgttttataacaagactcccaatttcatgagatgacaacatcgcacgacagtcaaacaaaattctaagagaacatcaaataatactagaaacggacatattagtgaaggagctcttaaaggtagccttgaaccagagatgggaacgccatcaaatcaatctcattcatcaatcatggagttaattgttccgatggtggaattcaaaatcggtcatagatttttattttaagaataaaaattaaaatggaggctcctgaagctaacatgccaggtgcaaaagtttgggagaacgcgttcacgtcaacattcgaattcagtctcataaatggaaattgtagcaatgacgaaaaatattcatagttgttagtcacggaaatcggttccataatacaccaccgtcgaatgcgagctaaaacgagcaccaaattacgcagtcaggttaaaaaataactaaccagccgggtcgagacagtgctgtgttcatcagaaattcccggaaatacagtctccacaaccattgcaggcatcccagaatcaaggtgctcagtggcggtttcaccgccatcagccgcagccgcagtaacagactgccggaaatcgacgcctccgaccagagaaagccgagacaagtcattctcgtacgtccggacacagggaagaatcttctcatcggactccagcacagcttgaagaacctcttcctcggtcgtcggaattggcctcccagcagagcaagagaaggtagaaaagcaatgccttgagtttttcagaacaattttgggagtataaattaagggtatagcaaacagttggcgagctggtatagcctgtataggagaataatggataaaagacaaactcaacgccattggagaaatggccataaacctctgagcgagtgctagggttttcgttttatagtgctactagctgtgcgtcgccggagaagcgatgtctcgtgcccacatctttttccctccatttcttttcggggttatttctctatataccctcccaaaatattacaattaaaacagttccattttgttttaaaaaaataataaaaatttatttctcaataattttttttgaaaattgaccgtcaatttcgtacaatctacttttaaagaaatgattacttcatggatggtttctaaagggaatccaaaatttaaaagtttaattaatttagattatgttttatataacattgattaaatgaaatatgaaataaggtgtaagttgatattagccctaatatcaaagatgagggtaaaagtaaaataatagtgaaaagatatccaactgattcttgggtaccggttcgggtagggtttgggggaatccggttggcgttttttgagcacagagagatgtaaacgggacgggaagaaataaaggccaacacaactataaattctcctctcggcggaaaggcggagcagcgtccaacttcgcctttcacaaaatttactaagagggggcttccattctacgtcgattctgctcctcttctactttttcccttctgctttttgtcgacg

SEQ ID NO: 41 - DNA, 2000 bp, Cucumis melo
  source 1..2000: mol_type = unassigned DNA
ggtctcagacccataggaatagatcatttattgccttatgaattagtttatgagtacataataattgtcaaccgtatacaaatcaacatgaaagaatataatgttgtacatagtcattccaagtttactaatattatattttaggagtgttctacaccgaacctgtcacatgtacactgggtatgctacccctgaattcaatatcataaagcaactttaattgtcaagcattctcttgaccatttgtgacccatttgctcctactttttcaatcaataactatcacaaaaagctagataccacatgtgataaactcactgaaatcaggtatatggttacctgtgcttggtcagcactcaatcagattcgaaggcctagtctttgtatttccccccctctgcacactacaaatagtcctccacgtaaagacccataacaaaacgcaaaccaagtacagaaaatctagccgaaatccagaccactcaaacataacaaatcttctggtaagtttgaataaaatataaaactaacctaatttaatcaaataatacaataaaatggaagcaactaacataacatatctaaatatgatcacgtagtaggaaaaaaaaaaacattccaaaactattaacaatcattcttaatggtatgggtcaatccccattatttaggactataacaagaattcctcatacctaatgccacatcctatgtccaaccctcgagattacctcgtgagtaatcaatcttattcatccttatttcaaattatgtgaaatttctcatcaggttgatcatattgactttcaatacaacttatgattaatctttcccttgatataatttcgtatgaaaaggaagttgacattatgtgattttctcataaggtaaaccaagtaaacttgacatgacgtcttaacaagtcttggtttctaagtgtaatttactgcagaaaaaatcctaaattctatgacttttcctatgagattgaccaaatcaactttacgagaaatcttgggaagccatacctacaaagtcttcccccaagaaattacaatttctagtaaagattgttgaaatttaccctccaatttttccgtgaaatttgacaaacttgtaagaatatcaaatttgggttggatattgacattccaaaataagtagttttaaaaaggatttatccaacaataatagaagaaaaaagataggaaataacatacccacgtaaatggaatgtaaatatttatatattaggtgtcttgaacgaccgtcaaacgaaaataaattgttcatccgaagttgaaactctttaagtgtacatttatcttttcgtaagaataaaatgtaaaattaacgtgtgaaaggttgggttaaataagttatgtagagataatattgaagatgatagaataatcacgatcgatgaattagtatagtcccagagcggatttaaacctctctcactttcatgctttctatatatatcaaaataatttcaagtagttggtttagtcgtaaaaaagtcaaccaatctcttttagataaaccttgagttattaaaaaattagatcaaagataatcgttgaaattgaaattttaagagtataattataacaaattggaaagttctaaagtagtttttgcaatattctccatcaaaatagagtagaaaaatattttagtaattttcttatcttaattttagttttgtaatagttattaggatggtcctaagttctcaatccgcttttagtccataaaaagaaagaagagagagaaaaaaagtccccgatccgcgacacataccaatccaaccaattatgcacaatccatgtgatatcgaacggtcacaagaataaatgctttctacacacggatcaccatccaacggcttttccttccatctcatcctctatataatctaccaactctgtcatcttcgacacacttcaattatctcagcttttatttcatcggattttccatcaaacaaggcaaca

SEQ ID NO: 42 - DNA, 2000 bp, Cucumis melo
  source 1..2000: mol_type = unassigned DNA
actccattatttggtttgattaaagcttccatctgattaataaataataataattataaaataaaaaaaagcgagagttccattaagtaatattatctaccgaaagagagcaactatcacctcaaacttcaaaaagataaaatagagacgaaacttgaccaagtcaaacacaaaccacaaacaaccgatctgacagaaagtttgccagaatcttcaatgtacacgcgaagataaacaaataattaaatctcgttcgtctggataacataacacagcaaatgaatttttttaatacatattttaaaaaagaaatttaaaattggtagattttataaatcatttccaaagggtttttcttgttttaaaatgtttttttgtttaaaataggcagttcatcaccacttgagaagatccaaactgggcggcaccggttctgcgacgcttgagggccgtctccgactcttcgccgtaggaggccgatttacgcaaagaataaccggacaatgttggacagttttgacgagaagttaaaccgagtaagggcttatgcttcttctcaatgcgctcgtcgtcgtcgtcggcgacggcggcagcggtgatggggacttgctctgttgcggggtgaacattgggattccgacaagaaggtgggttcttagggttggagggaaagtggaaagcgttatggggttcttgatgctgttcctgcaacttttgctgtttgaggaagcgcttttggagatctaaaagagaagggcgaccctttttcttcttcttcttcatggtggatttagaaacctcgcccattgttcttcttccctttctcgcaggaacgaagcgcagggaggttaattgatttcagttttcacggcggagggtgcaggatttctaggcacgtgcgaatcgcatgaccctatcacgtgcgaatcagtgacggtataacgtgcatgcaaaggaatagaaacacaaaccgctcttacaattataaaactctaaactaaactacgaacgcatctcataatgggcccactccatcatcctatgggccttttgaattttatgtatactattttttttttttttttttttttctttaatcacaatcaatttttctggtatttttttaaatattcaacaaactttttgttttaatgttgtgtatatctaattaatttagttttattggatgtcattttttctatttttgaaaaaactcttaaaaaaaatataaacaaaaaaagaatggaaaaagaatatcaaacaaagagaggagagagcaaccatacctaaaaagtttgaaagtaaaattgaaaaaaagaatatacattgagggcagtgttgaaaatgaaattaatgaaaaaggaaagggtacgtaacaataaattacattttcttgcaggcttaaacgaaggcccatatatgaaaagggaagcttcgatttgggttcagttatgcgggcctggggttggtattgggcttaattttataaagaaggcccaaatgttggaaagacgggctttgagagagggtgttcggcttttgcccgaggggggtgggggagtggcaccgccaagcgaagacaacgaatattaggagagaaaacacaaagaggcggagagatggaagagaatgaggtggaccaatgagataagagtgcgcagattattgaggtggcaataaatttagaatcccgcctaaatcccagctttcatttcatgcgcaattgaatttcaatttgccattcccctccatagggacttaattatccccttttttttactctcataactccctctcttcccaccacgttcgcttcttcctcccccttcctcttcaaaccctaaacctaacctaacctaacctccttccccaacttcttccgtcggtacgtttcatccatctcctcccacttttcatctttttttccttctaatttcatctcttttctttgtttttccttccaattgttgctgatcccatactatactgcaggattcgaa

SEQ ID NO: 43 - DNA, 1115 bp, Cucumis melo
  unsure 46..60: note = unsure at all n locations
  source 1..1115: mol_type = unassigned DNA
  unsure 454: note = unsure at all n locations
aaagaaatcaaagcgaaaaaacgaggaggaaaagaagaaaaacgannnnnnnnnnnnnnnacataaataaagaatagaaaaataaggaagatgaggaaagaaatcgcagaaaagaaaaagagaaaagaataaagacaaaattgcagggaaagatggagaagatgaaataataggaagaagacgaatcgcgagaagaaaataaagatgagagggcaaacctgaaatatttaaaaaattgctaactttatgggttttgttacacgggccgtaaatagttttgttacatttatgtaaatttacaatcaattaattatacaattaatcaaatttccacaaatacaataattggatattttcccaaaatatctaataagtttcaatttctacccatcaaatatttcaaccattattaacaccaaaaaattcaaagattaaacttaagataattacaaanaaattaccttaaatttggggcattacacatttacattgaactatacaattgtttaccataatcaaaacgatcgtttttttatgatcgacatgataatttcctatgatcaacacgattttttatcatatcaacaccttcatttaaatttgaagtttttttcccatcgttaaaaagaagtacacgatcttttagaagaagattacttgcgcgggctgattaatcgtctgttgactgtgacattttttatatttttcatcatgagcctgtatgtcttttttgtttttataattgttttacatcgtgtaaatagtttgccgattagttatatttgttagaaaacactttttcaaatgtcgaaaatttgattttgatttattaaaactttagtaaaggatagtgtttattacgtatagaatcccaaattttcacaataatttttcaaaagtaatccaaaagaaaaaagcaacaataataaaaggctcaaagcgacgtcgtttagggcaacagctggggagaagaggacgatctgaaaaatcatttcttgagcgaagggaaaaggagctctactaaagcagtcgaaaaaagaaaactcaaacctcgctgcgactctcgacattgattctgttttcaattcattttgccaaagttaatcgatccgaac

SEQ ID NO: 44 - DNA, 2000 bp, Cucumis melo
  source 1..2000: mol_type = unassigned DNA
tatagtttgtaacaatctactctatgttctttcaattttgatacatttgaatcttaaactttattgagttacacaatatagtccttgtattttaaaatttataatgactctatttatattaatattatagaaatttttgttaaggtttaataaaaatttttctgtataaataaatcgaacacgaagtctatatttagactgcaatatagtaaaacctgacatctaagtttggtgaattttgttttgctttaaaaactaaactattacaattttaaaaatattttaatttagttaatgcacattaactttacggagtaaatttttacaagattgaatatacatagattaaatagttataaaaccaaagattagagtaaaaacatttaaatagaaagaactaagatttttttaaaacgaaaatgatactagatacatatatatgtatctatattataattactcattttaacatatagttttgaaagaacaaagattagttgcatgtgttgattgtttttaagaaggaaataatttttgaatggaaaattttcaaaagttttaaatttgacaataaactcatatttaaagtgtactacaaattttaacttttggttaaactccttgtttagttcaatcatgtaataaattctcattccaagaatcgttttagaaaattttattgtgcatttaataaaatatagaacatatatggcatataaaaattgattacttttttctttttttgggacgaaaaacacattagatataatcttttttgaaagtttatgaactttaaaaatgggttattttatacggtggtcaactttattttattgaaattattgagtttataaagattgttatatcattttcttcttctctttcactagaatacaatcaaacctatcaaactctctatgacttatttagaattctttttgttatatttttgaaattaataaatgaaaagcttagagtctaaattataacaattaaaattgaaaattttgcaataattttatttttagcaaaatgacgtttggtttttggggattgggaatggatcgatactatcccgattccggacaaagaaaccgacccgagattcgaattttttccattcccaaacagagcacttaaaatttaagcaacgttataacggcgtcaccgaactaaacggaaaaatatgaagaaaattagaaaaagaaaaacggaacagtcaaacgttacttcacgtcaatggcaatattcatttttttttttgtttaaataattgaatttaattaatttggtttataaaaatagagtcctcatatatcgcgaatgcgcatttgatcgtgaaggacagcttctcccttgtgttcaagagagagagatctatcattcttatttggggccgatctctctattctcctctcttctattccgtaagtttttctcattcattctcctctctcatttctctccgagatctgtttacaatccttttgattttcatttttcctgcttcgatctgtgctcctggtgattcccttttcctgttttatcttttgttgatcttggaattgattgttcttttgtgggttttcattgatttgtattttctgatctgggtttctgttttctcgccttgatgttttgtatttggatctgatctgacgtaccctttttttttttttttatttgaattgcttttccaatgtttatacctggatttttattgatgcatgggtttaaccgattggttggatgcgttttctttgtgctggatctaggtgtccttgtttttaatttgaattgtgggtaaaaatggcattattgtaatgtgtttggagtttgattttgaatcttggctagttgatttttgaattacaaagatcggatcctcttcttttttgggttgtcttaagatttttggctggtttaagtatttgatgtcgttgtattttaaggggtaactgatgccggcttgttgtgtttgtattcagtttacttgaaaa

SEQ ID NO: 45 - DNA, 2000 bp, Cucumis melo
  unsure 662..688: note = unsure at all n locations
  source 1..2000: mol_type = unassigned DNA
  unsure 1194..1198: note = unsure at all n locations
attatctaaacattaactgcaactaatacagattacagtaaatttgaattatgatgttatctagagtcatttgtcttcaatgatattgactcagattcaaactttatgaaaatgttaccctggaaaatattctaccgcaaaatttcaatccaaagttaaaggttgaataatttagaagttttctgcgacttccacccacttattatttagaagacctgaaatcaaaatgatagaagatgaatataaatatatttttttgctttaaaatttataaatcaaacatttgacctagtaattgataatacataatattatgtgactcgtaagtaaaaaagaaattgaaataatatatatatacggagatcgcaaaaaataaaaatgaaagtaatataaagtaaacgcaaagtaagaaagcaagcattttcaagtaagattgaaacccccgtccctgggggctccaagataacacgggtgcccaattacccggtacacgacttttgttgaacaacattgaataattagcccaaatgaaaatatttgtcgacatatctttcttataatatgtaaattagataccaacacaaacacttgtaacaatatcctaactaacttggttttaaatatatatatatatattatttttttttctatttatttatttnnnnnnnnnnnnnnnnnnnnnnnnnnntagataccaatatttagtggcgggtccataaattttatatagggttattatataataaacactaaaaatttagatattattattttcaaagttaggccacaagtaaaagtggggatataattattatactataaccatattttggtaaaattaagtattaaatatactttaaaattaatattaaaatataaaaatcgataatgtgtgggataaatttatggatgtaaatatcaatgttttaatgttcaaataaataaatagtaaatagaaacaaaacaagaagtcagtctttactactaatcgggactaaaatttgaatttgatttaaaatttaaaacttaaataggactaaaaatgttaggacaaaatagtaacaaacacgaaatttaggcaaagaaatataattttatttatttattatcattttttttatatatataattgaaaattgattactaaaaaaaacaaagaacggtaaaaccctagattaaaatcaaaatagaaannnnnaacccgaaaggagaattttgatttccagagctaaacataacacgatccaaacccataaatcccgcatcgagtggaaccgatatcttctccccttcgaagttccaactctccgtttccgtctttcttttcgattctccttcaaaccctcttttcttcgtcttcttcaaatctctacatttcaaaatcttcgctaatctcttcttccccttctcttccgatctgaccgtgaccccattcgaagcttcttctttcaccaagctttctctccgctatcaactttaactttcgtcctgtattccttagccttcccttgcttttgcagtctccgccaccgaacaattcctatcccgagataatcccacttttgggtcgtgtttctcacttattcaaatcgctggttctttgattttgggcttatttcactctgcatctgctgcgacttggaggttataacatctctctctcggtcttgttaggtatgaaggatttgagatattttctaatctatctgaactgggttttctttcgcttccgtttatgagatgtaatttgttgttctgggaagttttcagatcctttctaatgggcttctttaatttaatttaaagcttctttgtttgtacgagatgtcaagtcttaatttctagcaatatcagtatctgggttggtggtatttaggatgatcaagtcttttgttatttaatggatgagaacaattattgtcattgttattattattttttggaaaaaaaatcaatgggttttcactggttttgttgatctttttagataattgaagttc

SEQ ID NO: 46 - DNA, 2000 bp, Cucumis melo
  source 1..2000: mol_type = unassigned DNA
cttctcgatcgcagcaattcaacttcataaacaagtccaagaacgaagagtttgattcccctaatttaacttaattttctggtgaaaatggaccatacttttaattacatattattttggttattgccttttaaatggtctattttaatctctaattttttttattaaacaatgatggtgaatcttttctaaaagaaagaaaaaacttctttacaaactatccaactctaataacaacactaattataaactagtctactacctttattataacagcaattaaaagaaaaaatcgtattcactgacaaaaattcgttcttttgaatgcttatcgaatgttttaatttttttaaaaaaatatataaatatttgtaagggaaggatcagaattaaaactctctcccctcaatgaaattgaattatttgtttttcttgtttttctttttttaaaataaacctatggatttagttggtcggtcgaattaaaatcgtgaggtcgcacacgcggtgtcttgtggattcaaaattatgattatttccatcaccccttggctttttcgctccattcggccatgccttacaaatttcgctccactcccattcttctcttcctctcctctttcaactgcattgaggccgatcctttaggtaaatggttctctcccatttcatctctaattcctctgtttctttttattttacttgttctttttccagccggatcctccatttctgtggtgaaactgaattgttcttatcgatttcttgtttgaattctgtttttctctgtttgtgtctgtgtgtgtttttaatttgttttggcatgttgaagtttaaagataccaaaagttgcgcttcactactttccagtttcgatggtagctgctagttgtaacgcttacgttcttggttttttagttaaaatttttttgcttcttgttgtttactgtttagcaaaaagcatggggaatactaccaaagtcccgaacttaatagatagatgatcatgtgctaagaagtgcgatactttccgtagctgatacgtgacacagtgtctgacatttgtttgacacatattagaaacttgttagtataacatatgtgttaaacaggcatagaacacctgttgtactaaaaaaaatatttgtatgataataataataactttgaagtgtaaaatatatccagctaagttttttcaagtatacaagtgcattaactcatttcctcttgattttcttttggtataaaaattatatatattttgaaaaccgtatactttaataaatgtatccttgtgcattatgtcctagatttttagaatatggtgtgttgttgtgtctatatcgtgtcgtatcaatatctcgtattcgtatctgtgtttgttagatcatatgtataagcgaggacagctatttctgatgttacaagaccttcttcaaattttacaggaaatcattcaatttgaaaattcaagattacaaatgcaatctaaaccaaacttcaagaacaaaaagtgttatttgttaatatccttgcgacctcacccaaagtatctattacaaacttcagaaaaaacttcataataagggttgggttgaaaaaaaacatgaagaagtcccaccccaacctaactctaaaaagcataaaaaattcaacccaacccaaaccttacaatttgggttgggtagtccgtgttgttcgggttgtcgggttatttgaactcctagttttagctaagtgtaaacttatttaaggatgttgaaggttagcattgatctttctctctcaaatttggtcaagaggaattattttttgagtcattcatatagttccattttgcttttgagcatttgaattgtttgttaactacttctttgattaatatattcgaaagtgaaatttccttggtttactttattgatcagtgtcccattttaccagttatttcagttctcctaataaccttcattggacttgagttcggttaacacaaaaca

SEQ ID NO: 47 - DNA, 2000 bp, Cucumis melo
  source 1..2000: mol_type = unassigned DNA
aatgtatcgtgggcatttattatgacaatagtgtaaaaatgttgttaacatatctgtgtattgtcgatgatgtagatctactacgccaatagttatagtttccatcggtatatctatgaaatgtcaacgccttaaaatagttgcatgggcatagacttgtttttttagaaaaatatgttttatatttgtatttttttcactaacatccttttggttttgtatctaaacacaactcaaaatatatcaaatactgaaattcatttcctaaaaaagttacaattgtttcaaatataagtcacctgaatgaagttcttaaaacacaaagaattgttccttacaaaattaacataagcaaaatagtaagatcgtccaaaataacaaacattacataaactttagaccaacttctaatttgtttgccaggaagtgatctccattgaaagtttgtcttaaaaaacaaataaaaagaaaataatagaaacatattcaataaactagtacattttgcaccttacatatatatacaaaaactttacctactttaatttcttgaaaatctaaattttgaattaagaacttttcttacaacgccaaaacaataataacttataaatcttagtgatggaataataagttataattcattggttgattgtatcattaaccatttctttcttttggtgtgagaaacttatccaactaaaaatattcacaatagtagggcgggtttgtcgtggctttgtctgaatttctacaacatgtgtaaatattttcaactgttttatcctctaagacaattttcctaaaaacaatcatgttgttcacacgagatttccaaggaaatttaattcaaggagttgaacttgtatttatgttgatttgatgcctatgcttaattttaagatttgagaaagcacgttatatttgtaaagttggagtattggaagaaaagtttgtattttcaaaagaacctcaatattcgagatcgacggttggcttcaaaagttaggaagtctttgactcataggagaatcctatctagactttatgcaatataaagagagttctgttttatggacttagttggataaataattaatttgattcaacggtcataatgaaaacatgtgacatcattataattaaccaatttatatctttaatacacatatcaacttttaaatagttctaaattcaattatttgtatttatcatcattaattaaataaataatgtgacaatttgtgattgatccaaaaatttcatattcaatctatactatattagttaagcttaaaattttactaaatgcttaaagttttggattatcgagcttcctaccaaacaaaagcctctattgcacatttaaaatatagaatagtaggtttatataatatgaaagattgactcttaagaccatactctatgacctaatgaaatcgacatttatgtaattgataattaataattaataaaaaaagtgtgacaaaaaagtggacataataaaagaaaggaaattgtgaagcattagcatccgaatttcgaagaaaacaaagggcgccctcagatcaaagaggacatactataaagtctccacgctatttcaagaattggcgtgattctcaagcgacatttccgtaattcaccacaaaaattaaaaacaaaaaagaactcacagattctgatttgacttttgaaaccccaacccccatcatctcccaatttaattttccctcgatatttatccaaattcagaaacataatcttgacaattttatgctccattcttccaatctcagccgtacgtttcattcaaactccaattctcccccactgcgccttccactacctttttcctttctattaaagtgtcctcacaaactcacctcctctctctgtttctgtctgcggtaggatcgccgactccggatttacatttcaggggtcgaagatttgttctggggtttctttaatttctttatatatatacacacacaatc

SEQ ID NO: 48 - DNA, 2000 bp, Cucumis melo
  source 1..2000: mol_type = unassigned DNA
gccaaggaaaatgaattgtctaagaagaagaaagaaaagaaagaacattttttgcaaggctacaaatcaaaacttaaattatccacgtgacacaacaagttcagagaggaaagaaccctctaaaactcccatataccttggcaataaccatgacaatagcaataaataaacaagtccatgacataaaataaatattgttttcattaaatctcaataattcatatgtagtccgctccgattatgccacagtcatatatcaagttcagtattttaacaattcaagtagacatacataaagctactatggaaaacataaacaagaatggaagaaggagggttaaggaaacctttatccctgatggagtttcagtaaaactgagcttgtaggtattagtacgaaagctgtgaaatgaacaaccttggccaggtaattgaggcaccccaagatttcctttatcaacactatcaaagaaagtaaagaaagttatcacttcaaaacccaactcccaaaagcagctcatcattttccagtaagttaatactttgaaagatcaaaatcaaatctacaatcaaattagacttcttaatagttattgccacgaaccatgcatttgtcacgttattaagactatggtttgcaataatctcctatctggttggatcactacttatactaggcacagcataaactaaagtagtttcccagagaaggaagaaacatgaacctggttgggtccatcttggcagtaaaggatttaagggagaagagcaaaccaaacataagtttatggtcttgctggggattcaatgtccggagaggccgattccactccctgtagaacaagcaaactccattcctattgaaaatatacatcatatgcacattgtttccagtcgctgtcggtaccggaggcgagggactaatttctgacccaccaaagaactgcatggtttctggaataaactaaactaaatcaaatcaattgtcatataaaatgatctacgaatctaagattctaacaaacccaacatttcactcaactctacaatcagtaacctagcaaagcaactaataattcaatcattcctaataattcattgaggttaaaaataaaatagcgaattgtcaacaggtaaaatctaacccgacccaaatcaggaatcactaaagcaagaagctgtatgactcgatcaaaaataacccagatgcatttccctttggcctctctacagaaccactcaatatagttagaaacaaatctagtgtaaaattgggagtcctattcatacataattccaaggaaaatggattttacttatgcatcgtataagagactgtgagcaggggaaaatggagagataatcaccaatgagctggatggtgacagattcaagaagaagcatcaaaatcaaacaacggagagcagaaagatacctcaaagagcagagactgcaaagtaaaggaagcgatcaattcaacgacgaagctcttgattcgtcaggcaatgattgccggcgacaacaacgtcagcagatcggagccttacggcaccggagacccctccgacaaggacagagtgaacgaacgtcgtttgtgaacggtgtaagcaaatcgatctctcggagtccaactccaaagtactgtttcgatatgcattaatacatttgatttttgttatatcaaaaataaatattatattaaaatttataaacattaacaaaaaaaaattaatttcacataatttaaaggaccatttggtaatatatacaaaattgcaaaaatcaaattgggcctattttgttgttattggaggcccaagatgggtggtgataaatatgggcctccaaaagaataagcaaaaaaccctaatttcctctcttcctctctttcccaatactataaatcttcaccattttcctgattagggtttttgttcgttcttggccgtccccttcatcgttcccagagagagggagagagtaagttgcaatagtaaaac

SEQ ID NO: 49 - DNA, 2000 bp, Cucumis melo
  source 1..2000: mol_type = unassigned DNA
aattagcaaattgtatgtaacaacgtcatgaagaggcatttcatcgaacaatttaatagcagagtcaagatggcccagttttataagtttattgatttctcgattcttaaggtaaatgagatcgcgagcatggaaatgcagacccaaacgagaatatgggtgtggtaaatggaccggtgatgttgtggctacaaattcggattttacagcagtaatagttctgacgaaggaagcgaatttagtgacctttcgcatcacagtttaagcttatcagagacctgaacaagggctctagctctacaacttagaaaggtttgatatggtccgtgatcgggagggaccgaataacaggcgcttaaattgttgttcataaagaagaggattgtcgttgatgtattttaaccaagagatgattagttatccacatccacagagagaacagaagacggctttctaaatctacaccgataaaaataaccctaccactttgtttctttagaaaagggtcacattctttaaaaacattagcgtcgaggattaatagggtatattgactaatgctctgtttggatttcgagaaataccaatttacaattgatttcaaattaattatgttttgttgttgcacgaaagataaaaagaatttaaaattcaaaaggatctcaaatcttatttttaacttaaaaacttttatgacccaaacggtttatgtatgatttaaaagtagaatacctctgtgaattcttaatttttttttctttccaattaccacataaatatgaaattttaaatacatttattttaaattttatatccgaaacaaaataataatttaaaactatttctcatatatgaagtgtgattcgatctaaattataaaataataaaaatttacatctagttttgattattttttttcgttagatactaaattgttaagaaaataacatttttaatccaaagttttgaagaatatatgacttttaaaatggtatttatctttttagtgtctgatttttaaaaaatggatttcaaaagttcatcaaatagcattgtatttttattttaaataattttgacatttaaaattagagtaatggtttataaaagacacttgatctctaaaactattttcttagatataaatacgtatgattatttttaaaaatcaatcaaaataggtaaattgtaaaaaaaaaaaaaaatcacatgaatagtagttgtaattatgctctcaaactttcggttatgaaaaataaacattttaacttttagacgtgtcaaagttgagtcaagttggaccttcaaagttatgtagttatataaattgtaatatatgtataagcttgtggattcaattttatcatttatgggtccaatctctacaattatcgtaagtctatgggtcaattgtaacacatgtggagtttaagagctcaattttggacgtggatgtgttttgcaaccaactccacaccttaaaaaggtgtttttttttaatttatcaaaaaacaagaatttagaatctttaagtttatctttaaaaatcaacggacattttgaaaaccaattgaaactactgttataaacctaacaactaaaagtatattttttaagaccgaaagcataaatccataaaaaaaaaatccagaactgaaaatgtaacttttatagttgaaaatttagctaaattatacatattaaaattcaaggaccatataaaattaaagtacctgattaaataataacgaattaatgtttggtatttttaacctacattagaaaaaaaaaacaaaagaaaaacggcatactatttgtcaagcgtccgatgggaagaaaatccaacggtgagtgttagtattgaaatacgcagttctcgtgaatgagcctggcttagatttgggaacaagagccaacccctttcgaccgagaagccgtcgtcttcaccatattcgcctcaaccattcgatagccacgtttgaagaagaattaggattgcc

SEQ ID NO: 50 - DNA, 2000 bp, Cucumis melo
  source 1..2000: mol_type = unassigned DNA
agccaatggttcaattaacagctctagttctagcatggctaccgctggtaacctttctatgactagagacgtcttggaggtggagggtagggcacgaggattgaaaggtgagggtttggtgaaaactcaagcctttcaaattcaagaaagcatgcttgacctagtagcatctggtgatcttggggaatttgcaatggatactcatacccttagtcggcattcgtctcttggttctgctggtatatatttttttctcttgttttctagtgatattttcttttatcaatttccattatgaagatggaatcttatgttctattttttcatttggaatgtaggctttcacaatgaaaaaattgctaatacgtttccagaagaggttgctaaagacccgtaagttcttatttcttaacaatttcctcagtttaacaagttttatttactaacatatccttagttgtataaatatgaatctattatattaactatttcatttatctatcttttaacagggtgaccattcacaacaaagataatacttcattgaaacgccctcctgtctcacgcacttcggcatcccaggatggattgtctgtcctgattcctgatccggttgttagaggaaagaactcagatggtaaataataagtgatccattctgttatcttctttattcattttcaattttgtattttgtatatatttatataatattttagaaagataaaagatccatcctgaaactttgtttcaggtggaagaccggacccaactagtatcttggtgaaccaagaaaacatggcagccatgaagaaagagatgcgtttccggcgctcttcttcttgtagtgacagcgacgtgtcagagacttcttttattgatatgctgaagaagacagctccacaagaatcccatttgacaacggcgggagttccagagccatctgatggaatgcagggagggaaaggtgggaaaaagaaagggaagaaggggagacagatagatcccgcactactcggattcaaagtcaccagcaaccgaattatgatgggtgaaatccaacgcttagacgattgatccattaggcaagatatagaacagaaattgatttttttttttttttttccaatcatttttgtagattgtgcagttatttgttttcgtgtttgtttaaccctcttgtaagttgttgtatataggtttcttagagttgtcagctgcgttgaaacatgtggccggtatatgtattccaattcttttcttttttcccgcagttgtaaatgatcaaatttgagttggtcaaattaccaaacctttgtacaggaacttcgaagagagttgaaattttattctttttcttttttgttcttttatagagttcgagattatttgtatgaatataatcaaaagcaaagcatgtaaaaataaaatgatttgaaagggaggttttctatcccattcaatgtgacgaatccaacacttaaagtaaatttgaaaactgtctaatttatatgtatggaatgtaatgctcttcaagaaattatcttatcttctaatatttaatgggattcacataaatatgaaatttcaacgtttttcttttcctttttgttgtgagattaaggatactagataataaccgacctcaaccttttaggccaagaggtctggagtctttatacttgaaaaaagtttacacatattctaaaagattaaaaggttaattgtttggtaaaacattaatgatgacgatacttaaggtttcattaaaaaaatatttggaacaatttgtttataatttaataaaattgtaactttgaacattttgaattacattttgtttttccatttttacggtcctcgaactcatcgatactcacaatggagaaaaatatcacaatgccgaaaatacccttcttgttcccttcttatacaaaagcaacactattggccttatcaacggagcagcagctactctcctttagcacaaatctccatcc

SEQ ID NO: 51 - DNA, 2000 bp, Cucumis melo
  source 1..2000: mol_type = unassigned DNA
tggtgtacccacttggttttttctctttttttcttttagctttttgctcctaaatttcttgccttagttttcaaaagcttgtttttatttttgaaatttaaccaagtgaatagaaaaaaaaaagagaaaacaaaagcttttaaaagcttgtttttatttttgaaatttaactaagtgaataggaaaaaaagaaaaaagcttataaaatttgacgaaatttgctgtattttgtacattttactatttttctattttaaaaaatgtgtctgaacgaaaaacttatattatgagatttaattttcaaaataaaattataccaaacagactttagaattgtcaatcaaatttgacaatgattaggtgcattttttaaagttatcctaaagtttttttttttttcgtagtcttgcccttgcttttatcgttaacaaataaaattttccttatatatatatacacatttaactactcaaggtctgtattttttccacctgatttatttaatattttttttttttgcagaaaatctatttgtattttaggggaaacaaatgagtgaagagatcatcaagcaacggttgcgatgttgcagcggaaaaatctttggtttgtcatttcttgtgatgggggtttatagggtagtatggttattgtattttaggatgttgatttttattttaatgagccaagagagagatgtggattctaaaattgatgattgatattattgatgtgatataaatatataattttgtgcgaaaattgctattttattttctgtatgctcattcagatcacacaataatatttgatgtagctttacttattgacaaaatataggttttaatcttgtgctcatacaaacaacagctatgggtgaaattattttctgattttatttggcaaagatgatgtcagcattgtgtaaatttaatgtgaattacacttctgatttcttcccaatgtgccctctcaaatattggcaccaagccatttaattgtaaatacggaaaggtcataaatttccatgcaagatttatttcatgtttaaaatgattgtgtgaaacaaaatgaaaaacaagaaattcttacctccaacctcaaagtagtcgatatgtcaaggttcaatatcaattttaaatatccatgaatagctttgatatcttttataaatgcttgtaatatatatatactaatagcaatgtctataagttagttttgagagtaatacttgttatagataacaatgttactctatttaccactctactattgaaagcttctttttcttccatttatgaattaataacggtcaagatccaattgcatgagttacttttaattaattacaatctaaaatgttaatataagtctaaaattgtccaatatatgtgattttttttttctctctcaaaccttcccttcttttcattgaacttgtggttcaaatttgatggaggacactgggaaacagcacaattcaaagagccaaagattgagtaattttttgatttcagagttttcatctcttcttcattctacacctttcacttctcatccacaactatccaatcaaccattgccacgtggcatcaaaaatatccaaaactgaatgagatccaccacaaagttcctctcatcactgtttgtcatcaactcatcaagaacttcatcatcaatcagaaatccaacatttcaacttctcttaggaaatgacatttttaccagtctccaatgtcaaaaactcacacaaaatccctctttccaatctaaattttacaaagataacaggggtaattgaagaaacttagcagtaagttaacatattatagctttcatcaacccaagtttttttggttcctttctaaactgtagtttgttttcttgatccattctaaatatttcctctgcatgaaaagaagaaaggaaaagtgaaggcgaaacctgttttatgcttcagaaaaccaattcagagtaaccaaagatctgaacttcagacc

SEQ ID NO: 52 - DNA, 2000 bp, Cucumis melo
  source 1..2000: mol_type = unassigned DNA
cgctcaaattactaacatccttctctttcttgttcccattcgactagagagacactatcttatccacctcagttggctgggtgaaatcattgaaactaacggttgattgtccagattgttaaactaccctatgttttatcatcttggttacatttataggattgtcagaataataattccttttgaaatcatattctaattggcacaggactaaaataatgcctttcttaagctgtaataattagaatctaaacagtgaagttagtaactgattgatgacatttccacgattttcatttatatcctgtgcagctattctgacatcacaaaacatttcttgattttcatttttacttgtcatccatcagtcaggacgatatcggcgcgcttgttgacgaccttgtcctaaatacaaagaggcttatccgagctacttcaagggagattgacaagtggaaaagatgaaattactcatttgttattacattgtacaagtgatctattaggaagaaccacaatcaaaactgaagaaaaaagaaacgtgctggctgtctacgtggcttttagaggtagaatttatgtacaattgtttagaaagatgtatttaattgctctaaatctcatatgcattggattttgagcaatcttaaaatgccgaatacttaatgtattatcgtaggggtccctagatggcagatttatcatgtccattctccagaaagaaagaaaaaaaccctttttattatacttgttcattttaagctttttctggttgattataatgtcagtaatttaaaaaaaaaaaaaaattactgtgtattggcatcggttatatgttatatacaaccctagttaaaaggtaaagttttgttcattcggtcattagtcattcctatacgaacgtcacattgtgctttataatttcaataggttaaaagtattcaatatagttttttaagttacctagtagaggtgatcattggttgatcggaatcggttttttgacaaaaccgccactgaaccgatcatagtcggtttagtaaatgttcaaatcgaccttgacatcgatgagtaaagatcggtcggtcggtttttgtcggatgggccggtttaacacttggaaatactattttgaaatttttcgaaattaatccctcttgttttcctaccgaccgattttgggtttggtcggtcagttcgattttttcggcctatcttactcactcttattacctagggattgaatttcattttatccttagttttagggttctttttttatacttttgaaatatttatgtcgatgtctagagtttaaaaataacacttgaaattataatataattttttataattgttagctataattttacgtccaaatatcaactcactcgcaacttgtttaatcaaccaataatatgtgtctggaatagtaagtatataacttgtggaaaatgactttaaaagacttttttaaagtatttatttaatgccaaaatatctatatttatgtttatacattaacatacatatccaaagttacatattagatttgttaaataattcaaaatgagctaaagaaaaaaagaagttccatataccaaaataaaatataaaaagttgaagactaaaatagagattttgaaacaaggtaagttagatttacaaattgcaatatgggagaccaaaccaccacataacaaaaatcccaatgtccaaatggcgcaattttgtttaggatagctcacgttatccaaatcactcaatcggagagaccaacttaaaggccacatctgccacgtcaccatactccaccaatcacaacacagcattggatttctcagcttatgagaccaatcacaaacctgaatccgacgtggcatgtccacatccaccagtaccaaccatatagcttctacgttctccacatctaatcttcaccatttacacaatattcttcattcttctttcctcccttcaatccttcatcctctccgccc

SEQ ID NO: 53 - DNA, 2000 bp, Cucumis melo
  source 1..2000: mol_type = unassigned DNA
aattctaacaactccggaaccaaataatttagcatggattgaaatataaatcttcttgacttgcaaaaaaatcattgtaatggtcttatgttggttatagttagggtatcgaaacgccatacaggaatatgggattaaagttaacttttgttcatcaatttcagcttatgaacttctaaaatatcaattttacctttgaacttatatgttattacccctttcgattgtggtatgttaattaatatctgaatctcagtccttatgaaacttttttatactgtcacaaacatatgaagttttattgtaagttcttagaaatcatctaaaaagagtagtttgttggactatttattttattttttcttattaagttgttttcacgccatttcagtaaaataactatagtgaatagagaatcaaacttctaatcttaagttaaggtagtagggtatatgctaattcaataagataatccgtgatgcttgacatctgacttaattgttataagttttaaattttttattgtaatatttaaaatactagtttttggtttctaataaagaaataattgaacaattacaaatatttatacaaaattaaactagaatatatgatcattttccttcgtgttagaaaaagggaaatatatgtgtgtatttatacatattagatattgttttactatattccattttcctcacgggaaatggaggattgagtgggagataaacattgtccccaagagaattgggaatggaaatgcaaatgacatggccctccacaaaattgttcgcctaaaaatgggctttctcacttctcactccgcaagaaaaatatcgtttcccttcgaattattcgggcggcaagatctcaaaaccacatgtttttctttctttatttttcaagcctacattatttataaaaatataacttaagcagagaattatgtaaattcaagtccatttttcgcttcacttagctaaatcattaacaaatctgtaattttgttcataaattagctcaccaattatgttttagcccactaaggcccattagacatttttattagaaaaacatgaaccgttggatcaagatgtgtgttttcttttctttttctttttattttttttgggttttggtggggttttggtggatcatggtggatcaattcgtagctttagcaacctattattatatggagggaaagggcgtattaatctgttagcgccgtccgggagtttagctttcttccccgagcctcggtcttatcccctaactccaaaaccctagcccaaaggtaatccactccttccccctccgctcttcatctttttctattcatcatctttaatctgttctcccttttggttcttagattcttcttttgttggattcttttaatctttactcatggttggccttgtaagtttagacgacgtttttatacattggttaatcctgcttctctatctattcgcacgctagggttttcctattgttttctattctgctctacttctgcaaggttgtgttcttcttcgttcaggtccctttttttaaccgaaattaaattaatgcaaattcgtttgtgcttctaattaggaagccttttggaacatctcgacattttgattgctgcatttcatttcgggtatatttctatgattgaaggatgtgggtctgttcactgcatggtcattacttatgcagctatgcttatcgagtccattatgtttgtgcaatctgtttccggattcataattttttagtaattgatcagtagatgaaaaaagatattgtaatattccttgagtgttgcaccagtcttggtgggtatctgctcctgctctttgcttgtggattttacttttattatatctgtattattcgaaatgttctgttcttgttataacttatacccgaagatgtgttcctccccgcgtctagcgttgtgggttacttatgatggacatggttttgattctgtttggtttgtgcag

SEQ ID NO: 54 - DNA, 1547 bp, Cucumis melo
  unsure 656..657: note = unsure at all n locations
  source 1..1547: mol_type = unassigned DNA
ataatgtgttgatgttgatgatcatgcatggtatattaatctcatgattaaagacgttaagattaatattcattccatgtttatgatgggtgttcttagggttgtacccatatgggtgtccctcgggatcaccaccttttttatgactgtatggttctacgagaccaccagtctgtcatgatatgtttatgaatggtacgacggggtcacttacagcccaattgcttaagtgttccttcgggttcactgaagacctatttttcctaggttttcctttgacttcagcaaaaatcagttttgtcctaggtgttcctcgagttcactgaagactagttttgtcctaagtgttcctttaggtttatcgaagatcagatgtgttcctacagaatcattagattgcaagtgttcgggaacacatcggtttaggggtacttctttacatgaaccctaatggaaaattaacagacatctagcggaattagtagttggtcccttactgagtatatatttatactcactctttttatgtttaatatttcaggcaaaggttaaggtagaggaaagttgacgagtgatagaaaaggatctgtgacatgtcatatggggactcagtttcgtttctgcttctatgtatcagtgtttcagtattttgtttnntaatgaaaatttagtcttcctctattcaagaaagtgtctcttgttattgtttatttttagtaatgatttcaacttagtataaatagttggatcattacaaataatatattggtgatatactttgtaatgatacattgagttatattattcatatgtttaatatacaaaactgcaatattaaaaaatgaaaatcacgtaataagtatatcaacaaaataatacatatattacaagcacgtcacaacactaatatacaaaactaatataaagtaagatcaaagcaaaaccaacgtaaaaaataaaacaaaatcatttgaaattaaatttaactcaaaatacacatcgaagaaagtggagaaaaatcacaatagagttaaattactttgattaataaccattatatttcatattgaaaataatatgtcattagtattttaaaatcaagattaagataggaagaatgaattgctcttttcgtataaaaagggatgattggggccttacgaaaggagaaaaatacatatgttatcgaaaaaacaaattatttttcttgtaagagagaatgattatatccttaaaaaaatgaaagaaagaaacaatcatggcattaaaaaggaaaataaataaattattaaagggcagttcgataataataacaaattcaacgagagtattaaaagaaaatgagaatttgcaaaatttaaacaaatgtgtatattaagtacagccaatgcaattttcaaattttaatttatttggtttacccaaaattcaatttctaaattgagaggaggatatagtaaattcacacgcattatccccttcgagtttcatcatctcacccattcttgcatacagtgcagttacaattccttcattctggatagaca

SEQ ID NO: 55 - DNA, 2000 bp, Cucumis melo
  source 1..2000: mol_type = unassigned DNA
aaacacttatcatgttatgtatcccacatcgaaaagataaaagagacttcatgatctttacatgatatatgagttactccttcgactaccattggttttggagatggattcaacccaataatatgaatctgacccaacaatggtcaactcaaagagacaccatcttgagatacatgttgtgtacccatattagatgaatgactatacctcgcaatatttataacatacatgaattacttctcttactgtaatttggttttgacgtggagcccatgattatctaattaaccataactggtatatgatatattagtaggtaacccgaagaggttctaagataaacacagaattcaatagaatcagagcctttccaatgatatggctttagatgggaatgatttgaagtataatcattctaccacaccctttatatttgtctgtcaccagaaatctcatcttttcttgaggtattattcactcgaaaagagggaggcatttttgggttacccatctaatgcacgatgaactaagggaggtcaagttctgggaatacagctaggcaaccttcacagtggatacattcgaacaaatgataaatgtgaaaatgaatcatttcatgagtgtgactaacccaatcattcctccttctatatctttgaatcccacagtgagtcagagtaaaagttccagcaacaagtcctacaacccaaattctttagctatttcttccaccagaacaaaaccaagcaaaaaatcagccacaaacacagctcaacaatctataaaggccaaaatactaagacagtcaccattaccacattgaaagccgtattttccaacagactttgcctgcaaaatagatcacaaagacacgatttcacattggacagacgccacagctccacaatctcaatttcaatcaaataaaagtaaatcaaagctaaatagcaagtgtatggtaccacgaaagcagcatggctgacgccactgaggcctgtaagagagaaaacaaaataagtgtagaagataaagtgaaatagaaaaatcaatcgataagatagattttcagattaccatttttacgggaattgtacggacccaaacacaaaccccatagagcgccggcctgaagatgaacaggggcaggaaattcagaggaagaaattaaagaaaatgaatcatagtttgagaaattattcgtaaagtttaccgttccgacgcgaatgctggattcgacggcgagggaagaacaaggaacgacgccgttgagttcgtcttccatcttccaattctcaatttccttcggaggtccgtatgctgagagctctgtgtctaccaagttccaaccatactacgtcgttttggatttttatttttattttctttcctctcttttgccaaaaaagaaaaaaatagtattccaacctaaaacctcaaaataacatatttgttgtacaaattataattagtaaacatttgtcattgtgagcttggtatgtaatattaacacgaactttatcgctaataatttagacgttaatgaataatttgagcattgccttcttatattgttattgtgtttataataggattgcttacaatgtaacctagtatgttgttgagctcgttaacttttttgtttttcttgaatattcaaagttaaaaaattgtacaagtttttggtgacgttttcttactacattatcgggatgaagatcaaatatagcttagattagagaagataatcatgttgatttatcgttaaactttgactacaaaatccgtttaatttttttttggatgaattagttatacaatttaaacttaaaaggggtgaatgaagaaagaggatagttttacaaattcgaagtgaaatgagttatttctgcttaaagaaaacaaatctccttcgtgctttaaaacacaaactcaaaaccctaaattcagcgccgattcttcaatacatctctgcaggaagttagggcaaagcagaagcaaaa

SEQ ID NO: 56 - DNA, 2000 bp, Cucumis melo
  source 1..2000: mol_type = unassigned DNA
acttccaaaaatcagcctcatgggatatttaaagaaaacgtaaattaaaattagcatcatttcatattgaacaaactacaaaaattaactctaaaagatgatggtaactacaactaaaccttcaatttttcattgtaaaaatcgaactcttaaacttgttcaaatattaaaatttgaccctcaaacttaaaagagctaaaaaaagaccttcaaatagtaaaagtagaactctcaagcttatagaattattacggttatgattatagccatagatgattcaatcgattttcctccaagatgatggagtataattcttcaaatctagctgcttagatgttatcacgataatgaaatcatatgggaactcaacaaaaagaaagcacttaatgttgaaagacattattctttgggtgttgagttgggcgaacttgatttttattattaatccgcaaaggaccttttgagtaagttgtggcaatctttattggagtgctaagatttgttattcgaaatttcttgttttgatatttttccaactaaaactaatttttttaagaaatgcaccttcaactgatttcatgcgtgtccttttgcaagactcgcatgggacataacacatcatcttatatggcaaggcctatgtgtcagtggagatttgacgtcaatttctttccactgagagtcgtcctctttgtgatggcagaactttggagagtcatcaaaattggttctttgaaaatgtttcttattttgatttttttttttgaaagaaatgagaggaataagatatttttacgaggactctactagtgggtcaatttgcccgcatatggatatgcataagagtccttttggagagaaagggtatgatggaaagacattgcaaaggcccgtccactaactttctattatacaattaggtggaagccacccatagcaatgtcttggttgaacactgatattacttgaaaccatgcatttaagatgtgaaatctcgactagatgctttaggaatttggattgtgtctgttttgttgaattcaagttcattcctaaataccatgaagttaagatccttgaagcaatgaagaccatttatttagatccttaattcaaatctctttactaaagatgattgtttataaatgatcaatttgttgaatgatgttctacttgatatctctaaagcatctcttttcggtgagaagcccacaacttgaatagtattccataaatcatctatttttagtttctatcatgttctttaacatcaaaacattttagcgcactctcttataactaagacttagaaaaacacgaatcttcctttcttacgatatatatcctaaatggttttctatatttgtgccttacaatataatcaattctttttctatttgatattgtcataaaataatactgataacatagtttttatgttttattaacacctaacaagaaatatggaagacgttaatatatcttcaatgtcgatattgaatcattttatttatgaatatatccacgcgtcaaaaaatattttaatcattaacttctaggactaaattcaaacattcttggaaccatagacaaaagaacaaaatttgcaacctcaacaaacaaaattttatctttacatttgcggctacaattcacaaattcccaaaccatgatagaaaggccccaatctcccacgtgataaacacacatatggcacgtgaccaaatcaaaatcatccacatgatgaaaacttaatggacagctcggatcccaacacccaataaaaagcagccatgaagctgacgtggcagatttccccgaaaaccttttaaataataaacaataaaaaaatatatacataaccgttggcaacgtttttccctccacacattttcccattgccttatctttctttccctccaaacagcgagggaagaagaatccaatcatcttcttccaataatttctaaaacgaaattctgctcgattttccctctcca

SEQ ID NO: 57 - DNA, 2000 bp, Cucumis melo
  source 1..2000: mol_type = unassigned DNA
tgggtaacattatgagttttattataatttaaatgaagatcaaactttaagttgtagggtcaccataggaataaaatataatcaataagttagggccccttagtcccacctcgtaaaggagttctgtcatacatatacattatgatattaattctatctccatgagtcatacatgtgattttagtacttgtaattttcattctttttcctattataattcattcaagtactgtcaatatggttaggtattgaaattaattatagactcagatatcatcttcattaatggaaatggaatgttatattctacctctcatttttacacgttgatgataaattagaagaaaaaaaattattatttatattgttttaattgtgagatattagttcaaaatgtaattaataaaatgatacgtgtcttataataaaattaaacaagtataattaaatataaaacaacatacacactctttaactaaaagacacaactcacctaatgctcgacttaaaatcactttgtgtcgtaacttaaccatcaaagcatgttagggtaaacacaataaagatgatttttgagttatgcatgtcatataatgtcacttccaatttgacttatcttgcttgcttgattcatgtatataaacaaaaacatgaaaagtagtgtaaggataccaattacctactgattttttttaaaagtagtttgtctaagacgtgttaaattactaacttagtcacatttgagttttagttctaacttattaaacataaagtaggtatctcccttactcatgtgtgtttcgataatgtcaaattccaatgtttgattaaccaaattgggtaatttaacataaatattcataatataatattttttatggaataccgacatctaaaaagaaatcaaaatgaatattattaggaggtgagtttttaagagagaggaaaataataaaatatggcatcaacaagaacaataataataagaatagaaatccgacaaaggaagaagtggatgcgtgttagtactattgacattggcatatgaacggttgggttgggcctcaaataatttgcatttctaacttccaaacacctaattcctttttttttatccatacttgcaaatatatatttatatatattcaacaagtagtttaatttatttgatataccactttaagttttaaattgatggtagtgtataaataaataatttaggattaagcatgtctatgaaccttttgaaatttgatggagtatatataaaacagaatactcatgggttcattataaaaatctaatagtaaatgtattttttatttcatttaaacattttcaaacttttaaaaattaaaattatcttaaaaaacacgtgtggtttcgaaccatatggttaaaaatattgaggttctctattttgcaaaaaatttggaaaccttcatggaagttgatataaattgttgtaattagttagtattttttctttatttgtggcttaatcatgctatgattgatcattttatcatcatttctataatgtaaaacaatatatttgatgtgtattgtaaatttttatgcaagagtagaaaattaataaaaaaaaaagagagaaaaataattataaagtaatataaagctattaacattttaagaaaaataatagtgaaaatgaaagtttcggacaataattcaataaagaaatttgtagatttcgattaaaatttccaaaattaagattttcattaacacgtgtgcctcgcaaccgtctcctacgttatcccgtaagtagcccaatctatcccattcttacacaagccgtcggcccaaattgattgtaggccatcggcccactcaacacccacaaaccctagccccttgctcctcctcctcctcttttcacggctgctcactccctctctttttacaccttctccttctccttctccgtccctcttcccttttctgctactatcttcagcacttgctgagcttcaacc

SEQ ID NO: 58 - DNA, 1591 bp, Cucumis melo
  unsure 986: note = unsure at all n locations
  source 1..1591: mol_type = unassigned DNA
aatgttgatttacccttgctttgtttgaatttcgtcctcgtggacttgacctttggtctgcttcgtataggactatttacggctgccctcatagtccaagtccttgtccgccttaccctatactctctatctctagacaatgtgaagcgggcccttctataatattgggccttgaagttttgggctttggatctgccgaattgtgttgggtttctctcccaatttatttcatttctttattcaaataattataaatatggaattttattttatttaaaatataaaagttaaaattgaacgaatccaaaaataatggaatcaaatcgacgttttaacatatttttcaattatgtttttacattcattttcgtcctacaaaaatattcccacctttatttcctcgatatcggaggtcactttgtatgtttcattcgggtgatgtgatatagatcgagtttctatgcttgattgactatggaaatatattttaagaagatgttataaaagtaaaataaatgttttgattgtggatataattatattttaaacaagatgaggaataattagatccgaaccaataatcttgagtcaagagtgtacattgaaagtcgtatattaaataatggttgagtttataataatattgatagattgcagttaaccatattttctcaagttgttgaccaaagtacttattttataaacagtttagggaatgtttatgaagttttgccaagtgttttgaacctatatgagtattgacttaattggtatataagtgcattaacaatcaagaggtatttaatttgaatcgtcctacccctatcatgccaacaaaacaattatatgtttgtcatattttattgaaagtgttttcagcgcaatttagtttgatttgcgtacaaaacatgtctacacgtatcgagttagtagtaatggttgctagttaagactgtgaactaaaactttaaatttacattaaaaanaaaaacattatggtcgtttggtcctcatatgtgattgatagatattgattaatgagtatttgtggttgttgccaacaataaagatgtagacaagtgaactatgttggttgtcaaatcttgtttgtatttgttatgtgtggttttcaccaccaatgttgtagagtgtcagatccagaatagcttgatcatttttcatatatatctacagactcaattagtagataaaaactataagactttgacttatttctcttaaaatgtctcctcgttctgtacaatcctcaacaacgtttggtgactttaaaacatcacaagaatctaagaagaatgatgaattagatgcaatgcaaagatttggaccttaattttgttactttaaactttatatccgaacattggaagaggcaagcaaaaagcgcgctttagaatcgcggtttctttgggccgagtgggttgctcataacagcggaggtttgcttttctgccaagaaaacccctcaaagaaaaagggcttaataagcagctgctccatttctaagtgggtttagcctttagcacggaagcgccaattcgattcaactctgatacactgcaaaaattccgcc

SEQ ID NO: 59 - DNA, 2000 bp, Cucumis melo
  source 1..2000: mol_type = unassigned DNA
aaagaatggagcaagctgatattgctaagcaaaagcttctgcatgattgtgaagttcttcaccaccgccttcaagattctactgtcgactttttcattgagcaggaaaataaactaattttggaaactgcttcgacagctgacgacgcaatagatctgttggcaacatctgataatcacattaaccttcttttagcagaggtacttgcgtttgtttccattgaaatgcatttgcatattgaacttcttcatcctgaatggcacaagtttttgtccatacaaacaggcaaagcttctggctcatgatgctaacaacagcgatgacactgctggatcagcccgtccaaatggaactgataaaggggcagctgaccaagtattaagtaacatactagcaaatatgcttgtcgaaaatgcccgattaaggatgcagatgaacgccgtcatccgctgtgttctaaatgcaaatgggacaagtgagaaagatgaagatgaatctctcaaggaagaactgttctaagcaagtttttagaggaagagattcctgaatgcacatatacaatgaccttatactgtcgtggcaagaaatgggagagctgtagattttgaataaatgcaacagatgttgcccattaatttgcaagtcctgacaaatttggttgtcggaggtgtagaaatgatgtatcaattaaatatttaacaaagtgccttttggcttggctaatcatgggcatttgaagactttgcacttggtaagagctcaaacaaaatctgggtggctaaatttagtgttgattaaatggaatttcactgatattcatgatctgtctcttcttccttcattgatatattatcttctcagtaaactcctgggcctgatgcagaattgcttttaaccatctgcatacagagaagaagtaaaaactagctcacgtggataaagggaaatttctactgacatgttggcattagaagaaaattttgaaagagttctattaccataacatcatctacttccgtgtattattgaaactattatttctcttacccggagatattaaattaataaatttctatttacattttgaagatgctcgtgattattgataaaaatgatgaatcattattttgattacgttacaaaaaagtcaaagagagtaacaaagctatcaacaaaatattagtaatatatacaaaaaaagtgtaaatttaatattaacactgagaaatatacacttaagctaatgggttaaaatatttatccattgaattaaatatggtttttctgtatttgtgatattccaataaatatgaagctgttatactgtcaaattcatattctgcctatacaatcaatttcaagtcactcaattttgcaaaaccatatcatattgagttcaaataaaatttcatatctatatacataacgaaaatgttatgtttttgcttttaatgttttgggtatctttctaagctacaagaaaatgtaaaaatgataataagaaatagattatattaaaattattttacaaatcaaattgcggggatagctcagttgggagagcgtcagactgaagatctgaaggtcgcgtgttcgatccacgctcaccgcaaatttttttcttcttttttttcccttgtgtatcattttaaatgggctgttcttactttgaactgcggaagcccatgaaagctaggcccaatttagaaaccgaccatctcaagggtcggttcgtcatttatcaagatccgataacccgattcgctccattttagtctctgctctttcatctccctcacccattctcgcttccactgagcgggcaagggagcttaacccctcaaagccctagaaaccgccattggagaagctccactagcttcttcttctatcagcgaacgtattttcgtcttgtatagacctttcatctctggaaccgatcggaagtttggagtttcttggtctcagtttgtagattagttttatcttggcgtctcaat

SEQ ID NO: 60 - DNA, 2000 bp, Cucumis melo
  source 1..2000: mol_type = unassigned DNA
gtgcatttaaaataatctagttgcatgttctaggttcgatttattatttggggatttagttgtgtctgtatgattgaaaaaattaatgttgatcttgtaacacaattgtttttccctcgatgatttgagattatttcaacaatttagatccaatgtttaaaaagccaccttggcatcttgccttcctcattcgcaacctgcctccagttgaagcctcgaggctcaaagcccagtgccctaggacttctttattaattttacttaaaaataaagtttgtatccctaaatgcataaaatacccttgtgtttaaggctttctgtttcttcgcgtttcacgtcaggtcagaccatgctcagctatttttccaccattcttcttcttctctcccaaagtctatcaagtattttatttccacacatatattcacctacgccaatttctttttaaaattttatagatatatacagtgcacctcacgaaaacaaagtttgcacttcttcagttttttgtttcgcctcacacttaagctacaaaaggttattacgttttagtaacccactactcagctttaaaaacactatttgtatcatatgacgtcgcccttatggaataatttcacttgattatcgggttgtttcataaacaatcttactctgttgtacctttgacaggcctggagagcatgcaactcctctcttgcttgagtttgagtaacaataaaatcggaaattttactgcattggagcctctgagactgataaaattcttaaaagttttggatatatcgtacaacgagataggttcgcattcgatcgacacaaccagatatctcttctcatctccactgtcgcattccgaagaaattgatttgagcagtgatgaaatggcaacaaattttactgatatggcaagttactgggaagcatattttctattcaaagatataagcttgatgcaattggatatagaaggaaacacaatatctagtgaaagtttcaaagcatttctggtaaagattcttcccaaactccactggcttgatgggaaacgggtacaatagatatggcttaatttatctacatccaatcctctgtccattgtggttgttcatcccctgaatgtaaaaaggtacgctacgaactagcattgatcctaaattgaagacattggttttgatttcttcccaatgcaaggttaagaactaaggatttgatattgcatccaataagcataggttatttaagattttggtgatagtgaaaattaggtgacatgtctcgaaagcttaaagggatacatgaggtatggagatggagatggatgtggttacaacatggaaatgaatacggtgcccagttttttggactgctctaaatcaaattttatcatatacattatgatactgtgtgccaattgtatttaaaaaggtactgaactttacatttttgttgtcccaaattttgaaggattgtagttttaataattcttataataactatcaatgttaattaaaaacttcagtatatttacaatttttctaaaaatgtttgctatacgtttagttattatcttgatcaattgccccaagagaaaaattaccctggactatttcccaaaaacatcttctagtcgtccatcagctatagtttcaaatctgtgtgggcccagtcggcccagttcattgggcctgagaatagagatcatgaaccggacggcccaaacctttttcaggccccagccaagcctggcctacaaacttctaacctaaaaccttatccgttgaagcaatccaataaaacaaagccacgtaagcacccaggatctaaaaatgtatccaaatccaccaatctgaggccacaaatttagcctctgtggctgaatggatgtcgaattacaagaatctctcgatttcttcctcttaaatccatttacccttcaaacaaataaacacaaaataaagaaaaggagaagaaacaattgtcgtaattagcagcaagaa

SEQ ID NO: 61 - DNA, 2000 bp, Cucumis melo
  source 1..2000: mol_type = unassigned DNA
acctagaacttctaaacgataatctcggaaaaaaaattggaacaaatcataataatgacaattaagaagcaagaaccgttgacaaaagcaagatttagagggagtaaatttgcatggtttggtgatgattatttagttgaatttagcctactcttaggaagtatccaataatcatacgcaaatttcacgtagcatatgaagcaagtgcatcataataccgcataacctgcggggttttgtcatctcgattaaacacaatgtgaacatgatgatgtctatgtgtttccagcttttgttctaatgatgatagacgatagtgtggtatagttcatatccttgatttaattgtttccatgtatactatcgaatttttaatatataattaatgtatgaaatcaaatatcaaataatgattgtgatttaatggaataagatcatgtctaaaattggtaatagtaataacgaagaaggaagagaataataaactacgatttcttgtgaatctcctagataaattaggataaaaactacgagtaagaatagaataattatactatataaaataggagttacaaattttgtttcttaaaataccaagctctgttacaagaaaaaactttaggtattatatcttcaacattttgttaatttgttagagattttaggatagtttgtcaactatgggtcttctaagaaacttggtcatcaagcaaatctaatgactcgaattgtccttgatcgatgtgaaagatccaatgacttcgaattatctttatgcaatgtgtaagatctaattgtcataaattgatctcatgtgcaaagtgtaagatccaatgatccaaaattgtctccaacaacttcttgaacaataagataactctttgaagaatcttgaatattaattttgacatagatagattgatcttgaatattaggaaataaggaaattttcttatgtacatgcctgaactccttcaacatagcattttgaatcatatctcttctctagtaacttgtatagttgcaatatattttgcttctgttgttgatatatcaacactgattgaagttttgaaacccaacatatagctctagtggcaagagttaagacatatatgtggtgaatttacttctatcaagatcacaatctaagcagatatactttgaaaataaagttagattatccattatacaatgtaatatttacggaccatttaactcgcttagaaattagagttattttgcaaactttattgacaaatatcttcaaaaatttcatacaccgtatagacactatcataagatgttaaagaaaaaaaaaaggtgaattttccatacaattaaaaaaaatcttaaactataaaggtggtttcgatacctttaaactttgaaaagtttcattttaattctcgcacttattgttttaaaacaaacttagtaaaattttcgtataaatttagaaagaaattttatatttacaggtggggaaaattctaaacacatagatgaagataaataaaaacacgatcaactataaactatacctattattaccttcatccttaacaccatgcactcaaatattcattaattctctatatttttttctatcttagcctcaaaatttactttcatcctaaacttcgagccctcaaatttgcgttatttcattcacgatattccttttttacgttctttcatttatggtattcttctttacgttcttctatttacgatattcttcttgcttttatagtgttttagatttgttcataaacaacgtataaattgaaaactttataaatttagggcattaaggtataattgaaattaaaaccatatttatagtcattaaccacagtattatttatcctttatttattaaaaaaaaaatctacttttagttttaaatttaggcattttacgcaaagctaattacgacataaaacaccaaaaggagaccccgttcgatcttcacatcttctcggccagaaacgacc

SEQ ID NO: 62 - DNA, 2000 bp, Cucumis melo
  source 1..2000: mol_type = unassigned DNA
gcaatggcggaaataacgtagcagagggcacaaacaacgaagaggagcttcgttcttcggtgcgccatctgggaaagtgagaaatggtggacgaaacacaaacgggggaatcggattggatctctcagaaaagaaatggttggattcgatcacagatcaatgaagcacattactcggtttttcaagaagattacaagaacttgcttcttgaaacctctcttcttctgtttgaaatttttgggcgtagaattgaggaccggagcagtcgcctgaatttggtcgtcgaatcgattccacggaaacaaaaatatgaagaaaccaaatgaatgacttagcccactcactacgcttggcgacgtgttcttcttacgaacggaccaatcaaatgcgagcctgatgaatatgggccaatcatattatgccacgtaagactttacttttgcccctgacctatgggaagaaaattgtggtcttttcttatgtcaatagaagaaaaataaaattatatgaaagtcttaaaaggaaaaaaacaaaccatgttaatattactgtttaaaaccataacacaaaatcaattattgtttatgttttgagactcccttatggtgtttgctagatagtgtggattttgtttttgaaaattgtttttgaattttgttattcttaagtttttttatccgaaaatttcattctagaaaacaaaattatataaaaccattttaaaacataatatatcgtgttatagttttttaatgtaacgggattacacggcctattatcaattatataataagatagattaaataaacaaaaatgatttatatggcttttttaaaaataaaatttaatctctaccgcttataactataattaagtcattttggtttaataaaatcatattatatagtctcactcgtatgtattatttacaaaagatgtcgactttttatcaaattatagactaaactataattttcttcgaggctaaaattataatttaaccaaatttataaatgtaaaatgtatttataaataaacgaataatagcttgtcgtcaactatattttagtggataagtaagattagttttatgatttataaatatatagtataaaacacatttaaacatgttttgttcattgcgtttggttgatatttaaacctagtaacgaaaaagtattaggtattacattaaattagcatccacctacaatgttaaatttttaagtcagttaataatttaagagactctcttcaacattgacttcatgcaacataaaatggtagaaattttcacaccattgtttatcgacattactacgtaggagaatggcaaaactttcttatatgtatgtgtgcttttagatgtgtctttacatcccttatcaaaacgaaaacctaattctaaccaaatcaaaccaacccgggttgttgggttattcttacaagccatttgttggattaaaaaaccaaaatagaggatgttcggttcaagcattttaaagttttgggctatttagttcgaccactggtttgttcaaagtcgggtcggaccaaaccgtgagcgatgtaaacaacaaaggtctaaattgggccgggatcagatgggctgaagatccacgattctggtttccaacccaaggcccaatgaattacaacaaaaaagcgtactcaggaaatccgaatctggatctcaacgtactctaacctctcacagttcgccacgtcaagaaaacacgtcaatactttaggcgaaaatcaagtgaagaattccccacaataaggaatcgtatatccacgaaactatccaatcagcttacgccatcggaagattcggaacaaagcaacagttcaatggtatatcatagggtgagaataagtcggttccgcagactagtatttcttagtcaaactttacctgcttcaatcggccgccgatttcccgatatttacaacatttagttccgatttttccctcgaagctctgaagtatcgtaaaa

SEQ ID NO: 63 - DNA, 2000 bp, Cucumis melo
  source 1..2000: mol_type = unassigned DNA
gatcaaccttgaaattttcccacatactgtgttgtaaagttgtccccaatttcattcacaaattacctacttgaggaatcggcagtaagaagagatcataatgtatttttgctactacactcgcaagtctaatcagaggatttgattacaatatcttgctgctgtaatagattcgttcataaattaatccagattgaaaagtcaagctttacttcattttcatcgacaatgtagaaattttgtttataactttgtactattgaatctattgctcctcgatttgcccccttggtacgatatcaagccattatttttccaactcttactcgcaacttcaacgcatgaacttgaccagcttcaacctataatcttatgcatgtttttaatgattaaagctgaaatagattgtgaaacgtaccttattctcactaccgctgccaaagccaaccaagcttccacgggtacccataaggtccacaatcatggcactcttggatgacatgtattggttcctactgtctcttccgggttctcttattaatggccccgaaagcaacctctcccggcattctcgaaattcggctcactaatattctttagctactaaaacacatgtcctcaaatttctcatttaaatgtgatctgagaaagtcattcgacccattttagtttaaataagcatcaagtcaaaaaccatttaacgtgggcttaaaaatttacagcagcgcagcgtacactaaagtttatgaacgatgaaagtgggtggcagaagaaagcaagaagtccgagagacatgccaaaaagagtaaaagtcatttgttggggccttgacagcaaggttccatatgcatcggtccattgcagcatggcggctcaaaattaaattttcacccttgcttttgcttctctaacctacccttctacgcatcgtgtctatcttccttcacactcattttgtggtaagctttaacgcaacattttcttaatgtaatttaagcttggcccaccaatccctttgaaaagtttcctctagatggtgcgtgtcaatttcaaattaacaatttgaacttatagttctaacccccatattgtctgccctttttctcttcttcttcttcttcttctagttttgttctggtttaatctttttcggttttctctgtgcagggtagtagcttttaagcttagtgattttctcttgttaacaactctaagcagtgaattgttagagacctattatttcatataaatactagatgacttcgactcattgattaggctggaagctgtcaaaattaaagagtttgacaaatacccactaatttggtaaccaagagccagcaggaacatttgtatttattgagacaagtgaaagtttgttattttctttactcaaaatctctctttaattttatagatatagacattacttggataagaaagggagttcaccggccggaggttttccttcaaatttaacagtgactgaggtctctttcagctttgtttttttggtgttattactgtttgctcaatcctttgaacgagtggtgtaacttgttaaatgcccacaaattcatgggacgcaatcctttaggagaaaggttggccactagttattggtggttaccgtggctcttagcaacttagcatcagaatttgtcttgaacttctagtcgttgaaaattctcttcatacaaagctaagtctgcttatttgtaggatccataaacatgagatgataagggtgatgggcctaagaatgcttgatggaaacatggtcattggacttgcttattaattgaaaaaaccagccccgtctctggttagaaccctcattaggattgtattgtttcaattctttcagcttgttctggattttaaaggctccaatggtttgagatgatagtcatggaggtgggaaggaatggacaatacagttttgaagaactgggttatctcaaatgggaaggtgaaatgtttatgtcagtatttgcttg

SEQ ID NO: 64 - DNA, 2000 bp, Cucumis melo
  source 1..2000: mol_type = unassigned DNA
atcttcaccgttaaatcgccgtggttgttagcggcggcgaggagagagagtgctctttctctgagaactcctgccatagtagacctaaaggaagaaagtggtagtgaaacaaggaatggtgaggagggtgagaattgaggaagtggttagggctttgaaggaacggggaattttatttccgggaagggaaacaacaggggagaacacagccggagcggtgtggttgtgagaaaatttaagcaagcagatgagacgacggcctggcgccgaggacaggcatatgaatatcacgtggctatggctatgggaaattgaacgtaggccctttctcattcttataccaatcttcatttttctattttctagggtttcttttctttcttttttctttttcctttttcacatttttatatgtcattgaatttcgaagtttggagttaatatgttggagtcgtgtatctatttagcttcatgggttataacattattttggatgatgtatgatatttaatctcaatttaagaaggaaacgagtaaccaaaaaatcttataatgaggtttgtccatcttttatgtattattctccacttatcacatttgtttgaaataaataaataaataaatgttgtgtcacctcaaacacaaccatatggttcaaattgaaatttaacacttgatggtccctatgttccatacgacctaacaaggtcatcttttgattgtgaggttcatccaacataaagttgttataaactaagaatatttcacttatgagtgtttatgtgcacgttgttggtataggccataattttcaatcatttaaaacttttattaaccatgatttcacattatcttgatctctcccattcgaatatgattttggttcatctatattccccttataaactcaacgttacgtgcctaccagttttcgcttggctcatccccaacccatatcttactgtggaatgttttttctctgataccatttgtattgtttcacaccttcgaatcatattttagaatgttgatacagtacctaatgcatgtgatattcccctccatttgttgtgacatggcagcatttgttcttacttgtgtttgaattgttttctaagagaaaaaaatgatatctccacaaaccaacgcacatcattttagcatatcatgtgtctcattcacgtggttcttaaaaaaaaatcaggacattatccaataagacgtggtcaagggatgaaccaaatgaaaattaaaagggcatgtaatggccgagttcatgaatgcgtcataaatgaatcaatatcacactaaaataagaccgatcacaagggtgtgaaagcatagttaacaataatataaaaaaaactaaaagctcatatctatgccaacaacatacacattattttcgattgcttaatcgtatgaactttaaagttaaacgtgtttattttaaagttaaacgtgcttatcttaaaacaatcttatgttggacgacctccacaattttttccattacgcatgtgagaaacacattgaaaggactcgaattagcatgtagagaatggtgtagcccccattctataaaagcaactcaagatctgaacatgcattgaaatttcactcttcattcctgacacatacataaagagaagcaagtacgagaatcatcctctactttttattcacaagttttaagtcaaatttcaacttgatttgtatgtttcaaaacgacacacctactcatttaatcttgagcgttacttcaattgtttttatgtttcaaaatgttaaaaaagaaaaaaaaaaaaagttcaatagttttgtaaattgcaaaaaaagagaattacgagtatgcccctgtacatttagaagaagcgtaaggtccatatgggaatcagaacaatcaatcgacggccacatctcacgagacataaacagggggagttggaggaatcgacggagatcggaatctggtttagggttttagcaaaagaagaaca

SEQ ID NO: 65 - DNA, 2000 bp, Cucumis melo
  source 1..2000: mol_type = unassigned DNA
aactcagtgaatacgataagaaatttaattgaagttaacaaactcaaacttaaatattttttaacacggacaatttaaaaccaaattcatagtctccttgtatagtgtttagagtgtgtcgcttcattaaacctttctatcgtggaacaaatctcttctaatattttgtcaaaaacctatcatcacccaaaatatcatgataattatttgatgaggatcaaggcttagagaggaacaagggaacttttcacaagggtggagagatttagctattaggtttaactcgttgcttctaatggtggatgaataacgacaaattttaaacaatgaacgttatcacgttgaaactatctacttctctcaacctactactttatcataaggtttgaaaagttctatcgaaaattttaaatacataaaacataaaaaggaaaattttcattggagaattttccatatatgtttacccacaaaactaaggctaattaaaaagctaaccttaagactaaggctaaaatggtatcttatgctacatttttcagttgctatgttttgaagcaaaagctaattatttgctaataatgagataggcatgtgggtgagtgatgagcttagcctggcctgcctttgtgtttcttcttattctcttaaatatcattgttcaatcaaaatagttttgttaaatttagcccatcctcacttcaacctcttatatttggattggccttctttgttttttgggcttttgatatttgatgtaatggacttcaatcatttatagaagccttaccctacagaaacaaacaaacaaaaagaggaaaaaaaaaatggtgagttggttaataacaactttctaactcaaccaatatatggtgtgtgtatatatatatatatatcgaatacaaaatatgaatatgatatgaccacataaaattgttgaaagggttgaaaattagtgaattggacttttaaattttgtagtgtagtggtttacctatgatgctcgtaatgttatttaattttaaatgtgttttttttttttacaaaaaaaagtctcgcggtgcaagttcaataagttgatttaaaaacaaatccatcaaaataatgttcgcttgatatgatcgagtatagagccgaatgtgtatcaaacataaattcaaactttaatagagtgaaaaataaatgctacgcaaacaaagtttttgtattagcttcttaaatgtacatatatacttttccgattcaaacacctccaaaataaaactcaaaagttaaaatttagactcagaaaatgagagaaaaagaaaactaaaaacgaattctaaagataagcattttcaaatataggaaaatgaacaataaatatttacaaaatagaagaattgtaaaaaacgacaaattgacataatacttacaaaacataacaaaatttcagattctatcaatgacatacactgatatatctttattagtcatagaaagtctatcatttataaaatccaaatttttgttatatattgtaaatatttaaatttgttttaccatatttaaaaattttagatttatcaccaacaaataaccatagatttcaaattttgctataaatatttttaaccgtttatttaccataatttttctattataaaaacaaaaaaaacaaaaaacagataaaagcgaagaaaaagtaagagagcagaaatattttttgatttaggtttcatttggtaaaaaattgttattaaaaaatacaaactaatgggaaacaataataataatttaatttttttaaaatctaaaaagaaaatagttgacaacaataatttaataatttaatacacaagccagtgttattaatctctttttttctaacaacgcctttcacgagacatcctctcaatcctcgacatccagtggaaaaacagtatccctcaaccctcagctttccccaaccgccctccgtcgttcttctgatcgtcgccaccctactccgtcatcggaaa

SEQ ID NO: 66 - DNA, 2000 bp, Cucumis melo
  source 1..2000: mol_type = unassigned DNA
catatatttattatgttccacttgataaccattttgtttttgaaaattaagtttaaagacgacactaattccatcttcaactttcttcttttgttatcaacattcgaccaatagtccagaaaaccaattaagttgttgaaaactaaaaaaaaaaaaaattcttataaagttgttttttttttaaatttggttaaaaattttaatcattatacttaaaaaatatatacacgaatcatagtagaaaattgaaaacaaataaacttaattccaaatctactttaaaggctcactatctgtcaagaggctttggtatagttgtctgtactgattaagtgtgagagttcttttaatatttgtagctgaccaataaattctttcctttctttctaattttgctttaactccctatcctattcatacacaataaatatacaccatattctaattgacaatattgtttggatttgtttgttttctttacggtaggcaagaagttgcctagttgttgtctgacctcaaaaccctttgttgataagagcaaacaaagtctagttttccaaaaaaaaaatcaccaactcaaccaaatcttgagccttttactaatttccatcccaaactaatatctaatcagtgcttacatgtttgagccttcaactcaatttaacatcaaaacatcttgcaaccacaccttgacatgagtatgaaaacaatataggagagaactttagtattacattgagttccattatcattgtacattctcaaccaacgaaaccaacccaaaacaaaatagttttttgtaacatatgagattaggtatcgtcctagttaatgattttacaaagttatatgagtattcatttgttgatatagtttgaccggatcggacagttggctacaatggtatatttctataaactaaggtatacaatttttcatgtatgttgtttgatattgttttattattggcacatgtcttttgtgtccaatagtaataacaaggttgtttcttatctaaataaaataaactcttgccagataattgaagttagacttttaatcaaaacgtaatattaaatggggatgagaaataattgattattaggtaaacctaacaataaaatctttaaattgtgttagaatcatttagttagtcgagttctacactaaaaaaaattaaaaacactaaaatcatttataaataaaatattcaatatcttcaaaatgtactaaaacattgaagctcataaaactaatcatttttcttttgattaaatttctctctcatattaccaagaaacctaagataacattaccaacgattcataccaaaaaaatttattatcattgaacatatctcaaactagtgtattcaataatggttagagtagtagttatattaaggtgccatgagtttgatatttttcttttttgcctaaattaggttaagccgtagctagcttgaacaatgctaaagatcttcttaagagtttcgtagtttaacgtttatatgataaattttattacatccgaacttgatatttaatttttgtggctcttatctgtgtttagtttttcttattctcttttaacttgtagtaatcaaatgaaagccatttgcaaatgaggacaaatgcatctgcaagatatatattagccaatctcttgatatttttatgctctatgagacaatatattctgccatttgcccatcaaatggccataatttctcaagatttttccatttcgagtttgtttcaatcttctactccttttgtttttcctttgttcaattttttggacctttgatgaaatatcttcataactcctatgacgtgggcaccatccattggttgtcatttgataagaaatatgtgtcaatggcacaattcccattccatttatatattatatagttcctaaagccatatccccatgatttatatccttcttcaagctcacaattgaactttaacattacttcttccctacacaaagat

SEQ ID NO: 67 - DNA, 2000 bp, Cucumis melo
  source 1..2000: mol_type = unassigned DNA
aaataatttgtggattttatcatattatgtaccttagactttgtaaggtttataacacaagatgtggagaaatcccatgatgaacatggacgttattatatcctttgaaactaaaaacaaaggaaaaaaagacaaatggctgagtataagaaaaagagaagaaacaaccaaaaagctaaaatgtcatgctctcatatgtaacatatttgaattgggattgatttcaagatgtattttaaaataatttaacacacgataaaataattctaacaaactcaaaattactcttaaacatttacttgatatcttcgacataatgacttttgttttaatacaactcaaaacataattaaggttggttttaaatgacaacaaacgaaaaactcgattctaatttcaatactagtctttgaaagccctaaggagcgtggttttgaattgtatagcaaggtttcgaaaattttaatcatcaaacaataggtatatttacctgttcctcaagtacagttaattcatagctacttgtcgtgcttcctacgacaacattgtaagacttgccacacactaattgaagccgttgttgaaggtagttttccatgtatagcttgaatcgacggatgaccaaagaggttgaagaaggtttgaaaaataggggaagggatatacaaagtttgagagtgagagagggagagaaatagaaatagaagagactgaaaaatgtaaaagaaaggatgaaaaaatgtggggtaaacgcaaattggatttttatagtagtattttgaaaatgctatagaaaggctatgtatagtagtgcttccaaagttctatatgaatgagacaaatcaaaatatattttttttgattaattaaccccaaaaagactcataaaaaaatcttataaatcccactaagatattgcatgttatatgtagtaaaatttatcgttcaaataaaccttaaacataattcaaacgaagaaagtaaaaatttgaatttcttatcttacatcattcaaccaaacaatttccatataagagattaaacttctaactttaagagagagatatccagcatatgcaacctaaaccaacagtgactttgctatatatttgcacaaaatgtgggggacaaagttgtaatttcggaatatcaatgattaaagaaaaggtaaaatttaaaattcggaagcttgacgtggcaacacggaatggtgatgatatttccaactcctcgcgacttttagaagttggcctcaccaaccgcatatccgcccctttgccacgtgtcagactacaacaacttccaacaatttcctttaagaacaccaaattatatcaataaatatttaacttaaattaagaatatatttgttacaattttcctactgagttagatagatagacagacttgtcaattaactaataagtccaaagtcaatttactcaacatagatacaattctaatttgagtgtgaaatacatattcaaatcaaaattattattaagaggaaaaactgatttgctttctcaatttaaaatataatattttgaaaaagaaacacacatgtattatggttttcaatatatttactttcttagtcacctaatctcaaactaatttttgaagaaattaaatatatatattatcatttttattttcttggttatgatattggtatagaatcaacaaaactacaagtgtcctctcctctgctatcgatggggattagactctaaacttgtttagcgattagtaattatatattagaaaaagtttatgttaatgtggacccgacaatctcaccaataggctcttcacttcaccaacccccgacccattccctctaataattcgacacggctcatccccggttcgaaccgggccgacgtcttcctatttaacacacctccatttcctcttccctccgcaacacaaacagagacctcaccggaaaatcaagttaaagcaaaaccaaagagaagcttcatcactctccggaa

SEQ ID NO: 68 - DNA, 2000 bp, Cucumis melo
  source 1..2000: mol_type = unassigned DNA
taatagttgcaggtcttgtttaaaatactaatctaggtgtgtaaaacatagaaagtttaatgtggaatttcttatgagaacgattaaggctggtgaatctcgcttggttaaatttgaagtagtttcacttcgatggagactagacgtttaggcattcgtaatttcagagacaacataacgaggctacgttgggaaaatagttatgtcattttatcataactgcatactttgtggtaaggatcatactgattagtaagtacggtttccactctatagtgtttgagtcaacttctgtgcccctttactaatctcacgagagaatccgcctggtcctgtaacatttgggtgtcaaagaaacttgtaggaaagattccgaccaccatggattgaatcataagtcaagggccattagtaaaaacctctaacttgctcttgctttaaattttcctctattctccttattcgttaagcattgggtgtgggtgctatactaacttttgtgggttgttaatggcctttgtttctgtagatagtaaggacttctactgtaaacttgctttttgtttgcactttctcactctttcattttgttaaaaaatataagacaacataacagagcgacagagagaaagagagactaaccatagcaactggagctccttgtgaaatttatccaattcctcagaagtagacaatataggcttctttacagcaacttttctaccatccaaccttccttcatacacagtactctcggcccctgcatcaccattcgataaaaatccaatatgtcaacagaacctctcaggcaattgaaccggaataaattagtgcagcgttgagtgcttacctcgggcaattggagagagcagcgtgaatgcggaaggttgaagatgaagaggaatcgaattgctggagcagcagccctgatgcaggtgttcggatccataattcccaaatccatatccgttttcgtgaagaaatgttgaggaaaaattcattatgcgttcagtttataccattggagagtgggaaagttcgtattgttttgctaatttcgtcgattctcaggtcttggagtaaaaacgttgtacccgccacttcccattgggccattgtccaatattgtttgggttgggcgggtggatgacccaaattttggggaagatatgagatttgtccaactctgttatcaaatatgaccaaatgaacaaaatattgacttttttttttctatatttttttgaatgaagtataagtagttgttttattttgtttattttaactcaaaattaccaaatttggatttcacaaacataaaatagattttatactttttataattcaatcgaaagttgatcgtatatgaaaagaacaatgaataaagaaagaaatgtaaaatttatatcaacttaattaaaacctcgcaatacaaaaatcgagtgaaatagagggtggaggatgagaggaagagggagaagacatccataccctccatggacatgggtagatgtatgggttgggttgggttgggttgaattgggtcaacccatccactcggttcatatagacagcattcgttttataatttatccaaaataaaatataattaaaagaagaaaataaaagaaaaacgaaatctccaattcgcgtaggaaattaaaaaggaagagttaattcaattcgattctctcccatcttcatcataattttgcgaaaggatcgtaagttgtatacttctctccttgctgcttttcgaatcgggataagaatattttctttttgtcttccccattcctactcttcaaaattctctgcattttctacccatcacttttacttcaaccatttttgttgttgggagttccatattttgatttcctctacaacgcctaaactcttcttcttcttcttcttcttcttcttcttggagtgatttttcagttcaattttggggatttcatctatttctttgatctgcagcgttgctggaagttgcg

SEQ ID NO: 69 - DNA, 2000 bp, Cucumis melo
  source 1..2000: mol_type = unassigned DNA
agtttattctgttgtagcaattcaaagcagtgtgatccagatgagtacatatttggtagtagactcaattatcattttatggttgaaaaccacatcctcttctgtcattgttcatattattacgaggaaaaaagccacgtcctctttcaaaatttcttccacaaactctttcttaaaaggaggaattagagtttgaaagactaattagatcgagaattattcatattcagattggtacctaattgagtagcaaccatcgcagtaggttgaagagaaaggtcctcttgtttacgatgttttgcaagagcaaattcttcaaatttcaagagagtatgaagttcttcaaagattactgattgtgaacgagtgcgcatagagatgtgaaaatattgtattcatctagaaatcaaatgagagtatagatcaacaaatcttcatcgtttactattgaaacacacatttgcaagtttatctttgatttttttgcccctcctcatgtatgaattaatagattcatcaacttctttgaaatcgattgaagattagttttgagattaacaatatttgatcgtaaatttgagtagtgattttcaagtgcgacccagacttcctttgatgaagtacaaccaacaattagggtttttacagacatagtagcactgatcaaggtcataaaagcttgatctttagcaatttaatctttatatataaaagattcaacattgtcgtcgattgattttgtattgtagatgaactagtggtcgaagaaatcaaaatcgattttgaaatcaaaatcgattttgctagagctgtacttatgtcatcaacaaatccatataaatccatatagcttgtgtgctctcaaaatagtggagaattggaatttctaagaaacataatttgttgattcgagtctgataaatatgaggtacatatatttgtgaggaagatcaaaaaattgatttcttttgttcaagaagactcagcggaagccattgatggagagaacaaaaatcggaggggatggttaatcatgggtctttgatctgctctaataccatgtgatctttaccaagttgtgaaaaaataatctctcattttctcattaatttacaataatagaatatgggtatctattacaacccaatttacagaggaaatactagctgattacaacagaatcagtgccaaatcaattattaaaactaatactcaacactaattaccaaagaattagtggtttttttaccacgaatttatggggtaaaaaaagtgaacttttaccaaattagtaaaataaaaaaagaaagaaaaaaaaacgtaatattcaaatggatggtgaggcatgaagaagagtagcctaaagtacatgaagagctaaaagacttattatcttccattggtcccattgaagaccacaaagaaaatatcagtcctttttctctttagagacacaaacccaaagtagaaagaatctttcacaagaattaggaatttaatgcaatttttctttttaaaaaaaatctccaattttctatctcattatccaccctttccactctaaacttcactacaatttgatgaaatctgtttccaccaatcagattgcaccaaattccatcaaaaacgccccatcagataattatggatgtcttcttcttcctctcttctttcgtggctgaaattgaagctcaactcaaaaatacatttcattttcaaaattccctgatgacccaattcgccacgtgtcccttccactcaccactacccacacaaaacaactgcttctcttcctcttcctcttcttctccattaaattcccagacccatccctctgcaacttcgaatgcaacagaaagaaaacggaccaaaaatcccttgaggaatttctcatttttgaagcataattcaaagattaaacccgtattaaccctcttcatcttaccagaggtttgatttattgatcgaattgttttattggttttttttcaaggtcacca

SEQ ID NO: 70 - DNA, 2000 bp, Cucumis melo
  source 1..2000: mol_type = unassigned DNA
gcatcttatggatgtagtcacaacatatttatatggattttctgataatgatatttatatgaaagtcccaaaaggatttaagatacctaaaacatataaatcaaattcccataaactatgttcaataaagttacagagatcattgtatggattgaaaaaatcatgacgaatgtgatacaatcgcctgagtgaatatttagttaaaaaaataatatcaatataattcaatatgtccatgcgtttttataaagaaatcaccgtcaggatttgctattataactgtatatgttgatgatttaaatataattgaaattttgaagagttttcaaaggcaatagaatattaagaaagaatttgagatgaaagatctcagaaaaataaaattttgtcttgattttcaaatcgagcatctagtaaaagggatatttgttcatcaattaacttatacagagaaaattttaaaaagattttatatagataaaacacattcattgaacattctaatgcaagttcattcattaaatgtgaagaaagatatttttcgacgtcgagatgataatgaagaactccttagtccagaagtaccataccttaatacaattggtgcacttattttgtcaataatcaagaccagatattgcattttctataaatttattagctagattcagttctccaacaaaacaacattggaatgaagttaaacatatacttcgttattttcgaggaacaattaatataagattattttattcaaataaatcaaattttaacctagttagttttgcatattcttgatttttatctgatccacataaatctagatctcaaacaggttatctattcacatgtggaggaactgctatatcttaacgatcagtgaaacaaattaccataacagtcaactcttcaaaccgtgctgaaattcttacaattcttgaggcattcatgaggctagcggagaatgaatatggttaaggtcgatgactcaacacattcgaaaattatgtggtttgtcttctagtaaactccttccaacaacattatacgaagacaacacaacttgtatagctcaaataaaatgaggttatattaaaagtgatagaacaaaacacatctcaccgaagtttttctatactcatgatcttgaagaaaatggtgacatcacagtacaaaaaatttgttcaaaagataatttggtagatttatttacaaaattattacctactgcaacctttgaaaaattggtgcacaacattggaacgcgacgacttagatatctcaagtaatgttacatcttacttgccaagttaactatacatagtgacatttggtggagttgtaagaaacactaatattggagaaaaatcgaaagaaattggaaaatatggagaattgaattttttttagatttttcttattttctaattttaggtttccgtattctgattatgcctcattttcacaacattaataactttaataagatgatttcttgggttaagggaaaaaatcatttttttagagttgcacgtacaaaaatattatcataacatatcgattataataaaccaattcaccgtcaacctaacctaggtagagtttgagttaaatgttaaaagaatatccacccctcaacattgtaatcccaactaataaatcagcaacctaaagttttttttaaaaaactaaaaagaagagcaatatattttttttactattatttttttaaagagtggatttatttattaaattaaaaaatgaaaagaagaaaatttgttagtttgggtaatccgaaaacccgattatttgggcccgagaaaccgacgttttgtttattgttcctcacggcaataagtaatggcgtgaatcgaccgcgtgcgcttcaagctatctagacatttttatatcctccgattagaaaccctaattcagattctccgtattacccaccctggaacatctttgaaacgcgaaaaggtgacccgaagaaacttgaa

SEQ ID NO: 71 - DNA, 2000 bp, Cucumis melo
  source 1..2000: mol_type = unassigned DNA
taataaagtcgatgatatgaattaattagacggatgggttatactatagttattttattgtcttttatagagaatttaacattggtagcggggaaaatccgatagatattggtgaagaggaattcgttggtggatgtaaaggaaagttagtttcgccttggcacaacgaagggtttgaatggaaaatcatgataagttcgactgcctgttcaactaaacgaaagataccaagtcaaaccttagttttctttaaggatttatgatcttagtagtgtatctatttaaagattcaaaggtatcaatagactatttatttcaatcgttgtgttgaaatctaggagtatatttaacgattaacttaaaagattttgctatcttgttttgtgtttttcatttttttgggaaaacctagtgtctttttattttatttgatacaataagtattataaaaatgactagaatgactatatacttgatcattattttgacatatttgcaatatattaaaaatgactacttattttaattaccttcatggtctttttttaatttatgaaggggtgggctcgtgtggcagatgaggcctgtcataattagccttaccttaaataattgggccggttctttgggaaatatcggcccaacctaacttttcatgggctcaaatgatgctttatctaatacccatactttccattacctttgtatattgaattagaatgatagaaaaacatactacacagttgagttaggatataaataaatgcattgaactatgtattacatagttgagaaaaatgagaatgaagttttgtcttttgaatatatattctgtgaaagttagatgtatatagaaatgatgatacttcggcgtttgttgaagattgagtggggtgtcaacctaatcatagttggtttaagaaaagttttaattataagataaccgttttaagtgacttatgccatattttgattgcaggttcacaatgaaatgttttaatttggtgattagactttgacaatgtggtaatttatgttaagtgagttgttgtctcgtttaccttgatcatttgtctctactcatttctcattttgtttcatcccttgttatatggcatccattgttgttgtatttgtcattgttcacattcgatgcttaactaggtaagaacaacattttcattttagaattggaacgatagaaattcataagttttatttttgaggcacttggttcattttaatcatagaacattagtccacaatcgtttggaataaatttacactctatctagatatggaactcttgacaacctctaccaaggaaggatgaaaagcaaaaaaagagtagaaaaacgaaagtagacactataacaagccaattagcccattgacaaatattaccacgttattaaagttcattttaatcatcgtgtcaattatcaaccttataggtcaaataccatttataattattttcaaattcaattaatgaaacaagactcaaaaaaccaaacaaatatccaaacccaatatttgagtttagaatataataatttcatagttagacttggagacagatttgtacgtatatgttaaattaaaaatttaatcaaagtataaataaatgatttggagtggcaagaaaatattggccaaaatttcataagaaaaaggaagaaaataaaaaggtgtattggctaacaaaaacccaattccatggggaggagaaaatttgagtcctcaaaaaaggatttcagatagggaaccaaccaatcaaaacgaaggacgtctccacgtgtcgctacaagaggccatctttccaaaatgagatcgcggataaacaagccttttctgagcatagaaaaatggcgaattttaacaaaaagaaaaatctcagtaaagtcatcagctacagctgctctttgacggccacttgattcactatttccctctctttccggcgctgattctagtgtggttgaactttctgcaaagaa

SEQ ID NO: 72 - DNA, 2000 bp, Cucumis melo
  source 1..2000: mol_type = unassigned DNA
attacttgaacttaatccacatttatgtctttatataaagttcgaatactcataatatagccaagaaaccttgtttattggattttgagttttatcataagcaaatctcttatccaataacaaattattaaacaacacttcaacaataactttattcaacaatatattagtttaacattcacaaatcacgagtattagaacataaaacgcaacaaagaatggactaaggtactatattctaacttaggttgtttaggatttccatatgtcaatgcttttgtgatttttgaactagattttcttgttagattaattcaattctatttttaaatggcttaatatcttattttcggatgcttggggattgctagactaccgctttgttgaagcaataagttaaatttgtttgttacaggtattgatcaatctaacatagaattgaatttgtatgaatatttagttagacgcttgaaaactaattattctaccaatgagccgtagatcttaatgcaattgttattaatttgaactttgtatgcttctcatcgattaaatttatatcaagtagttaattaggacaaatctattggctttttcatttaattttgttaagtaaaagcaacttagaattttgaaaatgatgaacccatgatccaatacattgaaagagaattttgtttaactcaaactaggattcttctcacattgatttcgtataatttaactttttcaatttatatcaatccccccagggtgaaaaaaatttgtttgaagaattcatgtgctttctaaatctgatctagacttgccactaaaattaacttttgatatgtaatttggttaaatatttgattcggatttcgacgacaaacaattgatcaatgtggtattaaattctgatctccatgtaagaaatttacacattttcataagttcaatgttgacacaaagagagtaagagcattttaaaaaaaaagatacttttaatcttttctaaaaaaacaccaaaatgccattatgtaaatgtaacctaaataataaacatttaaacttagaattcatgcaattaggctttgtatgggacattgaattgattattaaaatcagtagttatagaccgtgagttataatggtttgtattagaagcataaattattttaattttgatcgtaatagcatgtatttgagatataaattaatttagtttgggtggcaaatagtaaacagtaaagcaaaatataaaaaaatgaatttaaaatagtaagatttgtaacaaatgattaatactataacaaacgtggttttaaaataacgttgatcgtagctaattgaacattatttattgtaaaattgagtgtttttaatatttggagcctcaaacttcgggtggatcaccacaatataatcatattcaaatttaaaattttattttttttattaattataaatattgattgttaatagatgctcattatgggccatctgtcactccctccgtgcatatcctacctgaaacatcatatatcttaaacaatgtccattgccatgtgtcactatttttacatcccatccacttgacaaatatgttgaagatgcctacttttttagggatcatgtaatctatctcatgcttgtcaaattgttcgataatagtgttacaaaaaatttagtaattattattattatatttcttcgatatttatgcttcatatgccattgtgctctccatttttaccatacttaaaaaaatttcttattataaattttttcaaaaaaaaatttactatatagtcatcatctttattaaaattaaaattgagaacctgatatttttgatattaataatttaaaatttgaattaatccactttaaaattattaataatttattcgaatttgggccttaaggaagagatacggaaacaaaccctagatcccatctatatataaatcgccacaaaaccctacctttctctcagtttctcgttttagccggcaaaa

SEQ ID NO: 73 - DNA, 2000 bp, Cucumis melo
  source 1..2000: mol_type = unassigned DNA
tgaaaaactaaattaaattgtccttacatgtgtataaaagaaaccttcggacatttgatctgagaactatgttaaataataagatccaaaaaactgaaccccaacatcttcgaaatcgatttgatttcaattcttaagataagctacattcaagttacctagatgatctaagaaactaatgattggacaaagttagaaactcccaataaaccaatgatcttcaaagcactctacgatcaagacagattaatttttagtttgaatgctttgaacactcgtgcattctatcacaagaacaaaaattatacgttttagaattttcaaatatcattcatcccaatttttattttaaacgtgaaaattacaactctatttatactaattaaaatattaattaacatgttacaatatttaattttatgtcatttcaactaatgtaataaataaaaacaaataagacaacgtaaatacacaatttcataaacatttaatttcacgacttttaagttttctaaataaattttcaactttttcatttgatttaattatgatttctcggatcatatctatatatatatatatatatgaagctgagttttagaaattgtaaattcaaatttctttaaatggtacaaattcaattagtaagaggaaaaacagctaattaaataatgtgtgatgccccactccctaaaacagtgggtttggatcgattaatcaactaaaactgaccacaaaacaatattcttctacaaccccattgatttttttaatcattaagtgccgattcaaagaaacaataaacaaaagaagttgaaaagattgagacttttaaattaaatctgcaagattctctccaaactcatgttgtattcaagtgtttaaagcttaaaatatcagtaattatgtgttatttaacggtgaaaccaatcaaatcaagcaagattcttcaatattcaattccaaatcctcaagtttccatgaaaacttcataacgcctttatccctcgaaagccaaaattcaatttcctccattcatcttgcagccctatctactttccaaaagccaacaaatacccttttaagcagtagccttttgtttggttgtagtaggatctttgtttctcttccattttaacacaagccacaggagaatctctatctctatcctgcaaccttcatccccacattgttcttcctccattatcggaaaaacccagtacagggtttgctttccggccactatccggttgttctttgtaagttttttgggttttcattatctgggtttgtggctgcttgtggattcagggtaatgtggccatgttttatagtccacagcctttttttcttcttttgacatgggattatttctgattctatttgtctattgttactttgtgctttttctggtttgttcttgtggtcatcatttcttatgcttggaagttcgaacatgaatcaattcaacaactaagttgagagtgttcgactctctcatctcattgaccctgatggtatatcttggcttggaagttagaacatgaatcaattgaacagcttacttgagactcgagagtgttcaactctttcatctcattgaccctgatgatatatcttggctttggagttatgaactatgagagcttggaggatgaactaaaaagaagggactattttttgagatggatatttagttttagtaatttagctttttttttttagtacatagtacattaactttgttcgcgaggaaatagtggtcttgttgacgagcatttcttaaacaatgtagtttttgtctcatctctttaaaagtttatggaggggcaaacaagtgagatcaatagttatagtatttcaatctataactttggaacagctgatttttaacttttcctttgtcttttttttattatagaacacattagagtgcgttagattcttcagttctgagattttgatctttgagtgctctcttttagcagtagaggcaaaca

SEQ ID NO: 74 - DNA, 2000 bp, Cucumis melo
  source 1..2000: mol_type = unassigned DNA
actttcagtagattttatctcataaaagagtcataaagataattagtaatgaataaagctttgtttgaagaaatgtttcattgcaactgatatttgtcattgatgtacaaatggctttgtaactctccactttttctaatctaaccatttacatacaaaatatctacgatacactaaaatgaataaagaaattttttttgtcaaaaactgtggggagaattgctccttgttctcaaatcattcatgaactttgcaatttagaagtaacatcaatgaaggcttcttccttgcagggaattctcaaacctccagttgggtggctgaatccaaactcttcttcagccttgttgagcaagtctatgaatgaaggctgactcaagtacgatattggaacgaaaaaccgctttctgtcggtttctcccacgtacactggaatgtggcctttgggaacaatggactgacatcttgctgagacagactgcatcttgagaacttgcttggcagcggaaagaagaaccgaaggcaaacgaattcccatggctaaattggattgaatctttttggaagtggtaaacttcaatgcttgaatgagaatatgtgaaagatttgaagttggagattagttgtttgtttagagtctatatatagaatgagaaaagagaaggtattgtgacatatgaatagaagatgggaaaccaagaaagttgggttcatcaatggctcacatgggttgctccattggttaaggtacattcattttctcattggcaccaatttctggtaagatggccccatatgtcataatacgtgaagtcatattgatctaaacaaaatgggacacaaaaattgtaactatttcaattagcattaaaatcatgtcaagaaaactacattaaatatagatatattagttaatgatgtaataatagtttcatgtgagatcaaactacgatttttttttataaataatgttacttttaaaaaaatgtcaaaaatatggtagaagaaaagctattacaaaaagttaagtcatctactcggttcataatgcgttatcgtggatcgggtacacgacaaggcaatgaagacatagacccagtctatgacttcgatgtaaaatgtgggtttttcctaattactcgtaaaaaaatatttttgaaaacttttctttttaacaaacttaaattttggttaattatatatataaataccatctttactttcttattatccaaaacaatttaccatatataattatatttattcaataaataataatataaaatatttagataaacaaaatcaattatttcaatcttatatattttaaatatacactaagctaatttaaatttacattctgaaaattttaattatatttctatctaatttaagattttaattatatttctatttaatttaaaattttaatggaaaattaaattgtaaataagaataagagtacaaacttactatttttatttcatttttaatttataaacttcatctcttttttcatatatttttaagaaatccaaccttatatttcgaaatttatttaaaaaaattataaaattttttaaactatatataaataaaaattgtaatttttgaaataatttattaattcttacaacaaacttataataataacaataataataataataatgagggtactcgattctcaaaaaaaccgaaccgatcaaacaacgttagatcaccaacacagaagtagggtttttcatcggcacataaaaaccctcacttcttcttcataaaaaccctcacttcttcttgacctaattcgcgccgttgatctccggttcgatcggtttctacgctgtaatctcaagctatctcctaccttatccttccctctctttttcttcttcttcttcgtatatgcatatcttcaaatttgctgctttttttgtctgattattcatctgggtttgtttgcaacaggaaggaggaagaatttcaaatcaagaagaaaa

SEQ ID NO: 75 - DNA, 2000 bp, Cucumis melo
  source 1..2000: mol_type = unassigned DNA
tttattaatctgaatcattctgtttcttctgagagttttattccttttaagattctaattttattttggatagttgaattttggtgtgctctctttgccccttctttattatacattcctttatcttaaaaaagccaaaaagttaaaaaacaaaaactaatcaaaattgtaacatttacaattttatgagcatgacatttaaaatatcgattttgaagttaagacgttgtattctcaccatcggtttttatctcttcccattccattagagtgataggctttatctttcatcactgtcaaaattcatccaacgtccaagatctcttctgcaaagagttacccacaattctctcagactcattggcccaccggataccgagtggatggatagaacctccaagattgcgagagcaaaagctcagccaaaacttgcacaaactcacccatggcttccctctcttgtactacctccattaatctcaccccaagatccttcaattctcgcccccattcaaattagcttcccattttcttggtcttcagtccaaccttcgatggctctcacccctctccattggaccctccaatgggtctagagcaacttgctggttcaatttaaggcaaaatgccgagggtgcaggcatttatggcagccagtcccgagatgatttcaacagagatgatgttgagcaggttcttttactaatttctctcttctttctttgtattttgttttgtgactttgattgttgaagagtggtgtcttttgtttaattgctggtttgggctgattcttatgggtttggagttgaaattgttcttaccctctggctgttctgttttcttttaagtattgtgaattttcaatggctcctttagtgaagatagatgaagaaatttaaattagtaatttttcgtaccgatgactctcttccagtggtgttaatgtcaaactaaccttttctttacgtcataaagcacttaatcggttggaactcagtagacgtctcactcatgtttgtagccctaacctaatgccatggcaatcgaaatttatatcgtatccctattgcgattattaaacatcaccataggtgagacattcctaacgtgatatactgagttctagatggttaagtgctctgacatttcacattaacgcctcatccgcactggttagtcgaaagaagaaggtgtttctgttatgagattgtgagaaaggacctccttaaacattataaccaacctcataacttgtgcatttgtgtatcaaactctgctttcacataaagaaactaaaacaaggtatcacattgccgttatgaaaagtgcatagaacttcctgcttccctcaaacaaaacttgcaaatattactgattggccttagcctttaggtaagggaagaatcaaaagtattccttcatccttctgctttaaaaatgtgctaaatgacgttgtccatagtttaaaaactcgaccaaatcgcatttgtcttacagtctctcaaccctttttaagcactctcagagtcaatccaaatagattcctagttcctaatatgtaacaagaagagtgatactatgaaaacccacaaaaaacccacaaacatgtgacttgagttaagatgactcccaatcccactgtatcaagcttttcaaatagaggaatcacgatgagatgaacaataatatcccaacgtgctgctatcccaaattagatacagaagtctacttgtggtgttcttaatccaataattcattatgaaattcttatataatttcttaatgagtatcttagaattaatgttacaacttatctcttattctatatgatagaatcttaacataagtattcatattaagagcaagattatgttgatacttctcgaatcataccaaaaacttggaaccatgacattaacttcattcgtggaaacaagttttgaaggaaaaagaaggattgacaaatgaacgttatggttgtgcagtattttaactac

SEQ ID NO: 76 - DNA, 2000 bp, Cucumis melo
  source 1..2000: mol_type = unassigned DNA
atctaaaactgcattttttactacatacagattcacctttaggtgctggggcttcccctatttcattttatcaatgaaatgtttcttatctagaaataaaaagaactacatacagattcacaccactgcagaaaggtcaaataaaacattcatcataattcaaggtaagtaagcataattttgtgaaacttatgtgatgcacttaatatatgaacgattgccccttgttctctcaaagtcagatcttctttttcctaacaattgaagaaagtggaaataagttaattaccacggccacgcaataatctcctgatggcctccaatgaaccccccaaacataatgctgtagggaatgtcttcttgcaatccttcaagacgcacaatgtgagacatgcaaaatattaaaaagtgacatcttcaaatatagcaaaagaaatcaaaatatttacaaaaaatatagcaaagtttcatattttatcaattatacacactgatcgacatattttgtaaatattttcaatagttttgacatctacaataattagttgagattttgtagtcaacaggatccagatttgtgtgttgaaagttgaaacccatgataagataaaatcccggttaaatatttcattttcattcttaagtttttgaaaaaggaatagcttggtaagctacattccgcatggtaaacaagcatacaacttttgtttcaaagaaccaacaagtactacaaacaaaagagtaattgatttaatccaagttaacaatgacaaattggtaatatttataggatattagtgagataatacaatcaagttccaaaagatgttatatttacaactatgagcattcatcttgttactaccaccaagaaaaagtagcggttttccaatctctgtcaagtatccatttgagttatgatttcatattcaagactgtcacaaaaatttcattaaaaggtgcaagtgcaacatttccttaagaaaaggataactgagagatcaatgactggaattcacaagttaaaatgaacacaacttcagaacatcacaagctaatacctccaaacggtccaataagttttctgcaacactgtcaacaagcgaatcctttgggcgcatcaaccaagcagctcggtcccgctgttaccaagaaacagcaatttcagcaagaacaaaatatagaaatcctccaagaaaaataaacaaacaaataagttcgaaggcaccacatatcagaaagcttatggaggagtacatgtagtacaaacgctcttgccatttagttttacttgttaaaagtgatttgctcagaataaacataaccaaagcagaatccgaacatatgaaccaatgaattaataaaccccatcacagaaagacaagtaatactcccagaattgtactctatacagacgaccactacaatttagccacacaatatcaccatgttctctccaaatatatttaaaaaaaaaaaaaaaaaccctcctattgttgcggttaacacaaatagatcaaaaagaagaaagaaaaaactaaaaggagacaaaggtgttaaatttggtttacctgtttacgcttaagatcacgatcaaaaaccttaacctttgagctgtgcattccatcaccgattcttccgtcataattatcatcaccagtagagaaagaacaacaaggaattgaatttggaaaatctcgccatcttgcacttctcaacactgagaacgatcttatgatcgtagacggcgacagccctctcatttcacagtcaccgattgaacctcgccggagagacggagggaaattttgtaaatttttaatgggcctgggccgtaaagtcgtgtccaaacactccttaaacggaccaaaaccggcgtagaaatgaaactatccagataagggacgtgctatacatttatccaaacgaggtctcttatcgtatcttgtacaagttcgttgcttttcacggctgtctctagaattttgggttgggcgaaa

SEQ ID NO: 77 - DNA, 2000 bp, Cucumis melo
  unsure 1501..1544: note = unsure at all n locations
  source 1..2000: mol_type = unassigned DNA
aaacctactctgtaaatgaaggtttacatctcttaaggcagtaccatttctgccattacttctaccattcccacgaaaccacctcttttctctttcattctccccgtcaggtatgcatttctcatctctaagtccgcccactgttttccgactgatttttcgatttttaattagtgagtcggttttattgtttcttattctaagctttcttttactctttatatttttagatatttaatttcggatccattcttcccatcatgcccaatccaaagactgtcgaaagttttgattgttggtaatgggacattaggtctggggtttctgtttttcttatcctataaattggttatccttcgtttcctctattttgactttattccgtagttaggttagaagaagaaactactgaataatgtttactatacaaacacctcaaaatagccaagcctgtcgaaacacatttagctgataagctagggatgaagagatcaagagatggttagctcagctgtattgcatctcatgggggacgggtgaaacgaaccagagaagtaatatacacgttttttttttaaaaaaaaaaccgaataatttacctgttcttgctacaattacaccgataagttttcaacttgagcaattacaccgtctaatttgcattgctgaagaaattggtctgttccattaccactgttgattaaaaagttctacttgtcagcacagcatgtccatgtgcccagatagttcttgatctttggaaaaagtgctatgtttgcatgcttcggtaagatgtgaggttaaaatgaggaggacataatgttggcatagggaggtcaaaatgtgttaattgagagaaaaaatgtggtggatattggagaggagacatggaagtagagagaaagagatgaggagggaggggtgaaggtaaagggaaatagacatacagaaataaagaactgtgcgagtaatgtgttgcgataagtgaaagagagaaagcaagagaaacagtggtagaaaattgaagtatagagagagatgtagagagggaaaatatggagaactacaagataaaatatctttattctttctctatctaagtatttatctctttagaagttatctctctttgtttctgagtttacccctagtattttcttttttctttctcaagcccttcctctctaacacaatttctctctctctcttctccctctctctctgtatctggctgtggcactttttttgacctcttcctttctgtctttatctcctttgaagacattttgattttcctacacccctcaattggtcttctactcaaactcatctacttgttattatattaaatgcatgaaatcctaatattttaggaagctggagactcattgtgcgtgcatctgcttgcttgtagaaagttttaaattgaaaggcaagccgaaggggcctaattattcaggccaggacaatgatgttggttttagttttttgtttttgaaannnnnnnnnnnnnnnnnnnnnnnnnnnnnnnnnnnnnnnnnnnnttttgaaactaatttttttcttagttttcaagacttggcttggcatttaaaaacattggtagaaaatggataacaaaaccaagaaacttacatgtggaagtagtatttataaagcttacttatgtgtggaagtagtgtttagaagcttaatttttaaaagtctataaccatatggtcatcagtagagtctcatgcaacttatgttgtgacagtggtgtaattgttctaattaaaaattttcgggtacaaatgtaaaaaacattatcgaacagtggtggtttgtgaaatatgcattaactttttgaaaatttgatgtgtcatcatattcattccatgccgtgccttgtttccctcccagctccttatccatgctaattagattcagaccattatccctttggaacagctatgcttaactctgttcttttctccctctgtacaacagtatatcaaaaa

SEQ ID NO: 78 - DNA, 2000 bp, Cucumis melo
  source 1..2000: mol_type = unassigned DNA
tagcttgttaattcttgtgttgaagacgtgtttcaacaaatctgatggggtattcatcttaagtgtccactgaagaatgggggttctgtggcagatctgtatgttatgtagtgaaaacaaatctgtaaagtttttttttacttcgaatttaacgttgcttaagcttctgtgtacagttttatcactgcctcgaggttatgattattattggattaaattacaatttagtttacgtttaccttggaactgtgtatttcttttgattgctcaacttttctcggggatttttcaagaattgtatttttaaaattttaatttatttggaacattaagaagttggttatttacagatgagatataacactgtgattggggtggaaaataaacacagcttcaaacacggagtgagatatagttaattacattacatagtactagagattatataaatcactccactcacatgagttttcatcttaaaagattggaatttacatcttaacagatgcaatcttttaatgtagagttcttaacgtgttctcttacggttgtatcttttcgttttcattattctttggtcaaatcaaaattagactttatagtttttaatgaaatattggacacactacgattcatcaaagtaacccatgatcttataaagttgtgaaatgtatgtatattgtctttgatcaaactttacgtttaattatatcttgaatttataattttgtatttaagagatgaatgaattttagaaaattctaaagttcctaggccaaagttgttatagaagggtaaagaatgctttaaatcatttattccataatcattagttttataatttttattcttcgtaactattttttaacaaaaaaaaaaaaagttatgcatctcttaaatactatcttttaaaagggaaattttcataaataaataaaaaaagacgatagtatacacataaaaaaaactcaaatgatttatagagagtttgatgaattttgctggatttataaatagtttagaaaaataagtattaacctaaaattttgcctatatctcaatggccttctatgtctatgttatttcttaactaaaatcgaaaggatataggcttatggattggcttaagctaaaaaatgtcggtccaaatagttgagatgtcaaaccttaaaagtactacgattatgtgattttcacatgacatagtgttctatggtcaaattttatagcgtacttattccaatccatcactttttatagaactaaaattcatagttcctattttaatatatatatatatattaaaaacacacattaaatgatgattttatctcttctaggttgattgaaaattactaactaaaaaacacggtgcctcaaacctccaacgtaaatacgatttctaagaactgtgttttttgtaaacgccaagtgactgattaaatctctccattctctgtttacttctatttggggttatttatgctaaaggatattattcattcaatagaataaatgtgagatagtcgagttatattcatagatgttacaatgaggtgattcattcctttgtcaaacaatgctttctcgactcgtattttactgtattggatcgaaatccttcttactcgcatggtttgccttcgttgattagttttggtatgaattgatgctttgtttaagggggaaaatgaaaatggttcaattggaggacaattgtccaaatttcgggacattatgggttaaacacaaagaagaagtccaacagtgtaattttgttaaagattgcgttacatttccgaaatataaatgagggtattttggggaaaggaaatcaatataggccttggccgggtgagatgcgaaaaagtctcaaaactgagtgagaagcgtttgagctgggctcgcagctattgaaaaagagagaacaaaccctttcgtcgctcttattttcttcctttgatctgaaatttcctgttccgatctcgctttaggacgca

SEQ ID NO: 79 - DNA, 2000 bp, Cucumis melo
  source 1..2000: mol_type = unassigned DNA
aattcattcgggattgttatgaggtaataaaaaatatctgagtgcgaacatgataattggtaaagtgaaaaaatgttcagctattctgttctagatacagggatggaagtgggaacaatgccaccttgcttattgacaataaaatgaggagtggcaatattttgtttctgaataaatattcactagcataacatattagtgatgattcaaactaaagtgcactaggtcactagtttcttgattcatcgtgtttggtagtaatggtaggtattgtatcttatagtattggacaaagctttaccgaccataaattgtggataatgtgcagagaagaattggcagttgaacgttcctggatattcaagtgatgagtggaagaatcacaacaaaaatgtaagaaaattatattaccctctctaaaacatcattctattctcctccctaaaaaatcattctgtttcaatttaactttcaaaattttgttttagtttaaccatattgagttttttttcttttttaattatcgtagttatcatcaagtgatgtccacaagaaacgtttggacatggtaagttggacttatctcttcaagtgtttgctccatttcttcttttatcatttgtctcaaattttctcttctggggtttcatcagatacgcctattgaaggaagcctcctgtgtcgaaacaaatgtaaacagccctaaagagatggtacgacaaggggttggaatgtcaattggtcccaacactctaacaaggccttcaccgagttcagaacaactattatcacaaacgtctggttcacagttgctgcagcaaatgatgaggttttagtgtattaactacgtttgaaactaatgcttggtagagatcccaactacttggtgaataaccaaccccagtgtcagttcagggatacaacaaataaaatgagatttagaggatgccatatcagagggaacctggactggacatctgtgtggagtggagtgtgatgatttttagtgatacgtctttcggaatcaatttttttaggctgtataatatgaagttgcattatctggaacacgggcgtaatgttaattgtacaaaatatttggcaggtcatattagtataggccttaagtattgttgttgtctaccatgaaggacattttccaatttatgattgataatctttacttacaatctcgagtcatatgaagtttgttgatcaggatcatagcacaattattacaaaaatgaaatagaagatatgatttttcaccccccccccaccccccccccccccccccctcccattcccatccccccttttaaactgttacattacaacttgttaactgttgattttccagatgagagaaagggcctacttgtcttgtacagaaaattcatccatgacgataaatgcagatgacctgaaccaaacgtgacagtaggggtttcttctatgccacaaagctccaagccattcatggtgcgcatgtggtacagagaggcttgatggagcctcttcaccttggtccttagctatctaaaaattggcttcttatgctgatatatctcttcccatgtgcatttggtccactccactttcttcgtcgaatatccttgggttaatcctgaatggtaagcacaacattcttgctaattaatccctctttttatcctacttgccaactgtacaagatgagcagaagaagaattgcccaatcatgaggtcattaactgcaaaaaagagaatttatttctttctttgagaatctgatcttcttgagagttcattgacagccacatgcatcacaaaatgaaatgctgtgtggccctcattcattcattcatcaatcttcctatcctgccatttgagtgaatgttactccaacttgcaggaagctaaattagtacttttttatataaaccctatgaaactcatcaagaaaccacaccatcccaaaaaggaaacgagtgaacaactagacaactcaccccgaaaaa

SEQ ID NO: 80 - DNA, 2000 bp, Cucumis melo
  source 1..2000: mol_type = unassigned DNA
cagcgatcttcgtagaaactaattcaatgctagctcattatttgacttttctcaggcaaggctccgcgaggaagagaaaaggtccaatttcaggaaccaagggcatggacaggttccggtcagaagaagctttttacgtaaaccctttgccagattgtttatgtcaaggagaattaccaaatggaagtacgacttccattttcagatagttcagtagaacatcaaagataaaatgctcttagagagctcaatgtatatgcaggcgaccactcaacgtgtcaccagctttgtacatccaataagccagctacgatggaacgacggagtgtttataacctgagttttggtagttggcggaggcggtgatggtggtatagaaggaaggtcgagggatggcaaaccctttacgccaagtagtggaagggagtagttggagatgaacacattttgagaagtttccaagatcactccatttgggggagaggggatgttggttatttagcacaattgttttcatgttttagtaattttatccaataatgagcgaggcattgaagcaattaaatttatttttaatgattttttcacccttccataggctttttcttttttcttttccttttagtttgcaaactttagctccttttatcggctgtcgaactcatttttgaagttattgaatgaaacacagtttgggctgtgtcagatgggtggtgaaattttatacattataattactacataaaatgaaatcatattgtaattttctatctatgccacaatttttttttattgcatcatgaggattaaattgtacgagtccaaatttgtacagtcatgtttttaaagctttcgagcattgttactaatgcatggaaaggatcgattatcaagtatcctcccaacttcatgaaagttattatttgtcttctaaatttgttttagaaaatgtttaattaattatttgagaagaaagtttaactaaatcctattggtttcctctaaggttgtcatacttatccaataacaattacgtttaaaatcaaaattattctaatggtataagactaatgttttaaaagcataaaattgatgaggaaggattggaagtaatactatttattttgaaggtaaacattcttgaatgtctgtcctaaaatcactaatgttttcttagtttgagactttgagtcgttgaacctctccatctttataaaatataatacgagtccttcacaataacttaaaatatatactaaatcctaattaatgaaaaataaataaataaaaggtacaaaatcattaaagcctaaaaatctattactcctttaaaacttttcaagggtccctacaaccaatgagaaactaccacgtcattttcacaatccgttcagtgtttagaaaagtcaaatcgcaccgtccatttatccactcgtaccaagtacggtaggaatctatctaccgtccgattaagcacaaagaagcacagtaaatgtcaatcgtgtccatccgccgccataccgcacatccttcgtccgaccggaaggccctatataaagtcctttggttttccgaatttctacttcattcgcttttgaaagatttcccaatctcttcgtcggccaaaattctctctcgcttctcaacccttcttcggctttttcatccaggtttgtttctcttctcttttttcttcctttgttgttcttggaatatgtttaatttcatttgtttttccattcaatttcatgctagattttacgattaggttgattttctgttcgtagattgtaattgatggttagggttagctttttctcccattccttctggaatctgtttcttgaccttcgaacttcgttgataaatctttagaaacatttacataaccaaacaataattgaacaactcgtgttgttatgcctatataatagcggttaggaaactggaaacgcccttataattgaaatcgccttagaaatttgttttgattcatacag

SEQ ID NO: 81 - DNA, 2000 bp, Cucumis melo
  source 1..2000: mol_type = unassigned DNA
tgtaatgactaaacatactatagcctatttggaccgggtcgaaaatccaaattaaccaatctcccctcagcctcacaccaaggataaatcatgtcaaccttctccatttgacatgctagctggacaaagagaaatactaactcaaattccatataaatatatctttacgactccttatcaggtaatttagactcaacaattagtaataaatttagtataatgaatgatagtttccatagatcaattatatcatttattgatttgctagatctagagtgaacttattgactaatataccaaatataaaatatatcaatgaacttacccaccaaacataaaaatgtaatatttatatctacatgaattttacaataaaaagtgtatcatataaaatacttatatacataaaccctattatatatatatatatataaaaggaaggtaagatggaaaaaattggaagagaataatttgacctaaaaaaatcgaaagagaaaagagtatttaatatataaataaaaagaaaaagagagaaagaaaaaaatcttgttcgtcgactcctcaaaaaccccagcgtgtagcggttgtgagagaaggagagctcgtttccatcacgataaaaccttatctttctccattcttctatcttctcttccggagctctctccatttctcagccgctccccacaatttcctctaaacacacacatacacgactatttttccattcaaattccttcacttcgttttccattttccttttctttaccccacccactcacccacctctcgtcgatggactccatggacttggcccaacaaccgtcgcaacagaattcagtctcctcaggttcttcttccacttcctcctcctcttttacgtcttctaccgttgattcccatgtcgatactccctctctcgatgaacctgagatgggggttgctgaaattaaaactagtgtagttgccgatgggggtggtagtgatggtgctggttccgaaactgaagggtttttgagtggggaggaggaatttgagtctgcttcagatagaccaattgtgggttatccagaggaagagtccatcgggaagtccgcccaaggggctgatactggtacttcttttgtgggttattctcaactttctgctccggttagtgttaggccaattgcgaaggtttctgttgatagtgacgttgaggaggaggatgaggaggaggaggaggaggaggatgaccttcaggtggatgagaacttgaggggaaaggaggaaattgaggataaagtgggtggagaagatgtttttgttgagagtaagaaggggaaggaagttgaggttccagtggaaaaggaggagactattgttgtatctgatggaaacaagaatttggatgatgtggtgaatgatgatgatgatgccagtcaagtgcaggaaagaacaattgagttgtcggggaactcaaaagagggcaatgtgcctgaaagcttagtagctgaagatgttggctctgtgcccgaggaatctgttgatggtgggaagcaggtgtcagaaggggatgaattgaatgatgtgacagttaaacagtcacaaaatgaggcttcagatggaaaaaagaagcagagttggataaagaaactctggcgtctgggaagcaggctggtaaagggattgacttgagtgagaaggtggttgctgaggatgtagagcaattgaaagaacaggaaacacctggttcttcttctgacgagaaagctgttttgggagaccaagcaagctctaagcttgtgaaactagcagatgaaaaacaagaagaggagacctctgcggctgagaagcaggtagatgtggaggtcaaattgaatgacacggtggctgctgctgaagatggagagcagttaaaaaatttagaaactgattctcctgttgacgacaaaattgttctagctgatgacgaaaactctaaggttttagaaccagcagatggaggacaagaagcagaa

SEQ ID NO: 82 - DNA, 1072 bp, Cucumis melo
  source 1..1072: mol_type = unassigned DNA
tttaatatggtatcagagcaaatggtccagagaggtcttgtgttcaagcccctgcatttacgtttccttcccaattaaaattgtttccacttgttgggcttttcaaatatttcaagcccacaagtgagggggagtgttagtgtatataattaaatttgccttcttcaaccactagctgaagtttgtgggtgaattggtggtttaatagtaactatatcatgcaattagcttttttgagttcaacaatatctgtggtggagatttgaaatcgagattatgatgccttaaccatgtgaactatgcttaggttgacaactatatcatgcaactatcgaaaacatcatctctaatttataggtcttttttaacatagttgaagtttcaatattctatatgaacacagctggctatttaaattaccatattgaaaagcagcacttgaaatgcttctaaaaattaatgccaattagaagtgtttatgattctaattggttaacattactgaacacagattagttatagttattgaaagaataaaaattgtaaaatgccgaactaataccaaatggatgggtagtctgcaaattttaccaaatggtactacagctggtgatgaacttagaaggggtaaaggtatagtgtaactgtctaagttaatgccataaaggtatagtgtaactgtctaagttaatgccattagcagatcaagtccgttgtattatgtactgaacacattttttaatcgtatagttctaaatcctataatctgtcgaccaagttttaggtttgttaggctgaaagttcatgcaaatctaggtgcttttttgtactaattgtttgagattcagaaattgtatctcaatgttctccatgattatgtgcgtgtatttgcaaacagctctttggttttttcttcttcttctgacaaggatagtcaaatcaattacaggacataatttcaagatttaaggagagaaagcaagggaaagattcacgggagtggactgagtttccaagcagagttgcagtgcaattaaatgatactcatccaacccttgcaattcctgaactg

SEQ ID NO: 83 - DNA, 1730 bp, Cucumis melo
  source 1..1730: mol_type = unassigned DNA
gttcaactccacaagtcaaatttttttggaaatctcgtgtgaacacttgtgaaacactttatttttatattaaaagaaacaagaagatttaagatgagaatcccgtatttgtttggttgaaggacaatgaaattggtaaatatatcccatcgaaaaataatcaaatctagacacaaaaatttaaagttaaaacttacttaataatcagctggagcatagtttaatttgaatgaaaataaaaatcctaaactagagaagtttcttatggtattgaaaggccagtttagaaagcccaatagcgtgggtttttcttggacccatgtgtatgtctcactcatgaaattaaattaattggcctccacattcacctctctcctcccaattcccataactcaattttagacctcttaaatgaaacatatcatattttcataaacttcttttttacgttacttatgagattaaaagactttaaataaagtgtcaatttatattatagtagatgagatggagtgtgtgtctttgtgccctccttggggcccaaggactaagtaaggatgaaagggcaaagaaatacaaaatagaagagagtagaaagaaaatgaaatggaatatatagtaagggttatcgtttatggttattatgagggaagggctgaaattgataatgaacctatccttatcttcccttcttcacctctcattttgcttgaaattacaaatgacttttttttcaattattttgtgtgtacatccaaatgtggtatgcacatatgggcctcccattaacttgtgatccaaattaattcttttgcaacctaagttgaaattaaacacttttacctctctttttttccctaacaattttactttcattgttagatggttgattatcttgacatgtaacaaaaagttctctcatgtcaagatagaaaaatcgaatatttgattttgagattgataatattataatatcagttgagctatactcattttaactatcagtaaagcttcattaacatattttttatttagtaaactaagattaatataaatagaatcttactttcattatatactttgacgagacttaaaacctatttagcgcatgatttttaaaagttggtaggattttaacccttgaaaaattggtcattcgggaatcaaaacattagtttccctttgagcatttatttttaaagcacttcaaaagctaaattagtagcattaaaaaaaaaagtcaaatagtatatatatataccaaaactttgtttttcaaaactatattttaaaccaacattcttttttttttattatttattactaattaagtgcagattatagtggttctcttttgtagttggatcaaatatttcattcttttttgacaataacaaaagttaaaatactcattaaatgctaaaaacttccatactaacattattgaaccattaaatatatgagcaacgaaagtataggtaagaatttatattgttgttgtttagtttggaaatagaaaatggaccaatgggtgagcttggtttaagttagggttcttgtggttggatgataatgaaataaaatggccaaaattttaatggagaagaagatccctttaagttcaaccactaatggagtcttttaggatcaattcacaacccctttctccttctgccacgtgtcatctcagctaatctcaactgtgtggttgttgagaaattttgaaactc

SEQ ID NO: 84 - DNA, 2000 bp, Cucumis melo
  source 1..2000: mol_type = unassigned DNA
aactagactagcgagtgcacaaccaaattacaaaatccttaacagagacaaccatctatctcctttaaagcaacaataacatcaaccgaattagaatccacaatcagtaaagacgatgccgacaccaatgaccaaaaccgatcaaatatagcttattacggaccattacttcaacagttacatcaacaaaaaaaaaaaattaaacattgctaataaaatctgaaaatgaggaaaaagagattaaaagttttgaagatagaaagaataaatctgaaatgttctaatttgatatataagaaatatgaggtaatatgacgaaagcattttgatagttttcaccaactccctttgtgaaaggatacatccaaccaattttacaatttctgttcaaattttgtccacctacccttctcttctgccccccaaggctgctttctttcttttattatttgctaaattaccaaaaactattttcgaattaaaccatctatttcaattatatacgtcattcgaattttaacttaattaacattagtatatgtttcggatcaaggatagtggtataaatcatcctaatttcaatttgtatttagaaaagttcaattatacttaaaacttctaaaaattttatattttaaatttggatataaattaaatttaagatttatggaaggtaaataattagagcaaaacaaacttcaaactatatggaaaatagaaaaggaatattttagccaaacaaaaacacttattatatttattttgttttttgttttttttaatttaacaatttttttttttattggttgaatgtgtttctccactggtgagtctccaactttgacctgcaaagggtctatatagcgagtttcacgagcacctaaccaatatctgtgtaataattcccatttttctttcatacccacttcatttgatcatctttttcacaaccccggatctctaattcttgggaatttgcctctttctcgatccatttccaccgtaattgaaaaatattcaggtttgatttcttctgggttttcattcaactgtctaacttcattatgccctttatgtgtttgttgaaagccccccacccaccatcgttcaatgcggtttctttaccttttgttcggtttcaacgatgatttagaagttatagatggatgctaattgtttcgttgttggtttgatccactgatctgcctttgattggcataaaaggagattctagatcttgttttgatgttgtgatttatggatattattgttatagtcgtggaagtttttcttgtcgttctgcggtatatggttgttttattttttgagtggtaaattgagcagattgtgaacttttgggttttatggtgaaagcatgaattagtaaatgtagagctgctgaaacaaaatggaggtttgctagacctctttgtgaattcttaatggtcagcctccatcttaagaggctaagtccaaaaatttaaggcagtcttttgttattgttacaaaggacaagaaataacagaggagttattttaattgaatcaagttggaaagaagtactacttcatgcttctttcaaaagcaggtcaaagtgctttaaagtcttcttatttatttattttttcctgaatcaatttaaactaatgatagaaagaagtgttttttaatgggttattataagtaacatcaatttttaaccattccaaaagttacatcaaattcatcatagtgtgagtttacgaattttggaagttgtaattttaagttaatacttcttttaaggaaatgtacactttgcatgttgtgttcataaggggtatttctttgacaaacgcagcaaccaccccttaatgaaaactacaccacggtggttggttttttcttgttatttttttacttggaatttacaataagttgttatattcggatatatggcaaagcagatatctgtttttatccgaaacctcataaatcttgaatgtgcagcaggtaaaaac

SEQ ID NO: 85 - DNA, 1020 bp, Cucumis melo
  source 1..1020: mol_type = unassigned DNA
tcattgatgaagaaggaaactttaagccaattcgacaacttatccattcaacaactttccaccttcagacattcagattcaactataatataacataaattgatagtcaagtcttttttgagacaaccataaatcatcttagcttcgagaactgtcacttccttaaattggtgaatatatcacattccatccattcaaaactttgttttcgaacttttactgtagttatgaatcaataaattgggagagatattgtttaaaaagagagagcatatttgtttctattatttactctctcctaagagagggttaattagtctataaatgatctattcttctcgtccattgaaattttgttatcctaaatttatgaatacttctacccaaaataaagacttttttttttgaaaagtgtcaaaaaaacataaagaaattgacaaaacattcatttttagtggatttttacggacgtaaatagtttgttttgtttcttttaataatacaatttttttactttaaaaaatatttttgttataaaaccaccgtatttttattcaattttaataaataaataaatgaaagaatataaaaaagaggaaggaaaaagaagccaacgaaccaacggttgccacgtatcaaaggtctaaagtgcgcaaaacgaggccttcggaaaccaaaatgcgtggcttcaattggagcaagtaaacatggaaaccacgtccattgtaacgcttcctgatctcttctttacaaccgttggattcgagtactttttctcaacgattaacgactgagtggacctccacttgcttctgttccacgcgcgtgggattgacgtgtggtccacgcaactcttctcgataggatcattcgagaacatcctttacttaaaccgcctctctctgcctcaatttctcgtcacttccttctccttctttaccctttccactgcggctgattcttcttcgccttttattctctcgtacgccgccatattcttcacttctttttccggcgaca

SEQ ID NO: 86 - DNA, 2000 bp, Cucumis melo
  source 1..2000: mol_type = unassigned DNA
aaatcatcttctcccatttgcatgtgttaacgcctaatgtagtacatttaccatgattcctagaataagaccgattttaccaacgagaagttgctttcaacttgctacaatatacataacatttctttggtacgttattgatgagaagaggtataaagcatttcacagtattctctcagcaactcattagtttaaaaaaaaattaaaggaatatttgaatatcgggggatgaattaagtatagcctcacaatttgccagctccttctccttagcggctgccaacctccgaagctttgcagcctgtgcaaatgtagacggtctacaagaacataaaagcaaatgaatacgatccccatgacagccataacagttgcaaacaatcatatagaatgaatgatttgagccttttttttttgtaagatgatttgagccgaattaacagtgtctaatgctgaatcgagctggaaaatactacttactgagataaggtgctagcctccctcagaagttgctttattgatttgcgcaactccaattccacctggctgttggaacctccctgaaaagtacacgcatgatggaaacatgattgtttcaaaacaaacaagttgacaagattgaacggataacaattataacatagcaaattcccagacattaaaactgaaaatgtcaatagatctccacattaaatgcatcacgtccctaaactaatcaaatcaaatgtcttcaatccaatatcgtaaacttaacgaagcacagttaggcatattgcattctcaagtctgtcaacgaaatactgaaacgcgctacagcccaaacctcaaaattttcaactataaataacaagctttgaattgaaaaacaaacggaatgatagaaaatacaaacacgaaaaaattccgacgggaaaaagaaaatcaaacgaaaaggcgaaccttcttcaggtgctccagccatctagcgagaaactgaaaaccgataacgataaagaaaataaatggagcggcaatggagcttccatgctctacgattccttccgcttccatttccatttccagaggacttttctgccacaacggtgaattaatcaaacaaagaaactccgttcatcgtcgcaattcgacggaggttattctggaagaagttgagatcgtaattgggctacgaatatcatcaaaggggcttcaataaaaggtctctcaaaacccaaggcccaaaaaaacgaaaagcccagcccaattagtggagaatcaaaacgctgcgttgtagatacaaatatcttaggaaagggaaccaagttacgaaaatacccctgagtagtgagatcaatgattacctcaacgacgcgttaatcgttttatcacgtttattgtgataagttccgcactaaggaagggacgagttgtaggaagggaggggtaaactggtgatttcgcattcaaacaacgggctttaactcacgtgtccggatctgttgagagggaacaattcacagcgaggaaattgcaaataacacacaaaggaaacacaaaagagcggaaagcaaatgtgaagagacgaagagtagccaatgagaaaaaaggacgaggatcgatgacatggcaaaagatttttgaaatcccgcctaaacccggagtttcaattgatatcgcgatttatctctccctctctttaacgaaaccgactcccttcatatccctctctctcgctccctcttcacttcaaagggcttttccttctttccacataaacacacgcactcgaagccaatctcaaaaccgcatcacacgaaccaaactaagcctaacccaattttttctcctcatatttcactctcacactctttccttatcttcttcttcccccaaaccctagagttttacaggtaaactcccaatctctccgccgctccctcgctcgattctccttcgtttctccgccttttttcttataatcattacctgttttctccttccctctatctgcaggattcatc

SEQ ID NO: 87 - DNA, 2000 bp, Cucumis melo
  source 1..2000: mol_type = unassigned DNA
gtgtagagtgagtgacggtggccgacagttcgtaacatttagttgttagtgagagacggtgagacgtttggtaacaaactttgtttttagttcaatcattgctttgttttctctttcttttccttaatgtctaatgttttcatcttcctttctttatttcttacccaatttccgaatcaaattttaatttctaaaaaagtatttaaaaaaaaaaaaaaaattagtcgctttattcgagaatttcataatcaacctaattttcaaaattaatcatcaatctggaaacttttttattttttttctcctttggattatcctgtatgaaagtcaacatactttgcactccttgagaatatttttagtggtgttttttttttctcttaataaataaaaaagtttacatctataataatcaagattccttggcaggtgtcactgtcaaaataattcctatttgttgaagttgaaaataatttaactataaactttatttgaacgtcaaaaaaagaaaaaaaaaagatatatgaattcacccattccataatttaactatataactttatttgaatgttgaaaaagaaaaaaatgaagacaaagcaaattcacctgttgccattacgacaaaatttcaaatgcgttttattttgtttttatgtccacaagattctctatttgtattctgcgaaattaaagtcacgggcttcgcacgtgtgtgattaatagtatttgtaaaagggcatgtagtcgaacaggatgggaattaaaggagattatgaatgggttgggtcgggaaggcccatttctataatgaattgatgggccgtcaaggacatttgtctacataaagggcatggaccatgaagttaagcccacttcctaaacgagttccttagtgtgtctacattcatatttaaatcatctttaattcagaattttcaccatcatcaaataatgtcttataaacctcccattttatagtttaattatggattctaataaaaaatctctaacttcaaagtggataatttttttttttttttaagttgaaccatgttcattcatttaattacatggaataaaaataacgtaatttaggttaaaagttgagaggataagatgaagttgaaaaattacaacaagttaagaagggaatatgaagaagaagaattcaaaattgagaacataataaaggaattaggtccaaagctgtaaagactaggagaaacgagtagagaagggaaggactcgtttttcaaagaaaagaaaagtgtggaaaaggaaaaaggttcattaggggtggtgaggaaatggatggatatggaatgatgatgatgagaaagaacagcacgggaagtttccgagtagttgccttttgcatataccaacaagttatctaataaaatgttttgattaattacattaatttattcaattgatttatcggaaatttccatactcttcacgtgatatgcacgtggtcttcccatgttctaatattttttgtttttgaaaatttgaattcctactctgttttgttattctgctcatttaactactcaaatattttagtttgtagatataactttgtaaatttttattataacattttgtaaatattttaaattgtgcccatagattatgagtagataaatttacgaattaaaaaaagtttaattctcacttcaatttaatttttttttattattatccaaatctatttgtcgcagtggggaaaacggggacgtacggccgattggagtccaattagtggatgtgaaacgtggacggtagagatgcaatatgaagctggacatcaactttgcgaaggaattgttccttctttccctctgacgcttgtcccgttattgctcgttttaaagcaattcgagctccgcgttgtctcttccctcacgttttcctttcaatcccactgctcctcctttcaccaataaaacaaaaacgcctcaaagaagaagaagcaacgaccagaaacctcaaaa

SEQ ID NO: 88 - DNA, 2000 bp, Cucumis melo
  source 1..2000: mol_type = unassigned DNA
gttcgagcatgtgaatgtcttctgttgtttgatgttagaaggaaagagatgggttagggagttcctgttgatgtctagtaggttcttttttttttctcttgtgcaatgtaacatagtaacttcgctgcaaagcagctcttatccttagaatacgaaaatcttctgttttttgttatgtttctaactttatcccttcttgattttaacttttgagttaaattccatctctctgactttgctttgtggtattctgtttctgttgtatgataattcttatggaactcctatgctctctctcattgccttctttttcggctgttacttaattactttcttcacttgaaatttatagcttctctcacaaatttgagctcattcaagtatcaaaattacacccatctcataccatatttctatctctgaaggaggatttttccccttttaaggagggtagattgacaaagctgatagggtgagacaatttaataactcaggtcagatgaattatacattgaagaactctcatccagggccagtgctttgtttataacaagatgattaatgtgttgctatcaaaactttgctggttcactaaaaaaaactcttggtccttgaaagtaggcttttactagttttagctttaatgcacatctgtatgtcaaccacgaactccatttttcttacttgatgcatgtgcaactttagcagctttctaagttcatatcaaagcaaatgtacctttattcctattgtaattccttttctgctttcctcttttatgaattgtcaaaaatatggacaggaaagtaagctgagcaccaacaggttgtacccctttttcatgtcttgaaaatgaactaccaggacacaaatcagatgatgattgttgggagaaggaatgtaagattattcgttctgtttgatataagagatgtaagttcacatgtcttacaactttttgaaatttgtgtgtcgcttatgtgcagattcctgtatgtcattagtggcatttgtaagctacaattgttgaatttttgtattattatcttaaaaggaaatgacaaaaggtataatcaaatcaagctgaacctaaaagaaggtacaggtttttagtattatgcatgaagaaggtttttcatgtctcttctgccatttggattttgtctgtgacaagggactaagacactacacatgatgctggaaactgcaagagtgtttttaccctaataagattaaaacgtgaaaagcaattagattttcgtgcatatctatctttttgtgcattccaccaaactgttcgatcataacttgtcaagatcttgctttttccttttttttataaatattttaatatccttctaatgtgaatggtgaaaagagatgcacaaagataagtgatactatagatgtatctaagtattacccttatacctttgccacgtaagattagatacgagaagagaaaaaaatctatgagttagtaatagggcaacaataaaccacagaaaaaccaattaatacctttcctcattgtctaataatatctaaaagaaacttcttttcatgttaatgaaccaaactatgttgtgctatagcatgagcacattatttctaccctttagacaagtgatgagaatggacaatatttcgactgagttcaccagaatgtaaccaacggttttgcatttgtaatatgaatttgaaagtttgagattccttatacgaggaccttttttcatgtatctaacaacacgagaaccaccaaaatgagaagggagttggtccaagccaaaagaattttgacctccatgaaaatccagatagtggggcatccttatctaaacaatcagaacctgaagtccgacgtagccttatccacatttcaacttcaaaaacactccctctaagatcctttcgaaccaccaaaatctaagaaaatttctcttcctcatcctcctccgacacaaaatctagcttcaatttcattcctctgtaaaaac

SEQ ID NO: 89 - DNA, 2000 bp, Cucumis melo
  source 1..2000: mol_type = unassigned DNA
attcgtcttcgcattatcagtaaatttatcattttaagagtttgttcttttttaaaaaaaattaatcatttcgataaagttggagaattcaaaaatttctccaaataatttataaaaactttcggttatatatcgaaaaaattaacatggtattaaaacgatcataactcaattaacataaacactccctctcaactttaataccaaatttctttattaacgcaaaatttaaaatttgtttttaaaattttcacataacataatagaaatacttttctttatggcaaaaatacaataatcaaaattgattgatggtgacaggacaccacacaatatttttaaattttgaatatacgaactatataataagatatttatgagattcccatcctaaagattcctagagatttccttgtgtacaatattacacaagtatcttggaagtccaaagtcctgagaaaaaagctatgtataaagtaatgtgtttgtcgtaggaaatttacttcattcgtgtcattagctttttattgaaaaaaaaaattaggtatatcttagtgaatctcacttaatcgttgtcgatagttattcttttaatatcattatatactaaaatataacaatattgaaaagctaaaactgtatataaaaaaaatgttacctctaaacttttatcgtttatttaaaagataaatatattctttcaaaacttacaatcaacatcctacgactatcattataggtacaaatcttttcatgtttacacaaaaattagatttttaaatggtgtaatgatgatatataacgaaattttgaatgattactatttgaggttaccattgtaattggtcgtgttgtttgaaatttaattttattagaaaatttgtcaaaagtagcaaaaatgaataaactatttaaactttaggataaaatcaagtgttatgagtttttgtctagtttatatatttttatttttattgaaaacccttttcctatcttttcattacttcaaaatagttttaaaatgtctattaaggctaaagttagtataaataaaatttcggaaattttttttcgaaaaaaattgataaattatttatattttatattaaagtcaaaatttattacgcgtagatgtttatcaaattttctttctttttgttgataattttccaaaatttggataattttttaaaatagtaaaattattaaaaaatgaaaacaaactatttataccttaagcaagaaatactaaaaaggcaaaaattcatttacttcatgaagcgtaaaaattaaatattttaccactttttgttattttttaccatctctatcaattatttgtaaaaagaaaactacaaaattagatgttttttcttttttaaggtttaatcaatattaaaatttcttaaattggcagacaagttggtgttggtaattacgaataaatcccgaattgactaaaaataaattcttctccaagtaaaatagacacgtggatgaagaaataagtgaatcaaaggcatccacagttcaataaatggaaaaaactactttctgctgactcattcataagttttcataaaatttcataagaaaggccaaagggcttatgaaagtgaatgtcatagcagtaaatgaagcacagcgccattgaaagacaactcaaattgcatgcaaacccacataattattcaacaaacccacatcaaatttcccataaagatcaattctttagggggttcaattacccaaaagtgaggtagttgaaaaccattaaacaacaagaaatcaacaattttgtaatttgtttgtacagaagtaagagataaaatcatcgttaaccattcctttatttcgtaatacaacccatcaaccatctctctctctctctctctctctctctcggcctttatctttctcttcctcaattaatttaagtactacccaagtgagctaaaagcaagttcagtggacagtgttgtaagaaccactacagaaaa

SEQ ID NO: 90 - DNA, 2000 bp, Cucumis melo
  source 1..2000: mol_type = unassigned DNA
aatcatcaggtctccttccaatgaaaccgacgacaacgacagtgtcggaaaagcgaggaagggatggcgaaggcgaggaaggagaaaacgaagtagagggttccggtaaagcagaatgaggagggagaggagttggggaaggtgaagaggaagaggaagtgggagttgataatggtggcggccggataagtactcggacagaggaggaattgggtacgtccatggatgagagaaaattttgagctttcagatgcaactgaaaactgcttcactgctttcacttccgatgaccgccgaggggaaacttattttttccttgccctttttgcctcctcaatattttccttttaccatttcctttccaaatttatttttctatgttttgattttatgttttgttatatttttgatttacttttacgttatttttaaatatttttgatttaattttgttatatttgaaaacaagatattcattatatactgtaaatcttactttattattgtttaaatgtcgttttggtaattcaaaattaagttgaataaacacaatattttaaatattattttagtaaaataatttttaggttggagaatggcaaaagaaacaaaggattgaaagactgaacccatatttgaggatagaagtcaaagccaatgtcaataagtgaaactcacttggaccaaaataccaattttagttttatatttttaattgttcaatcttagtttccatactttcaatgcatattaaacttatagttcattattctttttcaataaatcttaacattttactacaaatttttaaaatgtttcacatactttatttttttacatgaaaatgattgttattgtttaatccatttcaataaaattaaaatttgaaaagctaaaaattcaagaattatcgatagacaattacaattttgtcccattaaaattatcaaattgaagtggctacacaatggaatggtaaatcctttattcttgtattggtgtgatttggattgagatatgaaacattataatctaaaggaacatgtttaaaccgaacatcacgtattttgtctttcaaaatttcgtaagtttgtaggttgttttttttttgtcattttatatagttacaattatttaagtcagatcggataaattttgttatacaccaataggaaactaaaaattccacaaggagtatgaatgacctcctacgggagcattaatgaaaatgaccaagggttaaaaaatggtaagaaaaatgttcttcactaatgacaattcctcgtgaaagtactaacatgttcttaaaatgcttgcaagcatatatgtcaccaagaattctcattcattcctctggcttctttctctcatttctcatcaacattaatatgacacactttttccttcttctttttgtatgtgtttataatcttactcattccttattctcattgtcactcaacgattccaacaagcaatatgggaacaaacgaaggaagaagagaaaaatacactaagaagaagagatgaacaaagttgcattagaacaaggcgtagaatatcaaagaattcaaaataaaaaggaaaaaaagattactagacgagagagaacgagacttgaaaagaattagaataatttcggtaattttacattggacgacgaaagcaaatgacaaaaacaatttttttttcaaaaacatagctcaaatttcatttagatctttcatcccaaatggcataatttctctaatttcacatacaccacaaatataatgatgactgattaaacgaagtaaattacaataggactaaatatataattaaacttcttaaattgagtttgagataaaacctttgaagccacgaggggtcgggtcgggggcgaaagagacatgccatataagcagttggttgctgtaaagtggcacacgcatatctactggaagcctccatttccaatctcccattatcccattatcatcggcagttccccatagcta

SEQ ID NO: 91 - DNA, 2000 bp, Cucumis melo
  source 1..2000: mol_type = unassigned DNA
ctttcctgacccaataagagatcaaatcactgtctcctgtagcctttcccttgccgctctattattgacatttgggcctaccttccccccccccccttctcccgattcatcacccttgggccttggcccattaaaacattacccagctccttactactttttaataactatcacgtctattccttcgcaagtgggtggaagcgaatatttataccaattatcttttggttgatcatgtagccaaaatttggctcaccaaactcgtacaaagacatttacttgttttccactgtagattttaattttggaagaagagatcagttgccaatagattgaattaatgcatttatgtacactttcatacttaacttttggcaaagagttgaaagcaaggttttaaagaataaaatgaacttactttttttacaaatctcatgatttacgctagctcaaacttaggatttctttcgtttgaaaaattggaccaaatatatatacaatagattgaataggagtcttttaaaatactggcctcaaagaaatagacaagttagctaggtcgggataattgcctcactcattcttcacctcagagatgcctctcctcctaggcatgttttctaccctcataatttaattcactcatttttgcttccttattgattagtaaaagtaccgatttgccttcttttctatgttgacaagttcccactagaaaacaaattagattatgagtttataggaaagaattaaacacaaatacataagtcaaattgtgaagtatcaagataggctgttaggacagaaagttcaaatttggaaaacaaatatatatgttattgagttgtcatcttcttagataatgataaaatgtgaacttttgacacatataataaatagcatgttcttgataaatagttttccattaaaacaataagctattattggatgatagaaactcccctgggactacaagaaaaagctaaaatagaatcagcattaaaacttcctttaataggatcgttatcccaaataacaactccatctcaaaacacttctaaagaagtagttaaagaataacaatgtatattagttatggatgttgatgatagagaacttggattttagctaaatttagaatcttaaaaagggaaggaagaaaaaaggaacaaaataaaaagataacagtatgattactccaacttgtgatgaacagtaccactcatggtatgtcaaacatatacatagaatgagaacaatttagatcaattaatttactcatttatccttcttgctacagattgttgagaaaatagaaaaacaaattaaagtaggaaaaaaaagaataaatggggaattatggaaccaaaatatcaagaaaaaggaggggcaataaattaaagaggaatagtgtaggccttctcacagtggaagtattagcgtttaagtcagtaccttacctttatttgttttcatactaagttctttctctttcatgttaataaattttcaatcgatccatctattcaaaatggtgtgttttattaggaagaaaggtaatttcatacaagaaggctaaaaaatagttgacagctgtgggatttgaacccacgccctttcggaccagagcctaaatctggcgccttagaccactcggccaaactgtcggaattgtgagttgaataactaagatgatcggaaatgtgacgaaataaattgggctaaagaaaagaaaagcccaaacaatgaagaacaattcggcccacttaatttcacgcgcatggcacgtgtaaagaaatcccaatctgttctactaggtggtggtggtggcgaggcgaagcaaagcaaagcaagatcagccttatcaaattgtgtggtgaagaatgaagattgtataatgtagatagaaaaagatccccccattcccattcccattcccttttctgaatccgccattgttatctctctcagacctccataacctccatttctacccagcc

SEQ ID NO: 92 - DNA, 2000 bp, Cucumis melo
  source 1..2000: mol_type = unassigned DNA
cttctaaacatcctcaatgttcgattttgatcaaggtcgtttgcttctaaacatcctcaatgttcgattttgatcaagaggtcgtttctctatagtaaacatctgttacaccttccatttctgttattcaatttttccaattttattgagcagtttatttatttccgtaactactttgcatcggaggcgatcatcagtttttaaggtacaaaactagattatatataattatgaagcacagcaaagtataaaattttgaagatgaaattgattggaccttgtgaacagaactctaaagagaaaatgcatcagatagtctggatcgttagaatttgaaatttaaatttctatcttccactaaagatatctctgttttgcaaactaatgttcctcattctaaacagagaatgccagtggtattttgttcgttttttgcgaatatgattaaattacccattttatttgcatattttatttattctcatatcagctccaaaagaatatgatccctttttcctcgataagaaaaaatatttaatactttcaacttcatgcattgtgagactgcccatttgttttgtttaaagtagcaccaacttctcaattgtataagtttgtgattttttttctatctaaattgacttgaattatttttagatataattaaattaattgcttttaagagcaagttaaattaaggtttcgtaaggatatggattaaatttaattaagaattggcttcttgctctaaatacaaattagagtgagatttgaaataggaggaaaaagagagtatggttacaaaggatatgaaagatcaaatttcaaacctttgccaactgaggcttttcagaactcttaaaccatcacagttttttctttgcccaaatgaaatcaaacattaagaaacagtgataacgaaaacgaattatccctatgccaaccgtgacagatgataggcaagaaacccacgattagtctctcatccggattgttccaacaaatgaaaaagcgttttctgagactacacaaacaacaaacacagagttagatagttcaagcaaatgattctagcagattagaggataaggtttcttattaaatgtttgaatacattctaaccaaaaaccaaaaaccctatttgcaaatcagcttatgtaaaccaaaaacatatttactaagaattcagaatttcgctgcttgaaatttgaaggataccatataaaacaataatagattcccccaatcgtgttcagtagctcaatataggcaccgtgcaaaaggttgtttgttgtaagattaatgaacaaacacccgtgtctgattttaatgccaattcaaactctaattcaaaaaccctacaaagacctaattgcagataatgggattagaaattttaaaaaatgtcgaccgggcattgtatcttaaaactattaagtttcaaggatcttcctccggtaacaaaatatcggctccatgcggcagacggatcgccattaaaacggcgcctgctgctgactcgatgatagagccaattcagaataaccaacccatttcatcgaaatttttaaagagagagaaaataaacgattcaagatatcaaacgcatttcgcttctattgaaggagaagacaatgaaaatcaaaacaaatcggaaattaaagttaaagaagaaggagataatctcaggacggacggaagataattctaaaggtgcgattcggttgaaatttatagaggatttgtgaaggaaccctaaattctgattgtgaattttatcggaaagaccggagaggaagcccattgtgtgaggcccaaagtaactgatctgggccttttttagtttcagcccaaacggaagcgacacgtcgtttctatgtagagccaagagcgtgccacgtcaacagacgacgtcggttagtaggaataataccgatttgtggatttaagaattgttcatttcggtttgtatcggaagttctgaatcttgatccgtggca

SEQ ID NO: 93 - DNA, 2000 bp, Cucumis melo
  unsure 1064..1094: note = unsure at all n locations
  source 1..2000: mol_type = unassigned DNA
aagtagaaattcagcgaaaaatgcagatggtttcatagacaataaaaagcaggaacaagcgcagagaatggttaatcctccagaaaatgtgataaaaggcgccaccaagaccagtaatccctttaccaatcacagaatactcaacaagaaaagcgattccagcaaaaacgaagatgaaactctcacttacaagaagagggtcgacattttcccgcaaaacgatgagaatggcgagtgcccagaagaagaaaatggccagtgattgctgggagaatgcgaatctgtaagtggggtttccggaaaaagcgagaaaaaggaaaatttcagagaaggcgacgatggggaggaggaggatgagggaatataaatcgaaatttttccatttcggttctgataaataccaggtttttgatcggtaaagagatgggttgttgaggtaaatggaggaagaacagaggaggcgacgaaggccaatggggatgaggaaaagggaggcggagagatgcgttgctagtgatgccattgaaagggcttttgaatttgttgaagcattcagattcttctctgtctatggttccgtagattgttctccaattcttccattgggaagacggagttcggtggctgaacgttgaccctaacaagtttgatcacgttgatccgttcaatgttaaacagctcgatgattttcgtctaaaaaagaagtgattttttttttaacctttttattattgaacaaaaaaaagatctgtttataccatagtttacgttcttccacatgagaagttttataatagtttatagaatctatccaaattgtgttttattgggtttcgattttatagaaatgtcatatcaaaaaaaaatttaaaaatgataaaaatcattataattattttatgaaatttttactgtgacttaattagattataaaccgaccattctttaatcattattttggatgtctatcgtatgtgtatttatagatgtcaaacatgagagcatagatttaaaaaacaaatagcttaaacaaacaacaataactttttatctttcagaaaagnnnnnnnnnnnnnnnnnnnnnnnnnnnnnnnaagaaaagaaaagaaaagaagtcttgaaaaaagtattaaatttcacaataaattttttaaaataaaatacattaaatggggatgaggaagaaacaactaagagtccaagaagagaaataaaaaatgagaggtggtgttttttttggtatgttaatcaaattatggtctccacatacaagaaatgaagccacgttaatgacccaacaacactaacacatcaattcttaaaattcaattccttcttttcttcccttccaaaattatgggtcctccaacttacaaattaacaattgactttagctaactatgttttttaaatataaaaaacgaatacaagtcagtttaataggacttgaagattgtataaaccaatattagacaatcaaaacaatcaattttaggttcattcccaacgatacatcaatttggattagattaatttttcattatggtttgatagagtggatttagttttagtggaatgcagggagggaaaagtaatttgaaagaaaaggaatgaggttggtcaattccgaagcctaggtatccaaatacaagaatccatatcaaatttatgaacacctagaaaataatagtaattttaataataaaatggagaaatggggtccggtcgtcctcttcctcgcggcggagatgaagccaccgcgataagagaaagagacccttttcaatacaattcaacaatcacatgaattattccaattcacatctctgcttttgaaactaaactaaacgccaaaaacccttctgtggctcataagtttcctctctcaaatctccgatttccctcacccacatcccacatttcgcatccaaataaaaaagggacacggacaacaagaaggagtttttaattcagtagtgcctctggaagaagctgtttca

SEQ ID NO: 94 - DNA, 2000 bp, Cucumis melo
  source 1..2000: mol_type = unassigned DNA
ttagtgaaagttcaagatgtaattcactctctttaacaaggttgtttctttgcttcactacgcatcaattcaaatatttagatattgatgtttaagcttaatctcctatcttagctcagaacaaaattgtcaaaatctcattcttatttgtctacctggtaactttgctgctatagttatttgtgggagattgtagcaaatgactgtagatcgaaaccatttcagcatcaatttcgacccactcttctcgtcaacaacttgatcggcagcttcgacattcttcaagcgccagcttctattggatctttagctcaaccacatcttcgtctttgaattgcatgtgagctgttgggctcctttcttttgtgcttatcagttgggagattattactataaatacaaagcctcacgggtattttaagacacaacaaaaaattaaaagtctctcctctgaatcaccacttccattttctataaattttgttctgagcaacttttgtttgtttctatttcttattctgaagagtgcatgtttgagtatggggagtaatgttaaccttgaggaacaattggcaacacgattggcacctcggtcaatcatagttgcttttaggacagtggttcgtcacaacacaacaatttattttaagttcaacattctcattcttttcttctacagtattcaaagttatagtgtttatttctcttattgttcctttagttaacaatctaccctttaactaaagtaacaacttaaaagtaaaatggattattctactttttcttaattgttacttttaaaggtttaagaactgaattgttactccgatgaaagtctaaagaccaatagtggtttctatccttaaaaaactattcaatgaaatttatgctaaaaaaataatcactaattcatcgtgagcttccaaaccacttgaaattagctcaatgagattgtaacttggtcgggatctcatcaaagggatggtcttggctagattcttaaagatcattttagaaagtagatcatgaaaggttgcaaagatgctagaaacaactgggttgtcgacgttttggaagctaaagcggtgatgattgacgtaatagatatcactaaacattggcacaatcatacttggaaatagcttctatagatatattccattttgtaaggtcttaaagacaagaacaaagctacctataagcttgtatcttagtttcctcttgcgatcttcttgtcgagagatgactttccggttttgggttgtgtctttgtttgtttttctttataaaaaagtcaaaacaaaataaatttggattaattatcctcgtactgaaatcaattggtttggaactaagtaacaataggatacatgcggcgcaccggatcatgccattctccctctttaaatatcaaagcagatccctaaaccctaacaaagatccaaatatcaaacctcccctcttactacacgctccggcacctccaaaactccatctcgaggtttgtcacttttatgttcttgtttttctttatttagaatatgatgatgattagaccgatggctattttctttaaatgcctttactcctctgactagagtggtctgtactctgaatcagagggttcatttcgaatcttcgaacgttgtatttcgcttcaaaagctagacttttcccaatttacttgaacttattgtaattttagtgctagcccattgatcttggtctccaatgccactctctgttccgaataactgccgattattgaggggttttttttggacttcatgatttcgagttgttgtaaaatgattggggattcatttaaatatgaaatatatccatcgtttatctcaaaagtatatatcttaagataaaccatgaacaagaagtttccgatctaattcccatgggttgtctaacgagttattctcaacagattacgaactgataactagacgtttgaattttggcacagagagaaatcgcatcactttgaa

SEQ ID NO: 95 - DNA, 2000 bp, Cucumis melo
  source 1..2000: mol_type = unassigned DNA
taaatgggaaattggaaactaacttgaaacgaccacaaaccatggggacttaaaaaagtgataatctaacaaaggctttacactcctttttcataataaagaacaaaaagaaagctcaagagcaatcaagtttatcataactaattaaagtcaaacactacatttctcaaaagaatgatataaaatgaccaaacatctagctgctttacagtgtaatgaacacccaccattaaaggaaccaaggcaactgaataaattggtaacttaattgccctccaaatcagagtccccataccaacatcctcttccccattctcttggggcatcgaatcaacctccatcgctttacattccgataacaaacctctaaaacggacatttctgcacaaccccaattgcgttctacgactcccgcaggcaaatttatgagcatcagtcgacaaactcgatgaatttaaacgacccagatgaaagctgtgatagtagaagagtcaagaagataaatggggctaaacgataaggttttgaaagaagatgtagttgccattgtgaagtggtacttgccttggagtaatggtggtgaaggagaggtggtcgttgagtttgttctttagggcgccgagttgggtgggtatgcagactatggaggccattggcatcacatagctgaagatgaaactgcagagtgaagctgcttgttgaagcagaggatggattaattaaagtgggacgattttagttgtgtcttatcttcttcaactttatgtttcctcttggtttgacacggttttaccattatcgctaccattttaagtaacaatagtagtgatgaatgggtaaaatataaatcttattccattgttagaaccttcgacaagttttccattatgtgtggctgtgtttgacccaccaactcgagtagagttgaatttgtttggtctactatatttacaaactaatattaaataacaaaactctattaatttcatcggtgttcactgttgaaatatatacatttagtatgaatctttatctatttctctcttacccttcctctaacatttctagtgcctccatcatcaattgtcatcaacgacgaaatgtgacgataactatagtcaacgagtatttccaccttactttgacaatattcattgccacaatatgctcttgacgacctctagcactccacgtatgataaagactacatttgatgaccaattaaggaaatcgtatttgacaccacattccaatggctatctctagtgatcaatttcgactatcacttgtggttatcgactttcaaccatttctaacgactaacttgacgaccatcttaatcaatcatatactagaaaaacaaaaaaaaaaataactcatcaaatggaaacatttttaaatgcaattttgaaactaccacttctctgtatttaatagtaatttgacattaacaaaaacacttttaagtacataaaaaaccaaacaaacttgtatataaaacacttttgaaaaaaacggatgtaaccaaacacacaagtatttttcttttagattatgttttaaaagatagaaataaaaatattaaaagaaaagcacctttttacaaacatgtaaatccaaatcaaacatgctattttttaatactaaaagaaatagaaaaaacatgttaaacatatccattagcaaaataaagtgaaattccaagaattagaaagatggtttgaaaattgattttataaagcgaagaaaaacctttttccccaaaagaataatattcttattttggaaaaaacagaaaacaaaaaatgtgacaaaaagttacattcctgcggatttgaccctctggtggctgcattcgaatctttgatttcgaataactgaagtaaacattaaccaaagtccgtcgaaatcttcctttttttcatttgggattccctcaatcttcatcaccaccatcaccatcctccactttcactctgtttccctccaaacatcaaaaa

SEQ ID NO: 96 - DNA, 1373 bp, Cucumis melo
  source 1..1373: mol_type = unassigned DNA
tttaaatgttactttgatatgatctatgtttagatttgaagtatttttctcatcattaaaaagaactacacgatcgtattcatttagaagaagaattgtacgtacgcgtgtagccgattaatcacgtgttgagtgaaacattttttatatttttgctaatagacctatatattgttttcatttttaaaattgatatgtaaatattttggtttgttatatatatatattttttttggaaaaaaaactcctttatttatttgtcgttaagtattaatttctttttttagtacttttattaccattgtggccttgttttgctcctcaatttagatatttattatttgtggtttatttatttcttttgttttcgggacaagtgatgtttgggatattaaagtaaaggaaaaaaaagagagatattttgattgtcaaaatgtcagaaatatctaaacccggagcttctgccacgtaggcatcactttcattaccttttataaaaagtacgaattgaaccttcatgacactgctcccctgctcccttatataaaacccaatcctcttccatgctcagtattatcttcactctttgctcgaaccgcgtgtttaacagataagattcaactcacaagcattcatcgctaggttcttccaaacaaaaaccctacatcttttccatttcgcctccttaattctctcatatttctgtatcttaatccattctaaaactacattttaatgcactgccttgtgttctgtattccactatctgttatcgttttattgcgttttctttgatcagatcgctttgttgttgcatgaactgctgagttcgtttgatgattttgtttgcgcttcagttttcatcgtttgccgtccagattgtttgattggcgagagtgaagtgaaaattctgtatgatattggagcgtttcgtgtaaaatctgtcttgtttttctattatctgtattttagtgatttgtttttcgttgacgattttgtatgacgtaaagatattgtccattttaaaggattttcttccactggttactagagatcttagattgagctttcattcggctgtattttgatgatgctttttgtgtttttttttcctttctttctttagcttttgcggactcatggagtctttttctgaacgacatcttaagatgtttaagatgcttatttgcttttttctatttttggtatgacggggtcgagtctgattttgaacgacatgttaatatttatgatatttttgaagctagttgtgcttgattctgaaaattgcttttgatacacgagaaacttttttgttttcttcaatggtaggattttgaccattattattattattttttaaaagatcaaat

SEQ ID NO: 97 - DNA, 2000 bp, Cucumis melo
  source 1..2000: mol_type = unassigned DNA
ccgaattcgctattgggctgcataactttatcacttgctgggagactgcaatttgtttgtttagtgctatgtagttttcaagtttactaggctagtatgtttgtattgcctgagagtgtgcatcatgaggtggataaaattcttaggtcttatttttggaggggtaaggaggatggtagagggggtgttaaggtggcatgagcggaggtgtgtcttccttttgaggagggcaggcttgccatccatgatgggccttcttggaaatattgctatgtctatgaagattctttggtcgctattggcgaattctggttctctttggtggcttaggtggaggcttacattcttaaggggaggtcgttatggacgattgatagtgaggttggttgatattgtgtcttcgggctatcttgtgtaagtgggatagtttgaaagcacttgttcctatggaggtgggggatgggagaaggtgtagagtttggcttgatacgtagttgcatggcggtcctatccttgattaggttggggagagggtgctttatgacgcgacgagtcggagtgaggcttgactttctaattttcttggtcatgatgaggagtggaggtggccacgagtttctttggagttggttaacttatgggatacggttcagactgtttgttcgtgtcttagtgttagtgataggtgagtatgaattcctgacagtcatggtggtttttcgaccgcgaatgtgtgggatactctctgtcctcgaagtagtcaggttccttggactggtttattgtggggtaggggggaattgttttccaaaacatttctttttgagtttgacttgccatcaaagataggttgttctttttgtagttctttcttttggtgctttttgtttctatggatcctgtgagggttttctgctctcgtgccttaaactcaggctgtgaggtcctccttgttatggtataataatattaccttttcaaacaaaaaaaaaacaaattgattcagaatgatttttttttctttcttttgtatttattctatgtttccttattcaggctactagatttgaatatgttatttgttacttccttttctaacaaaattagttataattaattttatttggtttctttaaaaagtgtggggttgaagcttcttgcagaatataggatcacaaatgcctaatacacttctttctacttctttgttttgcagcagggtatgaaaaaacaaattaatatgtattttttatacttctttctcgtatgcattattcttcttttgtttctgttggctttgcattgtagccgttttcttgttcttgtctcattttttctctaccttttgtttcttctctaaattccttttatgttcatttttcataatgcggattttttcaaaaaagaaaattatagttgttagttgtgtttgatgagaaacaagaaaagagagtgaaaagagaaaagaggtagaagagaaaagaaaagaagaatctgagtagaggaaaaaaattgaacaaaaaagttggaattgtgttggatgaagtgagagcaagaactaaattttgtttgagcgtcaagcccccaccccacacgtttctaagaacaagatggtaattttaaatacaactaatataagcaaaatacaatttctcgaggaaataggaaacttcattccaggcttcaaaggaaaaaagaaaaaaaaagaaaaagaaagtaaaacgattagaacgtgaattgcacgtcactagacaaaaccatcttttggtagagaaaaacacgtgattacaaaaaacaaacgaaacccaaataaatatatatagaaaaaaaataaataaaagaaatagaaaaatctaaaaaaattgggttagcgggcaaacaagaaacccttgtttcgatcccccaaaacccccccaccctttctcccatcttctttcttcttcttcccttccccatttttgaagaaccaaccagcacctctgaccaacatttgcttaccc

SEQ ID NO: 98 - DNA, 2000 bp, Cucumis melo
  source 1..2000: mol_type = unassigned DNA
tagtttggttcataggttatagtttccaaatttgttaggctatcattaatcaaacacaatacttctcttgtaggatggctgccccctatagtacttttttaacttaggagaaggatataataattatattccttttagaaaatataataataattgtgtagtgctttgatataccttaaattagctactcacgtttttaggaggaagcttccgttgcttttcatggtgttatgatcttttttattttataaaggactgaactttaaaatttctctttcatctattttggattggattccatctattttatacgggaagtgaactctaagatttctcttcacctattgtgaatcggactccgtcatgtaggtcaagactacgacagataagaatagacttccacgaaagaaagtggtcaatcgagatggctatatttggctctttcagctcaatttcttcttttttccttgcatgttcttccgttggtacatttcttgcactttttttgttctcacatgactaatgtattccaagtttatcattggcattgtgcctcttttaggcttgtaaactctcgatccaaaattatctaggacatatgtttcctagtgaagaaatactagtatattccttatgtcaatatgtcaaaattttcaatttcttaacctttgagtaaatcaatattatatttttatggaggttatttataattggaaaaaagttacacccatctcaaccctaattaacaccaaatgaaattgtaccatgcggcacaatatttttttgtgagttttttgcaaagagaaacaaagtagcagacaaagaacaaacattcccccaaaaacagcagagaatacctaagagagaatgctctctcgtaaaaaataatacccaagaatcttcccaaaaagagggagtaaaagagtccaaaacaaacgaaccgaagattgacaagaagggcactctcgccctccactgcgccgctaaattgtaagaagcatattttcttgagttaacataggaataggtgtaactcaagagaaatgtaattcgtagaattgaactttgtatattaatttatatggtgttgtagatacaatctttagtatttactcatttggtgctttctctcaaatacaatttaaacttagaactttttgatcttcgattttcaggaagttggagttgcaaatcaattcgagtttcaatctctggaatttaataaaagtttgatcttccaagttttcaatctttcagaagacgatgatcttgatatggataaaaaattgcacatcatgagagctttttgaagtttaaatcttcaattctctagagcttaaattcttccttaaaccaaagatcaccaaatgaatgacaaatgtctctatttatcgaaaaatttcatagacttttagatgggcttaggcacattacttgttgggcttggacttgggcttatttgcttggcgggctcatgctcgagcccattatttctttggcctatttttcatgaggggcttgaacttggttgtatacgaaaaaacttgactacctaaatctaatcaaattataatcatcacaattttgacgtgttacgatttaattggccaaaaattcttgttcaacacttgtctctaatcattttcctatataatttaactaaaatatttaactttaagtaacttaaaagatatagtttaattcgaatcaaaatacaaatacaatttcgtctatctattcccatcataaatgttgattgagattcatattataaacttctttcaggaaaagaaagaggaaaattcacctaaaccacgttttcctattttggtaagaatccccaaaccataaatcattccaaaattattttttttagaaaaaagaaattcacatggcgtaaaatttcagccccgtgagatattttcgaacccccagatacaatctacaccgtgaaaacaaaatcggacggtggagattgctataatgtccgtttagaggca

SEQ ID NO: 99 - DNA, 2000 bp, Cucumis melo
  source 1..2000: mol_type = unassigned DNA
acactttgaaagtccatttgagagattagggtaaatttgagtgaggatggcgtgatgacaacgataaaagtgaaaaatgtcagatccaagagagactcaaaagtgaatgacgtgaagacaatcggaatcgaaattgaaaaatcagattttaaattatcttaaaccacatattaattaaatttcgattccagtttcaatttggtttgctgtgataaaactaaattcttaattgtacctaattttctattaaataaataggtaaaaaaagtatagtaaaaatattggcgtcgcccggactcgaaccggagaccttcagtgtgttagactgacgtgataaccaactacaccacgacaccgttttgttacatgagtaaaatgtttcctatttgtctaatattattattactactactacttcttcttcttcttcgagaaaaaccaatttctatgggtttaaatttccaaattgatgttgagtgtatcaataatatagcactcacatgctacttaacaaaaatcaattctttctttttagttaaaaccttttcttttatatttagtgaaaggattaagctatgttctacgttaaattgttataaacaaaatttgattgttacttatcgagattaatttatttaagtggatatgttggaatatgttactaaaatgataattgatagtgatacgtcgagtttatgctaaacacattttgatatggttttctttttcaatataataatttgacattaattacatttttttttcatatactctcaagaatgtttatttttattatgtacttttaaaaattaagattttttatggttttatccataaatttgtttcattttttaatcgaaattttagtattagactttagttgttaaagatcctaaaatatagtcattatattttattaaagagtctccgtcacgtgtataaattaaaatagtcttaaccgttaaaagtatagtgaacaaaatttctaacaagaattggatcggagtagaagggtgattgattcaacatgatccttgtgccattattgttgttactcaagggacgttcatcaatagataacttgaaatcaaaatggcataaactattgctcagttgaaaggttgtttgttgattgaagagttaggtttggatatttgggtggaagccaatggccttgtcgtggttaataaggtgctttcatttaattttgcactctctcctcatggggtttattacactaaagtggttcatttaattgagagcatattggacgaaaataaacaattaagactaaggacgaaagtaatatttaaacattattttaagaaaaagtcattttaattcctaagttcttttttagtataattttcatttgtttgctatattttaaaaggttacgcttttatcaataattctttagtttagttttcatttgacctataaattttaaaatatcacctttttccttttatattttgggtttaattttccttccttgcattttcatattttacactaatacctttaaacaactaaggcttactcctagtctttgaaggttaaacgttgagtttcaactaattgatttaatcatctaaaattttgagatttttttaaaagcaatgattaggtgcagtcttctgcttcccatttatttatcacgtaaaaaaattataaaaaaatcattttttaaaattgttacctgacaattttttgagtgcaactcgaactgcctatcgttgtaacccgactgtacctaaatattttcaatattttaaaacctttgattaaatgataaacaaattaaaactaagggggaaattacattttccttaatttaaaaacaattttgttgataagatggggcctggcccatgaggttttgggctgggccttttcgaatcgtctatttataatgagcaaacgagtctgagcttcgaagaaatcccctttttttcacttgcgaaagagacgaacaaacgcaaaacagtcgaaggaag

SEQ ID NO: 100 - DNA, 2000 bp, Cucumis melo
  source 1..2000: mol_type = unassigned DNA
tgttggcaatgatttctttcagaaacttttgccaccttaatgcttgcgtagtttcaaactaaatgctgattgctgtcagtaactgattaaattttgatttaagtatagtagctgccttattgtgttaacaagtttctccatcattttttgcattgacttgatgatttgacttcttttgggtcatatctttgattctttccatgtttgaaagttctaatttagatgttggtttgtatagccattgagaagtttaattggcaaaacattttatagcgaccttgacatagaagaagatgatatcttcgtttctgatggtgcaaaatgtgacataacacgacttcaggtatgatttgttttagtttggaacatctttcatccatgtaatatttttattttcctcattttttttgaactttaatgttggttactaaccttagttaaatatgtaagatagcctggtaatcgtatctttcatcttgttactatttaacttctcttcccaattttggcagcttgtttttggatccaacgtgtcgatggcagtgcaggacccatcatacccggtgattttctcttctcattaatgaaaactttcgatgattaattggtacactaataatattttgcctgtccacctatatatcagacatttacttaaatgatcatttgaaaaatatcaagctcttgggcaatcattttgtgtgtctcatctttactgttgtgcttgaatgagtgaccacgatggatagactttttgagaaagatccctttgttaatgggtcttttttgttgtattctttgtcggaaagcggaggaaacccagatcatcttatttaggagtcttagtttgtgaggtctatgtggaattttctttcaaaagttttgatgttgtacttgcttgctagagggatgttcatttgatgattagagagtttctcctctagttgcctttcaaagagaaatgacaactattgtgggttggcctagtactaaaataggagacatagtctcaataactaactaagaagtcatgggttctatccatggtggccacctacctaggaattaattttctatgagtttctttgacatccaaatgtagtagggttagacgggttgtcccgtgagattagtttaggtgagtgtaagttggtttggacactcatggatataataaaagagaaatgttgttttctattttgtggtttgtgggtgtgtcatgtgtgctttgttgtggaatctttaaggaaagaggaaccacaaaccctattgaggtttggttcttggtgaagagtgtgaggtttcatgttctgggcttcggtttcaaagactatttgtaattattcactctcacttagttgcaaacactttcttttgagggtttccgtgggcttggttttctgtatgctgttgtgtttttttcactttttccctcaatggatgcaattctttattcaaaagaaaatctttactcttgaatttgcatatgcaccctttgataacttttggtaggttagtcacttcagatcaaaccacaaataataatatattttgttttcgcaaaacttagaaaatatatttttgatatcagtctgttggtccattctcccacttattggtttatgtttttttggtagttatgaagtaacatccaaaggcctgtattgtttaggctgtagaactttatacaaacctctgctaagtcaattcctaatcaagaatttgtggaactgtaggcttatgtggactcgagtgtcatcttggggcagactggacagtaccagaaggatgttgagaaatatggcaatattgaatacatgaggtgtacaccagaaaatggattttttcccgatctatctaaggttcctcgaacagatatcatatttttctgttcaccaaacaatcctactggctcatctgcaactagggaacagttgacccaacttgtgcagtttgactaaaagaatggatcaattatagtctatgattcagcatatgca

SEQ ID NO: 101 - DNA, 1078 bp, Cucumis melo
  unsure 857..867: note = unsure at all n locations
  source 1..1078: mol_type = unassigned DNA
ataatattaatttcatttaaaaataacttgaattttttcctcctatatttatcatgcatttttacaaatccacgttcgaaaatcccattaatcataggagttaaattgtcatcacttgatttgaatatttatttttttttaaaattaataaataaataatgtcacgaaaatgataaaaatgcaaagtatcgaatttaaaaattaaacagaacaaaatttaaaaattaaatgataaaaataaatataaaatataggtggatgttaaagataataatttaaatctttatctatcatcaaatgacgatcctccaatggaaaaagaaaaaaaaaactttattctttacctcaaactcctcgctaaaaagtaacaatggtaagataaaactttattttaaattattcttccacttgcaagcaaagtaaatagttatttgattcttacacaaaagagaatttttactttttacttttcattagttatatataactttataatacatttccctctcatggaatttaaaactaccatttgagcaaaatattttaaactaaagaaaaatatgaaacttaaaactatgtgacagggatgataatgacgtttactccaaattttcattttaaattaacgtacgttattttataagtatatgtcaaaattttaaggatctattttattagacaattcaaattatatgttgtgctttcatattttgttaaattcaataaatatgcctttggttgattatactatttttctaattaactctggagacatttcaaaagattttttatttatttatttaagaaaatatattaatatggtcaatagatatgtattatgcacatgatataaaaannnnnnnnnnngtaataatattattacataattaaattctttcatcttcctaacagagagagaggatcgtcctctcagcgacgctgatcccaactgttccagtaccaaatctctgtgtcccaatccaacagatccttcttttaagctaaacccaccatttttttttttttctgaaacccatttcttatctctcgccggaccttcagattttacctcaaaacc

SEQ ID NO: 102 - DNA, 2000 bp, Cucumis melo
  unsure 1356..1385: note = unsure at all n locations
  source 1..2000: mol_type = unassigned DNA
cactatctatcatagataaataagtgatagatctaaacgatcatttaccaaagtctcaaagatcatgtaccaatatctaaacgagtttggtacaagattgtataccaaaatcatttgatttgatacaagatcgtgtaccaaaattgtttagatttgatacaatatcatgtacaagatagtgtatcaatatttaaacaattaatcgtctatcctagataaacaaagataaaccactaggaaatcgcacgaagagaaatagaggaagtgaagaaaaaaattactcatataaattgatgaaaaatgttatccttctctaatatggttttaatttttgcactaggaaatcacacattaatgattataatacaaagtcctacaaagagatctgaattgattcatttgtgaaactttacaattttaatcgatacaattattaacttaagagtgtaattgatttaagctacaaggtttaagcaaaaaactaaaacataaacagaagtcaaacttttcttaatttttgagtttagtgagctacttatttattgggtagctttagaaaagtcaaacttgaattgtcatttttaagtatgatcaaacttaatttaacccaaacttctgttgtaggtgaattagcagctagtttgtatatattgactgatttacaaattcttattttaattaattttaaccatccattaaaatggagagttatagttattcaaggattttaactactctcaaaatcatcaagatcacttgcatatttagtataagttcaaggacttaagtccttattgatattttcatcatcatctggaaaactaatcaaataatcatgttgatgcaacttagatgattaagattaaagctaagacttttgaaatgataaagaatataaataaaaaaggaagttttttaaaaatataacaaataggtaaaatatttacattatataaaacaattctagaaacgaaaaaaacccacggtctcacaatgaaaaatacaaaaaataccctagtcaatagcaattaatcagccagcttgcgcgaagaatattcttttaaacgactgtgtactacaatttcaacgaataatccagtattgtttaggtcatgacacgatcatgtagttctattttaacgatgggaaaaaaggttttgaatttaaatgatcgtattgatcatgaaaaacaactatgttgattacgataagcgatcatgtagtccaatgtaaatgaatttcaagtctaacgatcatgttgaccatgctaaacgattgtgttagctatggtaagcaattgtgtagatcatgtcaacacgatcgtttagatcattttaaacgatgtgaaaaagactnnnnnnnnnnnnnnnnnnnnnnnnnnnnnnccatgataaacgattgtgttgacaatggtaaaagattgtgttgacgatgataaacgattgtgttgataatggtaaagatcgtgttgacgatggtaaacgatcggctaaatcatgtcaaaatgatatttagacgatgtagatatttttgaatatgagaaagatgaagtgactttaaagatgaagtagcttttaggtcaaaagcaaataaaaacatataaaaacatacgaggaaaagttaacatatttttagtctattcagactcatccaaattttaattgtgtcatcaaatctcaatccacagctctcaccttgattaaatacataacatatctaagatcttataattaagttcatgaacgtatctaactttttaattcattgatctgccttgcttagttcaagttacataccctcttgcttaaaaaaaaaagttacataccctcttgcttaaaaaaaaaaagttatataccctctttgacaaatatcaaaggagaaaaagacaaaaactgacattggcttcccatcatccagagaaaagaaagaaaagccgcgcgggttgtttatccacttgtttcccttattatcctcatcgattccaagttttgaactcaaca

SEQ ID NO: 103 - DNA, 2000 bp, Cucumis melo
  source 1..2000: mol_type = unassigned DNA
ttcctatttaaattaactggttttcttagaaaataacagaattcctgtgtggaatcccggctctaatccaaatttcatagggatgaaaaatgaaatggggagtagacatggagatggggagtggcattcctgacctcaccccgtccccgtggacatctttggtgacagaatcatcccatccaaatcaatctgtgggcaaatttcaacactcacaacaagttgaaagactttgttttgtaatgtatttcgagttcaactcacatgtggttgtatagtctaccatttcaaacccactccaaataagaaaaaaatataaaaaaacatattttagaaccccacaacattttttttatttgaaacaaacaaatatctccacgtgtttctgtttgatctcaaattgtacaaaagggagacaaacaagagcaacttaatcgtgtggtcgaaagttcataaaaaacgttgtttttcattactattattacatcaaccaatgcgatctcaatcttgtgaagatttttcttccatgtgtgagtcatttcttctcgatcttaattatcttctcacaatccatttattatagcataatctaagttaatttagattcaaaactatacaataataataattaagaaaattacaaatttaaatagcaaaagaaccatttgttctttatagtttctacactaacttttgaaaaggttaaggttattggaaatctttttctggggcatttttctccaattctacaatagacaattttttttaattaattaattaattaaatttaaagtttaccttggagtagtcaataattaatttttatgcacatttgtcttttatatgattgaatgtaacaaacaataacttattcttcttcttttattctattgttttgatgcaaacccacaatatttaatgagctcatagttatgtgtttgctttactaattaattattttcttttcataaaataaaaaaacttgtacaatataaactctattatcattgaatttttagtacttaatttaaacgtactaaaataaaatacatcattctgactgacgatccatgtaaataaaatctaaaaataaaagaaaaatgtcagaaatagcaaattgacaaaatatttacaagccatagcaaaatttcatattctaccgataacaaacatttgatagacattgatattcttctgtcagtggtattggtagacagtgatagaagtctatcaatttctatcatcgatagaattcaaaattttgttatagatcgtaaatattttaatttatttgttacttttaaaaatgtctcaatataaaaattattaaataaacattaattttttatttttcaattttaatatctaagctcataaatattaactttacccattatttatttggtttcttaccgcttaaatgttgcaaaaatattttaaattttatttttgaaatttggttaaattcgtttttacttaaaaatttccgtgataaaaatattcgaattttttaggtttttataagatttaaaagtaaactacataaatgaaatcgttattttctaattctcaatttaacttttttatactttttaattaccaaatggaaacatgaaattttaaatatatttattttaaatcttactcgttacaaacaaaacaataaatttaaaattatttttccgagttttaaattacaagatttaaaattaatttttcaacaagaccaaaagaattgtaagtttcgaataaaaaggtttctttttgggctataaagtccaatttcctataaaagaatttgatcaattggagcccaaagtcagatccattaacttttgggcccaaatagaacaatgaaaagaaagcccaaaagctgacccaccaattaaccttattattagggttttgctctctcttttaacatccgaaaatcaggactctcttgccgcttttctcttcgccgtcgccttcttcgagcttcaagtctcccatcctcttcagcc

SEQ ID NO: 104 - DNA, 1974 bp, Cucumis melo
  source 1..1974: mol_type = unassigned DNA
tttgctattttcgtttcatgtgggaaaaatagtatagtatgtttacgtcttaaattatttcaaattcctagctaggaattaaaactttaatatatccaaaacgtttcttatttattataaagatctgcaatagcacaatgccaatttctcttctttgaaatccaggttcaaatcccggttgcggaataatgttttgctattttcgtttcatgtggaaaaaatagtatagtatgtttacgtcttaaattattacaaattcctagctaggaattaaaactttaatatatccaaaacgttccttgtttattacaaagatctacactagcacaacggtaggtagtttctcttctttgaaatccaaaatctttgctattttcatttcattttcaaattgaatgcatagctttagattgtagtaaacattgtatatatatgtttaggttgtgctaactttaaatgtacaaaattcaaaatgtaatagaattagatgtacatgataaagagttgcaatatttagattaaaatataagaatttaaatgtaagacttgcatatatcaaaaaaagatttctttataaacaatatttttttatacaatttgaaggcaacttattgttactcatgggcttgatccaaacttttgttgtcttcactaaaattcctctaaatagttcaacataaagttgttcatgagaaaactcattaagatatattccaacattatgaattgtttgtccttgtattttgttaattgtcattgcaaagtataaatgaatggagatttgttttcttttgaacttgaatagatatccattatcatttggtgggtttaatggtattcatggaagaaaaatttatttttctgcataatcacccattattatttcagcatgtataatatttttgctaaataattgacatactaatcttgtctcgttacacaatccattagatgaatccaaatttttcaataacaacgttggtaaaaaaaatcaagacagccttttatatagtaaaaaaaatgttacaacaacttttcaacgttcaagtctttaaatttgtattgttgattagaattaataagatatttgatttgcaacaaatttctaaaatgtaaataaaaaaccatttgcattcaaactctttacatccaatactttaattccttcgcatcctatactttaattccactcacttaaatataattaattaaaaatatagtggataaatgaaaaccaatttgcatttaatttttatatatgcatactttaattccactaaacttcgttagaattaattcaaaaagttgtgggagagaatgtgcattttatcatattacaagaaaaataaaattaaaaaagaatttaccataaagtcattaaacaaaattcaaaggttgaatggagagaataaaatttctgcacgctttgatatatacaagatatttaaaattaaaaaaatagttttaaagagaatgtttctaaattattattctaactttaaatataattactcataattatacttatttttttttaaatttagaaactaaaatgatacattctcgaaaactataatcaaacgagttaatgttataaactttgaaaactattttttgtttttaaactctgcatagcaaatagcatatagaggttttttaaaaaataataataattaaaaaaacattaaaggcaaaatctattattccttgatttgtgtatagggtgtaaatattttgttactgtgttatttttaaccatttgcgcactgatacggactaaaaggtaaaaacataattttctcgaattgttattagaaaactggggaagaaaaaggaaatcaaatcgcgcgaggtgggatttgacccgggaacctaagactagctcggtgtggtgttgaaatccacgcgttgttcaccgattttcttcataacaaacgcacccaggctacggcagtcttcgaagctctctcaatc

SEQ ID NO: 105 - DNA, 2000 bp, Cucumis melo
  source 1..2000: mol_type = unassigned DNA
gtcgtcgagcagagctctggcagttaccctacaacccggagcacgataactgcagtgatccctatcctcagcatcagttaaatgggccgattcaaaacttttatgggcctcagcccacttccacttacaactattacaaacatggatacgatagtcacgatcaggcccatcatcttaattactccacacattccaatatcttcggccgccaaactgcctccgtttttagcgacgaaaatgtccataattgctctattatgtaataaggctaaacactaatcatctatccctttaaatcttgaatttttgtaataaaaccaatctatactttttgccatagtttatttctagaaaagtttagaatacattgaagatttgagaaacttgtctacaaggcatcaacaaaactacgtgaaaatgacaaattggaaacaaataaaaatatcttatgtttgagtatatggaatgaagggattgatgtaataaaacttaacgtcaagtgttaatattacgctatagttattttcttgttgtagtaattttctcttagttaatttttttattattgaaataagtgataaattttctaataagaacgtaaagatttaaacctctaattaagttaaaaaaaaaaacttgaattattgtttgagttatgaggtaacgtaaaagacaacttaaattttaaagtcaaaccgaaaggaaaagagttaaatacccacaaatggatcaaagaagttaataacacacacgcacgttgggaagcttaaaaattagcaacaaacaagcaatcattggtgtgggacagtattgaaattccacaaaactacaagggtatattggaaaatccaatttatttatttattttttaataggaattaaatttactgtaaaaaaatgtaagaccgtcgattgacaattggtggactgtgaaacgtggcaaaagttaattggcgaaaaggagaggaaagattttctcttttcattataatgaaaaaaattaatgatagtacacgtggcaaaaaagtattggagagaaatttccgggaattatctctaatacgcggctaatttggatgtcaattttgcaaagaccagaatctttttgaacagcgaagaagaacaaatatatagacatacaataataaataaaaataaaaatatattaagcataagagaaaaagaagatttgaaggttatattgaagtgatattgttggtttctccatttctgtgggtctgactctgcctctctcttttcgagccagaaccaccaaaacgaaaaaacccacacactgtatagcaaaccctaattctttggtctcagatcgcccatggcttccactaaagaacgcgacaacttcgtttacatcgctaagctcgccgagcaggccgagcggtttattgtattggttttcctaccttcctttccactttttttttttgggtttgcttctcatttctattttatgcttttcttaatttgtgttttacttttcactctctctttgctcagatcgtatttcttctggttgtttaattttgtgtttatgttttttgacttcggatttaagccacgatcgcttgcctttttgtgtactattttcagaagtgttgttatgtttatccgtttacacgatctgtttgaaatttatggaaatttagtttgcttataattttgctacatttctattgtttagcttctcgagcagttttttttttttttggccgatccattgatttatactgtttttctgtctgatctgttttatttaatggagaatactctttttttgcgaagcttggtagctcatttttcactcatacttacacagactacttggtcattgtttttatctgtaagcacaaagcaaattcaagtttctgcctgttctttctttgcttgtcaaacgacaaaactatgtttgtagttgcttttggatgatagatggtgattctgattttaatttacgttttcctgctgttttttttttaaaagaa

SEQ ID NO: 106 - DNA, 1643 bp, Cucumis melo
  source 1..1643: mol_type = unassigned DNA
tccattggccctctcaaacctttatgtgcatatcactaatatggttgaatatgtatatcttctttctcgaatagatgattcttggtggtttcaataatcatttagcaaatccagaaattgggacctcaagttcggttgccgtggaaatagctaaattaactctgaaatcctcaaactgaaatgtgagaataatcacgatgaatctgaaccgtcaacggcggaacatagcaacagtaatcagaattaccaaatttcaattgggggaattcctttgtatgatccttccttgggcctaacagggattcttgatttgaacctctcttcttcgtaaaaattacacaaaatatttagctgctagagctagacaaaacaagatttagattggaaaaacaacaatgcagctcccaaattgcaatcctaattccactatttcttttttctttttcttttttttaatcttaggattcaattcatcattcatcaattttattgttactgctcattgatgaccaatgttttggattttgtgtgtcaaatattttagtttatatatggtgaaaagataaaatgaatagtttcaaattttgtgttttatgaattcctcactacctctttctttcactaatacgtatgaaatgtgtatggttgtttataaataagatggatggatgttttgattttgatttgaatgttaatgttacttaaattatagattttaaccatttgaatgaaatatggagagaacagtttttatatgtaaaaacaaattaatgtgagaaagaaataaatagcaatgcctttcttcactaataaatgtatttatattttttattaacaaaataaaatttatattaattattagtgtatgaggtgtgttcttgtacaaaggaaagtattgcaaaattacaaaaatggaaagttgaaattactgcactcatttgctaaaatcaaattagttaattatagaacgaaaaataaataaataagttgtatttgatgatcctagataaataacttttgaagaataaagatcaactatttaaaaaaaatatgtgtatcacaaaaaagaatagagaaaaaaatcacaaaaatcacatcccaaattataataattcatattataataatttatataccaaacataaactataataatcacgtattattataactcatagactataataactcactccacgtcccgtagttattaaataaaagaaagtaacggtaacattaacattataacttcgccctcatttatggcaaggaaaaattggggggattggcaagtattatatttgtttatggaaaacttttgtgaaggtggaaaatagagagagccaaattaacaaaaataataacaaaatcaaagggtgtagaattcatccagtttgagagcggaagattagatgggtgaagaaaggattattctagaaccctagcccacgtgtcataatccaaccctcaccttttcttcaaaaaccctttctttcttctccctcccctatatctccttcttcgaccaccaaactcttttctctcaatttcccagcatcttcttcatttttcattcttaattcaacccatttcttctctcttttcgtttctattttcatcgtttctctataatttctccctta

SEQ ID NO: 107 - DNA, 2000 bp, Cucumis melo
  source 1..2000: mol_type = unassigned DNA
ggatgggcaatcgtgcgacacttgttctactcgattaacaaattagccgtgtaaaatccaaaaattgtggacaatttacggtatgatgtagcccctcttcacgttcttaagaaattttttataaaatgagaaagggaaaggaaatcattgaaaagatcataaaagaaagcattgaagactgttaattgcaaagaaagcttagcttaaaaagagtgcaacaaggcttagttggggatttaactactatgtctcccttattgtacattttgaatatttttatccttggcagacttgcatatgaaaatgtcgaaacgtcacacactaggtcgacaacataaaaatgaaagcaatagagcaatagattaaactaagtagaaaacataaagacaaggtgatttgaaggtatttggatatgtggccataggcaaataacgcgctggacaagcatgttcatgacatatgacactttgcacgcatgctcaatgtggatatatcagcatggcgcacgtgcctcactcggacacataaacatggtatgcgcggcatcatgtgcgcacgccttacacgaccaacgagctaggtgtagtccaagcacacacgcgatgggcaaacgtgcctatggctgcccctggcgcagacagtctcgaaagatgcatgtccatcctaggcccatctagacacgtccaaaagttccaatgacggtccaaaaggatacaatacctttagaagtgtcatggtaggtctagaatgttctagagtcatttgtaaattgttaaactgccttatatcttctagatatacaggtcctcggccgaccttcaaagcacctaggtcggttaggaaagctataaatagatgtaaggtggcttatttgtaatcaccctaaaatcttggcataacctagccaagtaagacaaccttgcctcatcatttgtacacaaggtacctttacaaatggtaataccctggcaaaggactacactcatttgtatacaacttgtacacaagcaatcttggaacgcaaagtactcttccaagaagtgtcaagctaagctccatcattctcacaaaatgatctctcttgcctttcaactatcttaaatcttctactgccatattctttctcatagtgcttagtgcactaacctctcaaaggcttacttggctacgtgggcgttaatattagtcaagtgttgtacgtttggttagttgaaaaatctaaccacgtgacaatagacaaacatcaattttattttattttagagtctcaccaagttcttaaataaaatgtttattgtaagacaaacaaaaatgaaaatatgttattatagtgatatagaattttcactattagtacaagatataaaagcgaaaggaagaatgaatgaacactcaacatttagaaagtgttttgagtaaagaagtaaatagtgagaaataacgagtacaaatgtgtggaaagttataaacttctaagatctacagaacaaaagattgataagatataaaattgatgttaggataggagctacaaactcctttgaccaaatatcgagcaggattcacaagtcatactctcttactctaccaaattcattagaagtacataatgggcatgcatgtgaacgaattaaaaaattggtatttttatttttatattttaaaaaaattggatgaattggcaatggccatgaatgaaccagttgttaaagtttagaggacaaaacccaaaagagagaagtgtacctcataaaaaacaaatccaccaattgagaatcacataaattataggaagacgtgtcactctatcggccgatcctcaaactcttccaccaaatccacatgcacaatctccttctcttcccttccaccatacactcaaaatcccactgatcttcttcttcataaaaacccatataatcataaattaatttcctcaagtttttcttttccaattaaacaaacaactctgcaaaagaggcctttcttccaccatttcc

SEQ ID NO: 108 - DNA, 2000 bp, Cucumis melo
  source 1..2000: mol_type = unassigned DNA
agacgaagaagaagacagggtgtgatcatttaagaatatgcgttttaactctgccctttttagggcttttttcttttatttatttgccttttttctcgctcctagggtttttccctccattgaattagaaggatgactgggccacagacttatgatgggcttcacggtcattatttgaaagtgtgatattctaaaaaaaataaaaactcatttgaattaaaatagggtttccctccatgggagtatgaaagacttttaattgaattggggtttttaaaccctaaattgaactaaatatatttttatgatttttacaaaaattaatactacaaaacaaattatgattaataaaatttgttgatatttttcaaaaaacaattatataataaaacaaactaaatattcaatttggtatttttaaccatgctataggaaaagattcatatgggcatcaaaatgaagcaagaacatggcaaagcaagttgggtgaagataagtattgtcctaaatcgaaggacgaggcaataaactcgatatctcgaagagtctccaggtcaacatcacaacgcctgcacaaaccaaatattattatatatatagccatcgtttactataagactatgtatttacgaaaaattctatattgttttcgacgattacatttatattatataggaataaaatacaaacttttcgaaaagtcatatatcccaccatataaagatcaaacgtggtagattgaaaacattatacagtatattctctatttttttctcataaaacttattacgctttgtcaagttataaagattaatggttttggtatattgtgctaacttcgtccatttgttgtgaaattacattttcactacttttttccacattgcaccatttttcatatgttttatttccattatctcgtagaatatgagcaaagaaaaagattaaagatgaaatttttcaacgtgtgagagaaacatcattaaaggttacttaataaggaattagaaaaaagagcatgaacctagaacaacaagatacaaaatatcaaagacaaaagagttcgatggagagctagaaagataaatcaagattttgtaaaagaaaagtgctcggtggggaactagaaaaatgttatagaagctagaaagataagccgataatttgtaaactataagaggccgtttagaggaagagttgggttgtgaaatgttagtgttatgataaaactattgttatgtttggggaaagagtttaaaaaggtacttttatgataaaatatgtctgggataagagttgaaaaacgtagttttatgggagagttgaagatatagggttatgaagagttaaaaaaggtataagaaaaggagagagagagagggaataggggttatgatcatagtcttaaaacagaattatcataacccaatccaagtgataacccttggaccaaacgacctaaaatatcaaagagaaaactgtttggtgaggaactataaaaatgttatagaagctaaaaatacgaactagaaagataagcggagccaattataagggttggttaagtgtagggtttatttatttgaggggaatgataatttaagatataaattaatagaatggcaagttttgtaagaaaaattaataacaactcgataaacttttgtttgtgttggtagagaaaacatgggccacaaacatgagcccaaatgtggagaagcccagctgataatttaattttaaaaataataaagattagattatttttgttcgcccaaaattcggcgcggctaggaggttgcttataaatggaaataaatggaaagggtgttaggtctcgaacaagtgtgcgacggtattttaaaggtcggccacgttgaggcggcccttttcactcctttttcctcgctcgtattcaatctagggttttaggtttccaacttctcttcctccccttccccttccccttccccttccttctctactcatcactattctca

SEQ ID NO: 109 - DNA, 2000 bp, Cucumis melo
  source 1..2000: mol_type = unassigned DNA
atgggtagttttcaaattaatccgacctttgaagtactttggtttttaaaataattttttatcatctgaaatcactccatagacttatgttaccgtaaatcattattctttacaaatgatttgattttacttaaaagtatattatttcaaacacgttataggtattatgaagttttaccgtcaacaattatagttagtaagccaactatttataaaaatttaaaaaggaatatttgaagcatggtgcatgatgtatgttcttctctctcttaagttgactatcaaaacttaatcatgctcagaataacatacctcacatagcatgtgcaatttaatctaagcaattcaaaattcattaacaataattcatacacactacaaagtcataccacctatgtcacccaagaactactattattgtaacaagtcaaataagaagtccctatcctatccatcctaagatggagtaatttttcttttccttaaatttttggaaagaagaatattgaaattcaggacattaaatcaaagctgttcggagataaatgaaccattcttcaagtaaaattcatatttgtcatcatgcaaacaaatattgaaaacatgatatcaagaaaaagaacaaattatttaaaaacatcataccgcacatcaaacttaaaataaccttttgtgcatatcaaacttaaaataacttttctcaacaaattaaagcgacataaaattgataatttttgttttttttttaaatatatattcaagaaaatcgacaaatccaaatgacaagttgttcacctgtatattaaaaaaaacaataatgaaaatttgaaaggagagatgagaaaaaaaaaatcaatccatcaatccaacttgaatttttgggtcgacagcatatccctaattataataggaagcaccctactttttttacaaaagtatcgaaattattagtcgaaaatcttaattagagtccaaattggatgcagcaaggatagttttaaatccaattaatagcatgcctaatgctattacaaatatattttggattatacataaatagaaaaaaaaaagtgaacttccagactcaaatagattttactctattgttataaaaactatacattaaaattagatgtagagaatgagagctcaaaaccaagaaaagtaaatgataaaagggaaacaggaggtgaaaagaaaaggtgataccgcggatttgatgtggctctcggtttttgcctcccaagcaatccccattgcccatctcctctacaccaacccacttttctccctttctttctttctttctttctaaaacttttgttttccaattttgacctctcttcttgggcccacttactaacaaatcaaaaccaattttcatttttttttcttttctctattcccttccacaaataagaaaaaactttatataaatcaatacaagaatgacatttattataatagtatatactataaggtgggagggatggcaattgccaattgtataagtaactattaatagacagtaaaactttgaaatagaaggtatttagatgtctgagggtaaacttataacattttatgaaatctaaaaactaaaattaaaattattgggacaatataaactttagacataagaagaaaacatatttttgttaataatttaacaaagaacacaacaagaatggtagagtgttgattaagagtgagcataatagacaaaaaaaaatatagtcaatcagccaaaatagacggtggttggtcggagatgaagagagtttcaatctaatcagttggtaaaaaattcaaacatcgtcacattctttaaaacttttaaaggtttaaatctgctaagatttatcgaacaatgacctatttgtactactttatgattgacatcaatttaaatatttaccggtgaatttagtaacgattagggcgagagcggttttaaaaacaggagtggagtagtggacaaggagggggcggaccaaccg

SEQ ID NO: 110 - DNA, 2000 bp, Cucumis melo
  source 1..2000: mol_type = unassigned DNA
ataatactataaaacaaataaattttaattaagttgtttttactttcatattatactaacaagaacagtttgttaagttttatatgtatttataaaagatatatgagttattggttaatctataatatcaatgtcgaatctctaacaaatattttagtgtctagaccttatgtaaaaatcagaagtgacgctcaatatttggaaacaggaatcattgggatacgtggaaatcaatctttctgacattgttacaaacaaaagaataaacgaaaagtatcaccttatagactcaaagaatggaaggattcagattgagttgcaatggaggacttcatcctgaagtacctatatatttttcttcttggttagtttttcagtcttctcttctatggatcaagtggggaatacagcaagagacaagaaagacattttcctatacaaattcattttattattctttcattgtctctataaataaagaggcatttaaatccttttcctaatttaggtttggtatcaatattttgtttgtaacagagtaatagaaccaaaatatttcattatgttacttgaaatgttgatttttttgtgcccattctcttctgagtcgacaagtgagagtagatatgaaagtagcttacatttatattttaagagtttggaatctcttaccttaaatattttctaaaagaatatcgttgtgggaatatgatttttctattttataaatttgacactatcgatcaatttaaacacgacgtataattttagttttatttttagaaaaataagctttttagtttaagtttttttttacgtaattactattgaatccctaaagttttaaaatgctatagctttactcttatactttgagtttagtttgtatatatggtcgataaattttaagattatgtaccgtaattctaagttaaaacattgctcacctcttgtcctcaaagttaatgtaaatgaaattataatactcatacataatagaacttttttttattcttaattatgcaaaaagaatagtgaaggttaatttagttataatcagttctagaaaattaacacaaacattctaaaagtagtttgaaattgagataaaatgaaagtcaaattcaaaacaacgaaataaagttataaatatgaaactttgaaaaatatagataaaattagaactacgcatgaaaactctaagacaaatagacaattctcgagatagaagtttgaaatcgaaatctggggaaggaaaaatctttacatttccattttattcctatatctactaataagttttgtattaaaaaagaacatcaaatagagtaaataactgcacactaaacaacactcacccaaccaccccatatctcaatgagaaatcttaatgtgaactacaaagctagggacagaaaaatgattcattagattccagaacaataataattatgattacattttggattcattagattccaaaataataataattatgattacatttggtgtttgacctatttatttatttattttatataaatattttgtcgaagagatagaaaaaaggatgcatttaaattaaaaaagaaaaattatattgataaagaagaagatggcgggctgacaagagaagaccgggaggctgatgtggcaatgggaattccaattttccaatcaaaaactaaaaacaaaaaagaaaaaaaaaaagcaaaataattttggttcactgaaaattttcatataaatacatgtggcggttgatggcccaaaggatgagctttgaaggtcgcattatcaaaagtttggggaagcagatttttgctaacttcgaggtactctcctctcctctcctctcctcactttcctttctctctatactaactataatttccattcctcctccatcaataatttcttcatcctcttccttagctaatctctctcttctctaccagtcaaacgccctcccttttggtgctctctagcctcctcctccc

SEQ ID NO: 111 - DNA, 2000 bp, Cucumis melo
  source 1..2000: mol_type = unassigned DNA
ttctctctctcttttaatctacaacgtatccaattatatggagaaagttgaggttgttggatttaattcattttttcacattttttagggttaaaatctaaaaacacatttcgattttgcgactgttcaaggcgtatatgtttctttaaatattatagttgaaattacgatcaaattcttacgctggttaagaaagagaaaatcgtaggagagaaattgtgagcatataagtgaaaataacctcctagagttttttggatttttgagcgaaacaaaagtaaggttgtaacgatttatgtctaaaatgaacaatgtcatatcattgtgggaatatgtgtgaatgataaattatcacaacttccaacaatgagtatcacaatacatatcattgatgggttttgagaaaatgagggttgacttggacatgacaacttgatagacttataactgtgtggtgtacttataagaaatcggaagttggtttaaaaggagaatcatttgcgtcgacaagatgattattattgcaaaagatgcaaccaaggtatgtcgatacataggctagataaaaagaagatcaagtaatgatataactatgtctctgttgatatgtttttaagtgaaataaaaaacaaaactattaatcctatacctaaaatgaaccatatcgtactatattagaaaagaataatgtacctcttgatagaaacttatagtaaaagtgattaataaaatatcactagagagatacgtaaatacttcgttatcataatttattttacttataaatgaactataacaaaaagtatttatatccacaaaatcaacgttaagaatattagggcgtcgaagaagacgccaaactattttaatataggttacgtttggtatctcatctacataacaatcttttgctttcaaatacactcaataaagtaaatggaatgattttttgttttctaattttgtcattaaaaacagtgttttacattgtacttgaatttgtgacatataatgatatatttctttttacaatacccaaaatcaacagtaaaaaaacaaatacttacttcttttcattcaaatttttcatatgcttttgaccgttattagcctttagtagtttatcgtaaatagattgtgatatttttatcaagaagttttattttttaaaataaatttcctttttcataaccacaaaaagcacccttgcaaaatcaatatttcattttggaccgggttgacattaggtgctttaaggatcggcccaatctagattcaataatctcagtaaggcccacttgtaaaccacaaaaaggcatggcccaccgagcccactatgccaatagttgggcctttctttcgcaaatgcacgcagctaattaaggcttcattacttaataatcagtaattaattttgctccaaaacgggtcaaagagcgacccgacccgcgaatatgtatctgggccgttgattattttagtagtaatctcaaccgttcagtcggtccttttatatgtctgtcctccctcgtaatcaattcttagggttttctagggtttttagttcttctctacgcttggttggaagtgcccttcctctcattcttcctgctttactacaggttttcaatcttcaacaatttaaatctcaacatttaatttgttttgcacattgattcaagtgtgttttttttctttcgtttgggcttttgttgattgaaattactcaagaaattgcagccacaaggatagtctaaaaatgcatttgatttagtgtaggtgctgctctcttttttgtgattgttcttagcttacttggagctgtatgttaatgctagagttcattgagtatcaatgctcaattacagatagttttgttttgtaatttccacatatattttcgtatttgtgaaattaagctcgtttgttttcattgtttgttggcaatttatgttttatgttatgccaacgattcatgatttgtagcttgactcgaaagg

SEQ ID NO: 112 - DNA, 1027 bp, Cucumis melo
  source 1..1027: mol_type = unassigned DNA
ggggggggttgtcttcctcaaactctgttatcgaaattaggtttacatttagtatctggttgacgttttgattgtattcctgggcctgaaatatgataaataaatatgaccattgaagttggtgtttattctgggtttctatcattaagagggtgtgaacttacgagggaaccaagaaagggatggaaataggaatatttagaatagtaggaagctcaaatgaattatttcgttcgataaggcggagtgaatataaataatatgcaaggagatgcggagaatattacccaccttatgagagagagccacaaatcagaaaacagtaacaaatataacaaatcaaacaacaccatttgcaagcgaattcaaacgtttttcgggtatgttgttccattaccacattcaaacatgaattgaaacctgagctcttgggcactttaattttattttcaacacattacgtttaaattgccgagtggtcaatatcatgtattgcttagtactaggtggatacaaaccttacatataaggtcaaagtattgtgggcatgatataaatgctctagcatattggtctcatagagttttttatacttttacatatccattaatgagataagttaatgtttcaacattaaatttttagttaatatgaattctagatgcatttgttatacaaatggtctgatgtatttgaggttctgaatgtcaaataggattgtagtttattcacgttgaatattgtaaagagttaggacgtttttttaagattagatgatgggtgccatatgctaccccatacgccaacaattataatgaaaattatattttgtcatttggtatttaacaattttttttaaaaaataagctaataacgcatagaattcctgagatttaaacaactttctgtaatttcttttctatgtactaattgttatagaacctgtgatgtgcttgtccatcatgcagattacaacgactttgaacataacttcaaaattgttgataatggtagccgagtttttgcc

SEQ ID NO: 113 - DNA, 2000 bp, Cucumis melo
  source 1..2000: mol_type = unassigned DNA
agccaaagtttaatatgattcaatcttgcacaaatgcaggttacccaaatggttaaggttaagacaggcaacaccactctagcagttggtgatggtgcaaacgatgttggaatgatccaagaagcggatatcgggatcggtattagtggtgtagaagggatgcaggtaaatttaaaacagatcccagctggagtgataaaatatagctttcattcatctcaattttgttttatacttcttatctttctgaatttcaggcggtcatgtcaagtgatattgcaatcgcacagttccgatacttggagcggctgctccttgtgcatggacattggtgttacagaaggatctcttccatggtacattatgatctataaatattactttatattagcttcttagtgagaatcatccagattaatgtgcaactatacagtctcaacatcattttcagcttgaaaatctttgaaatatgtcgaactcatcgcattttatatatggcagatatgctatttcttctacaagaacattgtttttgggttcactctattcttctttgagatgtatgcatcattctccggccaaactgtatacaacgactggttcctttctttgtataacgtcttttttacttctctccctgtgattgctttgggagtgtttgaccaagacgtctcatcccggtactgtcttaaggtaagttcaactttcctttatttcattggtgcaatcttttgccttccttaagtacaatatcaaatggctcattgccctcaacatttttggattttcagttctcacttttataccaagaaggtgtccaaaatgtgttatttagttgggttcgaattttcggatgggtgttcaatgggctactcagttctgtcatcatattcttcttttgtgttggggcaatggaccatcaagctttccgcaacagcggagaggtcgtcgggctggaaattcttggtgccaccatgtacacttgtgttgtttgggttgtaaactgccaaatggcattgtccatcagttacttcacctatattcaacatctcttcatctggggcagcatcattctttggtatttattcctcatggcatatggagctataaacccagccatatccaccacagcatttcaggtattcattgaggcctgcgccccggcaccatcattttggatcctcacactattggctcttggagcttcccttcttccatacttcgtcttttcatcgatccaaatgcgattcttcccaatgtatcatcaaatgattcaatggataaaagctgacggacaatcgaacgatccagaatactgtcaggtagtgagacagaggtcattacgtcacacaaccgtcggttacacagctcggttcgaagcatcaaagcattttgaagaattctcagaaatcaagagtcactaggtttgatgattagatcgtagaaagattcaaaacattttttctacgtaaagtttcttctcagtgtatatatatatatacatttatatatttatacaacatgtgtacataagattcttgtgtagttttgatctccttgtagcttaagtgaccattcccaattcaaatggtcaaaaattttcttcctttcatgaaatttttaggaaataagccaattgtagtattcaatcgtatatttcaaatagtcattgagaagttctaactctactatagttctcaattataagtatgatggttttgtcttattcatcttgtagtagaaacaaaagaataaatttacgaaggatgaagcattgttattttaattataatttgggaaatttggagccacgaaatgaaatccaattttgtgccaagtagatgtgcaacaatgggcaaagaatcactttttctttttcataattttcccttccaacactcaccactaattcatcacctcaatcctcttcttcttcttcttcttcttcttcttcttcttcactcgcctttgggttgaggttggctctgttacagtcatcc

SEQ ID NO: 114 - DNA, 2000 bp, Cucumis melo
  source 1..2000: mol_type = unassigned DNA
aaaagagtcatagtgaaaaaagctgagatcataatagtttcaccctaaacacaacttataataacacatactatcataatatacacaccaaacacagactctatagtcttcactctaaacgcagattacaatagtctgcaccccaaacgtagactattataatcttctgactattataatactcttttcacttatcgccccaaacgtcccctaagagaacaaagataggttataaaagagagatgagggtttatattatgcaacaagtataaggttctagaacgatgtagtcttcaaagtaacgaaagcaataggctacacgagaaaaatatttttaaaatatagtgctttccctaaactagatttcaatgacaaattattataaaaaatagaatcattaatccaacatggcttgcatgtcacaccttggccaaaactgaagacggatgtcatactcgacttcaatatatttttttaattaattttcatgtgacaacacataaatatttaaaatttagattgggttggattttttttcaagtgggtcccaaaatactcttcaaacccaaaccaacccaacttgtttacccatctaataataacccaccaggttcaagaagacgaaccgaaccgaaccgatccggtctaactttgtttcatacttaagtcgaacttagcggtacttttggttcggttctcggtttccccaaacagagccactcaaaattagatttagggttccgttcgcaattttcagcgcatttttatttgaatcggtcgtttgttgaacacgttctctctcagctggtttagggttcatcgttctctcttctcgcgctatatctttctctctctcaggttcgtttctttctcttaggccattttatcagaaagatcctcttcgtttctccgattttctttccgtgttcgccctcggtttctcagcagacgtaggaagtttggtttccgtttagtgaatctgtttggggtattacgaatgatattttgtactgggctttccgcatagtctttttctttctaggaatatatgcatctgagaatttatttgtttggcttttctttataaagtatgaggacatatacatctcgattgctaatccttgattataatctttttttttctatgttgtttgaatctgtttttttttttttaatttctaggttttttgaatctaaaaatgtatttcttggatgaattgcatactgttgaattagaagtttattgattagattgttgatatttgccctaagttccatggataggtttgcgtctttcaccttttcgtttgctttttcttttggctgacgacatcttacatagcctctgctctaaaaggtgccatgattttttttcctggctttatctgagtttgcgcaatttagatttgaagtgatgatttgtctaaatataaatatctatcggccatactattttttgttattttgagtttttcaaggatgactgctagagaatgaaaaatcttgaaaacattgtgttttgaagttcaaggatcttgtagttttgttcttttctagactatctcatttgatatagccctttaaatttaatcaaaatttgttaatattcaaatcctcggacattttaattatttatctaaatagttgtttaggcattactcaggttgcccactattttaagcttagaagcctactctggttgacctaaagtttgcatgctatttgccttatttcgcacgactctaaactgttatagacatcttttttcagccttcaggtaaatgaacacaaaaaggagtgaaagtctgacttctgtgtgatggtcttttaatcaattatagggattaagatggtttttttattcattgtataaatattaaattagaatgatgacaaccaataatattaaaactgacaatggaaggttccttatattatttggagtgtacattacaacagcctgattcttggcttggcaggttcctgatcaccttgtaaac

SEQ ID NO: 115 - DNA, 2000 bp, Cucumis melo
  source 1..2000: mol_type = unassigned DNA
aatgtaaatagtttataaacttaagataaaattggtaattgtttaatacaaatacaaattgttaaatgaaatgacacaccttgagcaattttcttttctaatcttctcttatagattaattttatttaatcatgaaggttagaatttctttagcattatttatttatttatttattagaaaaagatagtttgtgtatattttatatcataaagtttcagaagaaaccataaaattaatggagaataataaaaggtggggatctctaacatttttgccataaacaaatcactaagttaagaatatgacactaaacttcttctaatttaatattatatacaaagattttaaaattataaagtaagagccttgaattgtagctaatttaagaatatgctctaagttttttaaaatcacttttgccctacggttattatttatttttttgttgaaatatgtttaatccaaatcaatttcaatcgaacatagtcaaggatatgactgcggattcgtatattagttgattttgaaacgattaaatgtttgaaatattgtagtttaggaacaattacaattataacaatcagattcaaaattttagtatatacagtaacatttaaaagaataataaatatatcaaaatctatcgacaatagacttctcttcatagataaattatcagggtctgacttctctcatagataaattatcagggtctattagcaatagactaaatccttgatggtttatcattggtagaccaaaagagtttattagtgtgatagactttactacataatttgcaatttgtttaaaatgttgttatacatttggttgctatccttaacattacaatccataacatttgtcgtgtctttaacttgaattgattgttatctgtgataaaaagagatgatcactttttgtcatgagatttgaacaattgatgttaaaagtggtaattaatgtaccattcactaaccaatgtcaatatttattttgtttaataaaaagaaaaaggagattgtgacattagttttatactcttttctaaacataggtttggtttgtgttagatttggcctacacttagctcaaatccactctttataaaattcccttacttattacaagttatattttcactccaatcataatcttttaaaggataatatttgtattagaagatacgacacatgtagaagataattcttttttaaccaaaacaacatacaatttcgaggatatgacaaattaccttttctatttttaactatttgatcttcaagtcccatctaaacatcaaatgaaagttgattaggttaaagaattggacaattagagaaggaatggagaatcaaacctctaacttttaaggaattaggtcattcacattttcattgagctaagctcacattaacaagatcaatattacttgtatgtagttaattcagatgtgaatccttgaggtttcaaaagtgacactttagttcgaggtttaaaaaatatttatatatatacacatgttacaacccaaatttaaggtatatatataaatatatataatttaattatcttgaattataattaccttaaattacttaaagtaaagattggtttatttatgattaagttatgatgaatgttaagtaatttgaaaatttgaagtttagaggattgttaattcacttcattgtgggcctcattaattggcccattaaatctccatatgggcctgtctagggcttcatttccccaagcttccaactgtaatggcggccacagttctctcctccatctcctctcttcttacctacttattatgttaatatctacgttttccagattcattttctttttatttgtattattctaaatctccagaactgcttagctgctctggtttttggggattttagggggctcgatctggtgggtttacggttaaattttgcagcttttcgaggtccttttcggcttccattttgtcggaagttacaaa

SEQ ID NO: 116 - DNA, 2000 bp, Cucumis melo
  source 1..2000: mol_type = unassigned DNA
acacttgtaatgttgagcagggtaacttatggtaaatttgacatgagctggcgcacaaaggcctagcatgctcggagctgtttttccatggagtcaatgcttgatcgcattattggctatattctaaatgaaactaaaattattgatgggttccatctgtttggataccaactttatacacaggtgtttttctatttatgagtgtaaagtttgatttgcttcatcatcgtatattcaacgtagagtttcttagttaatccaatccatatgcctcaactatcatgctcttttccctgtaattgaatgttttttttggtgtccacatggtcatggaggttttgttctgcactagcttcacgatgctactaaacatgatgatgaagcttgagtttatttatttcttagtactttgtgatgaaaaaaaagtagaagaaaacggtagaaaattggaatggatacggtacaatggatgggttgtgctaagtcacgtctcgtggatacaactacaattagttattttgttttgtagatttcatattagcatttccttctgaatagttgaaatcaccatagaatgtgtactgatgttttgtgattttagtgcttcggtataatttgaacgctttacaagtaaaaatttcctcaggtaaacgagtcttccgaagtacttgttcataaaatgttcttgtgtgggagagttgattggagaggatcatggtcaaattcttcttggtgtgttttatataaggttttaatgattctttgaaattgtaatgtttccttagtttttttaagtgatactggtgggttttccttggaataaatattaagggctgaaacttaggaattatatggatttgagggaggtttgtggattctcaaatcaaatcaaaccaaaaccagataattttaaattctagaattttgaagttactatttgtgtttagaaataaaaagaaagaatatcgcttctttgtccttccaatattctttagaaccaaaagagaaccaaaattatatataaaagagtcgataaaatcaaatatatatctataatatagtttattattatttttcatttgctatcaataagaattttgaaatgtaatatttgctccaaattatattaaaaacagctgttgaaatttcaacaaaatgagaatttgtactctggattttgttattagtttttttttcaatatcttaaactatttcttaaatattctcattgcgagtccttccatttacatagaactaaaaatggattgagtttggttagagaataatcccaatcttactcatatttttaggttgattagattggtaatttgattagcggttaagttattgggttgtattgtttcataaattcgatagattacatcgatggcaatgtagtgtggaacataaaaaataatgaaataccagcggaacacaatggagactgaaaaggatagacgatcgaagatgatgaaatgagaagctgacaacaatgaggggcgtgagttgagaagccgagacaagagggagagagtgagtcggaaagagatgtggggcgttacaagttgtgttgaacaaagtgaggtcaaatttaaatttactatttgctaaattaataataaaataaaatataaatataaacatataaatatatatatgattgggttgggttgatacaaaatttctaaccctaactcgataaagcaaaatgcaacccaaattttaaattaaccagatcgggttattcttatcctaaccttaggacagtgattacttaatctgtacgcaggggcaattttgacctttgataaactctcccattttgttttcttttttcggcaattttccctccctctctagtctcttctgttctcagttcagctctctagggttttgtcgaacagccatttctaagtgtacatctcctctcaatttccctcgctttattccattttttcacgtactatcggcggatcctttgagctccaactctctcatcc

SEQ ID NO: 117 - DNA, 1025 bp, Cucumis melo
  source 1..1025: mol_type = unassigned DNA
tttttcttacttcattatcgaacaataatttgatttccaagcgaccctttcaaattcaaacaaacccatttctcctctcagccagagcaagtgattgaactgctgcgctgcgcgtgacctgttatcttctccgtttttcttaccgccgcccgcccctcacggcggagtagtttcaccgccgacccatttcgccggccgccggcggtgtttcgattcctcttgttttgtcccttttcgtcttaccagttttctctctgtctacattgtgtgctcgttaacagccagctgtatagccactgctttttttattgactcttgaaacagagagataggggagattctgtatagtcccactgtttctgctcaactttttcggtttaatgtctgtttctatattcgattcttcgttttatgttcgtgattcgatattgcttttgcttggaatcgtttagaggcaagtgattgtctctgcttttgctatgtagttactctgttttttttccctttctctctctctctctctccccccctctttctcaaaagggggttggtttttttatcgtcggaggatgttgggttgatcttttgatagggtctgttgactaatttagctggtgttcttggtctgctgaatccgaacttctcttagttttagagttttcgatgttgttggtttacactgattcttcttcgtttgtttgggattatttttgacaggactatagtgtttaactgctagctgccatggaacatgcagaatctgcggtgagtttttagaataaacttgttcggttggtgagaaaagcatgggaaagaggagggggaggtttttctttatgtcaaatattttctcaaactcaggttttagaataaaaaagcctttgtttcttaaccaaatagtttatttgataatcagctgttttgttttagctccctcatctcattttcggaaatcttagttatcagtttaatcaactctgtgttctatgatgctcatttgtacttaggcaaaggttataaagaac

SEQ ID NO: 118 - DNA, 2000 bp, Cucumis melo
  source 1..2000: mol_type = unassigned DNA
tgcattttatcagagatgaaattgaaaaaggaagaataaacacgtactgtaaaatcaaaacataagaaacccagctgacttagcttgttaattaaccaaacaaagtttgagcattgtctaaattaaagttgttcaacttgactgttgtagggttattaatttttcttgaaaagaaaacgcagcatataatattaaaggagtattttgtctcgagggggaagattattggttaaaagtatatatggtgtgacataattaaatactttgtaactaaaaaataaaacataatgggaagttatctctaccaatttttttgttaaagggctgaatatataacctccaacattacttagttactgatatatcagtttctctagccgtcaacagtactacatagttgctgatcataaatagaagaaacaagttagaaattttgtgaagagaaaggcgagattatgtgatttttgctttgtataattttgaaaacccttgatataaggaagttccttgttgctgcatgccttcttagagatcagcagttactgtatgtctatatataattctctctctcaatatttttttctgttcttgagcttgattgtttactgcttcagaaatcttctttacaactactactgtatttggaagttttagttccatatatatttctatttttttaatgatttcaaatcttgttgtttcaaacagtactctcctaattacaaatacaataaaattatatctagcattacaattttacaaagtccttttcttgtgaaaaataaattacgtgagactttgtaaatggtattttgaatgtattaaggtactatatgacacttagaattgctttgctttagctctaaccatgggttcaaatgtaaagttaaaaataaaacaatcaactatttaaggttttacttaaaaatgtaattatttgtcaaaataagcataataattgagtagtaatttacatatattgcctccacatttgagatcaaaactagagatgttcattttcttagatatattattaagctaagaatgagagaatgggtgaggggaaaagtgaacggaggcaggaagaccaaatcacccattcctgaaaatggaaggattaaaattgcaattttccttgcaatttaataccaacatgattttgtatatatatatttgaagaggggttttaaaaaaatataacaaactgttaaaatatttacactatatacaacaatcgttaagataaaaaaactcataggtccacaatgaaaaatataacaaatgtcatagtcaacacgcgattaatcagccacactcacgttcgagtaatcttcttctgaatgattgtgtattacagtcaaaatacacaatcgtagagttcttttctaatgatgttgaaaaatacttcaaatttagggtttagggtttagggtttaatgatcgtgttaaccgtgaaaaataatcgtgttaatcaatggaaaacgatcgtgttgattatgataagtgatcgtgtagtccaatgtaaacgatcgtgtttgactatgttaaatgatcactatggtaagtgatcgtttaaatcatataaacacgacgatcatgtagttctttttaaaagatggaaaaagaattcaaatgcaaacgttcgtgttaacaatgacaaatcattgtttagatcatgtcaaaattaatatttaaacgatctattgatattcttaaataggaggaagatgaagtagttctaaagaatactgtcgaaaacaataaagatagaatatgatatttaaattaaaaaataaatgatatcggaagagaagatgaataaatcagagaaacagatataaaaggggaagtgactgatcctccaaatctaaaagataaaaatattttacatgactctgtaaactttggtttcttttgctaggcagtaaatatttgagggttttggtattgtatttgtggcggaatggagtaagtgggcctggcattgggccgtatacgta

SEQ ID NO: 119 - DNA, 2000 bp, Cucumis melo
  source 1..2000: mol_type = unassigned DNA
tcattccagaaaaggtaatctttgattttgagaagttaatttgaattttattttaagggaattcaggcagcaagattaatcatctggcttcctggaaaaaggtcaagttttctcaatcagaaggggggctaggtttgggcagtttaaaaaataaaaaaaataaggccctctttctttactaaataaatggtgttggaggtttttgaaagaagactccacattgtggggtaagttatcaaaagtatccatggcttcaaaaaaatttaattggcagactctaaacaaactagaaaatagccttagaagttcgtggatcatgggaaagttgagttggcaactttcaaaacagagaacgaaaggagagtaactttctggacagattcgtggattagtgatctccctcttaaatatccatttccaaatatattcagattagctcaacaacccaatgattcaattactgcgcactgggattatgtcactaattcttggtcattagtattttgaagattgctaaaagatgaagaaattcaagatttccaaaggcttttaacactcaaatcctagaaagtaatagacttggatgatagaagagtttggtcattaaaaacctcaggccatttttcagttaagtccctttcgaagcacctctctccttcttcacctttggaaaaagattactttaaagcaccttggaaaaccaggagtccaagaagaataaatgttctggtttggattatagcagtgggttctctaaactgttatgagactatataaaggaagcttcctaatatgtgtttactacctttagtgtgctccatttgcttgaaaaacagtgagctcctaatacacttattcattttttgtcccttctcatctacttgttggtttagcatattttctatgctcaaacaacttgggtctttgatggttcattaaacaccaacgttgttcctaattttttagggggtccttatttatatatatatatataaaaaaaacttttctaatttgggttaatttgataaaagcactcctagctgagatttggtttgaatgtaaccaatgcatcttccatgataaaagagagagagagagattgggttgacattgtagacaattctaaaagaaacgtggtagcttggtgttcttcaaatgcagaattcaaatgcaggatatctacttattggactaccttcatatgaagagattcaatgcagtttcccccgactactagtttagaatttgtgtttttgtagttttaatgggctgtaatatgtatttctacctttaagtttttacttttcagtcttgcttctgtctaccataggtagtattgttattttgggtatttacttttgtcttttcatgaccttagtcttgttcttgtattttggatataatgagggtgctatcggggtatcaacctagttgagatgttcgagtgcacctactgatccccttatttgtaggcttctctattattctcaatgtataactctcttgtactttgagtttatcaataataaagaagcttgtctcattctaaaaaaacaaaaaggaaaaggaagataattgctcctaatcgttgaaattactactaattactcttaattactccaaatgatcgtataacatacatttataatttttaactttcttttcctttttaaataccaacattaaattttaaatacatccattaatttgaaattagttttcaaattccaaatcgaaagatttaaagtcctttgaatccaaagggagaatgagcccatccaagcaagtttttgtgtcgtagttgcatattttaagtcgtttcatattagcctcgagtttggcttaatgacttggtggtgtctagtgcaggcttgtggcgactggcgagcgtggttctaaagataaggtttgcattcgctccttctccctccctttcactacttcatatccatttcctttctcgatttctcgtcttcccttctgaattccccattccagcc

SEQ ID NO: 120 - DNA, 2000 bp, Cucumis melo
  source 1..2000: mol_type = unassigned DNA
atttatttgtttagagataagacgcacatgagaatatgagatggattccactccactcacactccaatttctacctatcctattactgtttactattatcattccacccctcgacccctcattcttcttctcaccttacttttttatgatttactactacttcattttggatcacaatctgatcaatgctgggtgggctgccctcggcctgtcaccaggcccagcccacttccaaattaaacctcttggcccaccgcccattgtccccatcccattccatttaatattcccaaccttccctttttctttcccaatgcgatgcttctccaatatacctttcctgccctccatgtttcctttttactgctttcttatatttataacacaccttctacagtcttttggctgggaatgctgcgtatgtgaatgagattcaagatttcgttgatgttatttgagtctctatattcataagttttgttcttagttttctctagaccaactgcaagagttagcgttccatatgctcataagtttcagatttctgctgtgtggtttgaagacagtcatcgatccatgggtgaattcggctttttattattattattattattatttattgttgtcttacttttctatttgaatcttcctatctttttactcattgttggactctaataattcttgctaaacacaatctccatttttattggacattttaaatcccatctcaactcataattttagttaccttccaccatcaccatatccaaatccgaaataaactcaaataaaatccttcacgtgcatgtgctctccatatattttttctacatggtaaaaataaaatgaaaacaatctaaatttaataaaataacatatatggcagacttttattgatgtagagactgggtgttgtacaagaacagtgcagccaagaaaaaaaaaatacttccaatgaatcgtacattttaaggattatgaaactaactagttccaaccattttttcacgaccacgtgcttgttaaacacgcaagtagaatcaaaatgtgggcttcttcgctttatataactgtgaatcattctccaaaaagggaaggggatctcattccctaattcaataaagaaaaagaaaaatgctagcgaacttcatccatctcattccttttacctatttcatgagatgcccattgtatataagtattttttttttttttatttcattttacttagtttactcctcacctctaaaaaaaattaggagagtttgctaaatccattctcaaacttagctttatttttttaattttatttaacctcgtcgtggatgttaacctcaaatgtcagttctttttattctatttattgatgttataatttactttaggattccaattttataaaaataagaatacaaataaagataaagagtgtgaaagccagaaagaaaaaaaggaaatcgtaatatgggtaaaattggtacaaattgggtcccgttaaatattaactcaaaaaatgcgagaaaatggtagaaaaggaaatagggggtaagagcaaagtagtggaaggagagcattgaacatattctctagtttttgcacttggatctaaacacgaggaattataggtttattcatttactaattacataaataggattggattttaaaatttgaccgagtgattatgcatatttgatagagttagaaaatagtggtggggcaggtacaagttacaagtaatgtataagagatatgatgagcatattaggaaactatagatttaaattcgtccgtaaataaataattagaaatataatattcgagtggaagggtattagggttaggcgaaaccaattgcagttgcacctataaaaccccttttacgcctccacccgcttcaacagcggtctcggcgtctacaactacacactacacactacacactacacactacacagttgcagaccagaagcataacgtaacgccggtccacaaaa

SEQ ID NO: 121 - DNA, 2000 bp, Cucumis melo
  source 1..2000: mol_type = unassigned DNA
tgaagagccggaaaagcatggcaaggtcaaggcatttcagcaagaatacgaggatgaattctgggtttgtaagtccggttcttcttcggagttgtggttcgaatttcaaacggccatgaggagccctacagcttaggaagcttcaggtctgagactctgactccgacagaaattgctgatttttgtgtgtgttttgaataaccattgtatgagtatgattggttgtataggtttgaaatgagggaaagtgatccatctccttgttgtctttgcaggaaaggctcatattcgaccaagaacgctttgtcattgctttcgataatcatagaatccacaatggtttggcatattagcaaacaaatcttctttaggcattgcagacagaaaattggagagtaagttattataattaaggattctaaatgagtaagaataataagatcaggcaaagattggaagaactaaagcgtttagtattttgtgtggtacagaagaattgtaattagatacgtagtctagagaagcagatggtgagatttgtgaaagacaaatgttagtggagagtgaagagtgtttctcaacaaccgacatagaaggattctcagaaaatgagaaagatttttattgcgcaaaatagctcccatatcatcatatgccgttgccattccctctggggttgcatgtaatgagtaatggaaagctgtagacaggctaacttcaccctttgtcttgggtatagggtgcatttttggtcactccattttaagttttctaataataaaaggatgaagaaaagatattgaaaaacagctcaggttttaaagttgtcacacttgagaataatgcatttaacagttagaattttgcatcaacgtctttcaaatagaaaagtaaaggagagtctagtttgagctggatagctaaactggtttaatcatatcttctatcaagtggttagagttttagacctcccaactttatatgtcgttgccctaacaatgttgatggatgtttagtcctaagctctaacattgtccccgtatcatatttcataatagaatgtactgagtaatggaaaactagagaggtaggaagttggacgaactttgaatctatattgattttactatagtctttcttccaagtcttaatgagagctttagctaaagtttttcaaaaactcaaaagaagtttttgttttccaattcttcgatccatagattgtcaagatggctataatatccttgaagttaatagccttcaaagtttcatagcttttatcctatgatctttagaaattcaagagttatattctttagaactacagaactttgatcttcgattcttcacctcttccagaacctttatagtagagtttcttgaccttacatgggcttgggattgggcctggctacttatgggcttagagattgaccttgggtttaagcacattcgttttacttggcccaattttcttaaacttctgcaaaatcctaattaactaacacctcaacaaaagtccagtattaaatggggcatataaacaaaagttaaacaaaattgtcgtacagaatcctaatttgctaacctcagcaaattttatgccattcgtccttgtgtatctaattagtgttaatttgaggaaatataataatatagacgtaggacggatattggtatttggtgcaacatcctccgaaccaattcctggaaagtaattggggcaacaagataatgttatcgtcagtttcaagtgcaaaacttgccacgtggaacagcggcaacatgatatctcaaatatggacctctcacccggtcaagcttccaccaccaccaccacctccgtattctaaaataaagtcaggaacaagaacagacacaccttaacaaaaccaatattcttcatctctatctctctctcatttcagcatagaagagagctgagtaaaaggaaaaaacttcaatcaaatttgacagagaagagcccaagagaaaaccaa

SEQ ID NO: 122 - DNA, 2000 bp, Cucumis melo
  source 1..2000: mol_type = unassigned DNA
agatgaaccagaaagatggaaaatctacttggggttcagcagtgagtttggaaaagagagaactatttgaagaaagagcagaaaccatcttgctaatacttaaacaccgcttccctggaattccacaatcttcactagacatcagcaaaattcagttcaaccgggtaaaagaacgctccttccttgtctataatctcatctaaaattatcaacaatccaaacacaatttatacaaactaaaatgaaagcttctcaactttaggctacaaaaacagatgcttattataattctgcccaacaatatcttctcctaaataagatgatatatgttttttgcccatataatcaaataggaaataacaatcctgtgcccatttctttggagtgtgagatcataaaacactgtctaaaacaacatgtccaaacatatcgtaaatacctagtttcatagtgtgatgaaccaccacaaacaaacttactctttggtgaactgcaggacgtggggcacgccgttttagagagctactccagaatactggaaagcttagccttcacagtgatgtcacgaattgaagacgtactccacgccgataggttaactcagaacccatcacaaatagcaacaaggaggaaaccgacgagcgaacccccaatggagaaatcagaagagttgaacaacaacggcccagaaacgccagcttcaatgacgctgttggatttcatggggtggggacaggatcaaaacgagttggagatgaagaaggaatggtttgggaattcagatgatttaaacgcggattcagatctgaaacaagggaataagccagggaatatagtgacgaacaagagggtttcatacctggagaatttgagcgctgtgagaagtccaacggcgcgccattgaagaagaagaatagatagagagatgatttggaggcaaaattccatgatttcagttatatacattccttttgtgtaaataggaagaagaagaaggagaatgagatcaaccccatttttttctctcttctttttttaatttggattttggaatcacaactctttgtgtttgtgtaaaaccaaaattgttctatgtatcatttgtatcaattaatgtagtcattttagattcatacattcaaaaatatcaactccattttccaactactatcttcctccatctcacctctaatcataattcaaagcggatacaaattcatgttagaatgaaagattcgagtatagcctattccattgatcaaatgcatgtatctatactattgacacttttcaactcaagtcatgcttgaacaattgttttttataaatgttaattacaagagtgtacacaaatcgagttgggaaaaaatatgaaccaacccaaaccaaaaactttgagttggaccgaatttgaataaataattcaattttcattatttttatatagtttttcaaccaaattttttatcttttttttctcaaatttcaagtttacaataatgtccattcaaaagtttaaattttcatatttcgaacattgaagttggaaggtccaaacgaaagaactaaatgattatgacacatgtctagggtttatatatattgttgagttgagtaatccaagtttttgaaacaccactagttatttatgttaaataattaactgagtactcgactttcttagttcgagaatattttttagaaaaaagaagagaggatgtgtttagaaataatagcaagttagggtgttggtgagtgagaaatattttgggatcttcttgtaatagttattaagaagatttttcaaagaatttgagagttaaagaaataataataataaataagtaaacatttaatagtaaacgacatgtcgttttatacagcatcgtatttacttaccatgtgctcattcacacacgattcctcctcctcctcctcccgttcatctcttcttatcttcgtcttcttcatttcggataacacaaaaatccctaaaaaaa

SEQ ID NO: 123 - DNA, 2000 bp, Cucumis melo
  source 1..2000: mol_type = unassigned DNA
tcattttcgtttggttaaagaatatatcatcgtttcttttataaaatgtttttgtagaattaatcttcgagtacttctcataaaaacattttttttaattacatagagtcagtaataattagaactatctcaaaccaaagtactataacatttcaaaccataacactgtattttttagaaaagttattgtaaaggatagaattacaaaaatattatagcatatgaaacattattgttatattttataaatattccatatacaatataattgattctagatgtctctaaaaatagggaagcatatgtgttaataactaaatttaaaaattaaaaaattactcgattactgtttatttatttatgtgttgaggctatacatactatattttagtaattattttaaaattaaaaacaaaatcacatggctaatagaaacgatagatatctagtagtaaaattttgttatatttataataagttgtcttattttacttaatgtaactacaaatatctcactgttattttcctttttttttcagcttattggtttatatgtttagaaaatttggtaaaatatttgtgtagctgcggttatcatgtatcaacttaactatgtaatctatgaaaaaatagtcattctttaaaaaaaaaatgaaaagttaaaaaagaaaaaaaggataaatttataacaatattctttaattgaattttatcatttgattcaaagatattcttatacttttaaaagctgcaatgttatttatgaaattgttttaaaattacatttataatgaaaaaatctttaaaaatgtagaaaaatcaaggcttagaattgtattgtcatttccatcaaggagaggatgtaattttttctttatcactttatttgaatcctcaaattttcgataagtatatattttgacatttgagaatatttttgtttactttaaatttaaagttatttttaaaacaaatgaaacaaaatattcataacgtggatcaaatcaccataatttagaaagcgttcttttgaaacatgaccccaaaactttagaagataaattacaatttgaactattttgaaaatggtagcaaggagacaggtaaaaaaagaccacataaatcactttaggctttaaagaaacaatgttaattggagaaagattcattggcatataattttgaaatatgattgtattttatatttcaaatcatattccatgaatttatctatctttgcttgtagtctaaatcatgcaaactttgaaaataacaatgttattgtatcaaaatttaaaagtttaaaacatataattgattaaataaagaaaaatatttagaaatgttgtatgccaataggtattatgtaataaattataaatgatataatattaaaaacaataattcataccattttttaaacataaaaacatgcttagtagattagttataaacagtttcaagtaatatttaaaagagagtcataagtagttttataatttataaaatacaatatcaaacgtacttaaaactaattgcttttaacttcaaatctaaattaagaataagaaaagggagagtgggaaagagcaaaatgagagaaaatgtcgaaaatacgcccaacggttcggaccggtccatttttgtcccgcgcaatggtaaaaatagattaggttacgacaatcaccatgatgatgaggatgatgatgatcatagcaattcaagaagcatagggccccacttttggccctcttattttctcttctcttacttactttaaagaatctaactgtcctccattaccccgccgatcaatgctctatttttctctctcttttttcttttctttattaaacaataataacaaaaaccatcaaattttcaaaattttgaattatattcccttaacacaaaacactctcctcttttcctttctcttataaatacaagtggagctccacacacttgtcattttgtacccttcttccccaacctcccaa

SEQ ID NO: 124 - DNA, 2000 bp, Cucumis melo
  source 1..2000: mol_type = unassigned DNA
gggcttccattggcctccttcccgtcgccgtagtgagagaaaaaagaaagaaaggggagaggcagaagaatttgagagatggatcgaggagaggttttggaatgaatgggaaatttgaaggaagaggtttaaacataaaagtgaggcacgtgcgagaatgcaaatatttacggggctaaaaatgggagagccaacggattcaccccagtaaaaaggtaaattcaaacacgtttatgcctttttacctttttctttctttttttaacacctatagatgtaagatatttcatattcttaactttctctttctcttttcttttttgttttactatttccctttcgttggctaataataaaaattgatggatacagtatatttggtatgtcatcataaatttagagaaggtattaagattttgtgacataaaaacccaatttcttttaatgagattcttagaaattttattgaagagaattataaactttacgtaaattaggtaaagtctttccctccttctcgatagaagttgataataaacatagcatacctagataaaagtttgggaacatttttgttgtttggagggttgaaaaaaattaagaaatttcaatttggttaggatttgatgtcttgattttttgaaatataaactttcaattccaaatggtcggacttggaacctaacaaatcgtgttttcaattttaccttgatattttagatgtgtgagactccattaagtattctcttcgctctcttcttactatttctctgttttgctatcgaacgatattttttttaaaagatttattttttaattggtggaatgtttgtatgagagtatataagttaaggtaaacaaataataattggttatttagcaatcttcctagtcaataagcaaaacagacctaacatgcatcaaagaaacaaaatcaaaaccttaaaatatcatggttgggcgttgattttttttttcttttaatgtttgaaaatgtgggctttgggtgccgcagtcgtatggttgtagggatttcttttaagaaaattattttatattgtattcgttttgatctgaagatatcaattatacaataattggaatataaggagtaatttaactttgttcgtgattgttttctactttattcgatgtgtattttggaattaaatatgatttcaaatgattttgtttatttctttttattgattttgttttgattttactttgtatcaattttgaatatcaatgtagtgatgtgcttgtattaaatgtattggttgataaatttactatgcaaattttttttcaaaatttatgcaattcattgtagtattattaactatatcaacacatcagtaaagtgaatcattatcaagtatatcaattaagttacaaagtgtatatatcaataatgtatcaagtttatcagtagcactttaagcatataaagtgtatttaatcaattaactgtaccagtgaatcttactagatgtatttgcagtacatccgacgtatcaaacatatcatgtgtatcatatgtttaaatttgttgagtatattagtgaaacataacaagtttattagtagtgcatcaagtatatcaaatttatcagttaaacatttaagtctactaagaaaaaatgagtgcaataaaaattatttttcggatatataaaaaaatattgagtgtatcgaagagttccatggtgcatcaaatatataaagataaaaaaatatcaagaaatattaaatgtatatccatatatcaagaaacaaacctaacatgtatttcgtgatccaacaaccggactggaagacaaatttcggcccgggactttcatagtccaaataaaggcccattaaacttaacctgggcccaaattaatttgtaaattttaagtataaaaagaagagaaaccctagggtttccttcattcaccaggccttcctatccccttcccttccccccctccccattcccatttttgccggccgcc

SEQ ID NO: 125 - DNA, 2000 bp, Cucumis melo
  source 1..2000: mol_type = unassigned DNA
ggacttatggggaatgggttcaagtgatggtaactagctacttcagatttaatatcctaaattgccttggcaacccaattcaaatgtattaggattagataggtgttttgtgaggatagttaataaggtgcttgcaagttggtgtcgacattcccaaatgtgaagggaaaaaaaccccaatctttggtctcaactggactttggttcattgcagttgaaaataaattattttagttcaaaccaataaaacacattttttaaaatctttggatatttgtttcttaaagttcctgaaacagcccaccaagtccatagcaattaggaaggcataagttagagctagtatgcttggcatggttgggggtgggttaccttgttatgtaaattcatagaaatattcatatcttgtgctaaaagtcaaatggaaagagggtgattgctgtgatgctgtctaatacaaagtgctagaagccatatggagaaagggtatttctacagtgtctaataagttaattacataataaatttctaggttatgagaatccaatccgcatgaatttaaggactgcacacttgctccatttgcaacatgtgtaccactttagaatcatatttcacctgagttcattattcaactagattaatgtatctcttttggtgttacatgtttttaagaacataattattttagtttactgtcggagagaagcaagtactggttatgcatggttctagtgagcctaatagagtaaggctatggtttgggcatttggaagttttagtggattagaattttgaaggcaaagctaaggatcatacacgcccttcttcccttttgaccagttggagatctatcatgtaactctattgtcttgggcttcggccttattttataaatttcatatatcaatgaaatttatttcctataaaaaaaagaaaaaagaaaaaaagctaaggattttaatatcattgttagtttctttaatttttttctttgggaagtgtgcatgtagagctcctttgaaagagaaaaagcaaagaactcttgaatgtaaaatctctatgtttgagttttatagtagcgtaccacattcacttcatggtgatgtagttatagttttcctatggaatatggctattaatttttgcgaggctcttattttatagttcttttggggtgttctttcctgtaccccctcccctttttgtgagaaggggaggtttctgtggctagctgggttggtttagatttgtggaccttttttgtgagaggaaccatagaaccttttgatgaggacctcgagcactatttgatcatttataagtttccataggcttttgtaattacctttttggtcttattttaattggagtccccttcctccccttttgttggcttttttgttgtatggttgggcattctttcgttagggaagtttgataattcacataataaacatacaataaacaaccatcaatacaatcaacaagcaggattagtgtaatactgtaaatgtcttttattttctttactccttttttcttttgaggtctatgataattgatatccaacagtgtattggccaaaatgatttatcatggtcagtaccttaggggtttgacttccaatccaggatttaaggtttgagaccagatattctgtgcctcaaggccctcaacaaccttctcatggctttttcctgtatacatattattatataaagttataaccaataaaagggacaggtcaaatcctcttaatatatgcgaaaatcaacctaatgtctactgtataccttctcaatcgccaccttcctcctgctgtcatccaaggtagggccttattgtatcagctagctccctttacttatttatttattttttgaagtgcgcagtttgtttgtttaccttgttataggaaattcaatctattctcattttattggtgcattcgtctcagaaattcttgtacggtttcaggttatcatctacccttgtag

SEQ ID NO: 126 - DNA, 1397 bp, Cucumis melo
  source 1..1397: mol_type = unassigned DNA
tatatatattaacttttaaaattttgaaaacgtcatagataaattatatacaaaataaaaagtttgattatttacgaaagttaaaaagtttatccgaaagttgactcaacgataaaaacactaaatatcacttttagagatgatgatattatacataaacatacgaacttacgcgtcaaacttttatactaacacaagatcaaaacaactttgttgagtagtgagaattttatctgctgatatggttgaaacttgggaagcaagcagaggaagttccattcattaccaaaatccattttgtattcatcaaaatatgaagtttagcgacttgataaagtcaagtcaagtggtcctatcgatttgttaatgtcaatgtttggttttgaatttgatacctattagacaatgatatataattttaagtatggtttacactgtgatgctttatatatttttaaatgtaaaatattagaacttgtaatttcaataaattttaaaaatgattttgtgttatttccttttttaaattgaaatatcaatgtatcaatattgcgtcatagagtattgcaacacaaccttatgttaaattgtttattgcttattgctctaattcaactccttcatcaaatgtgcacagaatttaaacaagaaaaagagtaggtgcttttttactaaaatatactaaaagctttttataccaaatcttatgacaaaatcattccaacaaaatgactatttaaatataagatcgaatccctaatttaaaaaaaaaaaatcaaagatgttaatttctattattaaactcactttagcgtagctaacaaaaaaaggaaaaatgagaggctacaaagcttgagccctctgcctccctttattgcattgtttgaaattagatcaatactttgtatttttttcaaaatgaaaaatcgtacatagaattaattctatggacaaaaaatcagagaaggaaataatctagaataaaattcgatttttaacccaaaaaaaaaaaaaaaactcgattctgatttttgtaagcaatcacccaaattaccataaataaatggtattcaattactcaattatggatattttagaaatgataaatttttattcataaactcttttctttctctttcaaaaagaaaaaaattagcataaacttcaatgacatttatttattcttcttcgtttggagtcaaaagtttaaattgagcatcagtccagcccaaaagcccacgaagaagcccaagaatcttcagctttttcgttcaaacgtccctttttggtttataaaattaaagaaaataaaaactaaatttatttgttatttaacaaaacatttttggttaagacattctctttgattatttttcttccattcttcgtcgtcaatc

SEQ ID NO: 127 - DNA, 2000 bp, Cucumis melo
  unsure 1914..1916: note = unsure at all n locations
  source 1..2000: mol_type = unassigned DNA
tttatatttatgaaaatgaagtctctaaacaatttttctactcccaaatttgttgatttttctgcctattctttatcggtgctttaaaaaatgaaaccaaatttcaaaactaaaaaaaccaagcttttaaaaaaatgttaggttatttttgaaattcaactaaatgttgaactcttttacttattaaataggcaaattattgaaataaatttagagcaagtaagcttaatttttaaaactaatatacttaccaaatcgaggactaaaatattcaaatactctttaaaattaagattaacattaatcactttgttatgtttaaaaagttgcagtgtcacttgaacctttttaaattaatataatgaaaatgaatccaactcaatatatataatatctatattattaatctcgatgtcagatgtttgatacgcacatatctcaaaaattatacctcaactaacatcggtgcacgatgtattatttcgtgaggataaaaatcgtttttagtataaattgatggaaagattatttgaattactgaaaaatgcaccggtacattatttgaaacttccccttcatttaaagaggctaatattagaaaaagacacgctgaggctatttttacaattaatgtgggctgttgacttggcccagcccaaaacataaaccctaaagtagcacaaacaaacgcctctttcttcttgaaaccgcatattaagcgttttatcacttctcaccacctctcattcctcctcttccgcacgttgtttcgctcctcaacgggagtgccttccctttgctctccctccaggttcttcttccttctcatctcatttccaagtaatttcattccgtttcttttctcttaatcgtatcttgttcagactctttcgcgtttttttttagttgcgccttaccagatctgtgttttcactcgtttctattcgatacatgcttccaagatccatttcttaatcgcatgtattcggttgattggatgattgtctttttgtaagttttgattacttttttggaatggatcggttgaaccaacggggtttaagtcgatggaagtaggttatgttaaagatttgcttctttttttttatgaagatgtgtgtgttctttttctttgctagatgatgttattatttgattgttttaacagtcgtgttttgtttttctgcagtttatagtcctcggtcttttgaagacttgtcaagatggttagtacacctcttgtcatcgtgattttgattgagtgatgtgttaagtgcttctttaggttacagctaacgcgattttttatattcaattgtgcctgtgcaggtgaagtttacagcagaagagctccgtcggattatggactataagcataacattcgtaatatgtctgttattgctcacgtcgatcatggtaagctacttagtttaagtttatttatgccgagcgtctatttaagaagattaacatcttagctttcatttattgtttatttggtaagcatcgtttctttttctccgaggaactgtacatgtcagttcacatgacaataaaacgatcttccttggacattagtttttgaagttcaattagacgccaaattttgttggttaaaagatgcttgtggagcatatggacctaatggaatcagtactttttgatggatggacttgtcttttgttcttttattttcaaaagaaattgcatgtgcaattacatcatctttgatcgaaagattgggtaattgggtaattggggtaaagacatgttgtaaaaactaatgttaattatcaattaccattatataccttatttagtgcttatttatatcctttttccccatttcagggaagtccactctcacagattctcttgtggctgctgccggtatcattgcacaagaagttgcnnngatgtacgaatgacagatactcgtcaagatgaggcagagcgtggtatcaccattaaatctactggaatctccctctactatgag

SEQ ID NO: 128 - DNA, 1657 bp, Cucumis melo
  source 1..1657: mol_type = unassigned DNA
ggcaaaatggagagaaaaaagtttctccctattgccacatttatatatagtatatagatatatactatagacatgatggagaatcataagataaggtaaggctgaggaagattttgacgatatagaatggaaaattttgaagatataaaatggaagattttgaaaatatagaataagatcatcaaatgatagcaaaaaaatccaaatgagtcagatgaaacactacgccaaattttcatcactccaaaattgttgcaaaggagattgattaatagggtattatacacaatcatatttttcgtagcatgataattggttaataattagacataatggcaatcaattagttaactaatacaacattttaggtagcaatattaaaattggagatccggaaaaaaactaaaaactcagaaaaatcttgggcaaaatgagcacggtttatcaaatttttaggcttttttggtacaattttgtctaggatgaaacgagatccataattttctttgagaagataaaaaaaattaagatttggtgtaagatttgggaagatttgaataatttttttaaaagaaaaaataagatttggaaaatggtagaataacggtctaatgtctcccaagatgcaccgggaaagcaaaaaacaaccaaaacaataaataaattggaaaattttaatattttaggaaaatctcgatgtcaatttcgtctaagattggatcgagaaaaacagttttacgagtttttaaaaaatgtgttatatttaaaaataaaatcaaaattgtgctacttttgtcaatttcccaagataaaaatgtatgcttccacgtaaaaagtaacattactacccttctttcatttaatctctatatttggaaatgtcgcactagttcttggtagctaatatttggatactaattatcttatatgacaaaatatttaatgtactttttttttacaacaaatattgaatgaacttaaataatcttttcactgcaatgaaaaaagataaattagagcatcccaaaaagatgaaaagttcgaaagtctgctaactacattgaaaaacaaagcatttaattcttcaaacttgatagttcaattaaatttctaccaactaactcaagtaaatctattattagtgtttgagtgaggctatgaactctaagactaagcctataagtttggttaaatttaattataccagcccttttgtaagtaatttgatttgaaaggtaagacgtaataccgattacccaacccaaaattactgtgaatgagttaaaaaataaaattagttgaattttaaataaaaagcataccaataagacgatgacacatgtacaaaatcttagaaggagaagcttcatttgaggacaaaaaagagtgtgtggagtgagaagaaagaatagtcacgaatattgctgactgtgcaacaaatgtacatttggcaccaatcaaaacctataaaaccttatccaaaaatcaataatctcatcccttcttcgctgttcttccccaatccaaaccccaaccattctcctccacacacacacacactcacatacacaaatccttccaacattattctatacccacttcccaattctcattgcatttcacaatcattgttctaactcacttacaacctccatca

SEQ ID NO: 129 - DNA, 2000 bp, Cucumis melo
  source 1..2000: mol_type = unassigned DNA
atgaacgaaggagaatatcggataatgaagaggagatccatgaatcacagagaatgaatgaaggagacccacgtgaattaaatagaacgaaggagaatgaagagaaaggatgaatacttcttttctttaattttaacctaatcgggtgaatcaaactcaaatcgaaactggtttagttcgattatgtttggtaccattgtcttttaaaccgatcaaacctgaaccaaacgaatcggtacggttttttgcacccctaatttatatcatgtgaaaggttttaagttaagggtcagctagtgtcgtttagagggaatgatattggttgacttcatgtcgtctcttggatcaagagtaggagattcgggaggggtgtgacgaaatcaaccccgagattgtcctacagatggcatgtaaaatgcatcatatctcgggactccttctacaaactcgagaaaaatgtctcttgagattcttcttctacacagccccaaattgatgaaatgactgagattctttgaaagacaccacatgcattaactgaaactaatgttgtacatctaaaaaactacatcacgccaccaactaaaaagttttccatttgcctgatttcaaactaaaaacaaaagacttaaacgataaactaaaaactaaaccacaaacaatgaaatcgttaaaagtgcaccttgagagatttaagagagtaaatgagttcacatagttttttgaaggaaaaatcactaaaacaagttggattgtaggagcgaaattgttcactccttaaccgaaattagcaaaatgtttggagtttagcgtttttagagaatatgtaacgttatgaataataagggtattttggtaatttgatatatccctttattttcaaatttttaataaaaaacacacatcttggtgacacactcgactgaaaaggaccaagatatttccttgaaagatttttttttttaaattgggaaagaatcttggggtcgatctcgatcgagattgatcgagaaaaatagaattacgagttttctaaaactgtgcttttgaaatatcacaccaaaaaagcgttatttctcaaaatttcccaagtttatatgtgggggttattgcgagttagcttttgatgggtttgcttttgggtgtttgtatataggtttgaaatgtacctttaatgtcgatttttgaagaaaggtacctttattgtttaaaattgacattgtaccttcatatttgatttcagtttaaaattgatattaattatccgcattttaaaaaccaacatcaaacatccatgttcatttcttttcaaatttaagcttgaggatgacttcgtgaaactttttgagcaaacacgtttatcggttgttcaaagtaaatcaccttcacaaatttaagcttgaggacgactttgtgaaatttcggcaagcaaaaatcagacaaatctcttcaatcttttttgagcaaacacactttatctctgctgaaatgagcacaaggtttagggttttgagaatatctagcatttaggctttcaatggtattttggtcatttgagaataccatttattttgaaattttaaaacaaaaacctaccatcttggtgacgatcatttaggccgagatgtattgaaaaattatgttaaaatgagtttttcaaatttgattagaacctcgtgttgaggtcgaccgaaattgaccgagaaaaataaatttacgaattttttttcaaaatgtgctacttttaaaatataaaactaaatgggttacttctcaaaagctaaccgaaactattagttatattgcggaaatatcaatttcgcccaattttagtcatccagagcctgactcatcgaatttaggagattctagacgttgcattcaggagatttttatccgttgtcgccgactctctttactgatctacattgtacttcattgctgaactcaacgagtcaactcaatcgtttctagatttggaagaatctgcttcagcgacg

SEQ ID NO: 130 - DNA, 2000 bp, Cucumis melo
  unsure 1257..1275: note = unsure at all n locations
  source 1..2000: mol_type = unassigned DNA
aaaaggcgaaaaaaaagttagcttcccgagaaggagaagacgaggaagagtttgacttcccggggagataaagtttgtgtctcgagggaatctctaatctggagttgaccgtcgacttatgtgtcgagcctggatttagttgcatggtgcgacaaagcgataaggcggcatatgtaaagtagtaattcaaaactagcggttaaagaaataatcagccaaaaaatttagtacaaatacgggtggaggccctaagtgaagtgctgctattcagaggttttggcaaaagagtgcaaagagttgagttgtgcagagaaagtactaggtgaggagaggcgtttggaaaagaaaaggatcaaacatttgcatgagtgatattcttaaactaaacactcttgtgtgagtgacttgcctaagctaaacactcttgcatgagtaactttcttaaactaaacgttttgtaatgttttcttaatggattcttttcgagtctgagttatgcttaacacgttttgtttctctcgtgttattgttgttgttgtttgaaaagagaaaactattgttttctatgtgctgattgtgatgaatgtgtgcgaaccattagccttaatcctatcaagtgaatagtgattatgtggtgtgtgcacataatgtaaatgacattgtgtggatggccagtgcaacaagaaatgaatcagaaagcttcccaaatactgtgaatggagtgaacatcacactagctcaatggcaagatattggcgatagtgaatcacaataggcttgacaaggggaaggattcatgttcttggttgaaaggaataagagaggctaatgtgagatttctgtgatttgcaaaatgaggcgttggaagacacgtttgagaaatgaaaacgaattagtgcttgacttgtattcctaaaaaagttgtccaatatcttcaatcactaaatatttgatgtgcctaagttttccttccttagttgttgaggcgttgaggccgagtaaggaaagataagataattatgacgttgaaaagctggtcaagttatccatctttggatggtttaaagttattacatgtagggagggttgcattccaattttgtgtgtgagatgagtcttattttcgagatgggttgctaggcgatcaaggagaagtataagaaatgagttcttatactcttgaacaacttgacacgaagaataacatcctagtggatgaaggaaggtgatggaacttaaagtttaggttttattttnnnnnnnnnnnnnnnnnnnaaatgtaattgtaaagtattagatcaagtaataaaacagagttgtgttttctatttttgctgtgttgggttgtgtatctttattgtgcttatggcctagttgctaaagagttaaggttattacctaaatgttttacggtgtgttgagttgtaaagatctcctgagttaaagttggaattttgtattggagattgttttgagaagtttagcttactaattgtttaactcattaggtgtctaagcgacacgcctccttttggtcgcatgaagtggctagcagggtggggcggaccggggtggggtgtgataataaacctaaaaaatcacccagataagcctaaattatacgttgaagttaaacttacaatttgattagaagaagaaggaatatctgatttggacatgaattaattacaaatacggcgccaatcatacaaagcacatgtaagatcaacgcattctacactcaatctcagccgttgattgctttcaatccttcaaaaagaaaaaaagaagggcagttcgggcagagtcatacctacccgttgactataaaagcaactacaaatcgaaaacctccatttctccgttaccattacagagaaaatcaaagaaatttggcgttgagagattgggagagaggtttctctttctagggttgcttcttcttcttcatcctccattgttgcaaatttcacttccttctcctcttgttctcatctccc

SEQ ID NO: 131 - DNA, 2000 bp, Cucumis melo
  source 1..2000: mol_type = unassigned DNA
atagagtaaccaatatgcccttttcagcagccaaagttttctatgggcagacttaatcaattaaggttcctattgaggccccactcttagtgaaaagcctagacccttctttccaacatgtctcaattggtcacctccatcaaaagcttctatcatttaatctaaaagcatactcttttttcctttttaaatttcattttgatggtctatatttgaaaataataatcactacaacgacgacacgttgttttcaaactattattttgtatgaattaataatttttttaatagtatagttgttttacttatggaatctatacgtttaatcgattcggtcacatctatttactttgatgtttttgttattttatttagacgtggttgtaaagagtttaaagcaatggagaagaaattgatgctttccaaagcaatacaaatttatatataccttcaaatgagactaacattagacaatacataaactataataaacattttgaaagtacatagatcaaaatgaaccaaagtcgaaaaagtacaattatcaaattagtttttaaaccttggataaacttcagcattcaaactttgtatttcttttttttttcgatcgatatatatagtgatagaagattttttttttctgtttattatttttgacgatacgttgagtagaagaatcgaacatcaaacctttaaatcaataatatatattttacgactcaatatctagccatcaatattttaaaatagcaattattattcactaaattatgttagagattggatgtcatacaacaattgttaaagattatttgtctagtttgttcaattaatcaagagagcattaagcattaaagtcaattattgtgataagatgcttttgcactatgtaactaaaaatagttggatacaccatttaaggccctacatgcaaaccatgataggcccacaaaaaaaaatctctttttggaaacaatggtcaaataatttctttcaaataataataataattacaacaaataaatacataaaccaaattactaaactaatgtatcaagttctagagaaaacaaaattatgccctttcaagttgcaacatcccctactataatttttcttcaaattttccatttaatataatccaattctaaacatggaaaagaaatgtaacaatatttacattatttcaatctttcctatattcatcgactaattttaataagacgtgaaatcaacatttttctaaactcgttgatgtcataaaaaataaacttaaattatgtacaagatcgtctattaaattatgtataacacgtgtggtgtatgagtaatagaaactttaaactcttgatcaaggacatgtacctataaataaatagatttctttaagtcttgactattaaccaacttgtattcagtaaggttaaagtgatctattatcatactaaatacacaagtttatttcgagtatgaatgcaaagaatcaaagatatatggtttaaacaaaatctattataccaataaaaaaggttaaccatatgcaataaaaactaaaaagtctattgctcaaatctctttcgagaccatattaaaaatgttagtttaattgacgtatgtatttattggatttatctaataacattttaagagattgttgcaaatatagctattagattcaaaataattaagtatatagcaaagtctgtcaaattctatcgatgataggactatgttaaaattgttgttcgatcgttggtaaactataaaaatctacgacaataaactactattcaaaaattttttactacccaaattttaaatccatctcatggatcgagtcaagcccacttctaaattgggccacgaatattgattgtgaagctcaataatcccatacgtatggctgaaaacgcattacgaacgaacgcccttcaactatccaaatccgaacccacccaaattccggttaggcttcttctccgagatcgacgaccgcg

SEQ ID NO: 132 - DNA, 2000 bp, Cucumis melo
  source 1..2000: mol_type = unassigned DNA
tgcagctgcacaaaagattccaaatgatataacataatagtttatgaaaatttaatgcatttaatttccccttccacagaagacactatatttttcaactacccaacaataccaataattatcattattattatacctctaattagtaattagtcacaacataaacagctattctcattaatacatataatcaacaacttcataaattcttaaatttgtatgtgtacttgatgggtgtagatttaagaagtccaagagtttgacaccctttgttaaaatgatatacaaattcctgcaaattaaatttaccattggtatgattgttgttggagtggtcacaacactaatttactaattagcttcgtatttaacatagttggccatgcgaggaggtagcttttgaacttccaataacctggcttggaaggacgtcgataaacagaataacaactatgctaaattttgaataatatactttatatatattatataaagacgacaaagttgaggagcatccgtcccctacatttgttggtgctcatatcatcctattgcatatgccttttaccaatgaaaccctatctccttaattatttctactccacactcataattatcattcatttattttcatgcatgactttcttttaccaaatttagtttccaattaaactccattaactaccaacaatcaactccaataacgtaactcacattcattctaaccaattgtttggattgactcgagaaaaaaaaatgttttttctaactcatttttacttatacatttaaaaattcttttggaagtgatcgtcaaacattttgatatttttttccttttaaaatgacttattttttaaaaaacttaaatattcaaaaaggttttccaaatgaatgtaattaattactcaacatagatctccattaatcattattatatgtaacaatagtaattcaaagtaaaaaaaaaattatgtggagtgcaaagatgaaaattttgacctattttacatgatttgaactatatgtttatgcgtacctatgatttaactcttatatacacatatttttgtctcaatttaatttaattttacgattttcttgaataattttattctctaaccacttttgaaaaacattttttaaactttagaaaagaatatctttaccaaacttaattcaatatatgaaaatagctaaataaaatttaaaaaacagataaccaccctttgataactgtagctgatattattaattaattgtcatatttatatttgcaatatgaaaaaggagatgtcatgagtttttttttttttaatcaatctaatgcaattttcttaaatttaattaatgtgaaggtgagagagagaggcaatttcaaattttaggtaagtattatgaataaggttacttaacattattttaatttaattttacattatgttttatttgaatttttttaaagactctcatttttccattttggaacttttggaaaagaaaattttacttcaatctcttatgcaagcaagttaaaactacatttgtcttttcatgggatttttaaggagatgtgtggggaaatacaataagcctttttttatttgcaatttgctaaatgtgtattcttccaattggctaattattaaagtgaaatttagattgaaaaaagagataaaattgaattgaagttgtatagatgggttaggaatatgaaaattgtttgagatatagtgagtattggttttatccaatgccatgtcataggggtggaatccaaatgaaccaatgagaatcactcaaaagaaaacagatataatgcactatccaaacctaaaactaaaagccacacattgctcatccattcactcccattctcaaaaccacacaaaaataaatatcaaatcaatctctttcccttttccatatataccactttcccctctcttcgcctctttgattattacccaccaaatattcccatatatcttacaacaacc

SEQ ID NO: 133 - DNA, 2000 bp, Cucumis melo
  unsure 529..531: note = unsure at all n locations
  source 1..2000: mol_type = unassigned DNA
aagcttgtttgaccctatttttaatgtcttaacacaggattatgaacaaaagaagaaactagtgaatcacagaggaactcacgcaaagactaggtgagaagatcatatcaaaatgagagaataagttcgctagaagataaaagtggtagttgaagttgatgtgacttgaccaagaggcagcttctggtgttgatatattcagaagactagatttcctgcctaaatctacctatataaagaactccatctccattaagaaaatgaggcctgaatggaccacccaagtggtcgactgtgtgaagagccaaatgtttgtgaactgcccatgagtgcctgaaaggcccgatcctagagagtggtgggaaggagcagcctttccaccatctgtaaagtctttcttcatcttctccagttagtttaagagtgaaagtttgaggttgagtgaagaagattccattcctatctttttctaactggtaatgtcatttctattctttccatttttgtatatttctttgtaatgtatttnnncatattgtacagtggcctaagacctatattctttaatacatttcatgtttatatcttttcaatctatcacgtttgttattattcatctgtccttgtgctattggtagcttaagatttatgataagttcttgataagaaggttagcttatatttcttatgtgtgttagttgtgagctattttcatcacctggctagtgtatattgcaaactacctgagagggtaagtagcaaagatatggcttaggcgcacaaggaggagtttggagacaaaatccacattggcaagataacttccatcatttgtgtctcaaaaggagaacaagtgtgggtattaagcattgagatgttgtgacccttaaacgagaagctatataagtcttagtgaaggtcgtttggatctcgagaggtgagcaagtgtggtgtttaaagacaccgagaggtgctcgtcttaatcataagctcgttaactaagttatattgcattagggatattttattgcttaatttcttggtaatgcacgaactttttttcacccattcttttatgccagctagttcacaattccatctcgcatccattttaatccccctttacagattctccggtgtagataagtagatatagtttaaacttacatgctttcacactatatattttattcttttatactacctaaatgcctagtgaagcctagaactaagctttgatatcgattccctgcattcgactctaaatcgcccatataaacctattgtttcgcttacacttgggcaagcaataggaaaacttgtactcaacgaggacttatgagttacatgatgacgagatacatagagagcatctaatatgcattgaccatgatcattgactcttcatgtagatttaaatacctttcagcttaattagatagaagatatataataaagccattccattagtttaaaagaattaagttagaggtagttgaaatgctttataagtgggggttaattctattttagctgtaatgctgagctgatctcaagccaaggttgccttgagatatccccgagtttaaaaacagaagctaaaatggaaactaaaaactaagacatataaactttttagttacttttagggaaatatcttagctataaattaaagaatatgaccaacatggaagttcctccatcacttttccaccaactcattttattgggggttagtcattttaaggccaattagtttaaattaaagttcaatctcagtgatgcactaggccgagagagaccgagataaatcattcaaatatttttttaaatttgggaagaatcttgaggtcgagattgatcctgagaaaaacaaaattacgagttttttaaaactgtgaaatataaaacaaaaaagtgctagttttgtcaattcccatttatcttgctcattgttgatacaagatcattaaaagtttatggataaatgttggttgaga

SEQ ID NO: 134 - DNA, 1696 bp, Cucumis melo
  source 1..1696: mol_type = unassigned DNA
tatatatatatataaaaagaggaatacaattaagacatcccattgttaataaggggtggaataaattgggaaattaccattcgagaaatcattgacgagagcaaatatgtcaaagtagaaaattagtcatctcaaaagaatgtaatcgttacaaaaattaaaagtacgtaaatttaatcatcgttacaaaaattaaaagaatataaattacaccgttacacaataataccaacaatccatttataatatgttgtttttatttcaactttgaataaaatttgaactctttgataaaatttgtttaaaataaatttaaaaccatttcaaaagctatttttatattatccaaatacatatattcttttctttttccaaaatgacttgtttctaaattcgaacatccaaaaattaaaacataacatttttagtatattaagaattataaattaagagataaaatattcaatactattataataaaatcggtgttttcagtaattgtatttgtacaagtaaataaaattaatagtaaaatttttaatatataaacaagttttaaaagaaacttaaagatataaaaaataaattgaaataaaattcaaacccatcaacaaataaagaaaataaagatggttttattgaaatgaatgaactaaaatttgaaggaggcaaaagtaagtacaccaaaaatagaatactaaaatggtagaggacaataattgcatatgtttggtagatttttcattaactatcataccaattaacaataatgaaataaactttctcgttgatattgattacaatcgtaatagggcaacccactgtttaacttgtcaaagttttcttaactttattatttttgactttatttgtttgttttattgattagattgatagattatatattttaatcatattatttatagtacaacaactacgaggtaagtgattgaagctttagtctctaagaacaaaggttcgacctaattttttagtctgtttttatttgacatattttgtccattgatagaattactatcacttaagttaaatgtattattattgcaaaccactaattctacgtaaaatctctaagtagcaagtgttatgtcaataaaatagcaattttttttttaccaattacacacatcatggtgataattattatcatgcacgggtaaatttttaattataaaatttcaactttcaaaattataccaatactaaatttattacaaaagttattttaggtaaattataaaaacttgataacaattacaagtacattctaaaactttcaataataaagattgaatcatccaattcatccaaatgttaaatttataatccgatttcaagaagaaaattaaaaactcaatttttatgaaaatgtaactacaaccacaaccatattaaacaaaaactcacaatttgtccatattttttaagttaaaaatataggtttaggattcaaatatttataaaataaaataaaatgaaactatttgaaaacatagtttaaaaaagaagaagaagaagtgttaaataaagtcccattttttaaaaaaatatcaagaccgatattaatattatatatatatagaaatgtacacaaagttaaaaaaaagtatcctataaatatctaagtttctccccgtctagccttcgccaaccttatctcaaaaactcggaagcc

SEQ ID NO: 135 - DNA, 2000 bp, Cucumis melo
  source 1..2000: mol_type = unassigned DNA
tttacatatttatgaacattttcctatttttgtaaatatcttgattcaagatttttgttcgatatatttaaaaataaacttattttaaattcatacttctttctccttctatatgattatataagtattgtagttactatagattaaactcataacctcctagttagatattgagattattactttcttttattatcgggccagtacagaaacgcttttatgacgattacattcgtcattcgtcacttatttgtgcattaaagttggcattgtaatgtttgtttttacatgattctctattccatagatttcctttatccttttccttgcatttgagtggccctttcctaagatgtattcttcggactttcaaataaataaagattagaagcatttttctcttcaatattgacttcatccttaatccttaagccttaagcggaggctaaaaaggctttatttgcctcgaatcccaactaattctccctctcatgcccatttcaatctcttgcctaattgttaattaatgggtcaaatttcgtattgaatttcaattttggatcaatcctacgattatctcaattaggggtcaaaattaatggttgatgtaggagcaagtggaagacacaattttggtgtagcaattggagcttcatcatcaacaacatgagatttaatcccgtggttgcagttaaatggtgtagaagaagtagtcaacacaacccaaggtgaagaagagggagacaagagaagtggttgaggttgtggctctatttgcctatggcagccttcacctcttctctctcgctccctctccgtttcaatcccttatccccttcctctccccgccattttcttcttctcttcttcttccctccaccaatttcacctcccgattctctgccctaaccatctcttcctcctccttgcactccgcctccgacaatttcgatcatgccaaaagctcccctttttcatctaaggtctgattcatttctgttgtttgtttaactcaatttgtcttagttatattcaatcgggattttgcttgcttgtggaattaattttcgtttattaagtggaagatatgggtatgcttggtgacactgtatttactgttaaatttcaaacaatcctaccaaattttggtttaaattgagtatttttagttccttcttggtaaattggatttgcgaatgattaacttaactatgttggcacttcgttgtaagaccgttaactatttagcttccttacgggtaatgatgtttagaaggggggtgcttggtccactaagtggagttaagtctatggtaaacatgttggcattagtaagtttttggtaaacatgttggcattagtaagtttttggtaaacacgttggcattagtaagtttttggtaaacatgttggcattagtaagtttttgtttgtgatgtagagttgtaagattgagttctttaataatttgagttgtaagattgaattcttcgataactgtgaaaagtatattaagaaagtaagatagagttacttgataaatttgaatagtggagataggggcaagattgagttccttgataaaagtataataaagtaaatgtgcaactcttgcctatatacagcttagcaggaactcttacttttgtgtgtcatgtattcttattggttcgttcttattgcatttagtagatagtggatcccagtgaacttttttaatcgctagaatggcgccttaaaaagttagttggagcttctacttgttggttggtatggtgcggttgcaagtatttttcctttctatgattatgtttttagatctaaattttaaagcactcgatgaatgctgatgcttgatatgttttctgtgttaaattcttttgttgatgaatattatttccatttttcagaaatcagttctttcatctttgatacaagagatagagccgttagatgtaagcttaattcaaaaagatgttccacctactactgtggatgct

SEQ ID NO: 136 - DNA, 2000 bp, Cucumis melo
  source 1..2000: mol_type = unassigned DNA
ttcttgattagcttggtgtgctgttgtatatcatatttggtgcgagcataacttacccttggctaccttgcatctaccctaagtggtttagtcagattgtatgatttgaggtatttcgtttctttgttgctctaagtggctttgagcttctactgagggaacctaggacgtctcttctttttgggatcttttttctcgagtagttggatgcctagttggtttttttgttcctttactcaagtcctttgtttgtcatttgatcgtgtcaaagtccaaatgctttctattgcaattcagtatcttaaaaaactgttctttgttgatttatgtaaatgacatactgtatgtataaaaggacagaatgctaccatttcttgaagtttctggcacttaccctgataatcgttacggtaattattatgtgcagattgacggcaataacgcagctagcacatcatggtatgatatttgtacctcttgggtacacatttgggagtaagatgatggaaatgaatgaggtgaaaggtggctctccttatggtgctggaacctttgcagccgatggaactcgacacccgactgagttggagcttgaacaggctttttaccaaggtaagtatgttgctgagttaaccaagaaactcaaaaactaatgccatgtttgaaatgttgttgggtatttgaaaacgtgttattacactagcacacttttactgtacttccttccaacatctattattcagcttctcacatcatggctatataaataaaggttaatggaagttactaaaaatgatgtaaatctatcacattgttaatactcctgtaattatattgattgatgaacaattcgatcaccatcttttgttatttaaaattaaacttgtaatatgtattcgaacgtttttagctttattgcatgcttattatttcactgttttaaaactatctttagacttcaaatcaaattctgaaaaacaaaattaagttttcacatacattatgtcatgaatataaaattttagatattttagttcattttactatatttaaaaatgttttattattattaattttgtaaaacaaccatgatcgtttattaattgaattgtcacaattaagccattatttttttttttactttcctttttcccatcaatttctttattttctaaaaattattggcctcccagactctttgttatttgcaaataatgagtctaatcataatagaatttcattgataaaaccaatcatagcgagtcttaaaaccaatcatagcgagtcgtaattataaatattattgaattgctcttggtccagtttagctagaattatgaatttgatcaaattttctgttatcattaccgtataacaataaatgataaaattcaaaaaaaaaaaagaaagaaaattgatatgttaacgacaatggtaatgataaccataattgtaatggtaaccgtaactacaatacataatttttgaatccaatgagatgaatcacttacttagttgatttgcgtaccaaattatagaacaccaatcatttttgtaattaggattgatttactagcgttagattagagaaaagcttggcttatttctaattcctcctccctcttccactcattttgtccttaactaaaacatagtgatagttccctttttcttttagagaaaagaaaagaaaagaaaagaaaaagagtgttaattggtaatacataataacatatcacatacataaataaatcatgccgagttcgccttagaaacgacgccgtttaaagtaagtcaacaagtcaacactgacagctaatttccgcaataaatacgtaaaaatgaaaagaaaattaaaaaacgatataatataaatagaagcaagaggctcccatcacaagatcccattcgcaaccacattccggccttgaggcttcaaaaaatcgaaggaaaacactctctgtatctctcccctctacccaccgattccgtcgcggccg

SEQ ID NO: 137 - DNA, 1575 bp, Cucumis melo
  source 1..1575: mol_type = unassigned DNA
atatatatatatataatttaactaaataaacaaatgaaagaaaaaagtgagttcccattcggaaaatgcagatggatccatttcttcaaatattaaaaaataaaaaataaaataaaataacttaaatatgcaaatagaaagaattttaatttctggattatccatatgggacaatttttaaaactcatttattttattttttttatttatttgattttgatatatctatggggaaatttttcgtaataattttcgaaaaaatattgcaatatatcatttgatcagatcggtattattaaatctctatcacatttggtcttaaattatccaaagattcctttaagataatttagataaccatctacagatcactactataatcaacaaaaggaacaacttaaattatttaaacaaattcattaatattagactttgtgcttcattagaaaatgatcttatcacaaccacaaccatagtggtggtttaaaattttattttaaactcttattagtattattttaattcatacttaatcaaactaattactttaaaaaacatatatatataaataagttaaatcattcccccttatctaaataacataaaaaaaaattgtttactctacaagaagtttgtatatatatatgctcggtactatttagcatctttataataaaatttctaaatcaattttttatatctctttattaaatgtatagtcatcaaaaaatttaacgagataatgtgtcaaagatttattttattaacgttcataaatatcaaattatacttagcttataattgaaaacatgttcgataaatataagtaaataaaattttattttttttaaatattacaaaataaactaaataagttataaatatgacaataaacattatatattttattatatttataaatacttaataatttagtcgtttaaaataattttcttaattttcaaaacatgtttcatatgttaataataaataaatggaaaaccttccaaaagaagaaaaaaagatatcttaaaatttaaaaattgagattttgaggatcaataattaataaaagaaggattaataagggtgaaattaaatcccaaaaagaaaattgaaaatgaagaaaagaaaagtgaagaaataattgaacgtgggaagtggattcgatgtctccagagaacaagcgaaaggagacgaaatccacataatttgcacgttacgtgtccctatcaaccgtagacacgtgtcaacatctcaacaccctacgccgaattgcttcgctggatctggacggtcatcggataacagcggcaaccaattaatatttccccttatatttcacagcctggccatgtccaccaatcacgttcaactattaattcatttttcatttcctttttctttttttttttaattcccctcaattattaccgacaacctgttgtagccggttaaccctaccctccaacgttccattataaggcctagaaaatggacgtgaaaatggagtactacaaactacaattaattttaaagaattttaattttaaagttctctaattactattagcc

SEQ ID NO: 138 - DNA, 2000 bp, Cucumis melo
  unsure 1772..1812: note = unsure at all n locations
  source 1..2000: mol_type = unassigned DNA
ccgtcgggaacctctgctctgacataattaatattcatgtatttcctgatacccatgcaagtcgtcgggaaatatgttaccaattttcgacgccagacgagaatcgttaggaacaagtgtcaccatgcccaacttcttgtatggggcatcaggataagttaaatttcttttttagttgtgaactattcccgacgccataaacctagatgtcggaaatgtcttcttgtttttcgacggcttcgtgaatcttcgaaaaaacgtaagattaaaataatgttttcgacgagttccgacctgtgcaaaacgacatcgggaataggtatttattccaacgttctagcttctgacatctagaacccttcaatttcttgtagtgccagtgcaaagattgacactcttaaacgatgggacttgtcaaatagatgttgcgcagatatccataggttatctaaggttttgttttgttacctaagttatcatcaaacttcttcatgaattctcttagctatttctaagtacctaagttctcctctatccactaggattgtctctcttaaagtcaagggtggctgttggtaggatgtagactttgtcggcattggtctaccaatttaatctcttatatccctaaagacctagactccatggtctccacctatttccataaatgtacccataacatcattaaatgaaattattactcaagtacaaaaaaattgtttaattttattgataaaaaccatatgtgaaaaaatagatgacatttttaaaagcttgtaaacagtgtgtgaaataagtatcctaagtgaaggctattaatttaacttaaacacaataattattattgttttaatgatgaaaataattaacttatataaccaattttcatcaacacatacataccttttgtataaacatttatttgaacacaaatgagagacaaatagacatttttatttggtaattttctcagcattattaattatcattttcagatatcttaattgaaatttctgaataattttttatttttcggattttcacattataatattttgaattagttagttgaaaaccaaagccagcatcagtgaaaactcattaatacatgtaaaatactaaaattgtttttttaaacttctcaaagaaaaaaagtcttaatttttattttcttaacttgacataaaaatcattggtgttgtttttaataaagtaaatgttaaagtagactcagttaaaaacgaaaaaaaaagttaaagtggactcaacacttggagtaaacattttttttaaaaaaaattaatcctaaaattatgattataatttttatttggcttaaatatttcaaaatgtgttacacatggtttagtttcaatttagttgttacaaaatttattattgtatttgaatttttgatagactaattaaaatttgaaaatcaatttatttatacagttgtttttcttttaatgatgtaaatagaggtctaatgattttaacttgtaagggttaatttttcttatgatctaatgtaattcaatgagcattaattttagaagaaaatgtgtacttattttgtgtaaaaataaattataataacaattttttcattttggtataacgtatgattaagttccatgaaaaaacaaaataaaaaagaataaaatatttttccatttaaagaaaaacaataataaaaatggagggattcaataggaatttcggagggcccacttcccaattccaactccccactcactcactcactcacnnnnnnnnnnnnnnnnnnnnnnnnnnnnnnnnnnnnnnnnnttttttttattattatattagaaattaataattattgtttatttcgctgtcaaataaaaataaaattgtggggcaggtgcagctcacgtgcctcctcacattgacaccacatttaaacactttcattttcaaaggctgctgctttatattcttcacaaaaacttcctcttccctttctcacactacta

SEQ ID NO: 139 - DNA, 1006 bp, Cucumis melo
  source 1..1006: mol_type = unassigned DNA
ataataataataaatacataaaataaaaaaataataatagtaatgaaaatcaatagaataattttaaaatcgggaaggaagtcgtgtacaatccttgcacgttggagagtcaaatggcctaagtggtgatgtggaagtcgtgtaccgggtacacgattttcctacaagtcaataataataatatggttattttttttctagtttagggttcatgacaaaagattgttcagtcgactggatgtagacaaatctaaaaaataaattaaaatctaatatgaaaactagttttaatttccaaattattaagggttgaattcgaccaataaataataataatacggttattttgaaatttaggaaattgaataaagttgttaaaatcttcaagcaaattgttaagccccgagatattaagaagaggtaataatagaggattctatatttataacatgttaaaattaattgcaaactcataaatgcatcacacagattaacaacataggagggacttccgataaaagtgcaaatattgaaataattacagttcgcgaacatgagtattttaatattttataaaatagtatgcacgtgtatttttgccaaaagaaaaaaagaatagattttgccatttttcaaagtgactctcggttatatcttttatggcgattgtattttatagcgtatgttgtttgtagttaacccatttctcattggcaaattcaatcgtgggccacaacgtttgggcatagcttcaatttggattaactcaattatgtctgaatgggttggactagttcggactcttcggctgggccagaatcagattcgggccgcaatctgttcatttcacacctatatccaaacacccccaaaatcgatacccatcaaaccctaactctcaataacccccatatataaattccttctttagggtttttcatcctcatacactctcaaacctccggtcattctcattttccctgccgcttcttcaataaccctaatc

SEQ ID NO: 140 - DNA, 2000 bp, Cucumis melo
  source 1..2000: mol_type = unassigned DNA
aaggagtagactctcaagtccactattctaacttcttacccgaaagagccaaaacttttcattcaaattcaactagaaagttattattgatctatcaatttgattttaatctacaggcgtgcgttgcaatttgggaagggattgagtttgtaactggagtacgggcaacctcattgaattctcttcgatcaacgtggggatgaagttctttcaccagttggagtctggaaaaacttttgctagactaacctattgctactgccttttggtgaaatctttgtgctctaatattaaaaagactccaactttgaatcgttaattataaactagtgttatttgcttgtaaatcttacttatagtttgaaatgagtgcttggcgaaagtgttgttcaaatcggtacgtgtaagtttaaagattcttatttcagctttgaatcagatcagagtcttttaaacttaatcaaccgacaccaccacaccccactcttgttcttctccacgtgggagttcccaaattggttgatttgttatctctttgaatcatctcaaatcaagaaatttcagaacaggtttggggaaatttgataaactacactctcttgctcgaactttgcaaggtttttactgtttgttatatgattcaatattcccatttcttctaattggatgaactgttgaaaattggaaatgctcagctgccaagtttttttccgaaataggtataaattcaaagattcaatcagtgtgggtttacccaaaaaaccaatggggtaagtccattttggactcatgtggagggcacatgtttaggcaaagccttatctctttgccagtgggctcacaatcaatacggacaagacaagaaatgcttcctaacaccgtcattgtcagcgaccatgtgagctttcagcaaattggatccttcaagtaactcacgtgaaagatatttagtgattgacttaattactctccccttcctgtttatctaaattaggcgaatagatccaaagtgggtatttttggagatcatttatctgtttcctgttcttgtttatcgtttataattattgattgtttttctggctcaagtaaaacgaggactttgacatttcaatacccccttttttgttttctggtaggtagcgctaagtgggtttctgatatcgtactgaaaaagttatagttttgctagaacactcgatagattttagcttttgtattgatttttttgttgatatttcctggtttcagtgaatgaatgatattcttttatgacggttgttgtgaagactcataagtttgtctcagatcttcagttatactcttgaagcttcttcgttcatacttcaacagttcttgtacattttaccccctctgttcctctttccatcggcttgtgaatctgtgattgtaaattgtgctgatgattgtttttaagctgttgagatggcgttggggttgtgtcctaatttgagactggtcaacttgatcatttggggtagtgatggccttcttttctatatcattctgtgaagagtactttctaaccgattttgttaaaaacacatgtcggattgcttgcttgttttgtggtgtttctgatttgtgatatgatttgattaatctctgatcgagttgttatgaatttgattgacagcaattgggggaccatggaatcattgtggttcctctcatagattttgatttctgaggtgttgagaaggctttaacctttttgtcactgaaatggatggtggaagctctgaatccccagatatgggttgtaacaagaccatagtatggtttcgtaggacctcaggattgaggacaaccctgctttagctgctgctgctaggaatggttttgtatatcctgtgtacatatggtgtcctaaagaagagggacaattctatcctggtcgggtatcgaggtggtggttgaagcaatcccttgcccatttgaaacagtctcttaaatcacttggtgctgacctagtgctg

SEQ ID NO: 141 - DNA, 1556 bp, Cucumis melo
  source 1..1556: mol_type = unassigned DNA
ttttagtcattatacttcaacatctcgttggttttaggtttttggaaagcaaacctacaaaacacactctttcattcattggttttaagttttgttgacaactttttaggagtgctttgactaagatttcaaagtcttgtacttaaaatgatgcatactatcgtaaaattagtataagagactagatttttaaaaaagaagaagatcggtggaagtatgttctaatttctaagtttttcaacacttacaaatttattgaaaaacagctgtcggtacatgcacacatactatttatggatctacaattccaagcatagaagagtttagtatatatccaaattcttatttttaaggggaaaaaatgaacgaaagaatgcattgtattctcgcttttgtcgtgataacgtatgattttcaagctctttcgtcgaaaaacatcaacaaacaaacaagctaagtgtaatctaaataatcttcaacatccttggaaatttattgaaaaataaagatggctagcaatgcatactttttatggatctatatcccatttcaaccgtagaagattcaaagtattcgaattcttaaaaaaacaaaacaaactgccttgttaagataaaatggaattagaatgaaattttcaaaattgaagtggggccttgtaaaagaataaactttgtttgaaaattaatttccatcgttggttggtagatgtgtccttaattgaaaaagtggaagaaatgaaggatgaatatgaaagttctgaaaagaatatggacggaattggaaaaaacaaaaaacctaatttcataaattaaccagaatctaaacattgggggatgaagggagcggaggccattcatgtaattggccgtacagattcatggtttaacaaaagccacaacgactcccattcttccaccacagaaatttcctctcctcctaaattcacttatctctttctatataattgcttcgttccccaactttctatcttcgtgcagccccattcaatcccccattttacccacttcgtcttctcctttctccttcgtcttccagttccgttttccccatctgggttctcctgatttctctttaaaatcaactacccatgttcgactttgaggaactggtgcgttggaattgagctttcgaaggagatttattgtttttatcacaacccatctgctcgaggtaaggggtaaaacccgggttcgtcaggctgtagacatcacggctatacacgtagtttcccggtcgttctttcatgtccgggctgtacgacggaagggttgtataactccgacaaacccttcgccgcacggcggacgtggtgctttgccttccgaaggtggtagtccttctgatcttctttttctcgccggcggtggattctcttgcttcttctcttcttcgtattagctttgcaacgagtccgtttgtgttttagctctaccggtttaggatttgacatcagcaagtttctgttttgcgtttctttgttttgggtggggagattttggtgttgggtttggtttgaattagaagcagacgat

SEQ ID NO: 142 - DNA, 2000 bp, Cucumis melo
  source 1..2000: mol_type = unassigned DNA
gagtacctaatctaaactaattaactcgctcaccctcttatttactgccccatactaatgatcctaatagattgtttggttgggttgataaattctctttaaaattatcaagttttccaatttttgccacctaagttgttttcttacaaaaaataaaaaataaaaaaaggcaatgttatttctcgtatgcattaattgattgattttctcaactaacccttcaatttgactttatatgtaataatagtgtaaaatatatacgcacatacctacatatgaccaacaataaaaacgataacattaaattcagacagaaataaaaattacgattatgattttaataaatataaatgcacataaataaaatttacagttcatagaaaaatccgatgtaatgaagtttaaatcgttagttattttatttcgtaaaataccaatttatgatttgcatgacaaatttttaaaatataacttatgaaattaaaagttggttttgagaaaacattcaagactttattacaaccaaacaaaaattttattgagttttgtttcattaaaaaaattattaaattacaaatatttggacttacgtaatttgttttctttctttttagggtagaaaaatatgatagattaaaaggattcgaaatcaaactttatatcaatttccttttaaataattatttctttccaaatttagtttttatatgatagcctaagtctccatcataagaaacaacgttaattataataaaaaatggatgtagattcaccaatattttccaactatattattactttcacgtttacattaaaattaaatccacacaataatataatagttttctttgtttgattcaaagtttctcttggttaaaattaaatttcgaaatgataataaataaactcgtgattaataaactttaatttaaatttcaaacttaggtgtctaataaattcctatattttgtatcacaacttttcaattatgtgcaataaattttctaatgatttattattttttttaagaatgtaaagttgattatattcatattaaacataagattgaaaagagagagttgattatataccgagtagccgacagtcattggaagcattaacccattatcatctccggcgagcaaaagcaaggatctacaaacaaacatgacaattaatatgaaactcatcaatccacgtatccaaacattccatatgttagacatggaagagcaataattacaaagctctctcatcgtctccgatcactccatttatcgtacaaatccgtctttcttcaccttaatcattttccccgaaattcatcccactgtttcgcaacaaaatccaagtttggaaagatgagtttgtttttagtgatcaaggaaaggacaaagaatgtagcattggcaatgacgggcaaacaagagaggtgtggctaaacttatacatgcttttgtttggtgaaaggttaaagcgaagaacgccaaagacagaggaaaccgtataaaatatgagtaaatgtcaatgctaatgaatgggcagaggtgaagcggtcgtctatggctggagaagggcagatgtgaaacaatatgaggtagacgaaggtggagacaaaacaatttagtaaagtcaaaacaattcatccatatcctaatccaattatatttctttaaaaagtttaagtatcaaaattggactgcttgatcatctatcaagttatttttgaactttattttaaaaagtttaagattattaataaaaatgtaatgtttaaagtggttagtgctttggaagccattacgtcctatggattatgtggtgtgttgggctactctctatttggacatgttttgacgtaccgtgcgaagtcctgactctatttgtaaaacgtcacccggcaaaaacccaacttaaaaaacagaactttatttcatttaatttgcggggtttatccggaaagaattgtgagagctctcttgtgtttggtttgcttatct

SEQ ID NO: 143 - DNA, 2000 bp, Cucumis melo
  source 1..2000: mol_type = unassigned DNA
gtaatgcaatattagcaattattttggagcaatacaaacaactaggtttggatcaaatatcacgaaatacaggagcaataacattaacaacaaataaatgcacgaagtttttttttttgaacaaacacttaactctctccaaaccaaaacgagctaagttagacctaaaaaaacaaagtatcggaatacaatatagcttaaacaaaaatcatgtttagattattggttaggttcatctaaactagtggttagccatttttcaaaagaaaaatatgatttgtccttgctaattttccaaatctatattttaaaagtatcactctcgtcataattttccatagctcaattaatactaatctcacggtagcttttaattgttcttgacaagtaatggattaacttaaaacatttatataactttgtaggtattattttatagaaaaattagtttatacgtgaaaacttcttaaatatctaactacaatcaaatacctagattacataatgtatttttcataatatttatacattatatttgaaaaaggactctcatttcttttattggtatctacgcagaaattaagattttcgagttgcgacatctcaatcaacgaaccagctaagaagaccggcaaattccaaacgtatccttcgggaagcactgagtgtttccacgtcaataacaaaatattgacccaataaatttcagccacgtagaaacaaagcaatgaaagccgtcggattctccacatcggctaccgtatgccgttaagatcatcaagtagacttctaattcccatgtcttccgtgggggccagaaatggaaaattgaaatcgctttatccacgtcaagctaacaaaaaacaaccaataataattcgccacgttttctcattagaaaagtgcaccgttggatcatccacgttggcaacatagatcgatccgatggacttatataaatttgggtagctcgtcgagaaatcagatcagtgatcgaagctactggaagtttttgctaagaaccatgaggaagtggacgatcgcttctgctcttctccttctttgcattctctctctcgttcccgatgaaggtgtgatttcgtttcttccttcagcagtttgatttatttgttggaatgtaaactgaatgcattgcattatcttaatcacgagggctgatgctttaatttttgggggttcgaggagaaatttggatgagattcgagcttcgtttgaactgcgaaggtttgatggtgatatttctattgtgtttgaattttcaggtcctagatttcatgccaaggccgacggtgatgccgacgaggttgtagatccaccaaaggttgaggaaaaaatcggcgccgttccacatggtctttccactgattctgatgttgttaagaggttcgtgaatgtctaatctcgttgatacacgcttcaagtatagatttgtccacttcgggaaaaaaaattatcgaaccttcttttgaatgttgattcagagagtcggagtcaatctcgaagagatctcttcgcagtagcggggagaaatttgagttccaagctgaggtgtctcggctcatggatattatcatcaattccttatatagtaacaaagacattttcctaagagaattgatctccaacgcttctgatgtaagttcactctgcctcttctcacttcattagatctagtaatctcattgttagatttgtgttagttaataatggcgtctctgcatttcaggcgttggataagattaggttcctttccctaaccgacaaagagatattgggtgagggagacaactcgaagctggagattcaagtgagttcgaccttcatactgacatattgttttcttattacctcgctgaaaaaagctgctcgttctggttgatgaaccttgcatacttttattgttgtccataaatcaaatatcgcagattaagttggacaaagcaaacaaaatcctttcaattcgcgacagaggtattggt

SEQ ID NO: 144 - DNA, 1337 bp, Cucumis melo
  source 1..1337: mol_type = unassigned DNA
ttttttttaattttctttttgcagattgtggggctgatcgtccacgatatgattccactttggctacgaggggtgtcgggcaccttgtccgtaagggcactggtgggagatcgtctgttaggtaacctagccctagctttttcgtgtttggattcttctatttaattgttggcttgatgttgtagatgtaatgctgggtttgagtgctttgaaatgttgagggaaatttaagaaatttaatgggatgaagatgaacagtggtacttcaagcctcaaattgaattaaaattattttaaacatcctaaattggtatgactaagtattgctaaacatgatagtcatataaaagcgcaaaagaaaagaaaaatcacccctctactaggattggtttattctatggatttttgccttcagtgttcttgaagtcacaataataaaagtagtaatagttgcagtcacaactcaaacctttatatgttttttaagattgtggtaaatattgttttgatcattagacaagacatagagattttaagtctctgggccttttcacgaagccataagcctcttatggttcagcaaaggcatactcaaggctagaagttaaaaaagccttgccttgagatgtaattctgaatacctttttaaaacatttggtacttcaaatttataagtttattagtggaaaatataatctttcagtctcttttttagctgaaatacttataccttttttccccattgtcattgatttcttaattcatatgcagaggaaaggactaattagatatactttgttttattgagtaatctaaaagatgtggcactacccactatgaacattttgacgtcattccagcttttatgggatattgaagcaggcaattttaatctgagctggtttctctgtcgctgtcagataatccttgtttgtgcttatgtgttctctttcaagcatgcacattaggattctcaggcagatcagatcattgatatttaattcaatttgtggatttagtttgtagtgaatacactaaattctgtctctggtttctctgatcttactgttttattacaaaattgttttgcagtgggattgttgctactgtcttcggagctactggattccttggccgatatgttgtacagcaactaggtaataggtgaacataaatggtactagcattcgactttctttttgcttagatatgtaatttattacgtttctctataccttctactactagtgggttttggcagctttggctctattcttgatttttatatcaattttatgctaagcatgattttggaaatgaattgtgtttcagctaaa

SEQ ID NO: 145 - DNA, 2000 bp, Cucumis melo
  source 1..2000: mol_type = unassigned DNA
atatgtgacgattaattacctaaaaattaattatttacatagggttagtcaagttgtcccgtgagactagttgaggtgagcataagttgatccgaaagctcacaaaaatataaaatacgtcaatctccatgctttcataaaaataacaatttgattctcatgactactcattcacttatcgtaaactcttttaaagaatattaagagcgtattagtgtagtgggctagtttgttacaaaagttggcgacaaatagatgaaattagagttatctcgagattcgacgagggttaaaaagagcatttgctttaccctgtattttcatcgtagttcatatttatttatattcaaattctatcaagttaaggccacgtatattccaagaaaacataatccattaatggtaatatgaaaaatgagttttaatttgatcatgttgtcggcattatgtaatcacaaagatatctaaagctcaatgttaaatctaattaatggaggccgataatccaattatatttgaaaattaagtggaacctacggtgagatatttgtactatcacaattacaattactcttacttgttcggaaaagaaattttgtaaacatgtcaaaattatcgttactattccaaatattgtcactgacctgaacattgtcaaaaagaaataaataaataaaataatattagataatgtaaaataaaccacctaaactttaatctattatggtcgcaaatgctttgataacacataaaccgattgatccgtcaatgaaattttaccataatctttattatggatcgataaatatgacttaattttcttttaaaaaagtgttttttaatttaaaaaaaaaaaaggaaaggaaagggggaggggcaaaggttctagagtgttccaaataggacaatggaggagggtctccaatggagggaggagccaaatccaacggccaacaattgctggaagcttcaggagcctacatgattcttgggttcgtttttctctcctcttcctatccatccttttgaaatttgctataaagaaacctacttctcttctccttacaaaaaatccattttacactctctgtaatacccccagttttgcctcactcgcagcgctcatttctcaccctcttatccaaatcaatccttctccctctaaaccctaaaacccctttgcacctccgccgttttcttgtaagattccccctctcttttcattctgttggactttcttatccttttactttactgggtcatgcttacatttctatttgggttttgtttttgcttgccgattcagtcttctgtattgtgttttgagctttctgactgttttggctttctgggtttcaattgttggtgtagacttatcgattgattcgtttgttttgtgtcctttcatttctgggttttgatttctttaacattttcttcatgggttttggattttgggtcttcttcttgtgtgcatctctgtagcttgctgattcatttgtatctcgtgtttatctatttgtttgagttcctgacatgtgggtttttgttgttgtctgagaattatgtgtcaaatgtcaattgtcaattcctatgttcttgaatttgtttatgtcatttcctttctgggttttctctgttcaatcttgctacatgggttttgggttttcttacccttgttgtgtgtagttttagctgatttttgtttatgcttactgattcggttctgtattctcgatgatttgcttacctggttttttatgtcgtttgagaattgtgtgtcaattcctttgttgttgattttgtttgtcatttctggtttgacattccatccaatcctctctgctctaagtctacttggttttcaattcatgaatttccatcagacgcattgtcggccccctgctctatttgtttacaattctggttgtgaagttgtttcagtttgaactaattgatggtctggtgattacgttctgtatcagtttggaagagggtaa

SEQ ID NO: 146 - DNA, 1212 bp, Cucumis melo
  source 1..1212: mol_type = unassigned DNA
atatatatatatataatggaataggctatttgatttagatgaaagctattacgtcctggggtttacatcataatctctattataatgttaatcgagaaactttataaaggttaactcattatctctcttgtcttcagtttattattgttgtttttatatcggtggaattccacctttcaccaactctcaagctgtggtgtgaatctatgggattaatctagggcgaataagggagctgagtattttctatttgtggaattaaatctatagtacacaaaacatttgctcaactactaaggatatgaaaacccttggctctgccaacatggcttatagaaagtatctgaaaacgttcaccactttgcaatttcaacaataagtgtaaattcttttcctattgttgttatttagtcgatttgatcgttgtacaatatttgctgtaacatgtttgatttttggccattttagtgttcacaagaagatattgtttgttataagaatctacctgatccttttcaattgttattcaatatattgcctactccgttgacagcaggtccatgcagaggaacaagttctaaagttcaaactcgatgctgatattcttcaggtactacttttctgttttcacaagtttgttgtttcaatagttctaagacagtgacactcatccctttatctccgtaacccaattcattaacgatgacttttgatcggtttgaagaaaaaatttataacactttctcatctcgttccctttggattttcagtttttaaaattgcatctatatgtattcttttgttatcaaattttacttgataatgacttttaaattgtactaactcatttagatgtgaatattaataattttaaacttcatttctgacgtctaatactaataaaataataataacaattatccttcttaattaaatatggtttacctaccggtctattgttctgaactggatatattcaatttgttttatctgaataatcttttgaggttgagttatcaagagcctgtttaacttacctaaagcatttctaacctgaactatgccccatatgaatacttcattttctttattctattgtaaaacattgttgttattataatttgaaacgcctgtaatagtttttacgatgtcttgcaggagtctatcgttcggcatgtaaacgaacacccacaggctggctggaaagctacc

SEQ ID NO: 147 - DNA, 2000 bp, Cucumis melo
  unsure 596..603: note = unsure at all n locations
  source 1..2000: mol_type = unassigned DNA
acatagtattaataaattagggaatgacttagttatttaatttaagcggtagtaaatattattaacttttgttcgttgtgttattttactttcaaaacgttcatcttgatctttatcctttctaatatttatttattttagttaatatcaaaaaactaaatttaatttatacgttaagttacaacttcatttatttcaatctaaaacttttagaattacactttattcactaaaaaattactcgtaaatgcaaccattccaaaaaggtttcaatattatataaaatatcataatttttcgaacattcttaaaataaattaaacaaaatagtagttttcatatacataaaattcgaataaatcctcatacaaaaattttaaatttgaatcatcacattgttttattttagataatcaatcaaataatttaggaaaagagaagaaagaaaagtaaaaggaagttgaaggtattttatttagtgatagaattataaaatagggtattttagaaataaaaacacaaatatataaaaatacagaaattgatgcatttaatggaacactatttgacaatcaataagaaagaaaaaaaagaannnnnnnnaaaaaaagaaaaaagagaaaaggtttggtattgggtttgtgggattttattaataaatgaaataaaaaaaaaagaaagaaaaatttaattgattaatttggtgggagaatattacaatgaaaccccactttgtgaacaaatacattgcatttgggttgtaatcaagtgtacatgcatctacccaaacctttcttgaactcaccataaatccttcttttagaccgcttcgacttcccaatttttcttcactttttttcccccttctctctcttcctccgtttcccccccccttttttttccctatctcatagggtttccatccaccttcttcttcttccgttctctcatgcattgtcattcacaatctcattctgaattcctcttgatcttcttcatcttcatttcctccttattttttgctctctttcgagggtttttcggttcatttccgtccagattccaccacctcccgtggttttttcacccatactcatgtcgaagctcttcgccttttccggtaagtttatggatttttactgattttttttttttgtttgttttgcctttctttggatttgacttagattgggtagctggtagggttaagcgtcgtgttttgtatgggtgtttggattgttatttggatcgtagggaaagatttggaattattggttttagtttttgggggtttcttgattcgccaggtggcggatcatggcttggtatgaattgtgagggaatatggatttgggtttctttctattaggattgttttattgtgttgattgattggctattttattgtcttgaacagtccatgccagatgtaagtttcttgaaaagagatatcgtagtttgaagatgggtttaccttttaagtgatgtgtatgtgttgttgatctgtcgttcccgtacagatttagatttgaggtttagaataagagagcacatcaatagtaaatattaaagggtcaaatatagttttgcagagattgcttcttgtttttctctgttgataaattttcgatcttttgatctagaagttgaggggtattttggtctgaggatttatttgtgatgttggatgatgtatctaacttgtagttcttgttgttgaaatttcaggtagtgaagatttttgcactggagggtcaatatacccaaatcccaaggactccagtttattcttgtcccttcctcaccacgttgatgtctattttcctcctcgaaagaggtctcgcatcactgctccatttgtgtttggtggagaagaagttgaatcaaaagcaaatgtttctatcgagattcttccggatgagtgcctgtttgagattttcagacggttgtctggtggcaaagaaaggagtgcctgcgcaaccgtttctaaacgatggcta

SEQ ID NO: 148 - DNA, 1254 bp, Cucumis melo
  unsure 131..143: note = unsure at all n locations
  source 1..1254: mol_type = unassigned DNA
tcataaatatatatataaaaaaacaaatattataacctaccttttgcaaatgataaaattgtaaagtctcgtgccgataatgtgttataaaataaaagaacaaagaaactaaataagaacaatgcaacaannnnnnnnnnnnnaatagagaagagaggaagaaggggaaacaattaaaaactcaattgtagtgtgacttacacaaatgcaacacatatatctatttataggacatatcatggtatatgttatattatgaaattcaatgaaatgaatgttacaataaagaattgaatgagagttgtatgaaaattgtaaccttcataaattatggatatctactcttataatatatcattatatttataatgtatactatatgtttgtattttaataagaaaattatcccattggatttgcgatcttagatctaacctactaaacaaatattccaacgaagaggaacgagatgagaacgccgttctaacctacgcaatatcaatcgtttcttcgctgctactttacgcctcaagttcctacccttcaagtttcatcttcaacgatcaacccaacgattaacccactgcaccaccttatctcttgttggtgtcatctaatccatcttcttcctgcatcttctgcaaatgctctcaggttctttcctctctcttgtgcacaaactgatcacccatgttgttcgccggaaaatgattcagattcttcgtatcttgcctgcattgtctttgactataatatgattgaaattcacttgttgattggttttcaattgttaattaccgttggttttgctgtttagtgatagtatattatgaggtttttgttcgttttcgggtttttggatgtgatttcatcctatagaatgaagagtatgcaacgtatgctgtcaccttgcgggggaaatggtacacgtggacccgaaatggagctaggttttgatacgtgcagtttgagttttggttttgggaggatttggcattcgttatatgaattttgtaattaactatgccgtttgattgttatttataacggtgcattgctttttgaggtttagaatttggacttaacgcctctttctattcatggttattggttttatttcttcctttttgttgactgagattggtcgtagaactcgttgcctgtctatgttttaatgttggcctgattttgaatttctaatccatgactaagtatttctttattgtcttgatatagttgattgaatcatcaatc

SEQ ID NO: 149 - DNA, 2000 bp, Cucumis melo
  source 1..2000: mol_type = unassigned DNA
cattttaaattgacctttcatgaaaaatcgtatgtttttggtgtgattttgagtataataaaaatgattttaaccatttgaaaatcactttaaattacacctaatgggtgactgaggttttagctttcgtctttgtttagctctaaatttgcatggcaagttttccattccaatgattgatgtggcttgtaatagttgaaatatatatatatatatatgaggtatcaaaatccccagccttgtgttaggttgaatatggagggagtggggagttatttttcctgctcttaccccgttcctaattcccaccttgtttactatgtgttattgttattgccatatttactatgtacataatatttcgattagaaattttattgtttaaccattagacaattttatatgtctaaaccataggtttgaacaaaccatttagattatatatatgttgacaattagattgatagggcaattattttgtttatcctaaaaatggtaaataatgttcttaaacttggttctttgtgaaataccttcaactttcaaagtttttaataatattcttacgcttataaaaagaaaaaaaggataagttgaaaaaagaatacttctatgataagttttagatggaaactatttactttttcatttaaaaaatacttttcaaatttatgaagttccaaaagtatgacttaaagaaatagttatacccttattgataatatacgacaaaaacaacgcaatatttcgttacaaaaataaatctagctgcattactatcttactttaaagatactcttatcgtctatctaaactaccttactctagaattaataattaagttccttttactttataaatataacttattcctactattagtatatatttatattggtatctaatagctaattttgaattttgttccaaaaaaaaaatatcgctgagttttgttttgaagtctttttttttttttaaatatatattttcgattaaagctagatgttgcagttgatatgtagatttaaaagaaatgtgtgagatcgtttataactatatagaagattaagcatttattacttcaaaatatatcgttaaaattattcacataaccaatttttactcatcaaatattatgtcagagaaaagaaaaacgaaaaagaaaacctacttcaacggacaaagaagtccttagttcaaatcttcaaacctttatttgtattaaaaaatggcatataaattttttcaatttttacgcattacctgttgcgtgaaaaacattgatttaatagaaaagaactgtcctttcagttttgtttttttaaaaccaatttcgaaattcaagaatagaaacaaaactttaagtctagaggatcactaaaatctatcataaggctagaaatacatcttgtaatctgcagtaggcatttgccgggatgacaattttctggtgcttggattaagaaaaaagaaaaaaagaaaaaagaaaaaaaaatggtgaggacttagaggccataatgagtttggcattgggcccacagtaggatgagtaaattataattgggagaaaatgagcatagggtgtggaggggaaaaggagaaggctaaaacactatcacaaatcacacagtagaagatacacagaagaagtaaccacagccattcattgagtgagaggctatccataatctcatcctcttacccttctcatcattcattcaaagccattcaactcaacatcccactcttagttaaccaacaaaatatatatacatccttctcaatttcccttctctctactgctttaatcttttgcttcttcttcttcttcttcttcttcttctgctttctcaataccctcaaccatggctacggctactctatcagtagccaaaccatctattcaggttcctccattactaaacaccatcctctttcccttccactcttctttaattttttgtatctgataaacattactgcattttcttgcatagcag

SEQ ID NO: 150 - DNA, 1680 bp, Cucumis melo
  source 1..1680: mol_type = unassigned DNA
tttttatgaagggagttgttattttcctttgggatttggagggatatgatatatatcctttttttgcaatttgatgacagaattctgcttttagagacttttcaaactgtttcgtaatgaatttgatgggttgggggtggcttagttcaatactttgtgggttgaaaattttgatttgcaataaatgaaagccaaaaatgtggggaagctttcagttcaagtaagttaagggaaaactgcagaatatctggcttgaaataagagatgtcttcgaaggttaatagttttacattgacttttttaaaaaaaagattatattataagtacaaatatgggtggatgtgaacttatattattcaaagagactaatataagttttgggcgcttaatattttatattttcatttagcagtcaaagatgtataagaaaactttggtaatgcattttatactagtttatttatgtaggatgtaggatctatcgaataatacaacatatttttaaatgatgtgtacaattgtgaaaaaaaaaggaacatacagtattgtagaaactaaaatattttctaagatatatcgagatgtaaaaaaaatgaatggatgtcaattccagcataacttaattgttgaactaaaaacaaaaagaagaaataaagggggcaatggtttgatcctcatgccccacatgaaagtcaaagttatgtaaaggttccgtgtaggatatccttcctcctaataaggggagataggattttatgagggtgccaacagctcagaattccaaattcccaaaataccctcttgcttgaaaatttcaaactcttctgtttttgccttgtgtaccattcactattccgatgcgtacagttcattaaccacacaagttctccttttgcaggcaggtttagctaaacttattggacttgctggagagaccaatgttcaggtaagatcttatttgttataatgaactcacaaactaatttagattagccaaagaattctgtttctgaagaaagagaggatgaaaatcatctcataccaaatttctttctttttttggaattatgtcttcacatttattcattttccttgtcaacagggtgaagagcaaaagaaactggatgtgctctcaaatgaagtctttatcaaagctttggtcagcagtggcagaactgtaagctgctatctaatcatacaaatgacacgacaaaaatatctggtgacttactctaatagttgacaaattggtggcagtgtattcttgtttctgaagaagatgaagagccaacatatgtcgagccatctcggcgtggaaggtttgttttccattcttgatgatttttgtctaatgcttacaattatcatcagtatcaactcctcttactttgttttaattttaatgttatttcttcttattttccaatgacaaaggtattctgtggtgttcgatccactggatggttcctccaacattgattgtggtgtttccattggaacggtaacatccctatgctaccttctgaatgagatttcaaatatttttggtataatttctttccaataagctgagtgtatgattgtttgaatatctactttttcatgtagatttttggaatttatcacttgaacgacagccacgaacctaacctagaagacgtcttgcaacctggaaagaat

SEQ ID NO: 151 - DNA, 1524 bp, Cucumis melo
  source 1..1524: mol_type = unassigned DNA
tatatatatatatataggtaatgagtaaagaaatgaaaaagaatgagttgaagaatcacacccttaccattctatttgaaactcgtgagtcttgtagacttttacatgtcttctccttcacttaatatcattctggattttgattatatgtatctttatttctaaacagcttggacagatttattattgttagaataccttgaatatgttttctggtgcttagaacgatcatacatgggtttttctagggttagaggagtgcgctatacataaactttctagttctagaggcattgctgtaatcttaagtttaacagtttctctttaataacaaaaactgctcttcccctacggtttaagttttctccttatcttaacagttataattatgaaaaatgatggaaccaaaacaaagttctgttaaaatttgactaattgattgaatgaacttttgtttccaagattcttaatttgtaaagtaataatgttcttacaaatcattattttgatgtctgagttataacccttaagcttggtggttcatattccattcaggtgaagaagattgtgagtgaaagctgttcccaagaggttttagaagtggcgttaaactccatctcatccctaattaccatcctctcctccatgtcatcgtctaccaaactccattcttcactttgatggtataagaaagtgaattagatttgggattgagcttcaaaacatgtatgatgatgtgaatatttactcgtgtaaataatataacatgttgtattcttgcttgtttctctttgctcatcttcgttttgttaagagcaaagaaaagcttacgagcatgaacatgtgcaaatttatgaaggtcaatgggcttcgtaattttttttccccattgatttaacgatttatggaagatggatatagtaaatttaggttaagctgtacaaaaccagagaattttcattatagtaaatactttacaattttcaattagctacaataaacaccgtttcaaaatctccctcatttgctaccatatttactattcgatatttatcattttttttattcctgttgtaatgtctactatttttcttttaaactattacaccacaaacacatactattataattcaaattaaaataatcactagtataactcaactataataactcagatgatttcattccatgaaagtggtaattataaatatttaatatcttatatgataaggataactatctatttggtgaaaccaaatcacaatgatgcagtggtaagtgctttggactttgaatctcttttttatagtatttcattcttttttcaacgaagcagcatcatgggccttgaatggaggccagctagaacgagcccattacatttgacagagcatctcttcggcccatgagcccaaacactattcatcttgttaaaccacgaacaaatcgagactgccgagagtgtaagagaattgagtaatttttttcgagacacagggagtttagagagtaagtcggagaaca

SEQ ID NO: 152 - DNA, 1851 bp, Cucumis melo
  source 1..1851: mol_type = unassigned DNA
ttgggtcgttacaatatcactgtttaaacttaagtttatttttatttatttttttattttttaatctttttcccttcttcctttcatcttccattcatttatacaaaaataaataatgagaaaattacttttcacttttgagtttaattatttttaagttctaaaatctacattttaatctttaaatttaaaaaaaaaaggatttacaagattcttgagtaatttattattattattattttgaacgtaaatataaccttttacaaaatctaaatgagtgtttgggacaatgagttgattattataagtattgaattataataattttttgtggggtatagactattttaatttgaagaataataggtacgtgtttgaaatataaattatgttagttgggaaagaaaatagtaaatatcgtagaaaaaaataaaataaatgaacaataagaatataaaatatggtaataaattgggactttgaaataatggtaataattaattaattaattgaaagctacaaaacaatgttcacttcattgctatagttctaaacagactaacaatctcaatcaatgacctaatgggtcaggccattatattgggctcaaatagattttggcaaaacgaatcgaaagcccaatggggcctatattatgtagggccgaaatgaatttcaacgaaaggaacccaaagcccaataggcccaaattgagacttacaaaggcgcatgttagcatgaagagagaattgaaagcttaaacagcgccatcacaaaacatttgcattttcgtgttgaaatcgcatttgggccgtaaaccaatgaaacacaaaacaaacaaatcctggaatagcctcaacggttctggaagaagaagaatcttctggaacctccaatcccacaataaaaatcaaaccctaaactcttacattcagctctttgcttaccttatcccaacaaaccttcaccaacgctctaccggaactaaaacccctccgacctcccacttccgacttacgacctctgttgcctgaacatggcgtctgccaatgctctttcttccgcttctattctatgttcttctcacaaggtacttcacttataaccccctcatttcttccttgtatttttcacaattcctctttggaaatgatgatatctagattgtagtagttgggattgtatgttaggtagagattttgtggagttagctgagagcggctgagaatactaatatatcgtttccagtagcttacgttgcgtttttctaatgttgcagagcttgagaaaggtgaatcaaacgcagaacaacagagtaaattacagacaggctggtagtagatttgttgtgagagccactgcaaaggagatagcattcgaccagagttctagaactgcacttcagtctgggattgataagcttgctaatgcagttggtttgactcttggacctaggggtaactttctgtttatatttatttatgaattggttagtattggatgttgttctaatattgaaatccctacaggatatattcatcacatttatagattcgtgttatggttatgttgagaaatttgggttcttcacataattctcaatcttgttgtgatattttgtatttgaagggaggaatgtggtgttggatgagtttggtagtcccaaagtggttaatgatggtgtgacaattgctcgggcaattgagttacctgatcccatggaaaatgctggtgcagctttaattagagaggttggttttttatactttgttatgaagcaaaattttctcatctatcgattattgaagtcttattagttcttacattgcgttgacaagtattctatatgtc

SEQ ID NO: 153 - DNA, 2000 bp, Cucumis melo
  source 1..2000: mol_type = unassigned DNA
actaattaatagtaatttgtatgggatatatgtatatgtgtgtataacaggaactacagagatagagattcactttctagaaataaagttgactgccattggagtttatttggagccttcagttgtggagcatttgcaacaatggaagggaaaagctgctaaggacttagtggaagatgatgacttctttcaggctattgtttctggtatctccctagttatcctatttttaactatactatctcatcacattcctaaatgtgaattacttgacgatctgttcaaacatatatatttcattgtttgatcgtaatgtttcatatttatgatgtttcatatactacctcgtcacacgtgcaaaggatttagatccgttcaaacatatttcattattggatcgtaatgtttcatatctatgatgtttcttatactatcgcatcacacatgagatccattcaaacatatctcattgttagaaagattatacattatttcaattcaaatagctctaaccaatgacaaaattagattcgtcccgtttagcttattctatatatatagatagatagatagatagatagtatggatatgcttgtgataagtgtttttttcttcttttttttttctttttttgttttttttctttttttgtcactttctaaattatctatctcacagttagctagttggcggggtgatgacttttggtgtgtcagtctagtgagaagtttgggggttatttttattttcgaaagcttcctaattgaatgacttgtaaaggttaatgtttatgtttttgtacatgtttttcatgaactattggttttacaagagttacaattctatttatttgtgtaagaaagatcatatcacatttttacccctggtgtgttcgttttatgttcttgatttgctttttgtttttcaataatttacggggaaagagagaataaaattttctttctccgatctccgcattcaatttttttttttttgaaaggtgcattcaatttttttgtgcttattaaatattcacttacatcttttgttttgtttatttttttattttcatctttcttatatgaaaataaaatattttttagtacaacaatagaacctcttgttaccattgaaatgaattacaggaaattaaaacttttactttttatttgagagaattaaaagagtagtttttaaatataacaaaacgactttcgcaatagatccagatgatcatttattaacaattttctaattaaaattgttactaaattttaacaattattaaaaaatattaattgaaaaacacgtgtatatatataggaacattttcaattatagccaaaagttataattatttactctataaaattctttagagtctatttaacctttttgttaaattttgttaatagttttactttgccattcataaaaatttctcatattatatacagtgagaattttataagtctcaaaagtcaaagatttgattaaaaaaaaaagaaatgaaagcatatctaaatatattatttatactttgaaaattacttccgaagcaaaatgtaaaaccgttataagtgaacttagaatccaaaaacatatattaaattaagtttaaattatataacaacacctttggattttgtcattttctaaaataccttttatcatttcaataattgtaaaatgagtcctaaattttcacaaatgtttcaaaaatatttggaggagacaattccttgagaatttcaaagatatattaaagaggacgtattgacccaaatcttttgttctatgtcactatgatcacccttttatatcacaatttatttccatctacaattctaaagaatttataatttaaaagtagtttcaaaatgtttctaaattttcgagggtaatattttaacttttggaagtacggaaagttaatcaaatctttgtctcaaaatctcaactaataactggaattgggaaagctaaagtctagaccttaa

SEQ ID NO: 154 - DNA, 1288 bp, Cucumis melo
  unsure 284..287: note = unsure at all n locations
  source 1..1288: mol_type = unassigned DNA
cgagaagtacccggcgttggtcaccggatttttcttcttcatgtggtgggtttaattgttggtcacgtgttttgcacggcgaaggccgggtggtaattattacgttgcggcaagtttgagcttggttgtgtgaaattaccgtgttgtcctttctgttttgtaggtactttttgaacgtgattttcaatatcctcaataagaagatatataattacttcccctatccatagtatgtatttccaatttacattttcatccctgtatttttcttcttcttcttcttnnnnttttttttttaatattgttattaatatttgtttacgttccagttttgtgtcggtgatccatttagttgttggggttgtgtactgtttgataagctgggcagtgggtcttcctaagcgagcagtaagtcaactctttctatagcccaatatgccaattttgtctttttctttcattaaaattgttatttttaactttttcatacccaatttagtttttttagtctgtttattagtcttgttttcttcaaatttagtagtattggtagtctaattggtagggctgttttgaaagtaattacctaatataatgagtatttagattgagacagtactatagtctaaacgatgtcattgcagttttgattgaaattttttctcctttatttatttcgaaaatgacaatataacttctgtaatctttgtaaccatgtttatttgaagctacgttgtaaaggggaaaaagaaaaggaaagtgtaaaatggtcaaataaattatatttttaagtgaatagattatataatgtgcggtaaaatatagcactcacaagtaaatgcaacttagtaatctaaagattgaactatcaattcatgaatttttttataattagcatggttttctatagtttttgggagtctgtttttcaatgaaaatattgccagtatggtaatcttgtatgacaatgtattttctaaagtatggatataattaaattttctttaatttatcggcttaaacttttcgttgtactaaaaatctaggataggacgtttaatatttgaacctttgtaatgccatttaatcaccgattaatatagatctaactattgaaacttcttcaaaagttttaatccaacggatggctaagagtgttaaaatattgatacaattaaatttatcgtagcttaagctttgacagtgtcaaaagctaattcagttattttctctgcccttggtataagggtaactctgttctctctatttcacacaagtgattgctaac

SEQ ID NO: 155 - DNA, 2000 bp, Cucumis melo
  source 1..2000: mol_type = unassigned DNA
ttatgttttattaactccatagttttactaaccccatttcacgggaaagtacaataaaacatttgtgttaaaaaggagttgtttgtatagatcgaataaacattttgtactaattccaatcatattaccaaatgttttaagagcatgcgttcttgtgtgcttgaataggggtgatcatcaattgagtggcgtcagatttaaatttaaagtggcaccaatcatcgacttgttggtttagatcgatcggtagttgttgtttgtgggacaatcatagttatcatttttctccttctatatgaataatagtcaactagatagaattgacatttcttagtattaaaaaaacgaccactgacctgtctatatcactaaactgcttgacctaagtgagtttggttggacttgattgcttcgttgtggtctaattcacttaaccctacttgaaagtaaaggtagaatataacatgattctttccaaattggcaagttgtgacttgatttatgcatgcacgaagattcttccactctccacctaactagtactcgatcacattggaaaatggatctgtcccgtgaaggatcgcaatattacatgccttcctacttttttcttttcttccaccaaagaaaaagaaaacgggacacacaataactatacattatcaataataattaaacgaatcatcccaccaaatgtaaaccatgaatattgaaatcatcatgttttaagaatcattttaataattatgttatttcattgttttatatagaacacaccgttcatgaaatcaaacaaagagagagaaattataattttgtaacaaattaccaacattctttttctttctatttttcataaatgagcatatgtttgtgtatatatacatactgttatttgatctccacattgttgaacaaaaaagttggtgttcttgaaaatagctatcaccgaaaatacgtcatatactgggttgttatgtaccaaggcccagaagaaagcccaaaatcaccggcccatgagcagtgaacaaataattgggctaaaagcccaatatacgtgatgatgggccaacccagagaagtttatagttatgttattatagaattccagatcagggagtatcgaaacaaaagcctccattgtcctcgttctcttctccttgtgctctctctctctctctcttgctcttttctctttctctttctctccgacgacaggttcctgaagctcgaccagccaagggcattgatctaggtgagtccgctttcacttttccactttcctctgccgtttttcttgtcatttccaattctccattctttgttctggatttcacttctttacttcgtcgttgattagaagataatagtgagatcgaattctatgtctcgcataccttcagtttcaaggaacaagacaatgattcaaccgcgccgtccacgttatggatagagggttttgattctcacctttatagctgcataacaccgttcttagggttcggacctttgaatctgcgatatttctcacactgttttggacgtttttaccgttttcctatggttctttagccttaccttatcttgccttcagatcttcgattgcggatctgattcgttcatttctacttgttactttttcttggaagtcgaggattataaatcaacaacaaagcattcaaaatctctagtgcaattagtgttttccatctagttattggagatcgtttgtagctttgattttgtccactttcttattttgaacgtctggaagacgttttatacatgttctttgggtaaagttgcgtttgggcactgttcttcacctctgggttttcgttcttatgctatgtttcatgatttcttttgatatctttgttattgtttccccatcatcgagtatctgattcttattcggaagccgtcttcttgaagctgcgaaccggttttctttttctccctcatcaagtctttaattttacaggaaagcgctgaataag

SEQ ID NO: 156 - DNA, 2011 bp, Cucumis melo
  source 1..2011: mol_type = unassigned DNA
ttgttagaggagttagcttttcagggagtcggttacaacttacaagacagacaaccatactgtacaatcaatcactactgcctcaaagtcgaaaccatttaataccagttgagcctcattgtgactgcataattttgacccctaccacgaggtaatttcagttcaaatcaattgtatctctcttctggatatcagtcaagaatctcatgttctcaagttatagtacattgataattactgtgcatttttattaatttatgagttaaaatcctgctgattaattcaactaaaggaataaaatagttattggcatttggatgagaatagaatgtgaatcccatcattcatcgctagattggaccgtaattataagtgagagggagaaacttctgttgctattccctttttatttcttaattcatttataaattgtttttaggccttttatatatatatatttctaccatttttacatttaaaattcttttaactttattatgtatggactcaaactaacaagctttatttgataaaattgttcaaactattatattagttttatatttgtaaaccataaaacaaatccataaaattccacctgcatcactcactcatgttgttgaatggtacgaaataaacaatacatgtgaaagatgaaaataagaattgttctcttattaaatctaaaatctagattttctttttagtacatttaacacttcaatgaaaggtctaaaacattattgaatgacgtcacgaactattacaaaagattattccgatttatctcaaaaggggtctatttcactaattttggtgtcccacatctgtaaagagaattttcgtgatatgtgtagatatttaaatataattgaattcgatgaaagcaaagcaagaatcgatatccgtagttattttgatatagatcggtgataaataaaagacaatatgcataaagtttgtgggtacagagtcggctagttgcaatgaggggtatggccccaagacttttccccaaccccaacggtcaattaatcaccccaatggggcatcgaatcttccccaccattttccttttcttcgccgactcttctacccatctcttttgccgactctttctcacaggtttgattaaatcccattcatattcagatacactatttcaaaatactcgcaaattaatttgtttttttaaatattggtataataataaaattaattataaattgtgacctaaaaagtactttgtggaagaggggatatagtctgtctaatatattattgattaaatatataatagaaccataaatgcaaaccactagaattgttaataaaaatcaacgctcttttaagtctttccatagaaagaaaggcaaagacgcgcaacgtctagaattttctttataacggaagctaactctgttataacggccgtccacataatatggtagatttaaaatgacgtcagcatgtcgcttctaaatttgggtccaaaggggggattcatttatagggaagggaaacggcaaatgcaagcatagtgagtaaatacgtggcggcaaagtaatggataattctgcctctgccgtgacgacattgcccttccatttcactataaatacacctcctctatccctcgatttcttcgcaattgaatttctcttctcatctctgtcgtagcaaaccaaatcgatttcttcaaaggtatttcttcctttcctttttttttttttttttttttttttaaatcatgttgttcaaactttgagagatgaaatgattaggggctttcaaagtggttttcgtttgatatgtttcttagatcgatagggtttagaatcgagcatccttgtaggtatcctgaggtttggtggttggatctgcttaatttttatgtggttgcatggaaaattgggattttttttttctaattacgtgattctggaaatattgatctgtggttcagatggaattgaatctgatctttctgttttgttctgtataggtgggc

SEQ ID NO: 157 - DNA, 1698 bp, Cucumis melo
  source 1..1698: mol_type = unassigned DNA
ttgttagaggagttagcttttcagggagtcggttacaacttacaagacagacaaccatactgtacaatcaatcactactgcctcaaagtcgaaaccatttaataccagttgagcctcattgtgactgcataattttgacccctaccacgaggtaatttcagttcaaatcaattgtatctctcttctggatatcagtcaagaatctcatgttctcaagttatagtacattgataattactgtgcatttttattaatttatgagttaaaatcctgctgattaattcaactaaaggaataaaatagttattggcatttggatgagaatagaatgtgaatcccatcattcatcgctagattggaccgtaattataagtgagagggagaaacttctgttgctattccctttttatttcttaattcatttataaattgtttttaggccttttatatatatatatttctaccatttttacatttaaaattcttttaactttattatgtatggactcaaactaacaagctttatttgataaaattgttcaaactattatattagttttatatttgtaaaccataaaacaaatccataaaattccacctgcatcactcactcatgttgttgaatggtacgaaataaacaatacatgtgaaagatgaaaataagaattgttctcttattaaatctaaaatctagattttctttttagtacatttaacacttcaatgaaaggtctaaaacattattgaatgacgtcacgaactattacaaaagattattccgatttatctcaaaaggggtctatttcactaattttggtgtcccacatctgtaaagagaattttcgtgatatgtgtagatatttaaatataattgaattcgatgaaagcaaagcaagaatcgatatccgtagttattttgatatagatcggtgataaataaaagacaatatgcataaagtttgtgggtacagagtcggctagttgcaatgaggggtatggccccaagacttttccccaaccccaacggtcaattaatcaccccaatggggcatcgaatcttccccaccattttccttttcttcgccgactcttctacccatctcttttgccgactctttctcacaggtttgattaaatcccattcatattcagatacactatttcaaaatactcgcaaattaatttgtttttttaaatattggtataataataaaattaattataaattgtgacctaaaaagtactttgtggaagaggggatatagtctgtctaatatattattgattaaatatataatagaaccataaatgcaaaccactagaattgttaataaaaatcaacgctcttttaagtctttccatagaaagaaaggcaaagacgcgcaacgtctagaattttctttataacggaagctaactctgttataacggccgtccacataatatggtagatttaaaatgacgtcagcatgtcgcttctaaatttgggtccaaaggggggattcatttatagggaagggaaacggcaaatgcaagcatagtgagtaaatacgtggcggcaaagtaatggataattctgcctctgccgtgacgacattgcccttccatttcactataaatacacctcctctatccctcgatttcttcgcaattgaatttctcttctcatctctgtcgtagcaaaccaaatcgatttct

SEQ ID NO: 158 - DNA, 313 bp, Cucumis melo
  source 1..313: mol_type = unassigned DNA
tcaaaggtatttcttcctttcctttttttttttttttttttttttttaaatcatgttgttcaaactttgagagatgaaatgattaggggctttcaaagtggttttcgtttgatatgtttcttagatcgatagggtttagaatcgagcatccttgtaggtatcctgaggtttggtggttggatctgcttaatttttatgtggttgcatggaaaattgggattttttttttctaattacgtgattctggaaatattgatctgtggttcagatggaattgaatctgatctttctgttttgttctgtataggtgggc

SEQ ID NO: 159 - DNA, 2010 bp, Cucumis melo
  source 1..2010: mol_type = unassigned DNA
tagaaaaaaattgaagatcttcttaatgctaataataaaagcatatataaaaaggcttcatattacacaagcatcttaaaaccaaaccgaacttgatttgaaattatcccactaattctgtagatttcaccaaagtccttaacctttatactaacatctgcatttgactctttcatttgacctccaacatattctttctcattttccttccattcaccacaaaaaccaacaaatacaaaaaaccacaaatacatagaaaattaataaaaaaatcaaaatttcgagatgaaatcattataaactcatccgataactttgagatttgaaaccttacactatataaagaaactcatccgataactttgaattcgcatcgaaattacgtaagtgaagacgaaaatgtaaatgattagatgcgaataacaaaaaaaacataatttctaaacgtaaaacactatattcaccttattatacttgattattttggaaaagtatgaaatttgagtgtgggagaggagccaaagaattggaaacttgttattaggagtcgttatgaaacattttcaacaagccaatattctttcacatactataatgatatacatagaaataatacaataatatttttgaaattgaggcatttttgtcgtaatttatctaaaaatgtcagggtagattcatcatgtatacaaatctctcgctatcaaaactatataaaaatcttgtgaatgatttcaatcgaaatggaccgagaaaaaacatcgtaaccacctctaaaatcgataaatttgtttcaatttcaaatccctaaaactaaaggtgctactttgtacaatttcccctgattagggtgctaaagttaaaccctaaataaaggtgtgtacgtttccggaagtttctagaatccccagcgaagttctccaaatcgtggcttgcagaccaaggacccccattaaaattcgtttcttcctctaaacctcctcccttaattttggcattttggtattttggctcctataaattcaccccctccttatccctaatcctttgtcttccaaattttccttcaaagcctgcttttcccatttcgtcgtgctttttcttcatctaaaggtatatttcagttctagttttctttctctgttgatctcttggatttgagggacgtttgaagttggctttgtttaattctttgttattcaatctctttttttgttagagttgttgtttaatcgtttcccttgttgtttttctcccttctagttcgattttagaacgctttttgtgggttgattttaatttctccgttttcttacatctttcacaaagaaacgattgaaatcgtgtttgttttttttcccacggcatacgttattagatcttgtagataatgatctcaatctattgtttagtttttgcaaataagaagttggttttttatctccaacttttatatattcgattcgatgagatgttctacaccgttaggatggaaccaagaagtgaggtaagggtgtttgattgaaaaattgaactgagaagttaaagttccttcctaactttttaatggattgtataattcgttcaattccttgtcgttccatttttatttctgtttcgtttttcgtgttgctgcgtatcgcttcccttgttgttttcctcccctattgattttgcgtttcttggagtttctctgttttctctcttcatttttctacaaaaatcaattctatttttattcgttttcaattcccgagctccttggaatgttatccttttctcctgtgtaaataagaacccgtattcaatcccagttcatagtttggctttcccaaataagagcaaaaagattgtactgagaagttgaagatttcaaaattttgtacatgatttcttctaatttatcaatttgattggactttttgtatatagatttggttcttgagctatttatgttatgacgttttcatattgaggccatgcgttgaattggtttcttaacaggtgggc

SEQ ID NO: 160 - DNA, 1107 bp, Cucumis melo
  source 1..1107: mol_type = unassigned DNA
tagaaaaaaattgaagatcttcttaatgctaataataaaagcatatataaaaaggcttcatattacacaagcatcttaaaaccaaaccgaacttgatttgaaattatcccactaattctgtagatttcaccaaagtccttaacctttatactaacatctgcatttgactctttcatttgacctccaacatattctttctcattttccttccattcaccacaaaaaccaacaaatacaaaaaaccacaaatacatagaaaattaataaaaaaatcaaaatttcgagatgaaatcattataaactcatccgataactttgagatttgaaaccttacactatataaagaaactcatccgataactttgaattcgcatcgaaattacgtaagtgaagacgaaaatgtaaatgattagatgcgaataacaaaaaaaacataatttctaaacgtaaaacactatattcaccttattatacttgattattttggaaaagtatgaaatttgagtgtgggagaggagccaaagaattggaaacttgttattaggagtcgttatgaaacattttcaacaagccaatattctttcacatactataatgatatacatagaaataatacaataatatttttgaaattgaggcatttttgtcgtaatttatctaaaaatgtcagggtagattcatcatgtatacaaatctctcgctatcaaaactatataaaaatcttgtgaatgatttcaatcgaaatggaccgagaaaaaacatcgtaaccacctctaaaatcgataaatttgtttcaatttcaaatccctaaaactaaaggtgctactttgtacaatttcccctgattagggtgctaaagttaaaccctaaataaaggtgtgtacgtttccggaagtttctagaatccccagcgaagttctccaaatcgtggcttgcagaccaaggacccccattaaaattcgtttcttcctctaaacctcctcccttaattttggcattttggtattttggctcctataaattcaccccctccttatccctaatcctttgtcttccaaattttccttcaaagcctgcttttcccatttcgtcgtgctttttcttcat

SEQ ID NO: 161 - DNA, 903 bp, Cucumis melo
  source 1..903: mol_type = unassigned DNA
ctaaaggtatatttcagttctagttttctttctctgttgatctcttggatttgagggacgtttgaagttggctttgtttaattctttgttattcaatctctttttttgttagagttgttgtttaatcgtttcccttgttgtttttctcccttctagttcgattttagaacgctttttgtgggttgattttaatttctccgttttcttacatctttcacaaagaaacgattgaaatcgtgtttgttttttttcccacggcatacgttattagatcttgtagataatgatctcaatctattgtttagtttttgcaaataagaagttggttttttatctccaacttttatatattcgattcgatgagatgttctacaccgttaggatggaaccaagaagtgaggtaagggtgtttgattgaaaaattgaactgagaagttaaagttccttcctaactttttaatggattgtataattcgttcaattccttgtcgttccatttttatttctgtttcgtttttcgtgttgctgcgtatcgcttcccttgttgttttcctcccctattgattttgcgtttcttggagtttctctgttttctctcttcatttttctacaaaaatcaattctatttttattcgttttcaattcccgagctccttggaatgttatccttttctcctgtgtaaataagaacccgtattcaatcccagttcatagtttggctttcccaaataagagcaaaaagattgtactgagaagttgaagatttcaaaattttgtacatgatttcttctaatttatcaatttgattggactttttgtatatagatttggttcttgagctatttatgttatgacgttttcatattgaggccatgcgttgaattggtttcttaacaggtgggc

SEQ ID NO: 162 - DNA, 1235 bp, Cucumis melo; identical to SEQ ID NO: 22
  source 1..1235: mol_type = unassigned DNA

SEQ ID NO: 163 - DNA, 617 bp, Cucumis melo
  source 1..617: mol_type = unassigned DNA
aaattttaataattaaaatgaacaatttttcaagagtaatagagtttgagagatgtcagagaagtttgaggaagaagataacaagtgggagaagagaataagtttgttgtgtgaaagagaaggggaaatttcattcaagggtatattgaactttttactcaaattttgtaagtctattttttccgatcaatcctaaaatcacacacacccttaaaaaatggattatatttggcaattttccatgataaactcatttttaatttagagttattttttcaacgagatattaacagttttagttcatatactaattgtaagaatagtttcttttaagttgaatagaatttttgaaacttttaatagttcaaaaggtatttttgaaacaaaataagaatgtttttgaactttttataaaaagaattgagatttttttgaaatttttgataaagagaaaagaaaagaagaaagaaaaaagaaaaacaagtttgtagaactccgtgggaaaatcgtcgagggccctgtgaaggaattttgaaattataatgagggtattttcgtcaacaagggaatttagacatcgtatataagcatcctcaaaccctataattaagcccttc

SEQ ID NO: 164 - DNA, 54 bp, Cucumis melo
  source 1..54: mol_type = unassigned DNA
aatccaattgccattctccatctctcgccgcaagggtttaagagcagcttctct

SEQ ID NO: 165 - DNA, 545 bp, Cucumis melo
  source 1..545: mol_type = unassigned DNA
cctcaggttggggtttccccctatcttcttcattcttcctcttctcgatttctttcttctatttgctcgatagtctcttatttcttgagcttttgctgtttttctcctgtacatcctaacatgaattataacttggttttgattttgtcttttacttctgtattaaacaacttttcttacccttttattcttctcttcttcttcgtgtccctgcccttttgtttttatgctaattttatgtttctgtttatcaatctatcgaggcgtgacctgtcgttcttccaatagcgtagatctgcacttaatctattctagctgattggattggtcgtttttcgtttttttaatttattttctctgttctagttccgataaatttttttatatataattaacaagttctccagccaaaagggttaatattgcgttggatattttaatttttacgttatttagatgtgtgaatctaataaaattagggttattcataaatttcagtaatgatattttggttatctgttcttgctgttcctgtttcgcagttc

SEQ ID NO: 166 - DNA, 19 bp, Cucumis melo
  source 1..19: mol_type = unassigned DNA
ttttacctaatattcaagc

SEQ ID NO: 167 - DNA, 2003 bp, Cucumis melo
  source 1..2003: mol_type = unassigned DNA
cagtgtgctggaattcgcccttatccaaggagattaatgtcgagagattattatcgaggtttgaatttattttgtccaatcatatgattccaagagctgaccatcaattcaacagaacatgaaccggaacctcatacctattgtaatggttcacagcatcctaatacagaacatgaaccgaaacctcttacccattgtaatggttcacagcatctttatacgtattataggtagtaccattgaagatgcatttaaatgctgtccatgctctgttctctaaaaagttggacttggacttggacgtcagctgaaagtatgaaatgcactgtagccaacgaagctatgttttcaggcttcaacatggttttaggaaagtggaggctctttggttgaagggttgaatgaatgcttttctaattccagcatgatcttcaaatttcgacacaaaaagcttaagtattttgttccgttattcttttaatccttgtattgttatatattcttttctctgaactgaatgtacgatgattgcaggggtcgagagcaagtccgatataatgaaacacgtaaggacgtgattgaatgaaaaactatgagcagagatacaaagtctaacttacgggatgaacgatgagaggtttgaccaagagctgtgacgcctgtatatttcaacaaaagttgatgactaacatcacatgtcagagtaatcaaagaaatgcagccgcacatatatatatctatatatatatcgagttttttttttttttttttttttttttttttttttatctaatatattttaatctattttcctctgccctcctccccctcctcttcccccacccttcttctgcacatagtagccaaggattgatcggtttcttttgattcggggggaaaatgttgtacaatttttgcttccatagaagcttgaaagttttgcagattatgttgtaaaattacccttgtgtactcacactagttcttctcgtggaaacttatattacaatggttgagttttaaggggcatattcacactggtaactaccattttctaatttatgaatgccgagtttctctccatgaaagacctttcaaatgccctttcctccgcggtgcgtttgttgttgtaaatgtgcagtgtcgttggatacacgattgtgtgaaagggaaaagggaatacgattaactcttaaattcaacccctatctccatcagtatcaatcacatttcagcaactagctcttgaataacattgagattcttgtttaatccacgtactactactactattactactatttgacagttgatatctcaaataacatccatatttatcaaattggtattttaaggacttttaatttcttcgtacatatttcattataatttaactactctgaccatcattgaaaatttcacaaagaagacattttaaattgaattgagttgaattaagttgatataatggttgaacgttggatttaatttataatttagtggtgtatgggtccattgtaataattcttaaaaaaaatatcatattctgaattctaaagaaccatctaagaccaaaactaaggggtcaccaatgagtatggtaaagtcaacaaagtttgtctacttttcttatccttatcatcaagagtgcaatatgatatcaaagataaattgtacgtgggcgtcatccattgggtaagaccaagaagcaaaatatcatagagaagttgttttagtagccataggaaggaaggaagcaaaataataatatagatttgaaattgtggatgataaactgccaaatgggaattcaaaataaactaaataaataaaataaaaagagaaatcttgggagtttccattttagccaatgaggaaacagatagagatctcatcaagataaggaccctattctcttcttcatctataaaacaaaaacaaatcaaaccctcatttcactcattcaaaacaaaaagtactccaaagtcaaactaacaaatacg

SEQ ID NO: 168 - DNA, 2004 bp, Cucumis melo
  source 1..2004: mol_type = unassigned DNA
tggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtccttgtaccgttgagcttcgccttctaatagagctctggttcggttggcgtattagctcgaattctttctctcttccagatctacgctgccgatttcatcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagagg

SEQ ID NO: 169 - DNA, 1067 bp, Cucumis melo
  source 1..1067: mol_type = unassigned DNA
tggatcgaccatgacattcaaaaacctttaagatatggatcttataaaataaatgtaaagggtaaacaattcttccttgcttaacccaagctatatattttatgcactaatttaggtattagagtatattcagctgaacaccacctaccaatgctagtactttaatcagtcaattctaacttcgataatatatctcaaccaaattagtgaaaaagagtcgtaaatgaaaaactatgtaccaagatattctatttgttttttttatgtttaaatatctcaaagataatacctaaaacgttttctcctcgtacaaagattcctcatttactttttattgtcgtaaactctaatacaataaactaaaacaagtacaaatacactagctttagaaatctactttttattgaaaccaaaaccaataattcaacatttcattttcaccgacaaacctttgtaaacaattgaagtaatttttgttggtactatgaatagtaacatcaagtcttcaagtgcatcatatcaaccaagacatgttcttaaaagcgacactaaaagatttaaaaccaaaagcatttatgaaatccgaacttaatcaaatcctaaatatttttcacttaaaaaaaaaaaaataggaagaaaaattgacataaatgggatattttcgttttcaaactggcaagccagcatgcaccacgttgttgacgtgtccttccacgtcggaaaaaaaaatattaccacagtaaaaagagaataaaatgaaagtcgttgactctcccttagtcggaggaagcgcgtgaagctgaagccggattagaaatcggcaataaccccgacacgtcatcgaaatgctagtatcaaatattgtccgttggatcttccttcaccaactctatttgaacggccacgatcttccaggtccaacggttcggaagaatcttttcgaaattccatggctagtccctacactcctccctattggctccctagggcatcccgaccggttattccggttgccgggaaggtggctggacgctataaatacccgctttgttcatctcgtagtcctt

SEQ ID NO: 170 - DNA, 92 bp, Cucumis melo
  source 1..92: mol_type = unassigned DNA
gtaccgttgagcttcgccttctaatagagctctggttcggttggcgtattagctcgaattctttctctcttccagatctacgctgccgattt

SEQ ID NO: 171 - DNA, 845 bp, Cucumis melo
  source 1..845: mol_type = unassigned DNA
catcaggtttgcgagctctgttccaccatttttcttttcctgaagctttgagcatgcttgtgattcttcatttcctcatttctttgatggtttatgaaagaatttaggggaattttctctttttgtattctagtggtactggtagatttgtttgaagtttgtttctcttcttctgagaagtgaattcttccagatctgacagttgcttttgattttttctttgggaattagtgaatgatacttcgatactgttttttgctctctgagattctggatctcgggccttggggttttctattgtcttttggtagctatgtttcgtttgtcagcttgtatttgtcattgttgaatggttcgatccggtttgtaaataaaataaattttgtaggcgcacttgttttccacggttttcgtgttacggtttcatgattccctagatctctggttagaactaagttttttgtcggtaattggatttggtaagggactgttactgtggttgaattgtagatccagtcatcttctacatgagtgtagggttccttagggcagatcttgtgttttataattttaattttgttgtttccctgattttgaacctgtttggttgttcagattcgtcgagtcatttccattcattaaaagtttctataattttatttgaatcttctgaatctgtgcttgtattacccagatttctataaacctatcttgatttcaagtgtgctatgtggtaactgttgatattttcaagcttaagcaatactgatgtgactaaaacttaactaatgaactgaatgttttttgtacacgaactaatatggtgttttgttatgtttcagagg

SEQ ID NO: 172 - DNA, 2018 bp, Cucumis melo
  source 1..2018: mol_type = unassigned DNA
actattggagacgacgaaggaaaactctaatgtgagcttagaattgaagatggtgaacaattgggagtcttttttaaaaatctttcgtcggtatattgaaatttccttttacactcaaataaccccccttttcacgcgttcaacgtcttcaaaccttcctttttcaattattttttcgtttacattaatgacatttaggtaataagataaattagtggattcttttgtgaaaaatgttagcccatttaagacttttatgaaatatattagaaaaaaatcatatagcaatttatattattctagttaaaagcccataatagaaactaacccaacctaaagtaacgtaaacattcaataagagagacaaattttaaaataatttctaattaaaaaaaaaattgtcaagaccgtccgggtcggatccaaaatataacccgaaccggccggtttagcatctatataaatacacctttagggtttttattctctcaagacttcaccgcatttccacttctctgaggccacggtcagccattggagcagctcaataatcctttgactccctactacggtaagtcgaccttactgctttcggcttctagttttttcaatcctgtcattagtcctttggagttcttctgtacatttatgacgttttcggctcgtgttttgtttcgcctgtatgtagtgggtttttcgagttttgtttttacttttttttatacttgcaggaattagttgaaatctatgtacttcatgccttggataatactcttgatctgttgtgttattcaaaaatgaattgttttaagatggtatttgagaatggtcatgtgagttttgcctacttggttattaaaatgaattgttttaggatggtatttgagaatggtcttctgggtatttggttggaacctttgtgctctgctatgaattagggtgttctccccgtttttttttttttttttcttttggttattaatatatcttttatgactacttattcatatatgatatcttttactcgtaaattttgactcatttgaaagttttatccttagtcctttctcattcagggtgtaaaggtatgttgttagggttaaaatagcctatgcaggaaagttctgtatttgttctaattattgcatttgtgtgcatttgtatctagtttatttcttgctgagagtatgcttcattttttagtacacatcacttgtgccactttattatagttgcacatttttgtttatggagaggatgaatagcatttagggatgtcaattttttattgagaaaaccctctctcctacttaagcttggggaatttttgttctaaatgtggtaaacataatacttcttcttattttaatttgaatggaaggggaagacgaatactaatattttcaacgaaccttcacaacttttttttcttatttaggaagccatgtttttcaaaattgtactgtgtgatccacatatttatcgattattagtgaatcgaataataattagagttttattggtataattttgaagttcagacttattacatttgtggaaagtttggttacaattttcaattttattggaatcctaagaactttgtgttaacatatattgagttttcttctctttttttttactcattaagttctctattaggaatgtttggttcaatgtcacatagtcgatagctaagaccagtgacccacaaagctatgattgaacgaaaaacaagcctttcacatcttggtaggaatttgttatttctcaatagatttacagagctgtttcatgtgatcacaatttttttctatttttctgaagttctctattaggaatgggctatctggttagttgcttttgagagaacatgtggattggtgttgctcggtttccttgcctttgtaattttgtccttggaaaaagcaaaatgattaggtatcctgatatgcataacatgtttaagccaactagttctcacttttttagtgcaaataattgatcttcaggaatcg

SEQ ID NO: 173 - DNA, 565 bp, Cucumis melo
  source 1..565: mol_type = unassigned DNA
actattggagacgacgaaggaaaactctaatgtgagcttagaattgaagatggtgaacaattgggagtcttttttaaaaatctttcgtcggtatattgaaatttccttttacactcaaataaccccccttttcacgcgttcaacgtcttcaaaccttcctttttcaattattttttcgtttacattaatgacatttaggtaataagataaattagtggattcttttgtgaaaaatgttagcccatttaagacttttatgaaatatattagaaaaaaatcatatagcaatttatattattctagttaaaagcccataatagaaactaacccaacctaaagtaacgtaaacattcaataagagagacaaattttaaaataatttctaattaaaaaaaaaattgtcaagaccgtccgggtcggatccaaaatataacccgaaccggccggtttagcatctatataaatacacctttagggtttttattctctcaagacttcaccgcatttccacttctctgaggccacggtcagccattggagcagctcaataatcctttgactccct

SEQ ID NO: 174 - DNA, 1453 bp, Cucumis melo
  source 1..1453: mol_type = unassigned DNA
actacggtaagtcgaccttactgctttcggcttctagttttttcaatcctgtcattagtcctttggagttcttctgtacatttatgacgttttcggctcgtgttttgtttcgcctgtatgtagtgggtttttcgagttttgtttttacttttttttatacttgcaggaattagttgaaatctatgtacttcatgccttggataatactcttgatctgttgtgttattcaaaaatgaattgttttaagatggtatttgagaatggtcatgtgagttttgcctacttggttattaaaatgaattgttttaggatggtatttgagaatggtcttctgggtatttggttggaacctttgtgctctgctatgaattagggtgttctccccgtttttttttttttttttcttttggttattaatatatcttttatgactacttattcatatatgatatcttttactcgtaaattttgactcatttgaaagttttatccttagtcctttctcattcagggtgtaaaggtatgttgttagggttaaaatagcctatgcaggaaagttctgtatttgttctaattattgcatttgtgtgcatttgtatctagtttatttcttgctgagagtatgcttcattttttagtacacatcacttgtgccactttattatagttgcacatttttgtttatggagaggatgaatagcatttagggatgtcaattttttattgagaaaaccctctctcctacttaagcttggggaatttttgttctaaatgtggtaaacataatacttcttcttattttaatttgaatggaaggggaagacgaatactaatattttcaacgaaccttcacaacttttttttcttatttaggaagccatgtttttcaaaattgtactgtgtgatccacatatttatcgattattagtgaatcgaataataattagagttttattggtataattttgaagttcagacttattacatttgtggaaagtttggttacaattttcaattttattggaatcctaagaactttgtgttaacatatattgagttttcttctctttttttttactcattaagttctctattaggaatgtttggttcaatgtcacatagtcgatagctaagaccagtgacccacaaagctatgattgaacgaaaaacaagcctttcacatcttggtaggaatttgttatttctcaatagatttacagagctgtttcatgtgatcacaatttttttctatttttctgaagttctctattaggaatgggctatctggttagttgcttttgagagaacatgtggattggtgttgctcggtttccttgcctttgtaattttgtccttggaaaaagcaaaatgattaggtatcctgatatgcataacatgtttaagccaactagttctcacttttttagtgcaaataattgatcttcaggaatcg

SEQ ID NO: 175 - DNA, 1999 bp, Cucumis melo
  source 1..1999: mol_type = unassigned DNA
ggtctcagacccataggaatagatcatttattgccttatgaattagtttatgagtacataataattgtcaaccgtatacaaatcaacatgaaagaatataatgttgtacatagtcattccaagtttactaatattatattttaggagtgttctacaccgaacctgtcacatgtacactgggtatgctacccctgaattcaatatcataaagcaactttaattgtcaagcattctcttgaccatttgtgacccatttgctcctactttttcaatcaataactatcacaaaaagctagataccacatgtgataaactcactgaaatcaggtatatggttacctgtgcttggtcagcactcaatcagattcgaaggcctagtctttgtatttccccccctctgcacactacaaatagtcctccacgtaaagacccataacaaaacgcaaaccaagtacagaaaatctagccgaaatccagaccactcaaacataacaaatcttctggtaagtttgaataaaatataaaactaacctatttaatcaaataatacaataaaatggaagcaactaacataacatatctaaatatgatcacgtagtaggaaaaaaaaaaacattccaaaactattaacaatcattcttaatggtatgggtcaatccccattatttaggactataacaagaattcctcatacctaatgccacatcctatgtccaaccctcgagattacctcgtgagtaatcaatcttattcatccttatttcaaattatgtgaaatttctcatcaggttgatcatattgactttcaatacaacttatgattaatctttcccttgatataatttcgtatgaaaaggaagttgacattatgtgattttctcataaggtaaaccaagtaaacttgacatgacgtcttaacaagtcttggtttctaagtgtaatttactgcagaaaaaatcctaaattctatgacttttcctatgagattgaccaaatcaactttacgagaaatcttgggaagccatacctacaaagtcttcccccaagaaattacaatttctagtaaagattgttgaaatttaccctccaatttttccgtgaaaatttgacaaacttgtaagaatatcaaatttgggttggatattgacattccaaaataagtagttttaaaaaggatttatccaacaataatagaagaaaaaagataggaaataacatacccacgtaaatggaatgtaaatatttatatattaggtgtcttgaacgaccgtcaaacgaaaataaattgttcatccgaagttgaaactctttaagtgtacatttatcttttcgtaagaataaaatgtaaaattaacgtgtgaaaggttgggttaaataagttatgtagagataatattgaagatgatagaataatcacgatcgatgaattagtatagtcccagagcggatttaaacctctctcactttcatgctttctatatatatcaaaataatttcaagtagttggtttagtcgtaaaaaagtcaaccaatctcttttagataaaccttgagttattaaaaaattagatcaaagataatcgttgaaattgaaattttaagagtataattataacaaattggaaagttctaaagtagtttttgcaatattctccatcaaaatagagtagaaaaatattttagtaattttcttatcttaattttagttttgtaatagttattaggatggtcctaagttctcaatccgcttttagtccataaaaagaaagaagagagagaaaaaaagtccccgatccgcgacacataccaatccaaccaattatgcacaatccatgtgatatcgaacggtcacaagaataaatgctttctacacacggatcaccatccaacggctttccttccatctcatcctctatataatctaccaactctgtcatcttcgacacacttcaattatctcagcttttatttcatcggattttccatcaaacaaggcaaca

SEQ ID NO: 176 - DNA, 2004 bp, Cucumis melo
  source 1..2004: mol_type = unassigned DNA
tatagtttgtaacaatctactctatgttctttcaattttgatacatttgaatcttaaactttattgagttacacaatatagtccttgtatttttaaaatttataatgactctatttatattaatattatagaaatttttgttaaggtttaataaaaatttttctgtataaataaatcgaacacgaagtctatatttagactgcaatatagtaaaacctgacatctaagtttggtgaattttgttttgctttaaaaactaaactattacaattttaaaaatattttaatttagttaatgcacattaactttacggagtaaatttttacaagattgaatatacatagattaaatagttataaaaccaaagattagagtaaaaaacatttaaatagaaagaactaagatttttttaaaacgaaaatgatactagatacatatatatgtatctatattataattactcattttaacatatagttttgaaagaacaaagattagttgcatgtgttgattgtttttaagaaggaaataatttttgaatggaaaattttcaaaagttttaaatttgacaataaactcatatttaaagtgtactacaaattttaacttttggttaaactccttgtttagttcaatcatgtaataaattctcattccaagaatcgttttagaaaattttattgtgcatttaataaaatatagaacatatatggcatataaaaattgattacttttttctttttttgggacgaaaaacacattagatataatcttttttgaaagtttatgaactttaaaaatgggttattttatacggtggtcaactttattttattgaaattattgagtttataaagattgttatatcattttcttcttctctttcactagaatacaatcaaacctatcaaactctctatgacttatttagaattctttttgttatatttttgaaattaataaatgaaaagcttagagtctaaattataacaattaaaattgaaaattttgcaataattttatttttagcaaaatgacgtttggtttttggggattgggaatggatcgatactatcccgattccggacaaagaaaccgacccgagattcgaattttttccattcccaaacagagcacttaaaatttaagcaacgttataacggcgtcaccgaactaaacggaaaaatatgaagaaaattagaaaaagaaaaacggaacagtcaaacgttacttcacgtcaatggcaatattcatttttttttttgtttaaataattgaatttaattaatttggtttataaaaatagagtcctcatatatcgcgaatgcgcatttgatcgtgaaggacagcttctcccttgtgttcaagagagagagatctatcattcttatttggggccgatctctctattctcctctcttctattccgtaagtttttctcattcattctcctctctcatttctctccgagatctgtttacaatccttttgattttcatttttcctgcttcgatctgtgctcctggtgattcccttttcctgttttatcttttgttgatcttggaattgattgttcttttgtgggttttcattgatttgtattttctgatctgggtttctgttttctcgccttgatgttttgtatttggatctgatctgacgacccttttttttttttttttttatttgaattgcttttccaatgtttatacctggatttttattgatgcatgggtttaaccgattggttggatgcgttttctttgtgctggatctaggtgtccttgtttttaatttgaattgtgggtaaaaatggcattattgtaatgtgtttggagtttgattttgaatcttggctagttgatttttgaattacaaagatcggatcctcttcttttttgggttgtcttaagatttttggctggtttaagtatttgatgtcgttgtattttaaggggtaactgatgccggcttgttgtgtttgtattcagtttacttgaaaa

SEQ ID NO: 177 - DNA, 2005 bp, Cucumis melo
  source 1..2005: mol_type = unassigned DNA
aattagcaaattgtatgtaacaacgtcatgaagaggcatttcatcgaacaatttaatagcagagtcaagatggcccagttttataagtttattgatttctcgattcttaaggtaaatgagatcgcgagcatggaaatgcagacccaaacgagaatatgggtgtggtaaatggaccggtgatgttgtggctacaaattcggattttacagcagtaatagttctgacgaaggaagcgaatttagtgacctttcgcatcacagtttaagcttatcagagacctgaacaagggctctagctctacaacttagaaaggtttgatatggtccgtgatcgggagggaccgaataacaggcgcttaaattgttgttcataaagaagaggattgtcgttgatgtattttaaccaagagatgattagttatccacatccacagagagaacagaagacggctttctaaatctacaccgataaaaataaccctaccactttgtttctttagaaaagggtcacattctttaaaaacattagcgtcgaggattaatagggtatattgactaatgctctgtttggatttcgagaaataccaatttacaattgatttcaaattaattatgttttgttgttgcacgaaagataaaaagaatttaaaattcaaaaggatctcaaatcttatttttaacttaaaaacttttatgacccaaacggtttatgtatgatttaaaagtagaatacctctgtgaattcttaatttttttttctttccaattaccacataaatatgaaattttaaatacatttattttaaattttatatccgaaacaaaataataatttaaaactatttctcatatatgaagtgtgattcgatctaaattataaaataataaaaatttacatctagttttgattatttttttttcgttagatactaaattgttaagaaaataacatttttaatccaaagttttgaagaatatatgacttttaaaatggtatttatctttttagtgtctgatttttaaaaaatggatttcaaaagttcatcaaatagcattgtatttttattttaaataattttgacatttaaaattagagtaatggtttataaaagacacttgatctctaaaactattttcttagatataaatacgtatgattatttttaaaaatcaatcaaaataggtaaattgtaaaaaaaaaaaaaaaatcataaaacatgatagtagttgtaattatgctctcaaactttcggttatgaaaaataaacattttaacttttagacgtgtcaaagttgagtcaagttggaccttcaaagttatgtagttatataaattgtaatatatgtataagcttgtggattcaattttatcatttatgggtccaatctctacaattatcgtaagtctatgggtcaattgtaacacatgtggagtttaagagctcaattttggacgtggatgtgttttgcaaccaactccacaccttaaaaaggtgtttttttttaatttatcaaaaaacaagaatttagaatctttaagtttatctttaaaaatcaacggacattttgaaaaccaattgaaactactgttataaacctaacaactaaaagtatattttttaagaccgaaagcataaatccataaaaaaaaaatccagaactgaaaatgtaacttttatagttgaaaatttagctaaattatacatattaaaattcaaggaccatataaaattaaagtacctgattaaataataacgaattaatgtttggtatttttaacctacattagaaaaaaaaaacaaaagaaaaacggcatactatttgtcaagcgtccgatgggaagaaaatccaacggtgagtgttagtattgaaatacgcagttctcgtgaatgagcctggcttagatttgggaacaagagccaacccctttcgaccgagaagccgtcgtcttcaccatattcgcctcaaccattcgatagccacgtttgaagaagaataggattgcc

SEQ ID NO: 178 - DNA, 1978 bp, Cucumis melo
  source 1..1978: mol_type = unassigned DNA
aattctaacaactccggaacaaataatttagcatggattgaaatataaatcttcttgacttgcaaaaaaatcattgtaatggtcttatgttggttatagttagggtatcgaaacgccatacaggaatatgggattaaagttaacttttgttcatcaatttcagcttatgaacttctaaaatatcaattttacctttgaacttatatgttattacccctttcgattgtggtatgttaattaatatctgaatctcagtccttatgaaacttttttatactgtcacaaacatatgaagttttattgtaagttcttagaaatcatctaaaaagagtagtttgttggactatttattttattttttcttattaagttgttttcacgccatttcagtaaaataactatagtgaatagagaatcaaacttctaatcttaagttaaggtagtagggtatatgctaattcaataagataatccgtgatgcttgacatctgacttaattgttataagttttaaattttttattgtaatatttaaaatactagtttttggtttctaataaagaaataattgaacaattacaaatatttatacaaaattaaactagaatatatgatcattttccttcgtgttagaaaaagggaaatatatgtgtgtatttatacatattagatattgttttactatattccattttcctcacgggaaatggaggattgagtgggagataaacattgtccccaagagaattgggaatggaaatgcaaatgacatggccctccacaaaattgttcgcctaaaaatgggctttctcacttctcactccgcaagaaaaatatcgtttcccttcgaattcgggcaagatctcaaaaccacatgtttttctttctttatttttcaagcctacattatttataaaaatataacttaagcagagaattatgtaaattcaagtccatttttcgcttcacttagctaaatcattaacaaatctgtaattttgttcataaattagctcaccaattatgttttagcccactaaggcccattagacatttttattagaaaaacatgaaccgttggatcaagatgtgtgttttcttttctttttctttttattttttttgggttttggtggatcaattcgtagctttagcaacctattattatatggagggaaagggcgtattaatctgttagcgccgtccgggagtttagctttcttccccgagcctcggtcttatcccctaactccaaaaccctagcccaaaggtaatccactccttccccctccgctcttcatctttttctattcatcatctttaatctgttctcccttttggttcttagattcttcttttgttggattcttttaatctttactcatggttggccttgtaagtttagacgacgtttttatacattggttaatcctgcttctctatctattcgcacgctagggttttcctattgttttctattctgctctacttctgcaaggttgtgttcttcttcgttcaggtccctttttttaaccgaaattaaattaatgcaaattcgtttgtgcttctaattaggaagccttttggaacatctcgacattttgattgctgcatttcatttcgggtatatttctatgattgaaggatgtgggtctgttcactgcatggtcattacttatgcagctatgcttatcgagtccattatgtttgtgcaatctgtttccggattcataattttttagtaattgatcagtagatgaaaaaagatattgtaatattccttgagtgttgcaccagtcttggtgggtatctgctcctgctctttgcttgtggattttacttttattatatctgtattattcgaaatgttctgttcttgttataacttatacccgaagatgtgttcctccccgcgtctagcgttgtgggttacttatgatggacatggttttgattctgtttggtttgtgcagggtacc

SEQ ID NO: 179 - DNA, 1263 bp, Cucumis melo
  source 1..1263: mol_type = unassigned DNA
aattctaacaactccggaacaaataatttagcatggattgaaatataaatcttcttgacttgcaaaaaaatcattgtaatggtcttatgttggttatagttagggtatcgaaacgccatacaggaatatgggattaaagttaacttttgttcatcaatttcagcttatgaacttctaaaatatcaattttacctttgaacttatatgttattacccctttcgattgtggtatgttaattaatatctgaatctcagtccttatgaaacttttttatactgtcacaaacatatgaagttttattgtaagttcttagaaatcatctaaaaagagtagtttgttggactatttattttattttttcttattaagttgttttcacgccatttcagtaaaataactatagtgaatagagaatcaaacttctaatcttaagttaaggtagtagggtatatgctaattcaataagataatccgtgatgcttgacatctgacttaattgttataagttttaaattttttattgtaatatttaaaatactagtttttggtttctaataaagaaataattgaacaattacaaatatttatacaaaattaaactagaatatatgatcattttccttcgtgttagaaaaagggaaatatatgtgtgtatttatacatattagatattgttttactatattccattttcctcacgggaaatggaggattgagtgggagataaacattgtccccaagagaattgggaatggaaatgcaaatgacatggccctccacaaaattgttcgcctaaaaatgggctttctcacttctcactccgcaagaaaaatatcgtttcccttcgaattcgggcaagatctcaaaaccacatgtttttctttctttatttttcaagcctacattatttataaaaatataacttaagcagagaattatgtaaattcaagtccatttttcgcttcacttagctaaatcattaacaaatctgtaattttgttcataaattagctcaccaattatgttttagcccactaaggcccattagacatttttattagaaaaacatgaaccgttggatcaagatgtgtgttttcttttctttttctttttattttttttgggttttggtggatcaattcgtagctttagcaacctattattatatggagggaaagggcgtattaatctgttagcgccgtccgggagtttagctttcttccccgagcctcggtcttatcccctaactccaaaaccctagc

SEQ ID NO: 180 - DNA, 715 bp, Cucumis melo
  source 1..715: mol_type = unassigned DNA
ccaaaggtaatccactccttccccctccgctcttcatctttttctattcatcatctttaatctgttctcccttttggttcttagattcttcttttgttggattcttttaatctttactcatggttggccttgtaagtttagacgacgtttttatacattggttaatcctgcttctctatctattcgcacgctagggttttcctattgttttctattctgctctacttctgcaaggttgtgttcttcttcgttcaggtccctttttttaaccgaaattaaattaatgcaaattcgtttgtgcttctaattaggaagccttttggaacatctcgacattttgattgctgcatttcatttcgggtatatttctatgattgaaggatgtgggtctgttcactgcatggtcattacttatgcagctatgcttatcgagtccattatgtttgtgcaatctgtttccggattcataattttttagtaattgatcagtagatgaaaaaagatattgtaatattccttgagtgttgcaccagtcttggtgggtatctgctcctgctctttgcttgtggattttacttttattatatctgtattattcgaaatgttctgttcttgttataacttatacccgaagatgtgttcctccccgcgtctagcgttgtgggttacttatgatggacatggttttgattctgtttggtttgtgcagggtacc

SEQ ID NO: 181 - DNA, 2000 bp, Cucumis melo
  source 1..2000: mol_type = unassigned DNA
aaataatttgtggattttatcatattatgtaccttagactttgtaaggtttataacacaagatgtggagaaatcccatgatgaacattggacgttattatatcctttgaaactaaaaacaaaggaaaaaagacaaatggctgagtataagaaaaagagaagaaacaaccaaaaagctaaaatgtcatgctctcatatgtaacatatttgaattgggattgatttcaagatgtattttaaaataatttaacacacgataaaataattctaacaaactcaaaattactcttaaacatttacttgatatcttcgacataatgacttttgttttaatacaactcaaaacataattaaggttggttttaaatgacaacaaacgaaaaactcgattctaatttcaatactagtctttgaaagccctaaggagcgtggttttgaattgtatagcaaggtttcgaaaattttaatcatcaaacaataggtatatttacctgttcctcaagtacagttaattcatagctacttgtcgtgcttcctacgacaacattgtaagacttgccacacactaattgaagccgttgttgaaggtagttttccatgtatagcttgaatcgacggatgaccaaagaggttgaagaaggtttgaaaaataggggaagggatatacaaagtttgagagtgagagagggagagaaatagaaatagaagagactgaaaaatgtaaaagaaaggatgaaaaaatgtggggtaaacgcaaattggatttttatagtagtattttgaaaatgctatagaaaggctatgtatagtagtgcttccaaagttctatatgaatgagacaaatcaaaatatattttttttgattaattaaccccaaaaagactcataaaaaaatcttataaatcccactaagatattgcatgttatatgtagtaaaatttatcgttcaaataaaccttaaacataattcaaacgaagaaagtaaaaatttgaatttcttatcttacatcattcaaccaaacaatttccatataagagattaaacttctaactttaagagagagatatccagcatatgcaacctaaaccaacagtgactttgctatatatttgcacaaaatgtgggggacaaagttgtaatttcggaatatcaatgattaaagaaaaggtaaaatttaaaattcggaagcttgacgtggcaacacggaatggtgatgatatttccaactcctcgcgacttttagaagttggcctcaccaaccgcatatccgcccctttgccacgtgtcagactacaacaacttccaacaatttcctttaagaacaccaaattatatcaataaatatttaacttaaattaagaatatatttgttacaattttcctactgagttagatagatagacagacttgtcaattaactaataagtccaaagtcaatttactcaacatagatacaattctaatttgagtgtgaaatacatattcaaatcaaaattattattaagaggaaaaactgatttgctttctcaatttaaaatataatattttgaaaaagaaacacacatgtattatggttttcaatatatttactttcttagtcacctaatctcaaactaatttttgaagaaattaaatatatatattatcatttttattttcttggttatgatattggtatagaatcaacaaaactacaagtgtcctctcctctgctatcgatggggattagactctaaacttgtttagcgattagtaattatatattagaaaaagtttatgttaatgtggacccgacaatctcaccaataggctcttcacttcaccaacccccgacccattccctctaataattcgacacggctcatccccggttcgaaccgggccgacgtcttcctatttaacacacctccatttcctcttccctccgcaacacaaacagagacctcaccggaaaatcaagttaaagcaaaaccaaagagaagcttcatcactctccggaa

SEQ ID NO: 182 - DNA, 2000 bp, Cucumis melo
  source 1..2000: mol_type = unassigned DNA
gcatcttatggatgtagtcacaacatatttatatggattttctgataatgatatttatatgaaagtcccaaaaggatttaagatacctaaaacatataaatcaaattcccataaactatgttcaataaagttacagagatcattgtatggattgaaaaaatcatgacgaatgtgatacaatcgcctgagtgaatatttgttaaaaaaataatatcaatataattcaatatgtccatgcgtttttataaagaaatcaccgtcaggatttgctattataactgtatatgttgatgatttaaatataattgaaattttgaagagttttcaaaggcaatagaatattaagaaagaatttgagatgaaagatctcagaaaaataaaattttgtcttgattttcaaatcgagcatctagtaaaagggatatttgttcatcaattaacttatacagagaaaattttaaaaagattttatatagataaaacacattcattgaacattctaatgcaagttcattcattaaatgtgaagaaagatatttttcgacgtcgagatgataatgaagaactccttagtccagaagtaccataccttaatacaattggtgcacttattttgtcaataatcaagaccagatattgcattttctataaatttattagctagattcagttctccaacaaaacaacattggaatgaagttaaacatatacttcgttattttcgaggaacaattaatataagattattttattcaaataaatcaaattttaacctagttagttttgcatattcttgatttttatctgatccacataaatctagatctcaaacaggttatctattcacatgtggaggaactgctatatcttaacgatcagtgaaacaaattaccataacagtcaactcttcaaaccgtgctgaaattcttacaattcttgaggcattcatgaggctagcggagaatgaatatggttaaggtcgatgactcaacacattcgaaaattatgtggtttgtcttctagtaaactccttccaacaacattatacgaagacaacacaacttgtatagctcaaataaaatgaggttatattaaaagtgatagaacaaaacacatctcaccgaagtttttctatactcatgatcttgaagaaaatggtgacatcacagtacaaaaaatttgttcaaaagataatttggtagatttatttacaaaattattacctactgcaacctttgaaaaattggtgcacaacattggaacgcgacgacttagatatctcaagtaatgttacatcttacttgccaagttaactatacatagtgacatttggtggagttgtaagaaacactaatattggagaaaaatcgaaagaaattggaaaatatggagaattgaattttttttagatttttcttattttctaattttaggtttccgtattctgattatgcctcattttcacaacattaataactttaataagatgatttcttgggttaagggaaaaaaatcatttttttagagttgcacgtacaaaaatattatcataacatatcgattataataaaccaattcaccgtcaacctaacctaggtagagtttgagttaaatgttaaaagaatatccacccctcaacattgtaatcccaactaataaatcagcaacctaaagttttttttaaaaaactaaaaagaagagcaatatattttttttactattatttttttaaagagtggatttatttattaaattaaaaaatgaaaagaagaaaatttgttagtttgggtaatccgaaaacccgattatttgggcccgagaaaccgacgttttgtttattgttcctcacggcaataagtaatggcgtgaatcgaccgcgtgcgcttcaagctatctagacatttttatatcctccgattagaaaccctaattcagattctccgtattacccaccctggaacatctttgaaacgcgaaaaggtgacccgaagaaacttgaa

SEQ ID NO: 183 - DNA, 1989 bp, Cucumis melo
  source 1..1989: mol_type = unassigned DNA
attacttgaacttaatccacatttatgtctttatataaagttcgaatactcataatatagccaagaaaccttgtttattggattttgagttttatcataagcaaatctcttatccaataacaaattattaaacaacacttcaacaataactttattcaacaatatattagtttaacattcacaaatcacgagtattagaacataaaacgcaacaaagaatggactaaggtactatattctaacttaggttgtttaggatttccatatgtcaatgcttttgtgatttttgaactagattttcttgttagattaattcaattctatttttaaatggcttaatatcttattttcggatgcttggggattgctagactaccgctttgttgaagcaataagttaaatttgtttgttacaggtattgatcaatctaacatagaattgaatttgtatgaatatttagttagacgcttgaaaactaattattctaccaatgagccgtagatcttaatgcaattgttattaatttgaactttgtatgcttctcatcgattaaatttatatcaagtagttaattaggacaaatctattggctttttcatttaattttgttaagtaaaagcaacttagaattttgaaaatgatgaacccatgatccaatacattgaaagagaattttgtttaactcaaactaggattcttctcacattgatttcgtataatttaactttttcaatttatatcaatccccccagggtgaaaaaaatttgtttgaagaattcatgtgctttctaaatctgatctagacttgccactaaaattaacttttgatatgtaatttggttaaatatttgattcggatttcgacgacaaacaattgatcaatgtggtattaaattctgatctccatgtaagaaatttacacattttcataagttcaatgttgacacaaagagagtaagagcattttaaaaaaaaagatacttttaatcttttctaaaaaaacaccaaaatgccattatgtaaatgtaacctaaataataaacatttaaacttagaattcatgcaattaggctttgtatgggacattgaattgattattaaaatcagtagttatagaccgtgagttataatggtttgtattagaagcataaattattttaattttgatcgtaatagcatgtatttgagatataaattaatttagtttgggtggcaaatagtaaacagtaaagcaaaatataaaaaaatgaatttaaaatagtaagatttgtaacaaatgattaatactataacaaacgtggttttaaaataacgttgatcgtagctaattgaacattatttattgtaaaattgagtgtttttaatatttggagcctcaaacttcgggtggatcaccacaatataatcatattcaaatttaaaattttatttttattataaatattgttaatagatgctcattatgggccatctgtcactccctccgtgcatatcctacctgaaacatcatatatcttaaacaatgtccattgccatgtgtcactatttttacatcccatccacttgacaaatatgttgaagatgcctacttttttagggatcatgtaatctatctcatgcttgtcaaattgttcgataatagtgttacaaaaaatttagtaattattattattatatttcttcgatatttatgcttcatatgccattgtgctctccatttttaccatacttaaaaaaatttcttattataaattttttcaaaaaaaaatttactatatagtcatcatctttattaaaattaaaattgagaacctgatatttttgatattaataatttaaaatttgaattaatccactttaaaattattaataatttattcgaatttgggccttaaggaagagatacggaaacaaaccctagatcccatctatatataaatcgccacaaaaccctacctttctctcagtttctcgttttagccggcaaaa

SEQ ID NO: 184 - DNA, 1463 bp, Cucumis melo
  source 1..1463: mol_type = unassigned DNA
ttttcttcttgatttgaaattcttcctccttcctgttgcaaacaaacccagatgaataatcagacaaaaaaagcagcaaatttgaagatatgcatatacgaagaagaagaagaaaaagagagggaaggataaggtaggagatagcttgagattacagcgtagaaaccgatcgaaccggagatcaacggcgcgaattaggtcaagaagaagtgagggtttttatgaagaagaagtgagggtttttatgtgccgatgaaaaaccctacttctgtgttggtgatctaacgttgtttgatcggttcggtttttttgagaatcgagtaccctcattattattattattattgttattattataagtttgttgtaagaattaataaattatttcaaaaattacaatttttatttatatatagtttaaaaaattttataatttttttaaataaatttcgaaatataaggttggatttcttaaaaatatatgaaaaaagagatgaagtttataaattaaaaatgaaataaaaatagtaagtttgtactcttattcttatttacaatttaattttccattaaaattttaaattaaatagaaatataattaaaatcttaaattagatagaaatataattaaaattttcagaatgtaaatttaaattagcttagtgtatatttaaaatatataagattgaaataattgattttgtttatctaaatattttatattattatttattgaataaatataattatatatggtaaattgttttggataataagaaagtaaagatggtatttatatatataattaaccaaaatttaagtttgttaaaaagaaaagttttcaaaaatatttttttacgagtaattaggaaaaacccacattttacatcgaagtcatagactgggtctatgtcttcattgccttgtcgtgtacccgatccacgataacgcattatgaaccgagtagatgacttaactttttgtaatagcttttcttctaccatatttttgacatttttttaaaagtaacattatttataaaaaaaaaatcgtagtttgatctcacatgaaactattattacatcattaactaatatatctatatttaatgtagttttcttgacatgattttaatgctaattgaaatagttacaatttttgtgtcccattttgtttagatcaatatgacttcacgtattatgacatatggggccatcttaccagaaattggtgccaatgagaaaatgaatgtaccttaaccaatggagcaacccatgtgagccattgatgaacccaactttcttggtttcccatcttctattcatatgtcacaataccttctcttttctcattctatatatagactctaaacaaacaactaatctccaacttcaaatctttcacatattctcattcaagcattgaagtttaccacttccaaaaagattcaatccaatttagcc

SEQ ID NO: 185 - DNA, 2006 bp, Cucumis melo
  source 1..2006: mol_type = unassigned DNA
cagcgatcttcgtagaaactaattcaatgctagctcattatttgacttttctcaggcaaggctccgcgaggaagagaaaaggtccaatttcaggaaccaagggcatggacaggttccggtcagaagaagctttttacgtaaaccctttgccagattgtttatgtcaaggagaattaccaaatggaagtacgacttccattttcagatagttcagtagaacatcaaagataaaatgctcttagagagctcaatgtatatgcaggcgaccactcaacgtgtcaccagctttgtacatccaataagccagctacgatggaacgacggagtgtttataacctgagttttggtagttggcggaggcggtgatggtggtatagaaggaaggtcgagggatggcaaaccctttacgccaagtagtggaagggagtagttggagatgaacacattttgagaagtttccaagatcactccatttgggggagaggggatgttggttatttagcacaattgttttcatgttttagtaattttatccaataatgagcgaggcattgaagcaattaaatttatttttaatgattttttcacccttccataggctttttcttttttcttttccttttagtttgcaaactttagctccttttatcggctgtcgaactcatttttgaagttattgaatgaaacacagtttgggctgtgtcagatgggtggtgaaattttatacattataattactacataaaatgaaatcatattgtaattttctatctatgccacaatttttttttattgcatcatgaggattaaattgtacgagtccaaatttgtacagtcatgtttttaaagctttcgagcattgttactaatgcatggaaaggatcgattatcaagtatcctcccaacttcatgaaagttattatttgtcttctaaatttgttttagaaaatgtttaattaattatttgagaagaaagtttaactaaatcctattggtttcctctaaggttgtcatacttatccaataacaattacgtttaaaatcaaaattattctaatggtataagactaatgttttaaaagcataaaattgatgaggaaggattggaagtaatactatttattttgaaggtaaacattcttgaatgtctgtcctaaaatcactaatgttttcttagtttgagactttgagtcgttgaacctctccatctttataaaatataatacgagtccttcacaataacttaaaatatatactaaatcctaattaatgaaaaataaataaataaaaggtacaaaatcattaaagcctaaaaatctattactcctttaaaacttttcaagggtccctacaaccaatgagaaactaccacgtcattttcacaatccgttcagtgtttagaaaagtcaaatcgcaccgtccatttatccactcgtaccaagtacggtaggaatctatctaccgtccgattaagcacaaagaagcacagtaaatgtcaatcgtgtccatccgccgccataccgcacatccttcgtccgaccggaaggccctatataaagtcctttggttttccgaatttctacttcattcgcttttgaaagatttcccaatctcttcgtcggccaaaattctctctcgcttctcaacccttcttcggctttttcatccaggtttgtttctcttctcttttttcttcctttgttgttcttggaatatgtttaatttcatttgtttttccattcaatttcatgctagattttacgattaggttgattttctgttcgtagattgtaattgatggttagggttagctttttctcccattccttctggaatctgtttcttgaccttcgaacttcgttgataaatctttagaaacatttacataaccaaacaataattgaacaactcgtgttgttatgcctatataatagcggttaggaaactggaaacgcccttataattgaaatcgccttagaaatttgttttgattcatacagggtacc

SEQ ID NO: 186 - DNA, 1664 bp, Cucumis melo
  source 1..1664: mol_type = unassigned DNA
cagcgatcttcgtagaaactaattcaatgctagctcattatttgacttttctcaggcaaggctccgcgaggaagagaaaaggtccaatttcaggaaccaagggcatggacaggttccggtcagaagaagctttttacgtaaaccctttgccagattgtttatgtcaaggagaattaccaaatggaagtacgacttccattttcagatagttcagtagaacatcaaagataaaatgctcttagagagctcaatgtatatgcaggcgaccactcaacgtgtcaccagctttgtacatccaataagccagctacgatggaacgacggagtgtttataacctgagttttggtagttggcggaggcggtgatggtggtatagaaggaaggtcgagggatggcaaaccctttacgccaagtagtggaagggagtagttggagatgaacacattttgagaagtttccaagatcactccatttgggggagaggggatgttggttatttagcacaattgttttcatgttttagtaattttatccaataatgagcgaggcattgaagcaattaaatttatttttaatgattttttcacccttccataggctttttcttttttcttttccttttagtttgcaaactttagctccttttatcggctgtcgaactcatttttgaagttattgaatgaaacacagtttgggctgtgtcagatgggtggtgaaattttatacattataattactacataaaatgaaatcatattgtaattttctatctatgccacaatttttttttattgcatcatgaggattaaattgtacgagtccaaatttgtacagtcatgtttttaaagctttcgagcattgttactaatgcatggaaaggatcgattatcaagtatcctcccaacttcatgaaagttattatttgtcttctaaatttgttttagaaaatgtttaattaattatttgagaagaaagtttaactaaatcctattggtttcctctaaggttgtcatacttatccaataacaattacgtttaaaatcaaaattattctaatggtataagactaatgttttaaaagcataaaattgatgaggaaggattggaagtaatactatttattttgaaggtaaacattcttgaatgtctgtcctaaaatcactaatgttttcttagtttgagactttgagtcgttgaacctctccatctttataaaatataatacgagtccttcacaataacttaaaatatatactaaatcctaattaatgaaaaataaataaataaaaggtacaaaatcattaaagcctaaaaatctattactcctttaaaacttttcaagggtccctacaaccaatgagaaactaccacgtcattttcacaatccgttcagtgtttagaaaagtcaaatcgcaccgtccatttatccactcgtaccaagtacggtaggaatctatctaccgtccgattaagcacaaagaagcacagtaaatgtcaatcgtgtccatccgccgccataccgcacatccttcgtccgaccggaaggccctatataaagtcctttggttttccgaatttctacttcattcgcttttgaaagatttcccaatctcttcgtcggccaaaattctctctcgcttctcaacccttcttcggctttttc

SEQ ID NO: 187 - DNA, 342 bp, Cucumis melo
  source 1..342: mol_type = unassigned DNA
atccaggtttgtttctcttctcttttttcttcctttgttgttcttggaatatgtttaatttcatttgtttttccattcaatttcatgctagattttacgattaggttgattttctgttcgtagattgtaattgatggttagggttagctttttctcccattccttctggaatctgtttcttgaccttcgaacttcgttgataaatctttagaaacatttacataaccaaacaataattgaacaactcgtgttgttatgcctatataatagcggttaggaaactggaaacgcccttataattgaaatcgccttagaaatttgttttgattcatacagggtacc

SEQ ID NO: 188 - DNA, 2003 bp, Cucumis melo
  source 1..2003: mol_type = unassigned DNA
aactagactagcgagtgcacaaccaaattacaaaatccttaacagagacaaccatctatctcctttaaagcaacaataacatcaaccgaattagaatccacaatcagtaaagacgatgccgacaccaatgaccaaaaccgatcaaatatagcttattacggaccattacttcaacagttacatcaacaaaaaaaaaaaaattaaacattgctaataaaatctgaaaatgaggaaaaagagattaaaagttttgaagatagaaagaataaatctgaaatgttctaatttgatatataagaaatatgaggtaatatgacgaaagcattttgatagttttcaccaactccctttgtgaaaggatacatccaaccaattttacaatttctgttcaaattttgtccacctacccttctcttctgccccccaaggctgctttctttcttttattatttgctaaattaccaaaaactattttcgaattaaaccatctatttcaattatatacgtcattcgaattttaacttaattaacattagtatatgtttcggatcaaggatagtggtataaatcatcctaatttcaatttgtatttagaaaagttcaattatacttaaaacttctaaaaattttatattttaaatttggatataaattaaatttaagatttatggaaggtaaataattagagcaaaacaaacttcaaactatatggaaaatagaaaaggaatattttagccaaacaaaaacacttattattttattttgtttttttgttttttttttaatttaacaatttttttttttattggttgaatgtgtttctccactggtgagtctccaactttgacctgcaaagggtctatatagcgagtttcacgagcacctaaccaatatctgtgtaataattcccatttttctttcatacccacttcatttgatcatctttttcacaaccccggatctctaattcttgggaatttgcctctttctcgatccatttccaccgtaattgaaaaatattcaggtttgatttcttctgggttttcattcaactgtctaacttcattatgccctttatgtgtttgttgaaagccccccacccaccatcgttcaatgcggtttctttaccttttgttcggtttcaacgatgatttagaagttatagatggatgctaattgtttcgttgttggtttgatccactgatctgcctttgattggcataaaaggagattctagatcttgttttgatgttgtgatttatggatattattgttatagtcgtggaagtttttcttgtcgttctgcggtatatggttgttttattttttgagtggtaaattgagcagattgtgaacttttgggttttatggtgaaagcatgaattagtaaatgtagagctgctgaaacaaaatggaggtttgctagacctctttgtgaattcttaatggtcagcctccatcttaagaggctaagtccaaaaatttaaggcagtcttttgttattgttacaaaggacaagaaataacagaggagttattttaattgaatcaagttggaaagaagtactacttcatgcttctttcaaaagcaggtcaaagtgctttaaagtcttcttatttatttattttttcctgaatcaatttaaactaatgatagaaagaagtgttttttaatgggttattataagtaacatcaatttttaaccattccaaaagttacatcaaattcatcatagtgtgagtttacgaattttggaagttgtaattttaagttaatacttcttttaaggaaatgtacactttgcatgttgtgttcataaggggtatttctttgacaaacgcagcaaccaccccttaatgaaaactacaccacggtggttggttttttcttgttatttttttacttggaatttacaataagttgttatattcggatatatggcaaagcagatatctgtttttatccgaaacctcataaatcttgaatgtgcagcaggtaaaaac

SEQ ID NO: 189 - DNA, 1024 bp, Cucumis melo
  source 1..1024: mol_type = unassigned DNA
tcattgatgaagaaggaaactttaagccaattcgacaacttatccattcaacaactttccaccttcagacattcagattcaactataatataacataaattgatagtcaagtcttttttgagacaaccataaatcatcttagcttcgagaactgtcacttccttaaattggtgaatatatcacattccatccattcaaaactttgtttcgaactttactgtagttatgaatcaataaattgggagagatattgttaaaaagagagagcatatttgtttctattatttactctctcctaagagagggttaattagtctataaatgatctattcttctcgtccattgaaattttgttatcctaaatttatgaatacttctacccaaaataaagactttttttttttttgaaaagtgtcaaaaaaacataaagaaattgacaaaacattcatttttagtggattttttacggacgtaaatagtttgtttttgtttcttttaataatacaatttttttttactttaaaaaatatttttgttataaaaccaccgtatttttattcaattttaataaataaataaatgaaagaatataaaaaagaggaaggaaaaagaagccaacgaaccaacggttgccacgtatcaaaggtctaaagtgcgcaaaacgaggccttcggaaaccaaaatgcgtggcttcaattggagcaagtaaacatggaaaccacgtccattgtaacgcttcctgatctcttctttacaaccgttggattcgagtactttttctcaacgattaacgactgagtggacctccacttgcttctgttccacgcgcgtgggattgacgtgtggtccacgcaactcttctcgataggatcattcgagaacatcctttacttaaaccgcctctctctgcctcaatttctcgtcacttccttctccttctttaccctttccactgcggctgattcttcttcgccttttattctctcgtacgccgccatattcttcacttctttttccggcgaca

SEQ ID NO: 190 - DNA, 2001 bp, Cucumis melo
  source 1..2001: mol_type = unassigned DNA
attcgtcttcgcattatcagtaaatttatcattttaagagtttgttcttttttaaaaaaaattaatcatttcgataaagttggagaattcaaaaatttctccaaataatttataaaaactttcggttatatatcgaaaaaattaacatggtattaaaacgatcataactcaattaacataaacactccctctcaactttaataccaaatttctttattaacgcaaaatttaaaatttgtttttaaaattttcacataacataatagaaatacttttctttatggcaaaaatacaataatcaaaattgattgatggtgacaggacaccacacaatatttttaaattttgaatatacgaactatataataagatatttatgagattcccatcctaaagattcctagagatttccttgtgtacaatattacacaagtatcttggaagtccaaagtcctgagaaaaaagctatgtataaagtaatagtgtttgtcgtaggaaatttacttcattcgtgtcattagctttttattgaaaaaaaaaattaggtatatcttagtgaatctcacttaatcgttgtcgatagttattcttttaatatcattatatactaaaatataacaatattgaaaagctaaaactgtatataaaaaaaatgttacctctaaacttttatcgtttatttaaaagataaatatattctttcaaaacttacaatcaacatcctacgactatcattataggtacaaatcttttcatgtttacacaaaaattagatttttaaatggtgtaatgatgatatataacgaaattttgaatgattactatttgaggttaccattgtaattggtcgtgttgtttgaaatttaattttattagaaaatttgtcaaaagtagcaaaaatgaataaactatttaaactttaggataaaatcaagtgttatgagtttttgtctagtttatatatttttatttttattgaaaacccttttcctatcttttcattacttcaaaatagttttaaaatgtctattaaggctaaagttagtataaataaaatttcggaaattttttttcgaaaaaaattgataaattatttatattttatattaaagtcaaaatttattacgcgtagatgtttatcaaattttctttctttttgttgataattttccaaaatttggataattttttaaaatagtaaaattattataaaaatgaaaacaaactatttataccttaagcaagaaatactaaaaaggcaaaaattcatttacttcatgaagcgtaaaaattaaatattttaccactttttgttattttttaccatctctatcaattatttgtaaaaagaaaactacaaaattagatgttttttcttttttaaggtttaatcaatattaaaatttcttaaattggcagacaagttggtgttggtaattacgaataaatcccgaattgactaaaaataaattcttctccaagtaaaatagacacgtggatgaagaaataagtgaatcaaaggcatccacagttcaataaatggaaaaaactactttctgctgactcattcataagttttcataaaatttcataagaaaggccaaagggcttatgaaagtgaatgtcatagcagtaaatgaagcacagcgccattgaaagacaactcaaattgcatgcaaacccacataattattcaacaaacccacatcaaatttcccataaagatcaattctttagggggttcaattacccaaaagtgaggtagttgaaaaccattaaacaacaagaaatcaacaattttgtaatttgtttgtacagaagtaagagataaaatcatcgttaaccattcctttatttcgtaatacaacccatcaaccatctctctctctctctctctctctctctcggcctttatctttctcttcctcaattatttaagtactacccaagtgagctaaaagcaagttcagtggacagtgttgtaagaaccactacagaaaa

SEQ ID NO: 191 - DNA, 4175 bp, Cucumis melo
  source 1..4175: mol_type = unassigned DNA
tagtttggttcataggttatagtttccaaatttgttaggctatcattaatcaaacacaatacttctcttgtaggatggctgccccctatagtacttttttaacttaggagaaggatataataattatattccttttagaaaatataataataattgtgtagtgctttgatataccttaaattagctactcacgtttttaggaggaagcttccgttgcttttcatggtgttatgatcttttttattttataaaggactgaactttaaaatttctctttcatctattttggattggattccatctattttatacgggaagtgaactctaagatttctcttcacctattgtgaatcggactccgtcatgtaggtcaagactacgacagataagaatagacttccacgaaagaaagtggtcaatcgagatggctatatttggctctttcagctcaatttcttcttttttccttgcatgttcttccgttggtacatttcttgcactttttttgttctcacatgactaatgtattccaagtttatcattggcattgtgcctcttttaggcttgtaaactctcgatccaaaattatctaggacatatgtttcctagtgaagaaatactagtatattccttatgtcaatatgtcaaaattttcaatttcttaacctttgagtaaatcaatattatatttttatggaggttatttataattggaaaaaagttacacccatctcaaccctaattaacaccaaatgaaattgtaccatgcggcacaatatttttttgtgagttttttgcaaagagaaacaaagtagcagacaaagaacaaacattcccccaaaaacagcagagaatacctaagagagaatgctctctcgtaaaaaataatacccaagaatcttcccaaaaagagggagtaaaagagtccaaaacaaacgaaccgaagattgacaagaagggcactctcgccctccactgcgccgctaaattgtaagaagcatattttcttgagttaacataggaataggtgtaactcaagagaaatgtaattcgtagaattgaactttgtatattaatttatatggtgttgtagatacaatctttagtatttactcatttggtgctttctctcaaatacaatttaaatttagaactttttgatcttcgattttcaggaagttggagttgcaaatcaattcgagtttcaatctctggaatttaataaaagtttgatcttccaagttttcaatctttcagaagacgatgatcttgatatggataaaaaattgcacatcatgagagctttttgaagtttaaatcttcaattctctagagcttaaattcttccttaaaccaaagatcaccaaatgaatgacaaatgtctctatttatcgaaaaatttcatagacttttagatgggcttaggcacattacttgttgggcttggacttgggcttatttgcttggcgggctcatgctcgagcccattatttctttggcctatttttcatgaggggcttgaacttggttgtatacgaaaaaacttgactacctaaatctaatcaaattataatcatcacaattttgacgtgttacgatttaattggccaaaaattcttgttcaacacttgtctctaatcattttcctatataatttaactaaaatatttaactttaagtaacttaaaagatatagtttaattcgaatcaaaatacaaatacaatttcgtctatctattcccatcataaatgttgattgagattcatattataaacttctttcaggaaaagaaagaggaaaattcacctaaaccacgttttcctattttggtaagaatccccaaaccataaatcattccaaaattattttttttagattagaaaagaaaaaagaaaaaaagaaattcacatggcgtaaaatttcagccccgtgagatattttcgaacccccagatacaatctacaccgtgaaaacaaaatcggacggtggttgctataatgtccgtttagaggcaatggcagggatgaaattgccaacgcaagataaggaacgaataagagaaggacacgtaagtacaagtttaggatgggcgggcccacagccacaagtgccgttcgtgcttatatacaagtcgctcatattcctagaagtgtctccaaataaaggaaagaaaagttcactcatagagagaaagagaaaaataaagcttcgttgccggcgatctgaaggcggcggccatttctctcgggagagagaaagagagagattgatagagcggagagttcgaggctctctcaaacttcggtcctcttcttctctttcaggtatcgttcttctctatcccttcgtattctgtttcctcttttctctttcttcgccatcatgctctttctcttgttttgtactcactcaatgtgattgactttatgttgtttttctgttttatttttccattaatgctcgttgtaatgtgtagatctatgataagatttgaattattgctcattaatgtgttgcatgcttttgatttcattttaaaaacagagattactttctctatattgattaaatcgttggattttaggttcttacagagtttgtaaacagtgatgttaaggattgctgagatttatgactgatgagagttagtgtttgtcttttagcttgtcgttttcctctttgaaatcacatggattcgatctggatatctgggtttggctcgtctgaaatggctacactatagcatatttgagtttgtgatgttgaagatttgttaatttcttggaaaatcgggagttcgttttgtttttcctctttttacaggttttattgattggtttattgatcggcgatatctcgttttcaacttccgaaatgctatttttcataagaagaaattgtggatgtctttttctactcgattagagatccttgaaactatgccaaaaaaaattggttctttcaccaaattgttttttgtcgtttgtgatattaatgcattttcttattcttaattaagttcaagtattcttttattattttttaatgatggttgttgtaatggttttttcccttttactaaaagctttttccatgtgattcaaaggtgtacttggggtttcccggtctttgttcccaagtcaattaggatgggcgccaattcgattttagcttctgtatcattggtgtatattctgttctggggaggaaaaaaaaaaagaaaaaaatcttccgtcctacagtgtgctgagtaacaatttgaccagccttttctgccgaaaactttttgaaattattttttaattgtgatttggtgaacttaaattgttttaataaataaggtggattgaatcttaacagaaacatcaaataaaatcgagttttaaaaaaaaaacatatttttagtgaatgtttattttatttaaaagatctccatcagtcctgatgtttcctagaaaacttatacatcataggtcttgattaacaaatttggaggaagtcaataggttattctttttttctttttccattctagtttgaaacaattttcttttcttttttaacttagaaaataatgggtagctagaaatatggaaatcaatgtattttgggcttctccttgaaactggagcagcggtcaatttctctttcgtttgtatagatgtgatagaaatagaatgtttccttcgcttacggcatcagagagttggaattggtctttctcaacctcaatatcaattaaatcaagtttcgtcataaacaggttttttttttcttcgtttcaaatgtttggtagggtcaaataatttgtaaaatacctagccgtccaatatgatacaaactggaggatttcacttgctcttttaaattacaaaaaatattttatcattgatgttgcctgtctgtgtttatcttttctctttccgcctcaagtaggcgtctaattgtcttggcaagttggttttttgtacttccgccccttgtcctttggcccttttgattaagtttttcatttaattttctggtcggcgtacgttgaattattaggtttgcatttaatgtggtacctggtgctttgactcttatttgataaggtattttgaagtctaaaacgttaaaccctttgtttgatgtttatttttttatcgttccaggacaatatcctttggaaaaa

SEQ ID NO: 192 - DNA, 1999 bp, Cucumis melo
  source 1..1999: mol_type = unassigned DNA
aaaagagtcatagtgaaaaaagctgagatcataatagtttcaccctaaacacaacttataataacacatactatcataatatacacaccaaacacagactctatagtcttcactctaaacgcagattacaatagtctgcaccccaaacgtagactattataatcttctgactattataatactcttttcacttatcgccccaaacgtcccctaagagaacaaagataggttataaaagagagatgagggtttatattatgcaacaagtataaggttctagaacgatgtagtcttcaaagtaacgaaagcaataggctacacgagaaaaatatttttaaaatatagtgctttccctaaactagatttcaatgacaaattattataaaaaatagaatcattaatccaacatggcttgcatgtcacaccttggccaaaactgaagacggatgtcatactcgacttcaatatatttttttaattaattttcatgtgacaacacataaatatttaaaatttagattgggttggattttttttcaagtgggtcccaaaatactcttcaaacccaaaccaacccaacttgtttacccatctaataataacccaccaggttcaagaagacgaaccgaaccgaaccgatccggtctaactttgtttcatacttaagtcgaacttagcggtacttttggttcggttctcggtttccccaaacagagccactcaaaattagatttagggttccgttcgcaattttcagcgcatttttatttgaatcggtcgtttgttgaacacgttctctctcagctggtttagggttcatcgttctctcttctcgcgctatatctttctctctctcaggttcgtttctttctcttaggccattttatcagaagatcctcttcgttctccgattttctttccgtgttcgccctcggtttctcagcagacgtaggaagtttggtttccgtttagtgaatctgtttggggtattacgaatgatattttgtactgggctttccgcatagtctttttctttctaggaatatatgcatctgagaatttatttgtttggcttttctttataaagtatgaggacatatacatctcgattgctaatccttgattataatcttttttttttctatgttgtttgaatctgtttttttttttttaatttcaataggttttttgaatctaaaaatgtatttcttggatgaattgcatactgttgaattagaagtttattgattagattgttgatatttgccctaagttccatggataggtttgcgtctttcaccttttcgtttgctttttcttttggctgacgacatcttacatagcctctgctctaaaaggtgccatgattttttttcctggctttatctgagtttgcgcaatttagatttgaagtgatgatttgtctaaatataaatatctatcggccatactattttttgttattttgagtttttcaggatgactgctagagaatgaaaaatcttgaaacattgtgttttgaagttcaaggatcttgtagttttgttcttttctagactatctcatttgatatagccctttaaatttaatcaaaatttgttaatattcaaatcctcggacattttaattatttatctaaatagttgtttaggcattactcaggttgcccactattttaagcttagaagcctactctggttgacctaaagtttgcatgctatttgccttatttcgcacgactctaaactgttatagacatcttttttcagccttcaggtaaatgaacacaaaaaggagtgaaagtctgacttctgtgtgatggtcttttaatcaattatagggattaagatggtttttttattcattgtataaatattaaattagaatgatgacaaccaataatattaaaactgacaatggaaggttccttatattatttggagtgtacattacaacagcctgattcttggcttggcaggttcctgatcaccttgtaaac

SEQ ID NO: 193 - DNA, 2000 bp, Cucumis melo
  source 1..2000: mol_type = unassigned DNA
atttatttgtttagagataagacgcacatgagaatatgagatggattccactccactcacactccaatttctacctatcctattactgtttactattatcattccacccctcgacccctcattcttcttctcaccttacttttttatgatttactactacttcattttggatcacaatctgatcaatgctgggtgggctgccctcggcctgtcaccaggcccagcccacttccaaattaaacctcttggcccaccgcccattgtccccatcccattccatttaatattcccaaccttccctttttctttcccaatgcgatgcttctccaatatacctttcctgccctccatgtttcctttttactgctttcttatatttataacacaccttctacagtcttttggctgggaatgctgcgtatgtgaatgagattcaagatttcgttgatgttatttgagtctctatattcataagttttgttcttagttttctctagaccaactgcaagagttagcgttccatatgctcataagtttcagatttctgctgtgtggtttgaagacagtcatcgatccatgggtgaattcggctttttattattattattattattatttattgttgtcttacttttctatttgaatcttcctatcttttttactcattgttggactctaataattcttgctaaacacaatctccatttttattggacattttaaatcccatctcaactcataattttagttaccttccaccatcaccatatccaaatccgaaataaactcaaataaaatccttcacgtgcatgtgctctccatatattttttctacatggtaaaaataaaatgaaaacaatctaaatttaataaaataacatatatggcagacttttattgatgtagagactgggtgttgtacaagaacagtgcagccaagaaaaaaaaaatacttccaatgaatcgtacattttaaggattatgaaactaactagttccaaccattttttcacgaccacgtgcttgttaaacacgcaagtagaatcaaaatgtgggcttcttcgctttatataactgtgaatcattctccaaaaagggaaggggatctcattccctaattcaataaagaaaaagaaaaatgctagcgaacttcatccatctcattccttttacctatttcatgagatgcccattgtatataagtatttttttttttatttcattttacttagtttactcctcacctctaaaaaaaattaggagagtttgctaaatccattctcaaacttagctttattttttttaattttatttaacctcgtcgtggatgttaacctcaaatgtcagttctttttattctatttattgatgttataatttactttaggattccaattttataaaaataagaatacaaataaagataaagagtgtgaaagccagaaagaaaaaaaaggaaatcgtaatatgggtaaaattggtacaaattgggtcccgttaaatattaactcaaaaaatgcgagaaaatggtagaaaaggaaatagggggtaagagcaaagtagtggaaggagagcattgaacatattctctagtttttgcacttggatctaaacacgaggaattataggtttattcatttactaattacataaataggattggattttaaaatttgaccgagtgattatgcatatttgatagagttagaaaatagtggtggggcaggtacaagttacaagtaatgtataagagatatgatgagcatattaggaaactatagatttaaattcgtccgtaaataaataattagaaatataatattcgagtggaagggtattagggttaggcgaaaccaattgcagttgcacctataaaaccccttttacgcctccacccgcttcaacagcggtctcggcgtctacaactacacactacacactacacactacacactacacagttgcagaccagaagcataacgtaacgccggtccacaaaa

SEQ ID NO: 194 - DNA, 2000 bp, Cucumis melo; identical to SEQ ID NO: 121
  source 1..2000: mol_type = unassigned DNA

SEQ ID NO: 195 - DNA, 1400 bp, Cucumis melo
  source 1..1400: mol_type = unassigned DNA
tatatatattaacttttaaaattttgaaaacgtcatagataaattatatacaaaataaaaagtttgattatttacgaaagttaaaaagtttatccgaaagttgactcaacgataaaaacactaaatatcacttttagagatgatgatattatacataaacatacgaacttacgcgtcaaacttttatactaacacaagatcaaaacaactttgttgagtagtgagaattttatctgctgatatggttgaaacttgggaagcaagcagaggaagttccattcattaccaaaatccattttgtattcatcaaaatatgaagtttagcgacttgataaagtcaagtcaagtggtcctatcgatttgttaatgtcaatgtttggttttgaatttgatacctattagacaatgatatataattttaagtatggtttacactgtgatgctttatatatttttaaatgtaaaatattagaacttgtaatttcaataaattttaaaaatgattttgtgttatttccttttttaaattgaaatatcaatgtatcaatattgcgtcatagagtattgcaacacaaccttatgttaaattgtttattgcttattgctctaattcaactccttcatcaaatgtgcacagaatttaaacaagaaaaagagtaggtgcttttttactaaaatatactaaaagctttttataccaaatcttatgacaaaatcattccaacaaaatgactatttaaatataagatcgaatccctaatttaaaaaaaaaaaaaaatcaaagatgttaatttctattattaaactcactttagcgtagctaacaaaaaaaggaaaatgagaggctacaaagcttgagccctctgcctccctttattgcattgtttgaaattagatcaatactttgtatttttttcaaaatgaaaaatcgtacatagaattaattctatggacaaaaaatcagagaaggaaataatctagaataaaattcgatttttaacccaaaaaaaaaaaaaaaaactcgattctgatttttgtaagcaatcacccaaattaccataaataaatggtattcaattactcaattatggatattttagaaatgataaatttttattcataaactcttttctttctctttcaaaaagaaaaaaattagcataaacttcaatgacatttatttattcttcttcgtttggagtcaaaagtttaaattgagcatcagtccagcccaaaagcccacgaagaagcccaagaatcttcagctttttcgttcaaacgtccctttttggtttataaaattaaagaaaataaaaactaaatttatttgttatttaacaaaacatttttggttaagacattctctttgattatttttcttccattcttcgtcgtcaatc

SEQ ID NO: 196 - DNA, 2019 bp, Cucumis melo
  source 1..2019: mol_type = unassigned DNA
tttatatttatgaaaatgaagtctctaaacaatttttctactcccaaatttgttgatttttctgcctattctttatcggtgctttaaaaaatgaaaccaaatttcaaaactaaaaaaaccaagcttttaaaaaaatgttaggttatttttgaaattcaactaaatgttgaactcttttacttattaaataggcaaattattgaaataaatttagagcaagtaagcttaatttttaaaactaatatacttaccaaatcgaggactaaaatattcaaatactctttaaaattaagattaacattaatcactttgttatgtttaaaaagttgcagtgtcacttgaacctttttaaattaatataatgaaaatgaatccaactcaatatatataatatctatattattaatctcgatgtcagatgtttgatacgcacatatctcaaaaattatacctcaactaacatcggtgcacgatgtattatttcgtgaggataaaaatcgtttttagtataaattgatggaaagattatttgaattactgaaaaatgcaccggtacattatttgaaacttccccttcatttaaagaggctaatattagaaaaagacacgctgaggctatttttacaattaatgtgggctgttgacttggcccagcccaaaacataaaccctaaagtagcacaaacaaacgcctctttcttcttgaaaccgcatattaagcgttttatcacttctcaccacctctcattcctcctcttccgcacgttgtttcgctcctcaacgggagtgccttccctttgctctccctccaggttcttcttccttctcatctcatttccaagtaatttcattccgtttcttttctcttaatcgtatcttgttcagactctttcgcgtttttttttagttgcgccttaccagatctgtgttttcactcgtttctattcgatacatgcttccaagatccatttcttaatcgcatgtattcggttgattggatgattgtctttttgtaagttttgattacttttttggaatggatcggttgaaccaacggggtttaagtcgatggaagtaggttatgttaaagatttgcttcttttttatgaagatgtgtgtgttcttttttctttgctagatgatgttattatttgattgttttaacagtcgtgttttgtttttctgcagtttatagtcctcggtcttttgaagacttgtcaagatggttagtacacctcttgtcatcgtgattttgattgagtgatgtgttaagtgcttctttaggttacagctaacgcgattttttatattcaattgtgcctgtgcaggtgaagtttacagcagaagagctccgtcggattatggactataagcataacattcgtaatatgtctgttattgctcacgtcgatcatggtaagctacttagtttaagtttatttatgccgagcgtctatttaagaagattaacatcttagctttcatttattgtttatttggtaagcatcgtttctttttctccgaggaactgtacatgtcagttcacatgacaataaaacgatcttccttggacattagtttttgaagttcaattagacgccaaattttgttggttaaaagatgcttgtggagcatatggacctaatggaatcagtactttttgatggatggacttgtcttttgttcttttattttcaaaagaaattgcatgtgcaattacatcatctttgatcgaaagattgggtaattgggtaattggggtaaagacatgttgtaaaaactaatgttaattatcaattaccattatataccttatttagtgcttatttatatcctttttccccatttcagggaagtccactctcacagattctcttgtggctgctgccggtatcattgcacaagaagttgctggtgatgtacgaatgacagatactcgtcaagatgaggcagagcgtggtatcaccattaaatctactggaatctccctctactatgagcagaagagctccgtcggatt

SEQ ID NO: 197 - DNA, 1999 bp, Cucumis melo
  source 1..1999: mol_type = unassigned DNA
aaaaggcgaaaaaaaagttagcttcccgagaaggagaagacgaggaagagtttgacttcccggggagataaagtttgtgtctcgagggaatctctaatctggagttgaccgtcgacttatgtgtcgagcctggatttagttgcatggtgcgacaaagcgataaggcggcatatgtaaagtagtaattcaaaactagcggttaaagaaataatcagccaaaaaatttagtacaaatacgggtggaggccctaagtgaagtgctgctattcagaggttttggcaaaagagtgcaaagagttgagttgtgcagagaaagtactaggtgaggagaggcgtttggaaaagaaaaggatcaaacatttgcatgagtgatattcttaaactaaacactcttgtgtgagtgacttgcctaagctaaacactcttgcatgagtaactttcttaaactaaacgttttgtaatgttttcttaatggattcttttcgagtctgagttatgcttaacacgttttgtttctctcgtgttattgttgttgttgtttgaaaagagaaaactattgttttctatgtgctgattgtgatgaatgtgtgcgaaccattagccttaatcctatcaagtgaatagtgattatgtggtgtgtgcacataatgtaaatgacattgtgtggatggccagtgcaacaagaaatgaatcagaaagcttcccaaatactgtgaatggagtgaacatcacactagctcaatggcaagatattggcgatagtgaatcacaataggcttgacaaggggaaggattcatgttcttggttgaaaggaataagagaggctaatgtgagatttctgtgatttgcaaaatgaggcgttggaagacacgtttgagaaatgaaaacgaattagtgcttgacttgtattcctaaaaaagttgtccaatatcttcaatcactaaatatttgatgtgcctaagttttccttccttagttgttgaggcgttgaggccgagtaaggaaagataagataattatgacgttgaaaagctggtcaagttatccatctttggatggtttaaagttattacatgtagggagggttgcattccaattttgtgtgtgagatgagtcttattttcgagatgggttgctaggcgatcaaggagaagtataagaaatgagttcttatactcttgaacaacttgacacgaagaataacatcctagtggatgaaggaaggtgatggaacttaaagtttaggttttatttttggccttgtgataaaaaaaatgtaattgtaaagtattagatcaagtaataaaaacagagttgtgttttctatttttgctgtgttgggttgtgtatctttattgtgcttatggcctagttgctaaagagttaaggttattacctaaatgttttacggtgtgttgagttgtaaagatctcctgagttaaagttggaattttgtattggagattgttttgagaagtttagcttactaattgtttaactcattaggtgtctaagcgacacgcctccttttggtcgcatgaagtggctagcagggtggggcggaccggggtggggtgtgataataaacctaaaaaatcacccagataagcctaaattatacgttgaagttaaacttacaatttgattagaagaagaaggaatatctgatttggacatgaattaattacaaatacggcgccaatcatacaaagcacatgtaagatcaacgcattctacactcaatctcagccgttgattgctttcaatccttcaaaaagaaaaaaagaagggcagttcgggcagagtcatacctacccgttgactataaaagcaactacaaatcgaaaacctccatttctccgttaccattacagagaaaatcaaagaaatttggcgttgagagattgggagagaggtttctctttctagggttgcttcttcttcttcatcctccattgttgcaaatttcacttccttctcctcttgttctcatctccc

SEQ ID NO: 198 - DNA, 1578 bp, Cucumis melo
  source 1..1578: mol_type = unassigned DNA
atatatatatatataatttaactaaataaacaaatgaaagaaaaaagtgagttcccattcggaaaatgcagatggatccatttcttcaaatattaaaaaataaaaaaataaaataaaataacttaaatatgcaaatagaaagaattttaatttctggattatccatatgggacaatttttaaaactcatttattttattttttttatttatttgattttgatatatctatggggaaatttttcgtaataattttcgaaaaaatattgcaatatatcatttgatcagatcggtattattaaatctctatcacatttggtcttaaattatccaaagattcctttaagataatttagataaccatctacagatcactactataatcaacaaaaggaacaacttaaattatttaaacaaattcattaatattagactttgtgcttcattagaaaatgatcttatcacaaccacaaccatagtggtggtttaaaattttattttaaactcttattagtattattttaattcatacttaatcaaactaattactttaaaaaacatatatatataaataagttaaatcattcccccttatatctaaataacataaaaaaaaattgtttactctacaagaagtttgtatatatatatgctcggtactatttagcatctttataataaaatttctaaatcaattttttatatctctttattaaatgtatagtcatcaaaaaatttaacgagataatgtgtcaaagatttattttattaacgttcataaatatcaaattatacttagcttataattgaaaacatgttcgataaatataagtaaataaaattttattttttttaaatattacaaaataaactaaataagttataaatatgacaataaacattatatattttattatatttataaatacttaataatttagtcgtttaaaataattttcttaattttcaaaacatgtttcatatgttaataataaataaatggaaaaccttccaaaagaagaaaaaaagatatcttaaaatttaaaaattgagattttgaggatcaataattaataaaagaaggattaataagggtgaaattaaatcccaaaaagaaaattgaaaatgaagaaaagaaaagtgaagaaataattgaacgtgggaagtggattcgatgtctccagagaacaagcgaaaggagacgaaatccacataatttgcacgttacgtgtccctatcaaccgtagacacgtgtcaacatctcaacaccctacgccgaattgcttcgctggatctggacggtcatcggataacagcggcaaccaattaatatttccccttatatttcacagcctggccatgtccaccaatcacgttcaactattaattcatttttcatttcctttttctttttttttttaattcccctcaattattaccgacaacctgttgtagccggttaaccctaccctccaacgttccattataaggcctagaaaatggacgtgaaaatggagtactacaaactacaattaattttaaagaattttaattttaaagttctctaattactattagcc

SEQ ID NO: 199 - DNA, 1023 bp, Cucumis melo
  source 1..1023: mol_type = unassigned DNA
ataataataataaatacataatagtaataataataaaaaaaataaaaaaataataatagtaatgaaaatcaatagaataattttaaaatcgggaaggaagtcgtgtacaatccttgcacgttggagagtcaaatggcctaagtggtgatgtggaagtcgtgtaccgggtacacgattttcctacaagtcaataataataatatggttatttttctagtttagggttcatgacaaaagattgttcagtcgactggatgtagacaaatctaaaaaataaattaaaatctaatatgaaaactagttttaatttccaaattattaagggttgaattcgaccaataaataataataatacggttattttgaaatttaggaaattgaataaagttgttaaaatcttcaagcaaattgttaagccccgagatattaagaagaggtaataatagaggattctatatttataacatgttaaaattaattgcaaactcataaatgcatcacacagattaacaacataggagggacttccgataaaagtgcaaatattgaaataattacagttcgcgaacatgagtattttaatattttataaaatagtatgcacgtgtatttttgccaaaagaaaaaaagaatagattttgccatttttcaaagtgactctcggttatatcttttatggcgattgtattttatagcgtatgttgtttgtagttaacccatttctcattggcaaattcaatcgtgggccacaacgtttgggcatagcttcaatttggattaactcaattatgtctgaatgggttggactagttcggactcttcggctgggccagaatcagattcgggccgcaatctgttcatttcacacctatatccaaacacccccaaaatcgatacccatcaaaccctaactctcaataacccccatatataaattccttctttagggttttttcatcctcatacactctcaaacctccggtcattctcattttccctgccgcttcttcaataaccctaatc

SEQ ID NO: 200 - DNA, 1654 bp, Arabidopsis thaliana
  source 1..1654: mol_type = unassigned DNA
tgatgattcttgttgttgtagttctttttaaaagtcccacctgagcctctatagactctgattctcttttaagttactattttcaccgctctctaataaggctcgtgattttttgggagccatactgtatactcgccggccctctcacgatgttgttcaattcacagactaaatttgatttatctatttcgccaaaacataacttcattaaaaaatgttctccaaataactaaacgaattaaataaaagaaacctttcatgtaaagttaaaggtatgagactttaagggcagttgctgaacattcgtaacacatgggagaacaatagagaaagttgaaaagaaacgtagcatatagaaaaattatctttgtaaccaagttgatttagaaaaatatcactatttgtgaaaaatactagatcagtttattattacttttttttttttgtatattcacaaatatcatattcatatagaagaaaataaacaaagttgtaaaaatctggcatttaaaataaaattgaacacttcaatttatttcctttcataataataattttggcataagatatttgcaaattgatctggttcggtatggtcgacaaaataattttccacgctacccttccagccgtccattcactatttgccctcaacgttaccaaataacggtccagattcctagggcaagatctaacggttagcaagtaaagtcgtaccatcagaaagaataacaattctttcacaaagtaaacataaccaacggttaacaagttcttagggttaaatcagtaagatccaacggatattaaattgcaaggcccaaatagtttttttgcagcagataataactcgtccccactggcgagtgacgaccgagactctgtgaccctatttttcgagacgataaaagggcaaacaatcgctcttttcaaagctcgcctcttcaccacagagaaaacttcgtctctcttctctgcttcgccctctcatttcctgtgagataaaggcggagtctctctccagttattttgctcatccatcgattcttaggtatgactcgtttctctcagatctgtgattctttataatctcgtcgttcttcaaatcattgttatattcgtttcttcgatctgtgttttttagatctgtaaggtaaatgagacgtttcgatctgtagatctgattgttatattgatagattatgttatctgctttgcttaaagtccgatcggaatgttttgtgctcattgtcgaatatctgatgtatcggtttcatagatctgcttctttttgtgcgtttcgttgatctgataatcttctagtgatcaaaatcgtttggatctgttgactttagtttaaaatgtatccgatctgatgtcgaggcttcattattggaagttgttattgttgtaatcctgatttaagttgctgttcttaaatttatatgatctttgcgttataatatgacatggtagatcttggttcatggttcactgttttccaataaacttggtttgtttggttggatagcgttctgtgatacgaccatgtcttgtgttggataagaattctctgaatttccttggctggtttgtagtatgttattcacgtctggtttctcatcaatgattatgtgattttgcagagttcacc

SEQ ID NO: 201 - DNA, 712 bp, synthetic construct
  misc_feature 1..712: note = Chimeric transcriptional regulatory expression element.
  source 1..712: mol_type = other DNA
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacactctagacagaaaaatttgctacattgtttcacaaacttcaaatattattcatttatttgtcagctttcaaactctttgtttcttgtttgttgatt

SEQ ID NO: 202 - DNA, 938 bp, Arabidopsis thaliana
  source 1..938: mol_type = unassigned DNA
caaaagccgagccgataaatcctaagccgagcctaactttagccgtaaccatcagtcacggctcccgggctaattcatttgaaccgaatcataatcaacggtttagatcaaactcaaaacaatctaacggcaacatagacgcgtcggtgagctaaaaagagtgtgaaagccaggtcaccatagcattgtctctcccagattttttatttgggaaataatagaagaaatagaaaaaaataaaagagtgagaaaaatcgtagagctatatattcgcacatgtactcgtttcgctttccttagtgttagctgctgccgctgttgtttctcctccatttctctatctttctctctcgctgcttctcgaatcttctgtatcatcttcttcttcttcaaggtgagtctctagatccgttcgcttgattttgctgctcgttagtcgttattgttgattctctatgccgatttcgctagatctgtttagcatgcgttgtggttttatgagaaaatctttgttttgggggttgcttgttatgtgattcgatccgtgcttgttggatcgatctgagctaattcttaaggtttatgtgttagatctatggagtttgaggattcttctcgcttctgtcgatctctcgctgttatttttgtttttttcagtgaagtgaagttgtttagttcgaaatgacttcgtgtatgctcgattgatctggttttaatcttcgatctgttaggtgttgatgtttacaagtgaattctagtgttttctcgttgagatctgtgaagtttgaacctagttttctcaataatcaacatatgaagcgatgtttgagtttcaataaacgctgctaatcttcgaaactaagttgtgatctgattcatgtttacttcatgagcttatccaattcatttcggtttcattttactttttttttagtgaa

SEQ ID NO: 203 - DNA, 633 bp, Pisum sativum
  source 1..633: mol_type = unassigned DNA
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaac

SEQ ID NO: 204 - DNA, 315 bp, Gossypium barbadense
  source 1..315: mol_type = unassigned DNA
tgatcacctgtcgtacagtatttctacatttgatgtgtgatttgtgaagaacatcaaacaaaacaagcactggctttaatatgatgataagtattatggtaattaattaattggcaaaaacaacaatgaagctaaaattttatttattgagccttgcggttaatttcttgtgatgatctttttttttattttctaattatatatagtttcctttgctttgaaatgctaaaggtttgagagagttatgctctttttttcttcctctttcttttttaactttatcatacaaattttgaataaaaatgtgagtacatt

SEQ ID NO: 205 - DNA, 315 bp, Gossypium barbadense
  source 1..315: mol_type = unassigned DNA
accatatgacactggtgcatgtgccatcatcatgcagtaatttcatggtatatcttaattatatggttaataaaaaaaagatggtgagtgaataatgtgcgtgcattcctccatgcaccaatggtgaatctctttgcatacatagagattctgaatgattatagtttatgttgtagtgaaattaattttgaatgttgtttttaaattttaatgtcacttggcttgatttatgttttaacgaagcttatgttatgtattttactttaatgatattgcatgtattgttaatttaacattgcttgatcagtatactct

SEQ ID NO: 206 - DNA, 2001 bp, synthetic construct
  misc_feature 1..2001: note = Codon redesigned coding sequence.
  source 1..2001: mol_type = other DNA
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 207 - DNA, 1653 bp, synthetic construct
  misc_feature 1..1653: note = Codon redesigned coding sequence.
  source 1..1653: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 208 - DNA, 936 bp, synthetic construct
  misc_feature 1..936: note = Codon redesigned coding sequence.
  source 1..936: mol_type = other DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 209 - DNA, 253 bp, Agrobacterium tumefaciens
  source 1..253: mol_type = unassigned DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 210 - DNA, 622 bp, Cauliflower mosaic virus
  source 1..622: mol_type = unassigned DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatctagacgcaagacccttcctctatataaggaagttcatttcatttggagaggacacgctga

SEQ ID NO: 211 - DNA, 1446 bp, Cucumis melo
  source 1..1446: mol_type = unassigned DNA
tcccttcagccacttaacacttaaaaatcttaggaaactccatgggctcctctttctccaatgaaattttgacatctgtgttgttgatagctcctatatcctttgagaatttgatatacacctgtcatttgctgatttgtggataactgttagccttttgatattgatacgttcctttttttatgaatttttgtaaatccattcaattttaatgctgtcgtaaatgaaaagccctttcattaatgttgtttatatacatattttaaaattaattcaataacaagtttagttctgttagcttctaggtttgtatctattttatctattaaaggtatgtttgggcttcaggttggaatggagtagaattgaatgggttggggagtaaattttccattcaacaagttcaatttcaaaatggctaataagttttgaactcaattttattttcaataaattccttaattttttgttccttgtttgtaaactattgacttattcgatatattttaaaattgaggtattttaaaaaaataatacaatattaaaattatttataaaatataacaaaatttatgtatagtttatttgaaaattttactatagtttcatttttatattattcctaaccatttccatttaaaattatttcaattatttcttttattaatataattgaaatttcatggatttattagacacatgatttgaaattttatgggtttattaagtattttctaacacaaaatcgcttccgcatcgttttcaattcattcagtaatagaagtaattttttaaaagaaccaaatttgccaaattttgagttccataaggactctgaaaactcattatgtctattactcttcactaattgtagagacttaaattcaagataagagacactaattgatgataattgcccaaaaaataaaaataaaaatgtttcttccccatcctcaacctccatgaattcacagagcccaaagattaattattgggccccaattcctactcatatataccttacagtccctcaaagaaatcttaggaagtaatcaatttctgtttattcaagatgtagcctcccaaaagaaaaatacatcacatcaaattcaaacaaaaatatctacagctagcaaaacctcaaaccgttaaaatttcaagccacataaatgaaattttcatctgaaaaaaggacaatctatctagacgttagatttcagccctaatatgaatctgaagcatttggtggacgagaaagagccatgtaggaatgcatcaaacaaaggaaaaatctttgaactccaatgggattgaagatacagataccaatggataagaatctgttctctttgcccactatttaaactcaccaaacccaccagtatcttcctcaccacaaaatacattccaccgttgatcacaagccttattccaccacctccaaca

SEQ ID NO: 212 - DNA, 2018 bp, Cucumis melo
  source 1..2018: mol_type = unassigned DNA
actattggagacgacgaaggaaaactctaatgtgagcttagaattgaagatggtgaacaattgggagtcttttttaaaaatctttcgtcggtatattgaaatttccttttacactcaaataaccccccttttcacgcgttcaacgtcttcaaaccttcctttttcaattattttttcgtttacattaatgacatttaggtaataagataaattagtggattcttttgtgaaaaatgttagcccatttaagacttttatgaaatatattagaaaaaaatcatatagcaatttatattattctagttaaaagcccataatagaaactaacccaacctaaagtaacgtaaacattcaataagagagacaaattttaaaataatttctaattaaaaaaaaaattgtcaagaccgtccgggtcggatccaaaatataacccgaaccggccggtttagcatctatataaatacacctttagggtttttattctctcaagacttcaccgcatttccacttctctgaggccacggtcagccattggagcagctcaataatcctttgactccctactacggtaagtcgaccttactgctttcggcttctagttttttcaatcctgtcattagtcctttggagttcttctgtacatttatgacgttttcggctcgtgttttgtttcgcctgtatgtagtgggtttttcgagttttgtttttacttttttttatacttgcaggaattagttgaaatctatgtacttcatgccttggataatactcttgatctgttgtgttattcaaaatgaattgttttaagatggtatttgagaatggtcatgtgagttttgcctacttggttattaaaatgaattgttttaggatggtatttgagaatggtcttctgggtatttggttggaacctttgtgctctgctatgaattagggtgttctccccgtttttttttttttttttcttttggttattaatatatcttttatgactacttattcatatatgatatcttttactcgtaaattttgactcatttgaaagttttatccttagtcctttctcattcagggtgtaaaggtatgttgttagggttaaaatagcctatgcaggaaagttctgtatttgttctaattattgcatttgtgtgcatttgtatctagtttatttcttgctgagagtatgcttcattttttagtacacatcacttgtgccactttattatagttgcacatttttgtttatggagaggatgaatagcatttagggatgtcaattttttattgagaaaaccctctctcctacttaagcttggggaatttttgttctaaatgtggtaaacataatacttcttcttattttaatttgaatggaaggggaagacgaatactaatattttcaacgaaccttcacaactttttttttcttatttaggaagccatgtttttcaaaattgtactgtgtgatccacatatttatcgattattagtgaatcgaataataattagagttttattggtataattttgaagttcagacttattacatttgtggaaagtttggttacaattttcaattttattggaatcctaagaactttgtgttaacatatattgagttttcttctctttttttttactcattaagttctctattaggaatgtttggttcaatgtcacatagtcgatagctaagaccagtgacccacaaagctatgattgaacgaaaaacaagcctttcacatcttggtaggaatttgttatttctcaatagatttacagagctgtttcatgtgatcacaatttttttctatttttctgaagttctctattaggaatgggctatctggttagttgcttttgagagaacatgtggattggtgttgctcggtttccttgcctttgtaattttgtccttggaaaaagcaaaatgattaggtatcctgatatgcataacatgtttaagccaactagttctcacttttttagtgcaaataattgatcttcaggaatcg